(19) United States
(12) Patent Application Publication
(10) Pub. No.: US 2025/0361297 A1
(43) Pub. Date: Nov. 27, 2025

(54) ANTI- SIGLEC-15 BINDING MOLECULES AND METHODS OF USE

(71) Applicant: ADAPTAM THERAPEUTICS, S.L., San Sebastian (ES)

(72) Inventors: Francisco de Asís Palazón García, San Sebastian (ES); Asier Antoñana Vildósola, Lodosa (ES)

(21) Appl. No.: 19/076,921

(22) Filed: Mar. 11, 2025

(30) Foreign Application Priority Data
Mar. 12, 2024 (EP) 24382261.6

(51) Int. Cl. C07K 16/28; A61K 39/395; A61K 40/31; A61K 40/42; A61K 45/06; A61K 47/68; A61P 35/00

(52) U.S. Cl. CPC C07K 16/2803; A61K 39/3955; A61K 40/31; A61K 40/4212; A61K 45/06; A61K 47/68031; A61K 47/6849; A61P 35/00; C07K 16/2809; C07K 16/2827; C07K 2317/31; C07K 2317/33; C07K 2317/52; C07K 2317/71; C07K 2317/92

(57) ABSTRACT

The present invention provides anti-Siglec-15 antibodies and antigen-binding fragments thereof as well as isolated nucleic acids, vectors, engineered cells, formulations thereof, and methods of use thereof for treating diseases including cancer and bone disease in a human subject. The invention is further directed to bispecific and multispecific antibodies, antibody-drug conjugates and chimeric antigen receptors derived from the anti-Siglec-15 antibodies and fragments thereof.

### REFERENCE TO SEQUENCE LISTING SUBMITTED ELECTRONICALLY

The content of the electronically submitted Sequence Listing XML, named 5337_0010002_Sequencelisting_ST26.xml, created on Mar. 11, 2025, is 166,312 bytes in size and is hereby incorporated by reference in its entirety.

### TECHNICAL FIELD OF THE INVENTION

The present invention provides anti-Siglec-15 antibodies and antigen-binding fragments thereof as well as isolated nucleic acids, vectors, engineered cells, formulations thereof, and methods of use thereof for treating diseases including cancer and bone disease in a human subject. The invention is further directed to bispecific and multispecific antibodies, antibody-drug conjugates and chimeric antigen receptors comprising the anti-Siglec-15 antibodies and fragments thereof.

### BACKGROUND OF THE INVENTION

Tumor-associated macrophages (TAMs) are a significant component of the tumor microenvironment and play a pivotal role in modulating immune responses against cancer cells. These macrophages are typically skewed towards an immunosuppressive phenotype, often referred to as M2-like macrophages, which contribute to the suppression of effective anti-tumor immunity. One of the key mechanisms by which TAMs exert their immunosuppressive effect is through the expression of surface molecules, such as Siglec-15, that interact with T cells and other immune effector cells, leading to the inhibition of their activation and proliferation. This suppression of immune responses by TAMs is a major barrier to effective anti-tumor immunity and is associated with poor prognosis in various cancers.

Siglec-15, a member of the sialic acid-binding immunoglobulin-like lectins (Siglecs) family, has recently attracted attention in the field of oncology for its immunomodulatory roles within the tumor microenvironment. Siglec-15 is typically expressed on the surface of myeloid lineage cells, including macrophages and myeloid-derived suppressor cells, and is involved in the suppression of T-cell activation. Its interaction with sialic acids on the surface of tumor cells contributes to immunosuppression, allowing cancer cells to evade immune surveillance.

The biology of Siglec-15 is complex and tightly regulated, involving various signaling pathways that promote an immunosuppressive environment conducive to tumor growth and metastasis. Its expression has been also observed in some solid tumors, including those of the breast, lung, and liver, making it a promising target for cancer immunotherapy. By inhibiting the Siglec-15 pathway, there is potential to restore anti-tumor immunity and enhance the efficacy of existing cancer treatments.

### SUMMARY OF THE INVENTION

Provided herein is an antibody or antigen-binding fragment thereof that specifically binds to Siglec-15, wherein the antibody or antigen-binding fragment thereof comprises a heavy chain variable region (VH) comprising complementarity determining regions CDRH1, CDRH2, and CDRH3, wherein the CDRH1, CDRH2, and CDRH3, comprise the CDRH1, CDRH2, and CDRH3, amino acid sequences, respectively, set forth in: SEQ ID NOs: 4, 5, and 6; SEQ ID NOs: 12, 13, and 14; SEQ ID NOs: 20, 21, and 22; SEQ ID NOs: 28, 29, and 30; SEQ ID NOs: 36, 37, and 38; SEQ ID NOs: 44, 45, and 46; SEQ ID NOs: 52, 53, and 54; SEQ ID NOs: 60, 61, and 62; or SEQ ID NOs: 68, 69, and 70.

In one embodiment, the antibody or antigen-binding fragment thereof further comprises a light chain variable region (VL) comprising complementarity determining regions CDRL1, CDRL2, and CDRL3, wherein the CDRL1, CDRL2, and CDRL3, comprise the CDRL1, CDRL2, and CDRL3 amino acid sequences, respectively, set forth in: SEQ ID NOs: 7, 8, and 9; SEQ ID NOs: 15, 16, and 17; SEQ ID NOs: 23, 24, and 25; SEQ ID NOs: 31, 32, and 33; SEQ ID NOs: 39, 40, and 41; SEQ ID NOs: 47, 48, and 49; SEQ ID NOs: 55, 56, and 57; SEQ ID NOs: 63, 64, and 65; or SEQ ID NOs: 71, 72, and 73.

In one embodiment, the heavy chain variable region (VH) of the antibody or antigen-binding fragment thereof comprises the amino acid sequence of: SEQ ID NO: 10; SEQ ID NO: 18; SEQ ID NO: 26; SEQ ID NO: 34; SEQ ID NO: 42; SEQ ID NO: 50; SEQ ID NO: 58; SEQ ID NO: 66; or SEQ ID NO: 74.

In one embodiment, the light chain variable region (VL) the antibody or antigen-binding fragment thereof comprises the amino acid sequence of: SEQ ID NO: 11; SEQ ID NO: 19; SEQ ID NO: 27; SEQ ID NO: 35; SEQ ID NO: 43; SEQ ID NO: 51; SEQ ID NO: 59; SEQ ID NO: 67; or SEQ ID NO: 75.

Also provided herein is an antibody or antigen-binding fragment thereof that specifically binds to Siglec-15, wherein the antibody or antigen-binding fragment thereof comprises a heavy chain variable region and a light chain variable region, wherein the heavy chain variable region comprises the amino acid sequence of: SEQ ID NO: 10; SEQ ID NO: 18; SEQ ID NO: 26; SEQ ID NO: 34; SEQ ID NO: 42; SEQ ID NO: 50; SEQ ID NO: 58; SEQ ID NO: 66; or SEQ ID NO: 74.

Also provided herein is an antibody or antigen-binding fragment thereof that specifically binds to Siglec-15, wherein the antibody or antigen-binding fragment thereof comprises a heavy chain variable region and a light chain variable region, wherein the light chain variable region comprises the amino acid sequence of: SEQ ID NO: 11; SEQ ID NO: 19; SEQ ID NO: 27; SEQ ID NO: 35; SEQ ID NO: 43; SEQ ID NO: 51; SEQ ID NO: 59; SEQ ID NO: 67; or SEQ ID NO: 75.

Also provided herein is an antibody or antigen-binding fragment thereof that specifically binds to Siglec-15, wherein the antibody or antigen-binding fragment thereof comprises a heavy chain variable region and a light chain variable region comprising the amino acid sequences, respectively, of: SEQ ID NOs: 10 and 11; SEQ ID NO: 18 and 19; SEQ ID NO: 26 and 27; SEQ ID NO: 34 and 35; SEQ ID NO: 42 and 43; SEQ ID NO: 50 and 51; SEQ ID NO: 58 and 59; SEQ ID NO: 66 and 67; or SEQ ID NO: 74 and 75.

Also provided herein is an antibody or antigen-binding fragment thereof that specifically binds to Siglec-15, wherein the antibody or antigen-binding fragment thereof comprises a heavy chain variable region and a light chain variable region having at least about 90% sequence identity to the amino acid sequences of: SEQ ID NOs: 10 and 11; SEQ ID NO: 18 and 19; SEQ ID NO: 26 and 27; SEQ ID NO: 34 and 35; SEQ ID NO: 42 and 43; SEQ ID NO: 50 and 51; SEQ ID NO: 58 and 59; SEQ ID NO: 66 and 67; or SEQ ID NO: 74 and 75.

In one embodiment, the antibody or antigen-binding fragment thereof specifically binds to human Siglec-15.

In one embodiment, the antibody or antigen-binding fragment thereof further comprises a heavy chain constant region. In one embodiment, the heavy chain constant region is selected from the group consisting of IgG1, IgG2, IgG3, and IgG4, or a variant thereof. In one embodiment, the heavy chain constant region is IgG1.

In one embodiment, the heavy chain constant region of the antibody or antigen-binding fragment thereof comprises a silenced Fc region. In one embodiment, the silenced Fc region comprises one or more modifications selected from the group consisting of NA, AAA, L234A/L235A, IgG4-PE, RR, GA, FES, IgG2m4, L234AL235AP329G, L234F/L235E/P331S, E233P/L234V/L235A, IgG2c4d, and FEA.

In one embodiment, the heavy chain constant region of the antibody or antigen-binding fragment thereof comprises an Fc region with increased affinity for one or more Fc receptors. In one embodiment, the Fc region comprises one or more modifications selected from the group consisting of AAA, DE, DLE, G236A, ADE, GAALIE, GASDALIE, LPLIL, VLPLL, S239D/1332E, A330L, Asym-mAb1, and reduced core fucosylation.

In one embodiment, the antibody or antigen-binding fragment further comprises a light chain constant region. In one embodiment, the light chain constant region is lambda or kappa.

Also provided herein is an antibody or antigen-binding fragment thereof that binds to the same epitope of Siglec-15 as an antibody or antigen-binding fragment thereof provide herein.

In one embodiment, the antibody or antigen-binding fragment thereof binds the D2 domain of Siglec-15. In one embodiment, the antibody or antigen-binding fragment thereof binds the D1 domain of Siglec-15. In one embodiment, the antibody or antigen-binding fragment thereof does not bind the D1 domain of Siglec-15. In one embodiment, the antibody or antigen-binding fragment thereof does not prevent Siglec-15 from binding to a ligand.

In one embodiment, the antibody or antigen-binding fragment thereof is a monoclonal antibody, a fully human antibody, a murine antibody, a humanized antibody, a nanobody, a single-domain antibody, or a chimeric antibody or antigen-binding fragment thereof. In one embodiment, the antibody or antigen-binding fragment thereof is a monospecific, bispecific, trispecific, or multispecific antibody.

In one embodiment, the antibody or antigen-binding fragment thereof is a bispecific antibody comprising a first binding domain that specifically binds to Siglec-15. In one embodiment, the antibody or antigen-binding fragment further comprises a second binding domain that specifically binds to a cell-surface molecule selected from the group consisting of CD3, PD-L1, PD-L2, Siglec-9, Siglec-10, SIRP1a, CD47, TREM2, and a second Siglec-15. In one embodiment, the cell-surface molecule is present on T cells. In one embodiment, the cell-surface molecule is CD3.

In one embodiment, the antibody or antigen-binding fragment blocks the binding of Siglec-15 to a ligand.

In one embodiment, the antibody or antigen-binding fragment is conjugated to an immunomodulatory agent, a cytotoxic agent, a therapeutic agent, a nucleic acid, a radiolabeled agent, a linker, a prodrug, or any combination thereof.

Also provided herein is a pharmaceutical composition comprising a antibody or antigen-binding fragment thereof provided herein, and a carrier. In one embodiment, the pharmaceutical composition further comprises an additional therapeutic agent. In one embodiment, the additional therapeutic agent is selected from the group consisting of an anti-checkpoint inhibitor antibody, an immunotherapeutic agent, a chemotherapeutic agent, a radiotherapeutic agent, a CAR T-cell therapeutic, and tumor infiltrating lymphocytes (TIL).

Also provided herein is an isolated nucleic acid molecule encoding the heavy chain variable region (VH) of an antibody or antigen-binding fragment thereof provided herein.

Also provided herein is an isolated nucleic acid molecule encoding the light chain variable region (VL) of an antibody or antigen-binding fragment thereof provided herein.

Also provided herein is an isolated nucleic acid molecule encoding the heavy chain variable region (VH) and/or light chain variable region (VL) of an antibody or antigen-binding fragment thereof provided herein.

Also provided herein is a vector comprising a nucleic acid molecule provided herein. In one embodiment, the vector is selected from the group consisting of a plasmid, a lentiviral vector, an adenoviral vector, or a retroviral vector.

Also provided herein is a host cell comprising a vector provided herein.

Also provided herein is a chimeric antigen receptor (CAR) comprising a Siglec-15 binding domain comprising complementarity determining regions CDRH1, CDRH2, CDRH3, CDRL1, CDRL2, and CDRL3 comprising the amino acid sequences, respectively, of: SEQ ID NOs: 4, 5, 6, 7, 8, and 9; SEQ ID NOs: 12, 13, 14, 15, 16, and 17; SEQ ID NOs: 20, 21, 22, 23, 24, and 25; SEQ ID NOs: 28, 29, 30, 31, 32, and 33; SEQ ID NOs: 36, 37, 38, 39, 40, and 41; SEQ ID NOs: 44, 45, 46, 47, 48, and 49; SEQ ID NOs: 52, 53, 54, 55, 56, and 57; SEQ ID NOs: 60, 61, 62, 63, 64, and 65; or SEQ ID NOs: 68, 69, 70, 71, 72, and 73.

In one embodiment, the CAR further comprises one or more of linker sequences, a marker sequence, an extracellular spacer, a transmembrane region, an activatory domain, a co-stimulatory domain, a suicide gene, a secretable immunomodulatory factor, and/or a signal transduction unit. In one embodiment, the signal transduction unit is selected from the group consisting of CD3, CD28, 4-1BB, or OX40. In one embodiment, the secretable immunomodulatory factor is a cytokine or a chemokine.

Also provided herein is an isolated nucleic acid molecule encoding a CAR provided herein.

Also provided herein is a vector comprising a nucleic acid molecule provided herein.

Also provided herein is an engineered cell comprising a CAR provided herein. In one embodiment, the cell is a CD8+ T cell.

Also provided herein is a pharmaceutical composition comprising an engineered cell provided herein, and a carrier.

Also provided herein is a method of producing an antibody or antigen-binding fragment thereof that specifically binds to Siglec-15 comprising (i) culturing a cell comprising a vector provided herein under conditions that express the antibody or antigen-binding fragment thereof; and (ii) recovering the antibody or antigen-binding fragment thereof.

Also provided herein is a method of producing a conjugated antibody or antigen-binding fragment thereof that specifically binds to Siglec-15 comprising (i) culturing a cell comprising a vector provided herein under conditions that lead to expression of the antibody or antigen-binding fragment thereof; (ii) recovering the antibody or antigen-binding fragment thereof; and (iii) conjugating the antibody or antigen-binding fragment thereof to an immunomodulatory agent, a cytotoxic agent, a therapeutic agent, a nucleic acid, a radiolabeled agent, a linker, or any combination thereof.

Also provided herein is a method of producing a chimeric antigen receptor (CAR) comprising a Siglec-15 binding domain comprising (i) culturing a cell comprising a vector provided herein under conditions that lead to expression of the CAR; and (ii) recovering the CAR.

Also provided herein is a method of producing an engineered cell comprising a CAR comprising a Siglec-15 binding domain comprising (i) culturing a cell with a vector provided herein under conditions that lead to expression of the CAR thereby producing an engineered cell; and (ii) recovering the engineered cell.

Also provided herein is a method of blocking the binding of Siglec-15 to sialic acid comprising contacting the Siglec-15 with an antibody or antigen-binding fragment thereof provided herein; a pharmaceutical composition provided herein; a vector provided herein; or a cell provided herein.

Also provided herein is a method of increasing the anti-tumor properties of T cells comprising contacting the T cells with an antibody or antigen-binding fragment thereof provided herein; a pharmaceutical composition provided herein; a vector provided herein; or a cell provided herein.

Also provided herein is a method of depleting cells expressing Siglec-15 in a subject comprising contacting the cells expressing Siglec-15 with an antibody or antigen-binding fragment thereof provided herein; a pharmaceutical composition provided herein; a vector provided herein; or a cell provided herein. In one embodiment, the cells expressing Siglec-15 are macrophages. In one embodiment, the cells expressing Siglec-15 are tumor cells. In one embodiment, the cells expressing Siglec-15 are osteoclasts.

Also provided herein is a method of treating, preventing, alleviating a symptom of, or delaying the progression of a cancer in a subject in need thereof comprising administering an antibody or antigen-binding fragment thereof provided herein; a pharmaceutical composition provided herein; a vector provided herein; or a cell provided herein. In one embodiment, the cancer is a bladder cancer, a bone cancer, a breast cancer, a carcinoid, a cervical cancer, a colon cancer, an endometrial cancer, a glioma, a head and neck cancer, a liver cancer, a lung cancer, a lymphoma, a melanoma, an osteosarcoma, an ovarian cancer, a pancreatic cancer, a prostate cancer, a renal cancer, a sarcoma, a skin cancer, a stomach cancer, a testis cancer, a thyroid cancer, a urogenital cancer, or a urothelial cancer.

In one embodiment, the method further comprises administering an additional therapeutic agent. In one embodiment, the additional therapeutic agent is selected from the group consisting of a chemotherapy, an immunotherapy, a radiotherapy, a CAR-T cell therapy, a TIL therapy, and a checkpoint inhibitor.

Also provided herein is a method of treating, preventing, alleviating a symptom of, or delaying the progression of a bone disease in a subject in need thereof comprising administering an antibody or antigen-binding fragment thereof provided herein; a pharmaceutical composition provided herein; a vector provided herein; or a cell provided herein. In one embodiment, the bone disease is osteoporosis or osteogenesis imperfecta.

Also provided herein is a method for depleting tumor associated macrophages in a subject comprising administering to said subject an antibody or antigen-binding fragment thereof provided herein; a pharmaceutical composition provided herein; a vector provided herein; or a cell provided herein.

Also provided herein is a method of detecting, diagnosing, monitoring the progression of, or predicting risk of a disease or disorder in a subject comprising assaying the expression of Siglec-15 in a sample from the subject using an antibody or antigen-binding fragment thereof provided herein. In one embodiment, the disease or disorder is a cancer or a bone disease.

Also provided herein is a method of selecting a subject for a treatment comprising assaying the expression of Siglec-15 in a sample from the subject using an antibody or antigen-binding fragment thereof provided herein, wherein the treatment comprises administering to the subject an antibody or antigen-binding fragment thereof provided herein; a pharmaceutical composition provided herein; a vector provided herein; or an engineered cell provided herein.

Also provided herein is a method of determining prognosis or duration of survival in a subject comprising assaying the expression of Siglec-15 in a sample from the subject using an antibody or antigen-binding fragment thereof provided herein. In one embodiment, the sample comprises cells, serum, plasma, blood or tissue.

In one embodiment, the method further comprises assaying the expression of a second target. In one embodiment, the second target is PD-1 or PD-L1.

### BRIEF DESCRIPTION OF THE FIGURES

FIG. 1A shows the comparative growth curves of YUMMER1.7 melanoma tumor cells in wild-type (Siglec15^{fl/fl}) and Siglec-15 knockout (KO) mice. Each line depicts the average tumor volume measured every two weeks 2-3 days using calipers following subcutaneous injection of 1 million cells in the flank. WT mice (n=9) are indicated by the black line, while Siglec-15 KO mice (n=6) are shown with the grey line.

FIG. 1B shows the comparative growth curves of YUMMER1.7 melanoma tumor cells in wild-type (Siglec15^{fl/fl}) and conditional myeloid-specific Siglec-15 KO (Siglec 15^{fl/fl}×Lysm^{cre}) mice. Each line depicts the average tumor volume measured every two weeks 2-3 days using calipers following subcutaneous injection of 1 million cells in the flank. WT mice (n=9) are indicated by the black line, while Siglec 15^{fl/fl}×Lysm^{cre} mice (n=6) are shown in grey.

FIG. 2A shows absorbance values (OD) of an enzyme-linked immunosorbent assay (ELISA) performed to assess the binding of monoclonal antibodies from hybridoma supernatants from the indicated clones derived from mice immunized with the ectodomain of recombinant human Siglec-15.

FIG. 2B shows bar graphs depicting the binding affinity of anti-Siglec-15 monoclonal antibodies to HEK 293T cells engineered to express only the truncated second domain (D2) of Siglec-15, as measured by flow cytometry. The graph compares the binding of clones 62E5 and S15B1 against the benchmark anti-Siglec-15 antibody clone 5G12. Each bar represents the mean fluorescence intensity (gMFI) indicative of the antibody's binding to D2, demonstrating that clones 62E5 and S15B1 exhibit significant binding to D2, whereas the 5G12 antibody does not.

FIG. 2C illustrates the hypothetical binding mode of the monoclonal antibodies to the Siglec-15 ectodomain.

FIG. 3 shows flow cytometric analysis showing the binding of anti-Siglec-15 monoclonal antibodies to Siglec-15 positive human macrophages. The clones 62E5 and S15B1 mark a distinctively positive population, demonstrating superior binding to macrophages compared to the benchmark antibody clone 5G12.

FIG. 4 shows the cytotoxicity of anti-Siglec-15 CAR-T cells against MOLM-13 target cells. The top panel displays the results of coculture with untransduced MOLM-13 cells, serving as the control. The bottom panel demonstrates the cytotoxicity against MOLM-13 cells stably overexpressing human Siglec-15. The effector-to-target (E:T) ratios used in the assays are indicated. CAR-T cells express NGFR as a transduction marker, with control CAR-T cells expressing NGFR alone. The differential cytotoxicity illustrates the specificity and efficacy of the CAR-T cells towards Siglec-15 expressing targets.

FIG. 5A shows macrophage phenotypic characterization post-polarization. This figure illustrates the phenotypic analysis of macrophages differentiated from human monocytes and subjected to various polarization conditions. Macrophages were categorized as M0 (untreated), M1 (treated with IFNγ), M2 (treated with IL4, IL6, and IL13), or conditioned with MDA-MB-231 tumor cell media. The expression levels of Siglec-15 (S15), PD-L1, CD206, and CD163 were determined by flow cytometry to assess Siglec-15 expression and the polarization-specific phenotypes.

FIG. 5B shows the normalized cell index corresponding to the cytotoxicity of CAR-T cells expressing an anti-Siglec-15 chimeric antigen receptor (clone S15B1) in killing Siglec-15-expressing human macrophages. The impedance-based cytotoxicity measurement reflects cell death in real-time, showcasing the specificity of the CAR-T cell-mediated cytotoxicity against macrophages treated with IFNγ, M-CSF (M0), a cocktail of cytokines (IL4+IL6+IL13) or conditioned media from MDA-MB-231 tumor cells (CM).

FIG. 6 shows results of enzyme-linked immunosorbent assays (ELISAs) evaluating the binding of monoclonal antibody S15B1 to recombinant Siglec-15 proteins from mouse, human, and Cynomolgus monkey.

FIG. 7A shows a scatter plot of antibody clones characterized by their binding affinity in a 2D ELISA format. Each dot corresponds to an individual antibody clone, color-coded to represent its binding property: Control antibodies are in grey, with trastuzumab, as antibody control, depicted in black. The assay evaluates the binding strength of each clone to Siglec-15 Fc fusion protein bioconjugated with streptavidin (Strep+Siglec-15 Fc bio) plotted on the y-axis against a streptavidin control (Strep control) on the x-axis. The distribution of dots above the baseline indicates specific binders to Siglec-15, distinguishing them from non-specific or low-affinity clones. Only hits and control antibodies are displayed.

FIG. 7B shows a 2D FACS plot of the screening results for antibody clones based on their ability to bind to cells expressing Siglec-15. Each dot represents a clone, with the color indicating the outcome of the binding assay: dark grey for hits, light grey for positive controls, and black for negative control (palivizumab). The y-axis measures binding to Molm-13 cells overexpressing Siglec-15, while the x-axis corresponds to binding to untransduced Molm-13 cells. The favorable positioning of dark grey dots demonstrates the selective recognition of Siglec-15 expressing cells by the hits, as opposed to the controls.

FIG. 8A shows an example of an ELISA binding curve of full-length IgG1 anti-Siglec-15 antibody (clone ZLA-1214-D08) with antigen immobilization and titration beginning at 10 μg/ml. Detection was conducted using an anti-human IgG Fab-specific secondary antibody, with palivizumab as the negative control.

FIG. 8B shows an example of a flow cytometry analysis of the same antibody (clone ZLA-1214-D08), titrated on MOLM-13 Siglec-15 expressing cells, with wild-type MOLM-13 cells as negative controls. Anti-human Fc secondary antibody was used for detection, and the calculated EC50 values indicate the antibodies' binding affinities.

FIG. 9 shows an example from a titration analysis of seven anti-Siglec-15 antibodies (clone ZLA-1214-D08 is shown). Starting at 10 μg/ml, the antibodies were tested for binding to HEK293T cells expressing mouse Siglec-15 (triangled line) and a truncated version of human Siglec-15 comprising only domain 2 (circled grey line).

FIG. 10 shows the impact of seven different anti-Siglec-15 monoclonal antibodies on the cytotoxic activity of anti-CD19 CAR-T cells against THP1-CD19 cells overexpressing Siglec-15. Each bar represents the percentage of cytotoxicity observed when CAR-T cells are co-cultured with THP1 cells pre-incubated with a specific anti-Siglec-15 antibody at 10 μg/mL concentration. The y-axis indicates the percentage of cytotoxicity, with higher values denoting enhanced killing of target cells. Negative values represent suppression of cytotoxicity. 19BBz (negative control) represents the baseline killing of anti-CD19 CAR-T cells in the absence of anti-Siglec-15 blocking antibodies.

FIG. 11A shows results of ELISAs evaluating the binding of monoclonal antibody S15B1 and benchmark antibodies (Benchmark 1: 5G12 clone, Benchmark 2: A2A5C7E8-3 clone) to recombinant human Siglec-15 protein.

FIG. 11B shows results of ELISAs evaluating binding of monoclonal antibody S15B1 and benchmark antibodies (Benchmark 1: 5G12 clone, Benchmark 2: A2A5C7E8-3 clone) to recombinant cynomolgus Siglec-15 protein.

FIG. 11C shows results of ELISAs evaluating binding of monoclonal antibody S15B1 and benchmark antibodies (Benchmark 1: 5G12 clone, Benchmark 2: A2A5C7E8-3 clone) to recombinant mouse Siglec-15 protein.

FIG. 12A shows results of ELISAs evaluating the binding of humanized sequences of monoclonal antibody S15B1 and benchmark antibodies (Benchmark 1: 5G12 clone, Benchmark 2: A2A5C7E8-3 clone) to recombinant human Siglec-15 protein.

FIG. 12B shows results of ELISAs evaluating the binding of humanized monoclonal antibody S15B1 and benchmark antibodies (Benchmark 1: 5G12 clone, Benchmark 2: A2A5C7E8-3 clone) to recombinant cynomolgus monkey Siglec-15 protein.

FIG. 12C shows results of ELISAs evaluating the binding of humanized monoclonal antibody S15B1 and benchmark antibodies (Benchmark 1: 5G12 clone, Benchmark 2: A2A5C7E8-3 clone) to recombinant mouse Siglec-15 protein.

FIG. 13 shows results of flow cytometry assessing the binding of humanized clones of S15B1 monoclonal antibody and benchmark antibodies to HEK293T cells expressing human Siglec-15 protein.

FIG. 14A shows an example of an ELISA binding curve of full-length IgG1 anti-Siglec-15 antibody (clone ZLA-1243-A01) with antigen immobilization and titration beginning at 10 μg/ml. Detection was conducted using streptavidin-HRP, with palivizumab as the negative control.

FIG. 14B shows an example of a flow cytometry analysis of the same antibody (clone ZLA-1243-A01), titrated on MOLM-13 Siglec-15 expressing cells, with wild-type MOLM-13 cells as negative controls. Anti-human Fc secondary antibody was used for detection, and the calculated EC50 values indicate the antibodies binding affinities.

FIG. 15A shows results of ELISAs evaluating binding of anti-Siglec-15 monoclonal and benchmark antibodies (Benchmark 1: 5G12 clone, Benchmark 2: A2A5C7E8-3 clone) to recombinant human Siglec-15 protein.

FIG. 15B shows results of ELISAs evaluating binding of anti-Siglec-15 monoclonal and benchmark antibodies (Benchmark 1: 5G12 clone, Benchmark 2: A2A5C7E8-3 clone) to recombinant cynomolgus Siglec-15 protein.

FIG. 15C shows results of ELISAs evaluating binding of anti-Siglec-15 monoclonal and benchmark antibodies (Benchmark 1: 5G12 clone, Benchmark 2: A2A5C7E8-3 clone) to recombinant mouse Siglec-15 protein.

FIG. 15D shows results of flow cytometry evaluating binding of anti-Siglec-15 monoclonal and benchmark antibodies (Benchmark 1: 5G12 clone, Benchmark 2: A2A5C7E8-3 clone) to HEK293T cells expressing human Siglec-15 protein.

FIG. 16 shows an example from a titration analysis of seven anti-Siglec-15 antibodies (clone ZLA-1243-A01 is shown). Starting at 10 μg/ml, the antibodies were tested for binding to HEK293T cells expressing mouse Siglec-15 (triangled line) and a truncated version of human Siglec-15 comprising only domain 2 (circled grey line).

FIG. 17 shows bar graphs depicting the binding affinity of anti-Siglec-15 monoclonal antibodies to HEK 293T cells engineered to express only the truncated second domain (D2) of Siglec-15, as measured by flow cytometry. The graph compares the binding of clones 62E5 and S15B1 against the benchmark anti-Siglec-15 antibody clone A2A5C7E8 (Benchmark 2). Each bar represents the mean fold change of mAb positive cells indicative of the antibody's binding to D2, demonstrating that clones 62E5 and S15B1 exhibit significant binding to D2, whereas the benchmark 2 antibody does not.

FIG. 18A shows the ability of S15B1 antibody to inhibit the binding of benchmark antibody 1 (5G12 clone) to human Siglec-15 in a competitive flow cytometry assay.

FIG. 18B shows the ability of S15B1 antibody to inhibit the binding of benchmark antibody 2 (A2A5C7E8-3 clone) to human Siglec-15 in a competitive flow cytometry assay.

FIG. 18C shows the ability of anti-Siglec-15 monoclonal antibodies clones ZLA-1243-E01 and ZLA-1243-A01 to inhibit the binding of benchmark antibody 1 (5G12 clone) to human Siglec-15 in a competitive flow cytometry assay.

FIG. 18D shows the ability of anti-Siglec-15 monoclonal antibodies clones ZLA-1243-E01 and ZLA-1243-A01 to inhibit the binding of benchmark antibody 2 (A2A5C7E8-3 clone) to human Siglec-15 in a competitive flow cytometry assay.

FIG. 19A shows the binding percentage of anti-Siglec-15 antibodies to HEK293T cells expressing full-length Siglec-15 (domains 1 and 2) compared to benchmark 1 (5G12 clone).

FIG. 19B shows the binding percentage of anti-Siglec-15 antibodies to HEK293T cells expressing only domain 2 compared to benchmark 1 (5G12 clone).

FIG. 20 shows the binding percentage of S15B1 to HEK293T cells expressing a mutant version of Siglec-15 (R143A) (Benchmark 1: 5G12 clone, Benchmark 2: A2A5C7E8-3 clone).

FIG. 21 shows the binding percentage of ZLA-1243-A01 to HEK293T cells expressing a mutant version of Siglec-15 (R143A) (Benchmark 1: 5G12 clone, Benchmark 2: A2A5C7E8-3 clone).

FIG. 22 shows the ability of S15B1 antibody to block the binding of human Siglec-15 to Jurkat cells in a flow cytometry assay (Benchmark 1: 5G12 clone, Benchmark 2: A2A5C7E8-3 clone).

FIG. 23A shows the percentage of Siglec-15 positive cells that internalized the indicated anti-Siglec-15 antibodies labeled with Zenon pHrodo iFL Green after incubation for 24 hours at 37° C. Percentages of Zenon pHrodo positive cells are shown after incubation with S15B1 and benchmark antibodies (Benchmark 1: 5G12 clone, Benchmark 2: A2A5C7E8-3 clone).

FIG. 23B Percentages of Zenon pHrodo positive cells after incubation with anti-Siglec-15 antibodies and benchmark antibodies (Benchmark 1: 5G12 clone, Benchmark 2: A2A5C7E8-3 clone).

FIG. 24A shows the internalization kinetics of anti-Siglec-15 antibodies over 1, 2, 4, 16, and 24 hours. Antibodies labeled with Zenon pHrodo iFL Green were added to Siglec-15 positive cells at 37° C., and internalization was measured using a MACS Quant 10 flow cytometer. Fold changes over the isotype control are shown for S15B1 and benchmark antibodies (Benchmark 1: 5G12 clone, Benchmark 2: A2A5C7E8-3 clone).

FIG. 24B shows the internalization kinetics of anti-Siglec-15 antibodies over 1, 2, 4, 16, and 24 hours. Antibodies labeled with Zenon pHrodo iFL Green were added to Siglec-15 positive cells at 37° C., and internalization was measured by MFI using a MACS Quant 10 cytometer. Fold changes over the isotype control are shown for the indicated anti-Siglec-15 antibody clones and benchmark antibodies (Benchmark 1: 5G12 clone, Benchmark 2: A2A5C7E8-3 clone).

FIG. 25A shows the binding capacity of S15B1-based antibody-drug conjugates (ADCs) with MMAE to THP-1 cells overexpressing human Siglec-15, as measured by flow cytometry in a cell-based binding assay. Trastuzumab-based ADC is included as negative control.

FIG. 25B shows the binding capacity of the indicated anti-Siglec-15 antibody-based antibody-drug conjugates (ADCs) with MMAE to THP-1 cells overexpressing human Siglec-15, as measured by flow cytometry in a cell-based binding assay. Trastuzumab-based ADC is included as negative control.

FIG. 25C shows the binding capacity of S15B1-based antibody-drug conjugates (ADCs) with Exatecan to THP-1 cells overexpressing human Siglec-15, as measured by flow cytometry in a cell-based binding assay. Trastuzumab-based ADC is included as negative control.

FIG. 25D shows the binding capacity of the indicated anti-Siglec-15 antibody-based antibody-drug conjugates (ADCs) with Exatecan to THP-1 cells overexpressing human Siglec-15, as measured by flow cytometry in a cell-based binding assay. Trastuzumab-based ADC is included as negative control.

FIG. 26A shows the cytotoxic effect of S15B1-based ADCs on human Siglec-15-THP-1 cells, as assessed by counting DAPI-positive cells using flow cytometry after 48 hours of culture (MMAE or Exatecan payloads as indicated). Trastuzumab-based ADCs are included as negative controls.

FIG. 26B shows the cytotoxic effect of anti-S15 antibody-based ADCs with MMAE on human Siglec-15-THP-1 cells, as assessed by counting DAPI-positive cells using flow cytometry after 48 hours of culture. Trastuzumab-based ADC is included as negative control.

FIG. 26C shows the cytotoxic effect of anti-S15 antibody-based ADCs with Exatecan on human Siglec-15-THP-1 cells, as assessed by counting DAPI-positive cells using flow cytometry after 48 hours of culture. Trastuzumab-based ADC is included as negative control.

FIG. 27A shows the cytotoxic effect of S15B1-based ADCs on human Siglec-15-THP-1 cells, as measured by an ATP assay (CellTiter-Glo luminescent cell viability assay) after 48 hours of culture. (MMAE and Exatecan payloads). Trastuzumab-based ADCs are included as negative controls.

FIG. 27B shows the cytotoxic effect of anti-S15 antibody-based ADCs with MMAE on human Siglec-15-THP-1 cells, as measured by an ATP assay (CellTiter-Glo luminescent cell viability assay) after 48 hours of culture. Trastuzumab-based ADC is included as a negative control.

FIG. 27C shows the cytotoxic effect of anti-S15 antibody-based ADCs with Exatecan on human Siglec-15-THP-1 cells, as measured by an ATP assay (CellTiter-Glo luminescent cell viability assay) after 48 hours of culture. Trastuzumab-based ADC is included as a negative control.

FIG. 28A shows a diagram of the differentiation process for obtaining macrophages from human PBMCs.

FIG. 28B shows the viability of MMAE or Exatecan treated human macrophages, as assessed by counting calcein-positive and Ethidium Homodimer-1 negative cells using flow cytometry after 48 hours of culture.

FIG. 29A shows the in vivo ADC efficacy results of ZLA-1243-A01-exatecan in a subcutaneous EMT-6 breast tumor model in balb/c mice in comparison to vehicle-treated mice.

FIG. 29B shows the tumor growth inhibition percentages (TGI %) of ZLA-1243-A01-exatecan in comparison to vehicle-treated mice.

FIG. 29C shows the in vivo ADC efficacy results of S15B1-exatecan in a subcutaneous EMT-6 breast tumor model in balb/c mice in comparison to vehicle-treated mice.

FIG. 29D shows the tumor growth inhibition percentages (TGI %) of S15B1-exatecan in comparison to vehicle-treated mice.

FIG. 30 shows the tested formats of anti-Siglec-15/CD3 bispecific engager antibodies.

FIG. 31A shows the binding affinity of anti-Siglec-15/CD3 bispecific antibodies, the S15B1 clone and an isotype control mAb to recombinant Siglec-15 analyzed by BLI.

FIG. 31B shows the binding affinity of anti-Siglec-15/CD3 bispecific antibodies, the S15B1 clone and an isotype control mAb to CHO cells expressing Siglec-15 by flow cytometry.

FIG. 32A shows the binding affinity of the indicated anti-Siglec-15/CD3 bispecific antibodies, the parental anti-CD3 antibody and an isotype control mAb; to recombinant CD3 analyzed by BLI.

FIG. 32B shows the binding affinity of the indicated anti-Siglec-15/CD3 bispecific antibodies, the parental anti-CD3 antibody and an isotype control mAb; to Jurkat cells expressing CD3 by flow cytometry.

FIG. 33 shows a luminescence-based reporter assay representing the activation of Jurkat T cells following incubation with anti-Siglec-15/CD3 bispecific antibodies.

FIG. 34 shows the in vitro cytotoxic activity of anti-Siglec-15/CD3 bispecific engagers against cells expressing Siglec-15 in the presence of human T cells.

FIG. 35 shows immunohistochemistry (IHC) results using serial dilutions of Siglec-15 primary antibody 62E5 on positive (top panels) and negative (bottom panels) HEK293T cell pellets. Bars in the images correspond to 50 μm.

FIG. 36 shows IHC results of anti-CD163 antibody, PD-L1 antibody and Siglec-15 antibody (clone 62E5) in a human breast cancer sample. Bars in the images correspond to 500 μm.

FIG. 37 shows IHC results of anti-CD163 antibody, PD-L1 antibody and Siglec-15 antibody (clone 62E5) in a human breast cancer sample. Bars in the images correspond to 1 mm.

FIG. 38 shows IHC results of anti-CD163 antibody, PD-L1 antibody and Siglec-15 antibody (clone 62E5) in a human breast cancer sample. Bars in the images correspond to 500 μm.

FIG. 39 shows IHC results of anti-CD163 antibody, PD-L1 antibody and Siglec-15 antibody (clone 62E5) in a human breast cancer sample. Bars in the images correspond to 250 μm.

FIG. 40 shows IHC results of anti-CD163 antibody, PD-L1 antibody and Siglec-15 antibody (clone 62E5) in a human lung cancer sample. Bars in the images correspond to 500 μm.

FIG. 41 shows IHC results of anti-CD163 antibody, PD-L1 antibody and Siglec-15 antibody (clone 62E5) in a human lung cancer sample. Bars in the images correspond to 1 mm.

FIG. 42 shows IHC results of anti-CD163 antibody, PD-L1 antibody and Siglec-15 antibody (clone 62E5) in a human liver cancer sample. Bars in the images correspond to 500 μm.

FIG. 43 shows IHC results of anti-CD163 antibody, PD-L1 antibody and Siglec-15 antibody (clone 62E5) in a human pancreatic cancer sample. Bars in the images correspond to 500 μm.

FIG. 44 shows IHC results of anti-CD163 antibody and Siglec-15 antibody (clone 62E5) in osteosarcoma human sample. Bars in the images correspond to 1 mm.

FIG. 45 shows IHC results of anti-CD163 antibody and Siglec-15 antibody (clone 62E5) in osteosarcoma human sample. Bars in the images correspond to 500 μm.

FIG. 46 shows the affect of anti-Siglec-15 antibodies S15B1, 62E5 and ZLA-1243-A01 on osteoclast differentiation. The results are shown as TRACP 5b activity (U/L) secreted into the culture medium at day 7.

FIG. 47 shows the affect of anti-Siglec-15 antibodies S15B1, 62E5 and ZLA-1243-A01 on osteoclast activation. The results are shown as CTX-I (nM) released into the culture medium. BL=Baseline (no added compound), C=Control (odanacatib, 1 μM).

FIG. 48 shows immunofluorescence staining of Siglec-15 at day 7 of osteoclast differentiation (clone 62E5) (A and B), and secondary antibody control (C). Bars in the images correspond to 100 μm.

### DETAILED DESCRIPTION OF THE INVENTION

Provided herein are antibodies (e.g., monoclonal antibodies) and antigen-binding fragments thereof that specifically bind to Siglec-15 (e.g., human Siglec-15). The anti-Siglec-15 antibodies and antigen-binding fragments thereof can, for example, block the binding of Siglec-15 to sialic acid and/or deplete cells expressing Siglec-15.

Also provided are isolated nucleic acids encoding such antibodies and antigen-binding fragments thereof. Further provided are vectors and cells comprising nucleic acids encoding such antibodies and antigen-binding fragments thereof. Also provided are methods of making such antibodies and antigen-binding fragments thereof. In other aspects, provided herein are methods for treating certain conditions, such as cancer. Related compositions (e.g., pharmaceutical compositions), kits, and detection methods are also provided.

### 1.1 Terminology

As used herein, the term “Siglec-15” refers to mammalian Siglec-15 polypeptides including, but not limited to, native Siglec-15 polypeptides and isoforms of Siglec-15 polypeptides. “Siglec-15” encompasses full-length, unprocessed Siglec-15 polypeptides as well as forms of Siglec-15 that result from processing within the cell, e.g., splice variants. A “Siglec-15 polynucleotide,” “Siglec-15 nucleotide,” or “Siglec-15 nucleic acid” refer to a polynucleotide encoding Siglec-15. In some embodiments, “Siglec-15” refers to the polypeptide of SEQ ID NO. 1

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, fully human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively.

The different classes of immunoglobulins have different and well-known subunit structures and three-dimensional configurations. Antibodies can be naked or conjugated to other molecules such as toxins, radioisotopes, etc.

The term “antibody fragment” refers to a portion of an intact antibody. An “antigen-binding fragment,” “antigen-binding domain,” or “antigen-binding region,” refers to a portion of an intact antibody that binds to an antigen. An antigen-binding fragment can contain the antigenic determining regions of an intact antibody (e.g., the complementarity determining regions (CDR)). Examples of antigen-binding fragments of antibodies include, but are not limited to Fab, Fab′, F(ab′)2, scFv, and Fv fragments, linear antibodies, single-domain antibodies, nanobodies, and single chain antibodies. An antigen-binding fragment of an antibody can be derived from any animal species, such as rodents (e.g., mouse, rat, or hamster) and humans or can be artificially produced.

The terms “anti-Siglec-15 antibody,” “Siglec-15 antibody” and “antibody that binds to Siglec-15” refer to an antibody that is capable of specifically binding Siglec-15 with sufficient affinity such that the antibody is useful as a diagnostic and/or therapeutic agent in targeting Siglec-15.

A “monoclonal” antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably to refer to the light chain variable region of an antibody.

The terms “VH” and “VH domain” are used interchangeably to refer to the heavy chain variable region of an antibody.

The term “Kabat numbering” and like terms are recognized in the art and refer to a system of numbering amino acid residues in the heavy and light chain variable regions of an antibody or an antigen-binding fragment thereof. In certain aspects, CDRs can be determined according to the Kabat numbering system (see, e.g., Kabat E A & Wu T T (1971) Ann NY Acad Sci 190:382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In a specific embodiment, the CDRs of the antibodies described herein have been determined according to the Kabat numbering scheme.

Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

As used herein, the terms “constant region” or “constant domain” are interchangeable and have their meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain. In certain aspects, an antibody or antigen-binding fragment comprises a constant region or portion thereof that is sufficient for antibody-dependent cell-mediated cytotoxicity (ADCC), antibody-dependent cellular phagocytosis (ADCP), or complement-dependent cytotoxicity (CDC).

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG1, IgG2, IgG3, and IgG4. Heavy chain amino acid sequences are well known in the art. In specific embodiments, the heavy chain is a human heavy chain.

As used herein, the term “light chain” when used in reference to an antibody can refer to any distinct type, e.g., kappa (κ) or lambda (λ) based on the amino acid sequence of the constant domains. Light chain amino acid sequences are well known in the art. In specific embodiments, the light chain is a human light chain.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, the Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the replaced non-human residues to refine and optimize antibody or antigen-binding fragment thereof specificity, affinity, and/or capability. In general, the humanized antibody or antigen-binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91 (3): 969-973 (1994), and Roguska et al., Protein Eng. 9 (10): 895-904 (1996). In some embodiments, a “humanized antibody” is generated by resurfacing (Roguska et al., Proc. Natl. Acad. Sci., USA, 91 (3): 969-973 (1994)).

The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

“Binding affinity” generally refers to the strength of the sum total of non-covalent interactions between a single binding site of a molecule (e.g., an antibody or antigen-binding fragment thereof) and its binding partner (e.g., an antigen). Unless indicated otherwise, as used herein, “binding affinity” refers to intrinsic binding affinity which reflects a 1:1 interaction between members of a binding pair (e.g., antibody or antigen-binding fragment thereof and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody or antigen-binding fragment thereof to an antigen, and k_{off} refers to the dissociation of, e.g., an antibody or antigen-binding fragment thereof from an antigen. The kₒₙ and k_{off} can be determined by techniques known to one of ordinary skill in the art, such as BIAcore® or KinExA.

As used herein, an “epitope” is a term in the art and refers to a localized region of an antigen to which an antibody or antigen-binding fragment thereof can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain embodiments, the epitope to which an antibody or antigen-binding fragment thereof binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giege R et al., (1994) Acta Crystallogr D Biol Crystallogr 50 (Pt 4): 339-350; McPherson A (1990) Eur J Biochem 189:1-23; Chayen N E (1997) Structure 5:1269-1274; McPherson A (1976) J Biol Chem 251:6300-6303). Antibody/antigen-binding fragment thereof: antigen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al.; U.S. 2004/0014194), and BUSTER (Bricogne G (1993) Acta Crystallogr D Biol Crystallogr 49 (Pt 1): 37-60; Bricogne G (1997) Meth Enzymol 276A: 361-423, ed Carter C W; Roversi P et al., (2000) Acta Crystallogr D Biol Crystallogr 56 (Pt 10): 1316-1323). Mutagenesis mapping studies can be accomplished using any method known to one of skill in the art. See, e.g., Champe M et al., (1995) J Biol Chem 270:1388-1394 and Cunningham B C & Wells J A (1989) Science 244:1081-1085 for a description of mutagenesis techniques, including alanine scanning mutagenesis techniques.

As used herein, the terms “immunospecifically binds,” “immunospecifically recognizes,” “specifically binds,” and “specifically recognizes” are analogous terms in the context of antibodies or antigen-binding fragments thereof. These terms indicate that the antibody or antigen-binding fragment thereof binds to an epitope via its antigen-binding domain and that the binding entails some complementarity between the antigen-binding domain and the epitope. Accordingly, an antibody that “specifically binds” to human Siglec-15 may also bind to Siglec-15 from other species (e.g., cynomolgous monkey, mouse, and/or rat Siglec-15) and/or Siglec-15 proteins produced from other human alleles.

As used herein, “cell engineering” or “cell modification” (including derivatives thereof) refers to the targeted modification of a cell, e.g., an immune cell disclosed herein. In some aspects, the cell engineering comprises viral genetic engineering, non-viral genetic engineering, introduction of receptors to allow for tumor specific targeting (e.g., an anti-Siglec-15 CAR) introduction of one or more endogenous genes that improve T cell function, introduction of one or more synthetic genes that improve immune cell, e.g., T cell function, or any combination thereof. As further described elsewhere in the present disclosure, in some aspects, a cell can be engineered or modified with a transcription activator (e.g., CRISPR/Cas system-based transcription activator), wherein the transcription activator is capable of inducing and/or increasing the endogenous expression of a protein of interest.

The term “chimeric antigen receptor” or alternatively a “CAR” refers to a set of polypeptides, typically two in the simplest form, which when in an immune effector cell, provides the cell with specificity for a target cell, typically a cancer cell, and with intracellular signal generation. In some aspects, a CAR comprises at least an extracellular antigen-binding domain, a transmembrane domain and a cytoplasmic signaling domain (also referred to herein as “an intracellular signaling domain”) comprising a functional signaling domain derived from a stimulatory molecule and/or costimulatory molecule as defined below. In some aspects, the set of polypeptides are in the same polypeptide chain, e.g., comprise a chimeric fusion protein. In some aspects, the set of polypeptides are not contiguous with each other, e.g., are in different polypeptide chains. In some aspects, the set of polypeptides includes a dimerization switch that, upon the presence of a dimerization molecule, can couple the polypeptides to one another, e.g., can couple an antigen-binding domain to an intracellular signaling domain. In some aspects, the stimulatory molecule of the CAR is the zeta chain associated with the T cell receptor complex (e.g., CD3 zeta). In some aspects, the cytoplasmic signaling domain comprises a primary signaling domain (e.g., a primary signaling domain of CD3-zeta). In some aspects, the cytoplasmic signaling domain further comprises one or more functional signaling domains derived from at least one costimulatory molecule.

In some aspects, the CAR comprises a chimeric fusion protein comprising an antigen-binding domain, a transmembrane domain, and an intracellular signaling domain comprising a functional signaling domain derived from a stimulatory molecule, wherein the antigen-binding domain and the transmembrane domain are linked by a CAR spacer. In some aspects, the CAR comprises a chimeric fusion protein comprising an antigen-binding domain linked to a transmembrane domain via a CAR spacer and an intracellular signaling domain comprising a functional signaling domain derived from a costimulatory molecule and a functional signaling domain derived from a stimulatory molecule. In some aspects, the CAR comprises a chimeric fusion protein comprising an antigen-binding domain linked to a transmembrane domain via a CAR spacer and an intracellular signaling domain comprising two functional signaling domains derived from one or more costimulatory molecule(s) and a functional signaling domain derived from a stimulatory molecule. In some aspects, the CAR comprises a chimeric fusion protein comprising an antigen-binding domain linked to a transmembrane domain via a CAR spacer and an intracellular signaling domain comprising at least two functional signaling domains derived from one or more costimulatory molecule(s) and a functional signaling domain derived from a stimulatory molecule. In some aspects, the CAR comprises an optional leader sequence at the amino-terminus (N-terminus) of the CAR. In some aspects, the CAR further comprises a leader sequence at the N-terminus of the antigen-binding domain, wherein the leader sequence is optionally cleaved from the antigen-binding domain (e.g., a scFv) during cellular processing and localization of the CAR to the cellular membrane.

While the present application often uses CARs to illustrate the different aspects of the disclosed subject matter, it will be apparent to a skilled artisan that the relevant disclosures provided herein can equally apply to other chimeric binding proteins. As used herein, the term “chimeric binding protein” refers to proteins that are capable of binding to one or more antigens (e.g., comprising an antigen-binding moiety) and are created through the joining of two or more heterologous polynucleotides which originally coded for separate proteins or fragments of proteins or multiple fragments of the same protein connected in a non-naturally occurring orientation. Non-limiting examples of other chimeric binding proteins include a T cell receptor (TCR) (e.g., engineered TCR), chimeric antibody-T cell receptor (caTCR), chimeric signaling receptor (CSR), T cell receptor mimic (TCR mimic), and combinations thereof. Accordingly, unless indicated otherwise, the term CARs, in some aspects, can encompass other types of chimeric binding proteins known in the art, e.g., those described herein.

As used herein, the term “reference CAR T cell” refers to a corresponding CAR T cell comprising the same structural CAR components but does not express Siglec-15 antibody or antigen-binding fragment.

The term “signaling domain” refers to the functional portion of a protein which acts by transmitting information within the cell to regulate cellular activity via defined signaling pathways by generating second messengers or functioning as effectors by responding to such messengers.

An “intracellular signaling domain,” as the term is used herein, refers to an intracellular portion of a molecule. The intracellular signaling domain can generate a signal that promotes an immune effector function of the CAR-containing cell, e.g., an anti-Siglec-15 CAR T cell described herein. Non-limiting examples of immune effector function, e.g., in a CAR T cell, include cytolytic activity and helper activity, including the secretion of cytokines. In some aspects, the intracellular signal domain is the portion of the protein that transduces the effector function signal and directs the cell to perform a specialized function. While the entire intracellular signaling domain can be employed, in many cases, it is not necessary to use the entire chain. To the extent that a truncated portion of the intracellular signaling domain is used, such truncated portion can be used in place of the intact chain as long as it transduces the effector function signal. The term intracellular signaling domain is thus meant to include any truncated portion of the intracellular signaling domain sufficient to transduce the effector function signal.

In some aspects, the intracellular signaling domain can comprise a primary intracellular signaling domain. Exemplary primary intracellular signaling domains include those derived from the molecules responsible for primary stimulation, or antigen-dependent simulation. In some aspects, the intracellular signaling domain can comprise a costimulatory intracellular domain. Exemplary costimulatory intracellular signaling domains include those derived from molecules responsible for costimulatory signals, or antigen-independent stimulation. For example, in the case of a CAR T cell (e.g., anti-Siglec-15 CAR T cells described herein), a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, common FcR gamma (FCER1G), Fc gamma RIIa, FcR beta (Fc Epsilon Rib), CD3 gamma, CD3 delta, CD3 epsilon, CD22, CD79a, CD79b, CD278 (ICOS), FcεRI, CD66d, CD32, DAP10, and DAP12.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some embodiments, an antibody, polynucleotide, vector, cell, or composition which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain embodiments, the polypeptides can occur as single chains or associated chains.

“Percent identity” refers to the extent of identity between two sequences (e.g., amino acid sequences or nucleic acid sequences). Percent identity can be determined by aligning two sequences, introducing gaps to maximize identity between the sequences. Alignments can be generated using programs known in the art. For purposes herein, alignment of nucleotide sequences can be performed with the BlastN program set at default parameters, and alignment of amino acid sequences can be performed with the BlastPprogram set at default parameters (see National Center for Biotechnology Information (NCBI) on the worldwide web, ncbi.nlm nih.gov).

As used herein, the term “host cell” can be any type of cell, e.g., a primary cell, a cell in culture, or a cell from a cell line. In specific embodiments, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

The term “pharmaceutical formulation” refers to a preparation which is in such form as to permit the biological activity of the active ingredient, e.g., anti-Siglec-15 antibodies, to be effective, and which contains no additional components which are unacceptably toxic to a subject to which the formulation would be administered. The formulation can be sterile.

The terms “administer”, “administering”, “administration”, and the like, as used herein, refer to methods that may be used to enable delivery of a drug, e.g., an anti-Siglec-15 antibody or antigen-binding fragment thereof to the desired site of biological action (e.g., intravenous administration). Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa.

Administration “in combination with” one or more further therapeutic agents includes simultaneous (concurrent) or consecutive administration in any order.

As used herein, the terms “subject” and “patient” are used interchangeably. The subject can be an animal. In some embodiments, the subject is a mammal such as a non-human animal (e.g., cow, pig, horse, cat, dog, rat, mouse, monkey or other primate, etc.). In some embodiments, the subject is a cynomolgus monkey. In some embodiments, the subject is a human.

The term “therapeutically effective amount” refers to an amount of a drug, e.g., an anti-Siglec-15 antibody or antigen-binding fragment thereof effective to treat a disease or disorder in a subject. In the case of cancer, the therapeutically effective amount of the drug can reduce the number of cancer cells; reduce the tumor size or burden; inhibit (i.e., slow to some extent and in a certain embodiment, stop) cancer cell infiltration into peripheral organs; inhibit (i.e., slow to some extent and in a certain embodiment, stop) tumor metastasis; inhibit, to some extent, tumor growth; relieve to some extent one or more of the symptoms associated with the cancer; and/or result in a favorable response such as increased progression-free survival (PFS), disease-free survival (DFS), or overall survival (OS), complete response (CR), partial response (PR), or, in some cases, stable disease (SD), a decrease in progressive disease (PD), a reduced time to progression (TTP), or any combination thereof. To the extent the drug can prevent growth and/or kill existing cancer cells, it can be cytostatic and/or cytotoxic.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with or suspected of having the disorder. In certain embodiments, a subject is successfully “treated” for cancer according to the methods of the present invention if the patient shows one or more of the following: a reduction in the number of or complete absence of cancer cells; a reduction in the tumor size; inhibition of or an absence of cancer cell infiltration into peripheral organs including, for example, the spread of cancer into soft tissue and bone; inhibition of or an absence of tumor metastasis; inhibition or an absence of tumor growth; relief of one or more symptoms associated with the specific cancer; reduced morbidity and mortality; improvement in quality of life; reduction in tumorigenicity, tumorigenic frequency, or tumorigenic capacity, of a tumor; reduction in the number or frequency of cancer stem cells in a tumor; differentiation of tumorigenic cells to a non-tumorigenic state; increased progression-free survival (PFS), disease-free survival (DFS), or overall survival (OS), complete response (CR), partial response (PR), stable disease (SD), a decrease in progressive disease (PD), a reduced time to progression (TTP), or any combination thereof.

The terms “cancer” and “cancerous” refer to or describe the physiological condition in mammals in which a population of cells is characterized by unregulated cell growth. Examples of cancer include, but are not limited to, gynecological cancers (e.g., breast cancer (including triple negative breast cancer, ductal carcinoma), ovarian cancer, and endometrial cancer), non-small cell lung cancer, pancreatic cancer, thyroid cancer, kidney cancer (e.g., renal cell carcinoma), and bladder cancer (e.g., urothelial cell carcinoma). Additional examples of cancer include, e.g., head and neck cancer, small cell lung cancer, gastric cancer, melanoma, cholangiocarcinoma, osteosarcoma, glioblastoma or glioblastoma multiforme (GBM), and Merkel cell carcinoma. The cancer may be a primary tumor or may be advanced or metastatic cancer.

As used in the present disclosure and claims, the singular forms “a,” “an,” and “the” include plural forms unless the context clearly dictates otherwise.

It is understood that wherever embodiments are described herein with the language “comprising,” otherwise analogous embodiments described in terms of “consisting of” and/or “consisting essentially of” are also provided. In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “about” and “approximately,” when used to modify a numeric value or numeric range, indicate that deviations of 5% to 10% above and 5% to 10% below the value or range remain within the intended meaning of the recited value or range.

Any compositions or methods provided herein can be combined with one or more of any of the other compositions and methods provided herein.

### 1.2 Antibodies

In a specific aspect, provided herein are antibodies (e.g., monoclonal antibodies, such as chimeric, humanized, or human antibodies) and antigen-binding fragments thereof which specifically bind to Siglec-15 (e.g., human Siglec-15). Siglec-15 is a type-I transmembrane protein consisting of: (i) two immunoglobulin (Ig)-like domains, referred to herein as “D1” and “D2,” respectively, (ii) a transmembrane domain containing a lysine residue, and (iii) a short cytoplasmic tail (Angata, et al., Glycobiology, Volume 17, Issue 8, August 2007, Pages 838-846). D1 of human Siglec-15 spans amino acid positions 40-158, and D2 spans positions 168-251. The amino acid sequences for human, cynomolgus monkey and murine Siglec-15 are known in the art and also provided herein as represented by SEQ ID NOs 1-3, respectively.

Human Siglec-15 (NCBI NP-998767.1):
(SEQ ID NO: 1)
MEKSIWLLACLAWVLPTGSFVRTKIDTTENLLNTEVHSSPAQRWSMQVPP
EVSAEAGDAAVLPCTFTHPHRHYDGPLTAIWRAGEPYAGPQVERCAAARG
SELCQTALSLHGRFRLLGNPRRNDLSLRVERLALADDRRYFCRVEFAGDV
HDRYESRHGVRLHVTAAPRIVNISVLPSPAHAFRALCTAEGEPPPALAWS
GPALGNSLAAVRSPREGHGHLVTAELPALTHDGRYTCTAANSLGRSEASV
YLERFHGASGASTVALLLGALGFKALLLLGVLAARAARRRPEHLDTPDTP
PRSQAQESNYENLSQMNPRSPPATMCSP
Cynomolgus monkey Siglec-15 (Uniprot A0A2K5UY47):
(SEQ ID NO: 2)
MESSIRLLACLACVLPTGSFVRTKIDTTENLLNTEVHSSPVQRWSMQVPA
EVSAAAGDAAVLPCTFTHPHRHYDGPLTAIWRAGEPYAGPQVERCAAARG
SELCQTALSLHGRFRLLGNPRRNDLSLRVERLALADDRRYFCRVEFAGDV
HDRYESRHGVRLHVTAAPRIINISVLPGPAHAFRALCTTEGEPPPALAWS
GPALGNGSAAVPSSGOGHGHLVTAELPALNHDGRYTCTAANSLGRSEASV
YLFRFHGASASTVALLLGALGLKALLLLGVLAAGVARHRPEHLNTPDTPP
RSQAQESNYENLSOMNPRSPPAAMCSP
Murine Siglec-15 (NCBI NP_001094508.1)
(SEQ ID NO: 3)
MEGSLQLLACLACVLOMGSLVKTRRDASGDLLNTEAHSAPAQRWSMQVPA
EVNAEAGDAAVLPCTFTHPHRHYDGPLTAIWRSGEPYAGPQVFRCTAAPG
SELCQTALSLHGRFRLLGNPRRNDLSLRVERLALADSGRYFCRVEFTGDA
HDRYESRHGVRLRVTAAAPRIVNISVLPGPAHAFRALCTAEGEPPPALAW
SGPAPGNSSAALQGQGHGYQVTAELPALTRDGRYTCTAANSLGRAEASVY
LFRFHGAPGTSTLALLLGALGLKALLLLGILGARATRRRLDHLVPQDTPP
RSQAQESNYENLSQMSPPGHQLPRVCCEELLSHHHLVIHHEK

In certain embodiments, an antibody or antigen-binding fragment thereof described herein binds to human Siglec-15. In certain embodiments, an antibody or antigen-binding fragment thereof binds to human and cynomolgus monkey Siglec-15. In certain embodiments, an antibody or antigen-binding fragment thereof binds to human, and murine Siglec-15. In certain embodiments, an antibody or antigen-binding fragment thereof binds to human, cynomolgus monkey, and murine Siglec-15. In certain embodiments, an antibody or antigen-binding fragment thereof binds to the second immunoglobulin (Ig)-like domain (D2) of Siglec-15. The amino acid sequences for D1 and D2 of human Siglec-15 are provided herein as represented by SEQ ID NOs 94 and 95, respectively.

Domain 1 human Siglec-15 (Ig-like V-type)
(SEQ ID NO: 94)
PAQRWSMQVPPEVSAEAGDAAVLPCTFTHPHRHYDGPLTAIWRAGEPYAG
PQVERCAAARGSELCQTALSLHGRERLLGNPRRNDLSLRVERLALADDRR
YFCRVEFAGDVHDRYESRH
Domain 2 human Siglec-15 (Ig-like C2-type)
(SEQ ID NO: 95)
PRIVNISVLPSPAHAFRALCTAEGEPPPALAWSGPALGNSLAAVRSPREG
HGHLVTAELPALTHDGRYTCTAANSLGRSEASVY

In certain embodiments, an antibody or antigen-binding fragment thereof described herein binds to human Siglec-15 and comprises the six CDRs of an antibody listed in Tables 1 and 2 (i.e., the three VH CDRs of the antibody listed in Table 1 and the three VL CDRs of the same antibody listed in Table 2).

TABLE 1
Antibody VH CDR Sequences

| Antibody | CDRH1 | CDRH2 | CDRH3 |
|---|---|---|---|
| 62E5 | GYWMS (SEQ ID NO: | EIIPDSSTINYTPSLKD | RDGYWFAY (SEQ ID |
| | 4) | (SEQ ID NO: 5) | NO: 6) |
| S15B1 | SSWMN (SEQ ID NO: | RIYPGHGETNYNGKF | VDNSDPYYFDY |
| | 12) | KD (SEQ ID NO: 13) | (SEQ ID NO: 14) |
| ZLA-1243- | SYAMS (SEQ ID NO: | AISGSGGSTYYADSV | DNEWTPGSFFDY |
| A01 | 20) | KG (SEQ ID NO: 21) | (SEQ ID NO: 22) |
| ZLA-1243- | DYAMH (SEQ ID NO: | GISWNSGSIGYADSV | GLWPSYDLDAFDI |
| E01 | 28) | KG (SEQ ID NO: 29) | (SEQ ID NO: 30) |
| ZLA-1214- | GYYMH (SEQ ID NO: | RINPNSGGTNYAQKF | VINDAFDM (SEQ ID |
| D08 | 36) | QG (SEQ ID NO: 37) | NO: 38) |
| ZLA-1214- | SYAIS (SEQ ID NO: | GIIPIFGTANYAQKFQ | DGGRDGYNPKKILEF |
| B07 | 44) | G (SEQ ID NO: 45) | DY (SEQ ID NO: 46) |
| ZLA-1212- | SYGMH (SEQ ID NO: | VISYDGSNKYYADS | DDSGYYDSSGYDY |
| E09 | 52) | VKG (SEQ ID NO: 53) | (SEQ ID NO: 54) |
| ZLA-1212- | SYAMH (SEQ ID NO: | VISYDGSNKYYADS | EGGSYYGYFDY |
| B05 | 60) | VKG (SEQ ID NO: 61) | (SEQ ID NO: 62) |
| ZLA-1214- | SYGIS (SEQ ID NO: | WISAYNGNTNYAQK | MSGYYGMDV (SEQ |
| H05 | 68) | LQG (SEQ ID NO: 69) | ID NO: 70) |

TABLE 2
Antibody VL CDR Sequences

| Antibody | CDRL1 | CDRL2 | CDRL3 |
|---|---|---|---|
| 62E5 | KSSQSLLYSRNQQN | WASTRES (SEQ ID | QQYYSYPLT (SEQ ID |
| | YLA (SEQ ID NO: 7) | NO: 8) | NO: 9) |
| S15B1 | RASKSVSTSGYSYMH | LASNLES (SEQ ID | QHGRELPFT (SEQ ID |
| | (SEQ ID NO: 15) | NO: 16) | NO: 17) |
| ZLA-1243- | TGTSSDVGGYNYVS | DVSNRPS (SEQ ID | SSYTSSSTRV (SEQ |
| A01 | (SEQ ID NO: 23) | NO: 24) | ID NO: 25) |
| ZLA-1243- | TGTSSDVGAYNFVS | EVSNRPS (SEQ ID | SSYTSTIPPFV (SEQ |
| E01 | (SEQ ID NO: 31) | NO: 32) | ID NO: 33) |
| ZLA-1214- | RSSQSLLHSNGYNYLD | LGSNRAS (SEQ ID | MQALQTQYT (SEQ |
| D08 | (SEQ ID NO: 39) | NO: 40) | ID NO: 41) |
| ZLA-1214- | RASQSVSSSYLA | GASSRAT (SEQ ID | QQYGSSPIT (SEQ ID |
| B07 | (SEQ ID NO: 47) | NO: 48) | NO: 49) |
| ZLA-1212- | TGSSSNIGAGYDVH | GNSNRPS (SEQ ID | QSYDSSLSGVV (SEQ |
| E09 | (SEQ ID NO: 55) | NO: 56) | ID NO: 57) |
| ZLA-1212- | GGNNIGSKSVH (SEQ | DDSDRPS (SEQ ID | QVWDSSSDHRV |
| B05 | ID NO: 63) | NO: 64) | (SEQ ID NO: 65) |
| ZLA-1214- | QASQGISNYLN (SEQ | DASNLET (SEQ ID | QQYDNLPLT (SEQ ID |
| H05 | ID NO: 71) | NO: 72) | NO: 73) |

In certain embodiments, an antibody or antigen-binding fragment thereof described herein binds to human Siglec-15 and comprises the VH of an antibody listed in Table 3.

In certain embodiments, an antibody or antigen-binding fragment thereof described herein binds to human Siglec-15 and comprises the VL of an antibody listed in Table 4.

TABLE 3
Antibody VH Sequences. CDR regions according to Kabat numbering schema
are underlined in humanized S15B1 VH sequences, extended CDR1 region shown
underlined beyond bolded CDR1, back mutation sites shown in bold italics.

| Antibody | VH Sequence |
|---|---|
| 62E5 | EVKLLESGGGLVQPGGSLKLSCAASGFDFSGYWMSWVRQAPGK |
| | GLEWIGEIIPDSSTINYTPSLKDKFIISRDNAKNTLFLQMRKVRSED |
| | TALYYCARRDGYWFAYWGQGTLVTVSA (SEQ ID NO: 10) |
| S15B1 | QVQLQQSGPELVKPGASVKISCKASGYAFSSSWMNWVKQRPGKG |
| | LEWIGRIYPGHGETNYNGKFKDKATVTADKSSSTTYMQLSSLTSE |
| | DSAVYFCARVDNSDPYYFDYWGQGTTLTVSS (SEQ ID NO: 18) |
| ZLA-1243- | QVQLVESGGGLVQPGGSLRLSCAASGFTFSSYAMSWVRQAPGKG |
| A01 | LEWVSAISGSGGSTYYADSVKGRFTISRDNSKNTLYLQMNSLRAE |
| | DTAVYYCAKDNEWTPGSFFDYWGQGTLVTVSS (SEQ ID NO: 26) |
| ZLA-1243- | QVQLVQSGGGLVQPGRSLRLSCAASGFTFDDYAMHWVRQAPGK |
| E01 | GLEWVSGISWNSGSIGYADSVKGRFTISRDNAKNSLYLQMNSLRA |
| | EDTALYYCAKGLWPSYDLDAFDIWGQGTMVTVSS (SEQ ID NO: |
| | 34) |
| ZLA-1214- | EVQLVQSGAEVKKPGASVKVSCKASGYTFTGYYMHWVRQAPGQ |
| D08 | GLEWMGRINPNSGGTNYAQKFQGRVTMTRDTSTSTAYMELSRLR |
| | SDDTAVYYCARVINDAFDMWGQGTMVTVSS (SEQ ID NO: 42) |
| ZLA-1214- | QVQLVQSGAEVKKPGSSVKVSCKASGGTFSSYAISWVRQAPGQG |
| B07 | LEWMGGIIPIFGTANYAQKFQGRVTITADESTSTAYMELSSLRSED |
| | TAVYYCARDGGRDGYNPKKILEFDYWGQGTLVTVSS (SEQ ID |
| | NO: 50) |
| ZLA-1212- | QVQLVQSGGGVVQPGRSLRLSCAASGFTFSSYGMHWVRQAPGK |
| E09 | GLEWVAVISYDGSNKYYADSVKGRFTISRDNSKNTLYLQMNSLR |
| | AEDTAVYYCAKDDSGYYDSSGYDYWGQGTLVTVSS (SEQ ID NO: |
| | 58) |
| ZLA-1212- | QVQLVQSGGGVVQPGRSLRLSCAASGFTFSSYAMHWVRQAPGK |
| B05 | GLEWVAVISYDGSNKYYADSVKGRFTISRDNSKNTLYLQMNSLR |
| | AEDTAVYYCAREGGSYYGYFDYWGQGTLVTVSS (SEQ ID NO: |
| | 66) |
| ZLA-1214- | QVQLVQSGAEVKKPGASVKVSCKASGYTFTSYGISWVRQAPGQG |
| H05 | LEWMGWISAYNGNTNYAQKLQGRVTMTTDTSTSTAYMELRSLR |
| | SDDTAVYYCARMSGYYGMDVWGQGTLVTVSS (SEQ ID NO: 74) |
| SISBI-VH1 | QVQLVQSGAEVKKPGASVKVSCKASGYAFSSSWMNWVRQAPGQ |
| | GLEW/GRIYPGHGETNYNGKFKDRVTMTADTSTSTVYMELSSLRS |
| | EDTAVYFCARYDNSDPYYEDYWGQGTTVTVSS (SBQ ID NO: 103) |
| SISBI-VH2 | 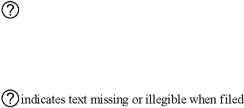 |
| SISBI-VH3 | 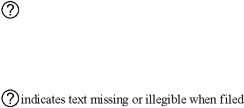 |
| S1SB1-VH4 | 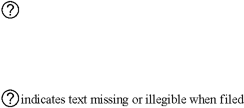 |

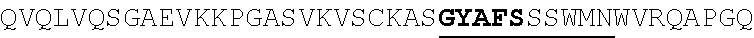

TABLE 4
Antibody VL Sequences. CDR regions according to Kabat numbering schema
are underlined in humanized S15B1 VL sequences, back mutation sites shown in bold
italics.

| Antibody | VL Sequence |
|---|---|
| 62E5 | DIVMSQSPSSLAVSVGEKVTMSCKSSQSLLYSRNQQNYLAWFQQ |
| | KPGQSPELLIYWASTRESGVPDRFTGSGSGTDFTLTISSVKAEDLA |
| | VYYCQQYYSYPLTFGAGTKLELK (SEQ ID NO: 11) |
| S15B1 | DIVLTQSPASLAVSLGQRATISCRASKSVSTSGYSYMHWYQQKPG |
| | QPPKLLIYLASNLESGVPARFSGSGSGTDFTLNIHPVEEEDAATYY |
| | CQHGRELPFTFGSGTKLEIK (SEQ ID NO: 19) |
| ZLA-1243- | QSALTQPASVSGSPGQSITISCTGTSSDVGGYNYVSWYQQHPGKA |
| A01 | PKLMIYDVSNRPSGVSNRFSGSKSGNTASLTISGLQAEDEADYYCS |
| | SYTSSSTRVFGGGTKVTVL (SEQ ID NO: 27) |
| ZLA-1243- | QSALTQPASVSGSPGQSITLSCTGTSSDVGAYNFVSWYQQHPGTA |
| E01 | PKLMIYEVSNRPSGVSDRFSGSKSGNTASLTISGLQAEDEADYYCS |
| | SYTSTIPPFVFGTGTKVTVL (SEQ ID NO: 35) |
| ZLA-1214- | DVVMTQSPLSLPVTPGEPASISCRSSQSLLHSNGYNYLDWYLQKP |
| D08 | GQSPQLLIYLGSNRASGVPDRFSGSGSGTDFTLKISRVEAEDVGVY |
| | YCMQALQTQYTFGQGTKVDIK (SEQ ID NO: 43) |
| ZLA-1214- | EIVLTQSPGTLSLSPGERATLSCRASQSVSSSYLAWYQQKPGQAPR |
| B07 | LLIYGASSRATGIPDRFSGSGSGTDFTLTISRLEPEDFAVYYCQQYG |
| | SSPITFGPGTKVDIK (SEQ ID NO: 51) |
| ZLA-1212- | QSVLTQPPSVSGAPGQRVTISCTGSSSNIGAGYDVHWYQQLPGTA |
| E09 | PKLLIYGNSNRPSGVPDRFSGSKSGTSASLAITGLQAEDEADYYCQ |
| | SYDSSLSGVVFGGGTKLTVL (SEQ ID NO: 59) |
| ZLA-1212- | QSVLTQPPSVSVAPGKTARITCGGNNIGSKSVHWYQQKPGQAPVL |
| B05 | VIYDDSDRPSGIPERFSGSNSGNTATLTISRVEAGDEADYYCQVWD |
| | SSSDHRVFGGGTKLTVL (SEQ ID NO: 67) |
| ZLA-1214- | DIQLTQSPSSLSASVGDRVTITCQASQGISNYLNWYQQKPGKAPKL |
| H05 | LIYDASNLETGVPSRFSGSGSGTDFTFTISSLQPEDIATYYCQQYDN |
| | LPLTFGGGTKLEIK (SEQ ID NO: 75) |
| SISBI-VLI | 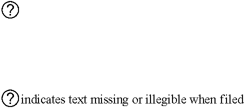 |
| S15BI-VL2 | 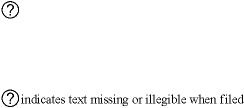 |

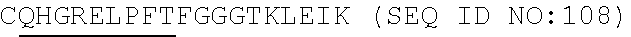

In certain embodiments, an antibody or antigen-binding fragment thereof described herein binds to human Siglec-15, comprises the six CDRs of an antibody listed in Tables 1 and 2 (i.e., the three VH CDRs of the antibody listed in Table 1 and the three VL CDRs of the same antibody listed in Table 2), and comprises a VH comprising a sequence at least 80% identical to the VH sequence of the same antibody in Table 3 and a VL comprising a sequence at least 80% identical to the VL sequence of the same antibody in Table 4. In certain embodiments, an antibody or antigen-binding fragment thereof described herein binds to human Siglec-15, comprises the six CDRs of an antibody listed in Tables 1 and 2 (i.e., the three VH CDRs of the antibody listed in Table 1 and the three VL CDRs of the same antibody listed in Table 2), and comprises a VH comprising a sequence at least 85% identical to the VH sequence of the same antibody in Table 3 and a VL comprising a sequence at least 85% identical to the VL sequence of the same antibody in Table 4.

In certain embodiments, an antibody or antigen-binding fragment thereof described herein binds to human Siglec-15, comprises the six CDRs of an antibody listed in Tables 1 and 2 (i.e., the three VH CDRs of the antibody listed in Table 1 and the three VL CDRs of the same antibody listed in Table 2), and comprises a VH comprising a sequence at least 90% identical to the VH sequence of the same antibody in Table 3 and a VL comprising a sequence at least 90% identical to the VL sequence of the same antibody in Table 4. In certain embodiments, an antibody or antigen-binding fragment thereof described herein binds to human Siglec-15, comprises the six CDRs of an antibody listed in Tables 1 and 2 (i.e., the three VH CDRs of the antibody listed in Table 1 and the three VL CDRs of the same antibody listed in Table 2), and comprises a VH comprising a sequence at least 95% identical to the VH sequence of the same antibody in Table 3 and a VL comprising a sequence at least 95% identical to the VL sequence of the same antibody in Table 4.

In certain embodiments, an antibody or antigen-binding fragment thereof described herein binds to human Siglec-15, comprises the six CDRs of an antibody listed in Tables 1 and 2 (i.e., the three VH CDRs of the antibody listed in Table 1 and the three VL CDRs of the same antibody listed in Table 2), and comprises a VH comprising a sequence at least 96% identical to the VH sequence of the same antibody in Table 3 and a VL comprising a sequence at least 96% identical to the VL sequence of the same antibody in Table 4. In certain embodiments, an antibody or antigen-binding fragment thereof described herein binds to human Siglec-15, comprises the six CDRs of an antibody listed in Tables 1 and 2 (i.e., the three VH CDRs of the antibody listed in Table 1 and the three VL CDRs of the same antibody listed in Table 2), and comprises a VH comprising a sequence at least 97% identical to the VH sequence of the same antibody in Table 3 and a VL comprising a sequence at least 97% identical to the VL sequence of the same antibody in Table 4. In certain embodiments, an antibody or antigen-binding fragment thereof described herein binds to human Siglec-15, comprises the six CDRs of an antibody listed in Tables 1 and 2 (i.e., the three VH CDRs of the antibody listed in Table 1 and the three VL CDRs of the same antibody listed in Table 2), and comprises a VH comprising a sequence at least 98% identical to the VH sequence of the same antibody in Table 3 and a VL comprising a sequence at least 98% identical to the VL sequence of the same antibody in Table 4. In certain embodiments, an antibody or antigen-binding fragment thereof described herein binds to human Siglec-15, comprises the six CDRs of an antibody listed in Tables 1 and 2 (i.e., the three VH CDRs of the antibody listed in Table 1 and the three VL CDRs of the same antibody listed in Table 2), and comprises a VH comprising a sequence at least 99% identical to the VH sequence of the same antibody in Table 3 and a VL comprising a sequence at least 99% identical to the VL sequence of the same antibody in Table 4.

Certain aspects of the present disclosure relate to bispecific antibodies that bind to one or more domains on a Siglec-15 protein of the present disclosure and a second antigen. Methods of generating bispecific antibodies are well known in the art and described herein. In some embodiments, bispecific antibodies of the present disclosure bind to one or more amino acid residues of a Siglec-15 protein of the present disclosure, such as one or more amino acid residues of human Siglec-15 (SEQ ID NO: 1), or amino acid residues on a Siglec-15 protein corresponding to amino acid residues of SEQ ID NO: 1. In some embodiments, bispecific antibodies of the present disclosure recognize a first antigen and a second antigen. In some embodiments, the first antigen is a Siglec-15 protein or a naturally occurring variant thereof. In some embodiments, the second antigen is also a Siglec-15 protein, or a naturally occurring variant thereof. In some embodiments, the second antigen is an antigen facilitating transport across the blood-brain-barrier (see, e.g., Gabathuler R., Neurobiol. Dis. 37 (2010) 48-57). Such second antigens include, without limitation, transferrin receptor (TR), insulin receptor (HIR), insulin-like growth factor receptor (IGFR), low-density lipoprotein receptor-related proteins 1 and 2 (LPR-1 and 2), diphtheria toxin receptor, CRM 197, a llama single domain antibody, TMEM 30 (A), a protein transduction domain, TAT, Syn-B, penetratin, a poly-arginine peptide, Angiopep peptides such as ANG1005 (see, e.g., Gabathuler, 2010), and other cell surface proteins that are enriched on blood-brain barrier endothelial cells (see, e.g., Daneman et al., PLOS One. 2010 Oct. 29; 5 (10): e13741). In some embodiments, the second antigen is a disease-causing protein including, without limitation, amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides. In some embodiments, the second antigen is one or more ligands and/or proteins expressed on immune cells, including but not limited to Siglec-9, Siglec-10, CD25, CD47, TREM-2, SIRP1a, CD3, PD1/PDL1, CD40, OX40, ICOS, CD28, CD137/4-1BB, CD27, GITR, PD-L1, CTLA4, PD-L2, PD-1, B7-H3, B7-H4, HVEM, LIGHT, BTLA, CD30, TIGIT, VISTA, KIR, GAL9, TIM1, TIM3, TIM4, A2AR, LAG3, DR-5, CD2, CD5, CD39, CD73, and phosphatidylserine. In some embodiments, the second antigen is a protein, lipid, polysaccharide, or glycolipid expressed on one or more tumor cells.

In certain aspects, the CDRs of an antibody or antigen-binding fragment thereof can be determined according to the Chothia numbering scheme, which refers to the location of immunoglobulin structural loops (see, e.g., Chothia C & Lesk A M, (1987), J Mol Biol 196:901-917; Al-Lazikani B et al., (1997) J Mol Biol 273:927-948; Chothia C et al., (1992) J Mol Biol 227:799-817; Tramontano A et al., (1990) J Mol Biol 215 (1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-HI loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

In certain aspects, the CDRs of an antibody or antigen-binding fragment thereof can be determined according to the IMGT numbering system as described in Lefranc M-P, (1999) The Immunologist 7:132-136 and Lefranc M-P et al., (1999) Nucleic Acids Res 27:209-212. According to the IMGT numbering scheme, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97.

In certain aspects, the CDRs of an antibody or antigen-binding fragment thereof can be determined according to MacCallum R M et al., (1996) J Mol Biol 262:732-745. See also, e.g., Martin A. “Protein Sequence and Structure Analysis of Antibody Variable Domains,” in Antibody Engineering, Kontermann and Dübel, eds., Chapter 31, pp. 422-439, Springer-Verlag, Berlin (2001).

In certain aspects, the CDRs of an antibody or antigen-binding fragment thereof can be determined according to the AbM numbering scheme, which refers AbM hypervariable regions which represent a compromise between the Kabat CDRs and Chothia structural loops, and are used by Oxford Molecular's AbM antibody modeling software (Oxford Molecular Group, Inc.).

In specific aspects, provided herein are antibodies that comprise a heavy chain and a light chain. With respect to the heavy chain, in a specific embodiment, the heavy chain of an antibody described herein can be an alpha (a), delta (8), epsilon (¿), gamma (γ) or mu (u) heavy chain. In another specific embodiment, the heavy chain of an antibody described can comprise a human alpha (a), delta (8), epsilon (¿), gamma (γ) or mu (u) heavy chain. Non-limiting examples of human constant region sequences have been described in the art, e.g., see U.S. Pat. No. 5,693,780 (incorporated herein by reference) and Kabat E A et al., (1991) supra.

With respect to the light chain, in a specific embodiment, the light chain of an antibody described herein is a kappa light chain. In another specific embodiment, the light chain of an antibody described herein is a lambda light chain. In yet another specific embodiment, the light chain of an antibody described herein is a human kappa light chain or a human lambda light chain. Examples of human immunoglobulin light chain constant region amino acid sequences are shown in the following table.

TABLE 5
Exemplary Human Light Chain Constant Region Sequences

| Designation | SEQ ID NO | Amino Acid Sequence |
|---|---|---|
| Human | 96 | GQPKANPTVTLFPPSSEELQANKATLVCLISDFYPGAVT |
| lambda v1 | | VAWKADGSPVKAGVETTKPSKQSNNKYAASSYLSLTP |
| | | EQWKSHRSYSCQVTHEGSTVEKTVAPTECS |
| Human | 97 | GQPKAAPSVTLFPPSSEELQANKATLVCLISDFYPGAVT |
| lambda v2 | | VAWKADSSPVKAGVETTTPSKQSNNKYAASSYLSLTPE |
| | | QWKSHRSYSCQTHEGSTVEKTVAPTECS |
| Human | 98 | QPKAAPSVTLFPPSSEELQANKATLVCLISDFYPGAVTV |
| lambda v3 | | AWKADSSPVKAGVETTTPSKQSNNKYAASSYLSLTPEQ |
| | | WKSHRSYSCQVTHEGSTVEKTVAPTECS |
| Human | 99 | GQPKAAPSVTLFPPSSEELQANKATLVCLISDFYPGAVT |
| lambda v4 | | VAWKADSSPVKAGVETTTPSKQSNNKYAASSYLSLTPE |
| | | QWKSHKSYSCQVTHEGSTVEKTVAPTECS |
| Human | 100 | GQPKAAPSVTLFPPSSEELQANKATLVCLVSDFYPGAVT |
| lambda v5 | | VAWKADGSPVKVGVETTKPSKQSNNKYAASSYLSLTP |
| | | EQWKSHRSYSCRVTHEGSTVEKTVAPAECS |
| Human | 101 | TVAAPSVFIFPPSDEQLKSGTASVVCLLNNFYPREAKVQ |
| kappa v1 | | WKVDNALQSGNSQESVTEQDSKDSTYSLSSTLTLSKAD |
| | | YEKHKVYACEVTHQGLSSPVTKSFNRGEC |
| Human | 102 | RTVAAPSVFIFPPSDEQLKSGTASVVCLLNNFYPREAKV |
| kappa v2 | | QWKVDNALQSGNSQESVTEQDSKDSTYSLSSTLTLSKA |
| | | DYEKHKVYACEVTHQGLSSPVTKSFNRGEC |

In certain embodiments, one, two, or more mutations (e.g., amino acid substitutions) are introduced into the Fc region of an antibody or antigen-binding fragment thereof described herein (e.g., CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody or antigen-binding fragment thereof, such as serum half-life, complement fixation, Fc receptor binding, and/or antigen-dependent cellular cytotoxicity. In certain embodiments, mutations in the Fc region of an antibody or antigen-binding fragment thereof function to silence the Fc region (i.e., remove Fc function). These mutations may include NA (N297/A/Q/G), AAA (L235A/G237A/E318A), LALA (L234A/L235A), IgG4-PE (S228P/L235E), RR (G236R/L328R), GA (S298G/T299A), FES (L234F/L235E/P331S), IgG2m4 (H268Q/V309L/A330S/P331S), XmAb® bispecific (E233P/L234V/L235A/G236del/S267K), LALA-PG (L234A/L235A/P329G), IgG2c4d (V234A/G237A/P238S/H268A/V309L/A330S/P331S), or FEA mutations (L234F/L235E/D265A).

In certain embodiments, one, two, or more mutations (e.g., amino acid substitutions) are introduced into the hinge region of the Fc region (CH1 domain) such that the number of cysteine residues in the hinge region is altered (e.g., increased or decreased) as described in, e.g., U.S. Pat. No. 5,677,425 (incorporated herein by reference). The number of cysteine residues in the hinge region of the CH1 domain may be altered to, e.g., facilitate assembly of the light and heavy chains, or to alter (e.g., increase or decrease) the stability of the antibody or antigen-binding fragment thereof.

In some embodiments, one, two, or more mutations (e.g., amino acid substitutions) are introduced into the Fc region of an antibody or antigen-binding fragment thereof described herein (e.g., CH2 domain (residues 231-340 of human IgG1) and/or CH3 domain (residues 341-447 of human IgG1) and/or the hinge region, with numbering according to the Kabat numbering system (e.g., the EU index in Kabat)) to increase or decrease the affinity of the antibody or antigen-binding fragment thereof for an Fc receptor (e.g., an activated Fc receptor) on the surface of an effector cell. Mutations in the Fc region that decrease or increase affinity for an Fc receptor and techniques for introducing such mutations into the Fc receptor or fragment thereof are known to one of skill in the art. Examples of mutations in the Fc receptor that can be made to alter the affinity of the antibody or antigen-binding fragment thereof for an Fc receptor are described in, e.g., Smith P et al., (2012) PNAS 109:6181-6186, Liu R et al., (2020) Antibodies 9 (64): 1-34, U.S. Pat. No. 6,737,056 (incorporated herein by reference), and International Publication Nos. WO 02/060919; WO 98/23289; and WO 97/34631, which are incorporated herein by reference. In certain embodiments, modifications in the Fc region of an antibody or antigen-binding fragment thereof function to increase affinity for one or more Fc receptors. These modifications may include reduced core fucosylation, afucosylation, and/or mutation including AAA (S298A/E333A/K334A), DE (S239D/1332E), DLE G236A, GAALIE (G236A/A330L/1332E), GASDALIE (S239D/A330L/1332E), (G236A/S239D/A330L/1332E), LPLIL (F243L/R292P/Y300L/V305I/P396L), VLPLL (L235V/F243L/R292P/Y300L/P396L), or Asym-mAb1 (one heavy chain: L234Y/L235Q/G236W/S239M/H268D/D270E/S298A, opposing heavy chain: D270E/K326D/A330M/K334E) mutations.

In some embodiments, the antibodies disclosed herein induce cytotoxicity in target cells. In some embodiments, the antibodies induce cytotoxicity in about 5-100% of target cells. In some embodiments, the antibodies induce cytotoxicity in about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of target cells. In some embodiments, the antibodies induce cytotoxicity in at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of target cells.

In some embodiments, the anti-Siglec-15 antibodies disclosed herein have a high affinity for human Siglec-15 protein as measured by the dissociation constant (K_{D}) between the antibody and Siglec-15. In some embodiments, the antibodies have a K_{D} value between 1×10⁻⁵ and 1×10⁻¹³ M. In some embodiments, the antibodies have a K_{D} value of less than 1×10⁻⁵, less than 1×10⁻⁶, less than 1×10⁻⁷, less than 1×10⁻⁸, less than 1×10⁻⁹, less than 1×10⁻¹⁰, less than 1×10⁻¹¹, less than 1×10⁻¹², or less than 1×10⁻¹³.

### Antibody Production

Antibodies and antigen-binding fragments thereof that immunospecifically bind to Siglec-15 (e.g., human Siglec-15) can be produced by any method known in the art for the synthesis of antibodies and antigen-binding fragments thereof, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Sambrook J et al., (2001) Molecular Cloning: A Laboratory Manual, Cold Spring Harbor Laboratory Press, Cold Spring Harbor, N.Y.; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press.

In a certain aspect, provided herein is a method of making an antibody or antigen-binding fragment which immunospecifically binds to Siglec-15 (e.g., human Siglec-15) comprising culturing a cell or host cell described herein. In a certain aspect, provided herein is a method of making an antibody or antigen-binding fragment thereof which immunospecifically binds to Siglec-15 (e.g., human Siglec-15) comprising expressing (e.g., recombinantly expressing) the antibody or antigen-binding fragment thereof using a cell or host cell described herein (e.g., a cell or a host cell comprising polynucleotides encoding an antibody or antigen-binding fragment thereof described herein). In a particular embodiment, the cell is an isolated cell. In a particular embodiment, the exogenous polynucleotides have been introduced into the cell. In a particular embodiment, the method further comprises the step of purifying the antibody or antigen-binding fragment obtained from the cell or host cell.

Methods for producing polyclonal antibodies are known in the art (see, for example, Chapter 11 in: Short Protocols in Molecular Biology, (2002) 5th Ed., Ausubel F M et al., eds., John Wiley and Sons, New York).

Monoclonal antibodies or antigen-binding fragments thereof can be prepared using a wide variety of techniques known in the art including the use of hybridoma, recombinant, and phage display technologies, yeast-based presentation technologies, or a combination thereof. For example, monoclonal antibodies or antigen-binding fragments thereof can be produced using hybridoma techniques including those known in the art and taught, for example, in Harlow E & Lane D, Antibodies: A Laboratory Manual, (Cold Spring Harbor Laboratory Press, 2nd ed. 1988); Hammerling G J et al., in: Monoclonal Antibodies and T-Cell Hybridomas 563 681 (Elsevier, N.Y., 1981), or as described in Kohler G & Milstein C (1975) Nature 256:495. Examples of yeast-based presentation methods that can be employed to select and generate the antibodies described herein include those disclosed in, for example, WO2009/036379A2; WO2010/105256; and WO2012/009568, each of which is herein incorporated by reference in its entirety.

In specific embodiments, a monoclonal antibody or antigen-binding fragment is an antibody or antigen-binding fragment produced by a clonal cell (e.g., hybridoma or host cell producing a recombinant antibody or antigen-binding fragment), wherein the antibody or antigen-binding fragment immunospecifically binds to Siglec-15 (e.g., human Siglec-15) as determined, e.g., by ELISA or other antigen-binding assays known in the art or in the examples provided herein. In particular embodiments, a monoclonal antibody or antigen-binding fragment thereof can be a chimeric or a humanized antibody or antigen-binding fragment thereof. In certain embodiments, a monoclonal antibody or antigen-binding fragment thereof can be a Fab fragment or a F(ab′)2 fragment. Monoclonal antibodies or antigen-binding fragments thereof described herein can, for example, be made by the hybridoma method as described in Kohler G & Milstein C (1975) Nature 256:495 or can, e.g., be isolated from phage libraries using the techniques as described herein, for example. Other methods for the preparation of clonal cell lines and of monoclonal antibodies and antigen-binding fragments thereof expressed thereby are well known in the art (see, for example, Chapter 11 in: Short Protocols in Molecular Biology, (2002) 5th Ed., Ausubel F M et al., supra).

Antigen-binding fragments of antibodies described herein can be generated by any technique known to those of skill in the art. For example, Fab and F(ab′)2 fragments described herein can be produced by proteolytic cleavage of immunoglobulin molecules, using enzymes such as papain (to produce Fab fragments) or pepsin (to produce F(ab′)2 fragments). A Fab fragment corresponds to one of the two identical arms of a tetrameric antibody molecule and contains the complete light chain paired with the VH and CH1 domains of the heavy chain. A F(ab′)2 fragment contains the two antigen-binding arms of a tetrameric antibody molecule linked by disulfide bonds in the hinge region.

Further, the antibodies or antigen-binding fragments thereof described herein can also be generated using various phage display and/or yeast-based presentation methods known in the art. In phage display methods, proteins are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with a scFv linker by PCR and cloned into a phagemid vector. The vector is electroporated in E. coli and the E. coli is infected with helper phage. Phage used in these methods are typically filamentous phage including fd and M13, and the VH and VL domains are usually recombinantly fused to either the phage gene III or gene VIII. Phage expressing an antibody or antigen-binding fragment thereof that binds to a particular antigen can be selected or identified with antigen, e.g., using labeled antigen or antigen bound or captured to a solid surface or bead. Examples of phage display methods that can be used to make the antibodies or fragments described herein include those disclosed in Brinkman U et al., (1995) J Immunol Methods 182:41-50; Ames R S et al., (1995) J Immunol Methods 184:177-186; Kettleborough C A et al., (1994) Eur J Immunol 24:952-958; Persic L et al., (1997) Gene 187:9-18; Burton D R & Barbas C F (1994) Advan Immunol 57:191-280; PCT Application No. PCT/GB91/001134; International Publication Nos. WO 90/02809, WO 91/10737, WO 92/01047, WO 92/18619, WO 93/11236, WO 95/15982, WO 95/20401, and WO 97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743, and 5,969,108 (each of which are incorporated herein by reference).

A humanized antibody or antigen-binding fragment thereof can be selected from any class of immunoglobulins, including IgM, IgG, IgD, IgA and IgE, and any isotype, including IgG1, IgG2, IgG3 and IgG4.

### Bispecific and Polyspecific Antibodies

Bispecific antibodies (BsAbs) are antibodies that have binding specificities for at least two different epitopes, including those on the same or another protein (e.g., one or more Siglec-15 proteins of the present disclosure). Alternatively, one part of a BsAb can be armed to bind to the target Siglec-15 antigen, and another can be combined with an arm that binds to a second protein. Such antibodies can be derived from full length antibodies or antibody fragments (e.g., F(ab′)₂ bispecific antibodies).

Methods for making bispecific antibodies are known in the art. Traditional production of full length bispecific antibodies is based on the coexpression of two immunoglobulin heavy-chain/light chain pairs, where the two chains have different specificities. Milstein et al., Nature, 305:537-539 (1983). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829 and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, C_{H}2, and C_{H}3 regions. It is preferred to have the first heavy-chain constant region (C_{H}1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only half of the bispecific molecules provides for an easy way of separation. This approach is disclosed in WO 94/04690. For further details on generating bispecific antibodies, see, for example, Suresh et al., Methods in Enzymology 121:210 (1986).

According to another approach described in WO 96/27011 or U.S. Pat. No. 5,731,168 (incorporated herein by reference) the interface between a pair of antibody molecules can be engineered to maximize the percentage of heterodimers which are recovered from recombinant cell culture. The preferred interface comprises at least a part of the C_{H}3 region of an antibody constant domain. In this method, one or more small amino acid side chains from the interface of the first antibody molecule are replaced with larger side chains (e.g., of tyrosine or tryptophan). Compensatory “cavities” of identical or similar size to the large side chains(s) are created on the interface of the second antibody molecule by replacing large amino acid side chains with smaller ones (e.g., of alanine or threonine). This provides a mechanism for increasing the yield of the heterodimer over other unwanted end-products such as homodimers.

Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exp. Med. 175:217-225 (1992) describes the production of fully humanized bispecific antibody F(ab′)₂ molecules. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bivalent antibody fragments directly from recombinant cell culture have also been described. For example, bivalent heterodimers have been produced using leucine zippers. Kostelny et al., J. Immunol., 148 (5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. The “diabody” technology described by Hollinger et al., Proc. Nat'l Acad. Sci. USA, 90:6444-6448 (1993) has provided an alternative mechanism for making bispecific/bivalent antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific/bivalent antibody fragments by the use of single-chain Fv (sFv) dimers has also been reported. See Gruber et al., J. Immunol., 152:5368 (1994).

Antibodies with more than two valencies are also contemplated. For example, trispecific antibodies can be prepared. Tutt et al., J. Immunol. 147:60 (1991).

Exemplary bispecific antibodies may bind to two different epitopes on a given molecule (e.g., a Siglec-15 protein of the present disclosure). Alternatively, an arm targeting a Siglec-15 signaling component may be combined with an arm which binds to a triggering molecule on a leukocyte such as a T cell receptor molecule (e.g., CD2, CD3, CD28 or B7), or Fc receptors for IgG (FcγR), such as FcγRI (CD64), FcγRII (CD32) and FcγRIII (CD16) so as to focus cellular defense mechanisms to the cell expressing the particular protein. Bispecific antibodies may also be used to localize cytotoxic agents to cells which express a particular protein. Such antibodies possess a protein-binding arm and an arm which binds a cytotoxic agent or a radionuclide chelator, such as EOTUBE, DPTA, DOTA or TETA. Another bispecific antibody of interest binds the protein of interest and further binds tissue factor (TF). In some embodiments, the bispecific antibodies bind to Siglec-15 and CD3.

In some embodiments, the anti-CD3 antibody comprises a VH comprising a sequence at least 80%, at least 85%, at least 90%, at least 95%, at least 97%, or at least 99% identical to SEQ ID NO: 109 and a VL comprising a sequence at least 80%, at least 85%, at least 90%, at least 95%, at least 97%, or at least 99% identical to SEQ ID NO: 110.

In certain embodiments, a bispecific antibody described herein binds to human Siglec-15 and human CD3, comprises an anti-Siglec-15 arm comprising a light chain comprising a sequence at least 80% identical to the light chain sequence of SEQ ID NO: 111 and a heavy chain comprising a sequence at least 80% identical to heavy chain sequence of SEQ ID NO: 112 and an anti-CD3 arm comprising a light chain comprising a sequence at least 80% identical to the light chain sequence of SEQ ID NO: 114 and a heavy chain sequence at least 80% identical to the heavy chain sequence of SEQ ID NO: 113. In certain embodiments, a bispecific antibody described herein comprises an anti-Siglec-15 arm comprising a light chain comprising a sequence at least 80% identical to the light chain sequence of SEQ ID NO: 115 and a heavy chain comprising a sequence at least 80% identical to heavy chain sequence of SEQ ID NO: 116 and an anti-CD3 arm comprising a light chain comprising a sequence at least 80% identical to the light chain sequence of SEQ ID NO: 118 and a heavy chain sequence at least 80% identical to the heavy chain sequence of SEQ ID NO: 117.

In certain embodiments, a bispecific antibody described herein binds to human Siglec-15 and human CD3, comprises an anti-Siglec-15 arm comprising a light chain comprising a sequence at least 85% identical to the light chain sequence of SEQ ID NO: 111 and a heavy chain comprising a sequence at least 85% identical to heavy chain sequence of SEQ ID NO: 112 and an anti-CD3 arm comprising a light chain comprising a sequence at least 85% identical to the light chain sequence of SEQ ID NO: 114 and a heavy chain sequence at least 85% identical to the heavy chain sequence of SEQ ID NO: 113. In certain embodiments, a bispecific antibody described herein comprises an anti-Siglec-15 arm comprising a light chain comprising a sequence at least 85% identical to the light chain sequence of SEQ ID NO: 115 and a heavy chain comprising a sequence at least 85% identical to heavy chain sequence of SEQ ID NO: 116 and an anti-CD3 arm comprising a light chain comprising a sequence at least 85% identical to the light chain sequence of SEQ ID NO: 118 and a heavy chain sequence at least 85% identical to the heavy chain sequence of SEQ ID NO: 117.

In certain embodiments, a bispecific antibody described herein binds to human Siglec-15 and human CD3, comprises an anti-Siglec-15 arm comprising a light chain comprising a sequence at least 90% identical to the light chain sequence of SEQ ID NO: 111 and a heavy chain comprising a sequence at least 90% identical to heavy chain sequence of SEQ ID NO: 112 and an anti-CD3 arm comprising a light chain comprising a sequence at least 90% identical to the light chain sequence of SEQ ID NO: 114 and a heavy chain sequence at least 90% identical to the heavy chain sequence of SEQ ID NO: 113. In certain embodiments, a bispecific antibody described herein comprises an anti-Siglec-15 arm comprising a light chain comprising a sequence at least 90% identical to the light chain sequence of SEQ ID NO: 115 and a heavy chain comprising a sequence at least 90% identical to heavy chain sequence of SEQ ID NO: 116 and an anti-CD3 arm comprising a light chain comprising a sequence at least 90% identical to the light chain sequence of SEQ ID NO: 118 and a heavy chain sequence at least 90% identical to the heavy chain sequence of SEQ ID NO: 117.

In certain embodiments, a bispecific antibody described herein binds to human Siglec-15 and human CD3, comprises an anti-Siglec-15 arm comprising a light chain comprising a sequence at least 95% identical to the light chain sequence of SEQ ID NO: 111 and a heavy chain comprising a sequence at least 95% identical to heavy chain sequence of SEQ ID NO: 112 and an anti-CD3 arm comprising a light chain comprising a sequence at least 95% identical to the light chain sequence of SEQ ID NO: 114 and a heavy chain sequence at least 95% identical to the heavy chain sequence of SEQ ID NO: 113. In certain embodiments, a bispecific antibody described herein comprises an anti-Siglec-15 arm comprising a light chain comprising a sequence at least 95% identical to the light chain sequence of SEQ ID NO: 115 and a heavy chain comprising a sequence at least 95% identical to heavy chain sequence of SEQ ID NO: 116 and an anti-CD3 arm comprising a light chain comprising a sequence at least 95% identical to the light chain sequence of SEQ ID NO: 118 and a heavy chain sequence at least 95% identical to the heavy chain sequence of SEQ ID NO: 117.

In certain embodiments, a bispecific antibody described herein binds to human Siglec-15 and human CD3, comprises an anti-Siglec-15 arm comprising a light chain comprising a sequence at least 97% identical to the light chain sequence of SEQ ID NO: 111 and a heavy chain comprising a sequence at least 97% identical to heavy chain sequence of SEQ ID NO: 112 and an anti-CD3 arm comprising a light chain comprising a sequence at least 97% identical to the light chain sequence of SEQ ID NO: 114 and a heavy chain sequence at least 97% identical to the heavy chain sequence of SEQ ID NO: 113. In certain embodiments, a bispecific antibody described herein comprises an anti-Siglec-15 arm comprising a light chain comprising a sequence at least 97% identical to the light chain sequence of SEQ ID NO: 115 and a heavy chain comprising a sequence at least 97% identical to heavy chain sequence of SEQ ID NO: 116 and an anti-CD3 arm comprising a light chain comprising a sequence at least 97% identical to the light chain sequence of SEQ ID NO: 118 and a heavy chain sequence at least 97% identical to the heavy chain sequence of SEQ ID NO: 117.

In certain embodiments, a bispecific antibody described herein binds to human Siglec-15 and human CD3, comprises an anti-Siglec-15 arm comprising a light chain comprising a sequence at least 99% identical to the light chain sequence of SEQ ID NO: 111 and a heavy chain comprising a sequence at least 99% identical to heavy chain sequence of SEQ ID NO: 112 and an anti-CD3 arm comprising a light chain comprising a sequence at least 99% identical to the light chain sequence of SEQ ID NO: 114 and a heavy chain sequence at least 99% identical to the heavy chain sequence of SEQ ID NO: 113. In certain embodiments, a bispecific antibody described herein comprises an anti-Siglec-15 arm comprising a light chain comprising a sequence at least 99% identical to the light chain sequence of SEQ ID NO: 115 and a heavy chain comprising a sequence at least 99% identical to heavy chain sequence of SEQ ID NO: 116 and an anti-CD3 arm comprising a light chain comprising a sequence at least 99% identical to the light chain sequence of SEQ ID NO: 118 and a heavy chain sequence at least 99% identical to the heavy chain sequence of SEQ ID NO: 117.

In some embodiments, the bispecific antibodies induce cytotoxicity in target cells. In some embodiments, the bispecific antibodies induce cytotoxicity in about 5-100% of target cells. In some embodiments, the bispecific antibodies induce cytotoxicity in about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of target cells. In some embodiments, the bispecific antibodies induce cytotoxicity in at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of target cells.

In some embodiments, the bispecific antibodies disclosed herein have a high affinity for human Siglec-15 protein and another target, as measured by the dissociation constant (K_{D}) between the antibody and Siglec-15 or the other target. In some embodiments, the antibodies have a K_{D} value for each target between 1×10⁻⁵ and 1×10⁻¹³ M. In some embodiments, the antibodies have a K_{D} value for each target of less than 1×10⁻⁵, less than 1×10⁻⁶, less than 1×10⁻⁷, less than 1×10⁻⁸, less than 1×10⁻⁹, less than 1×10⁻¹⁰, less than 1×10⁻¹¹, less than 1×10⁻¹², or less than 1×10⁻¹³. In some embodiments, the bispecific antibodies have higher affinity for one target than the other target (e.g., Siglec-15 and CD3).

### 1.2.1 Antibody Conjugates

A particularly innovative aspect of anti-Siglec-15 therapy involves the use of ADCs. These conjugates are composed of a monoclonal antibody linked to a cytotoxic agent or ‘payload’. The antibody component of the ADC specifically binds to Siglec-15 on TAMs, facilitating the delivery of the cytotoxic payload directly to the tumor microenvironment. Upon binding to Siglec-15-expressing cells, the linker connecting the antibody to the payload is cleaved, releasing the cytotoxic agent. This release not only affects the targeted TAMs but can also lead to a bystander effect, where neighboring tumor cells, irrespective of their Siglec-15 expression status, are impacted by the released chemotherapy. This mechanism is particularly advantageous as it allows the chemotherapy to affect tumor zones infiltrated by Siglec-15 positive macrophages, extending the therapeutic impact beyond the direct targets of the ADC. Such a strategy could enhance the efficacy of chemotherapy by ensuring localized delivery to the tumor site, reducing systemic toxicity, and overcoming the limitations of tumor heterogeneity.

Overall, the use of anti-Siglec-15 therapeutics, particularly ADCs, offers a multifaceted approach to cancer treatment. By targeting the immunosuppressive TAMs, these therapies can reinvigorate the immune system's ability to fight cancer. Simultaneously, the ADC approach provides a targeted delivery system for chemotherapeutic agents, maximizing their impact on the tumor while minimizing systemic exposure. This dual action-immune modulation and targeted chemotherapy-presents a significant advancement in cancer therapy, with the potential to improve clinical outcomes in several cancer types.

Anti-Siglec-15 antibodies of the present disclosure, or antibody fragments thereof, can be conjugated to a detectable marker, a toxin, or a therapeutic agent. Any suitable method known in the art for conjugating molecules, such as a detectable marker, a toxin, or a therapeutic agent to antibodies may be used.

For example, drug conjugation involves coupling of a biological active cytotoxic (anticancer) payload or drug to an antibody that specifically targets a certain tumor marker (e.g. a protein that, ideally, is only to be found in or on tumor cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cancer cells. Due to this targeting, ideally the drug has lower side effects and gives a wider therapeutic window than other chemotherapeutic agents. Technics to conjugate antibodies are disclosed are known in the art (see, e.g., Jane de Lartigue, OncLive Jul. 5, 2012; ADC Review on antibody-drug conjugates; and Ducry et al., (2010). Bioconjugate Chemistry 21 (1): 5-13).

An anti-Siglec-15 antibody or antigen-binding fragment thereof can be fused or conjugated (e.g., covalently or noncovalently linked) to a detectable label or substance. Examples of detectable labels or substances include enzyme labels, such as, glucose oxidase; radioisotopes, such as iodine (125I, 121I), carbon (14C), sulfur (35S), tritium (3H), indium (121 In), and technetium (99Tc); luminescent labels, such as luminol; and fluorescent labels, such as fluorescein and rhodamine, and biotin. Such labeled antibodies or antigen-binding fragments thereof can be used to detect Siglec-15 (e.g., human Siglec-15) protein.

In some embodiments, the cytotoxic agent for conjugation to the anti-Siglec-15 antibody or antigen-binding fragment thereof is a small molecular weight toxin (MW<2′000 Dalton, preferably MW<1′000 Dalton), a peptide toxin, or a protein toxin. Many specific examples of these toxins are well known in the art. See, e.g., Dyba et al., Curr. Pharm. Des. 10:2311-34, 2004; Kuyucak et al., Future Med. Chem. 6:1645-58, 2014; Beraud et al., Inflamm. Allergy Drug Targets. 10:322-42, 2011; and Middlebrook et al., Microbiol. Rev. 48:199-221, 1984. In some embodiments, an anti-Siglec-15 antibody of the present disclosure may be conjugated to a toxin selected from ricin, ricin A-chain, doxorubicin, daunorubicin, a maytansinoid, taxol, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, Saponaria officinalis inhibitor, glucocorticoid, auristatin, auromycin, yttrium, bismuth, combrestatin, duocarmycins, dolastatin, cc1065, and a cisplatin.

In some embodiments, a therapeutic agent is conjugated to the anti-Siglec-15 antibody. For example, the therapeutic agent can be a maytansinoid (e.g., maytansinol or DM1 maytansinoid), a taxane, a calicheamicin, a cemadotin, a monomethylauristatin (e.g., monomethylauristatin E (MMAE) or monomethylauristatin F), a pyrrolobenzodiazepine (PBD), an anthracycline, or a derivative of the highly potent anthracycline PNU-159682 as disclosed in WO2016102679 incorporated herein by reference in its entirety. Therapeutic agents also include vincristine and prednisone. In various embodiments, the therapeutic agent that may be employed in the invention can be an antimetabolite (e.g., an antifolate such as methotrexate, a fluoropyrimidine such as 5-fluorouracil, cytosine arabinoside, or an analogue of purine or adenosine); an intercalating agent an intercalating agent (for example, an anthracycline such as doxorubicin, nemorubicine, or preferably a derivative of PNU-159682), daunomycin, epirabicin, idarubicin, mitomycin-C, dactinomycin, or mithramycin, or other intercalating agents such as pyrrolobenzodiazepine); a DNA-reactive agent such as calicheamicins, tiancimycins, and other enediynes; a platinum derivative (e.g., cisplatin or carboplatin); an alkylating agent (e.g., nitrogen mustard, melphalan, chlorambucil, busulphan, cyclophosphamide, ifosfamide nitrosoureas or thiotepa); an RNA polymerase inhibitor such as α-amanitin; an antimitotic agent (e.g., a vinca alkaloid such as vincristine, or a taxoid such as paclitaxel or docetaxel); a topoisomerase inhibitor (for example, etoposide, teniposide, amsacrine, topotecan, or exatecan); a cell cycle inhibitor (for example, a flavopyridol); or a microbtubule agent (e.g., an epothilone, a tubulysine, a pre-tubulysine, a discodermolide analog, or an eleutherobin analog). A therapeutic agent can be a proteosome inhibitor or a topoisomerase inhibitor such as bortezomib, amsacrine, etoposide, etoposide phosphate, teniposide, or doxorubicin. Therapeutic radioisotopes include iodine (¹³¹I), yttrium (⁹⁰Y), lutetium (¹⁷⁷Lu), actinium (²²⁵ Ac), praseodymium, astatine (At), rhenium (Re), bismuth (Bi or Bi), and rhodium (Rh). Antiangiogenic agents include linomide, bevacuzimab, angiostatin, and razoxane. In some embodiments, the anti-Siglec-15 antibody is conjugated to MMAE. In some embodiments, the anti-Siglec-15 antibody is conjugated to exatecan.

In some embodiments, the anti-Siglec-15 antibody can be conjugated to a liposome, as described in Bendas, BioDrugs, 15:215-224, 2001. In such embodiments, the antibody can be conjugated to a colloidal particle, e.g., a liposome, and used for controlled delivery of an agent to diseased cells. In preparing an antibody conjugated to a liposome, e.g., an immunoliposome, an agent such as a chemotherapeutic or other drug can be entrapped in the liposome for delivery to a target cell.

In some embodiments, the anti-Siglec-15 ADC induces cytotoxicity in target cells. In some embodiments, the ADC induces cytotoxicity in about 5-100% of target cells. In some embodiments, the ADC induces cytotoxicity in about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of target cells. In some embodiments, the ADC induces cytotoxicity in at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of target cells.

In some embodiments, non-target cells remain viable after administration of an anti-Siglec-15 ADC. In some embodiments, 5-100% of non-target cells are viable after administration of an anti-Siglec-15 ADC. In some embodiments, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of non-target cells are viable after administration of an anti-Siglec-15 ADC. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of non-target cells are viable after administration of an anti-Siglec-15 ADC.

### 1.2.2 Polynucleotides

In certain aspects, provided herein are polynucleotides comprising a nucleotide sequence encoding an antibody or antigen-binding fragment thereof described herein or a domain thereof (e.g., a variable light chain region and/or variable heavy chain region) that immunospecifically binds to a Siglec-15 (e.g., human Siglec-15) antigen, and vectors, e.g., vectors comprising such polynucleotides for recombinant expression in host cells (e.g., E. coli and mammalian cells).

In particular aspects, provided herein are polynucleotides comprising nucleotide sequences encoding antibodies or antigen-binding fragments thereof, which immunospecifically bind to a Siglec-15 polypeptide (e.g., human Siglec-15) and comprise an amino acid sequence as described herein, as well as antibodies or antigen-binding fragments that compete with such antibodies or antigen-binding fragments for binding to a Siglec-15 polypeptide (e.g., in a dose-dependent manner), or which bind to the same epitope as that of such antibodies or antigen-binding fragments.

Also provided herein is a polynucleotide comprising a nucleotide sequence encoding a polypeptide comprising a sequence selected from the group consisting of SEQ ID NOs: 76, 78, 80, 82, 84, 86, 88, 90, and 92. In some embodiments, an antibody or antigen-binding fragment thereof comprising the polypeptide immunospecifically binds to Siglec-15.

Also provided herein is a polynucleotide comprising a nucleotide sequence encoding a polypeptide comprising a sequence selected from the group consisting of SEQ ID NOs: 77, 79, 81, 83, 85, 87, 89, 91, and 93. In some embodiments, an antibody or antigen-binding fragment thereof comprising the polypeptide immunospecifically binds to Siglec-15.

Also provided herein are polynucleotides comprising a variable heavy chain-encoding nucleotide sequence shown in Table 6, e.g., wherein an antibody or antigen-binding fragment thereof comprising the encoded heavy chain variable region binds to Siglec-15.

TABLE 6
Heavy chain variable region-encoding polynucleotide sequences

| Antibody | VH Sequence |
|---|---|
| 62E5 | GAGGTGAAACTGCTGGAATCTGGCGGAGGCCTGGTGCAGCCCGGC |
| | GGCAGCCTGAAACTGAGCTGTGCTGCTTCTGGATTTGACTTCAGCG |
| | GCTACTGGATGAGCTGGGTCAGACAGGCCCCTGGCAAGGGCCTCG |
| | AGTGGATCGGAGAAATCATTCCAGATAGCAGCACCATCAACTACA |
| | CCCCTAGCCTGAAGGACAAGTTCATCATCAGCAGAGATAATGCCA |
| | AGAACACACTGTTCCTGCAAATGAGAAAGGTGCGGTCCGAGGACA |
| | CCGCCCTGTACTACTGCGCCAGACGGGACGGCTATTGGTTCGCCTA |
| | CTGGGGCCAGGGCACACTGGTGACCGTGTCCGCC (SEQ ID NO: 76) |
| S15B1 | CAAGTGCAGCTGCAGCAGAGCGGACCTGAACTGGTGAAGCCTGGC |
| | GCTAGCGTGAAAATCAGCTGCAAGGCCTCCGGCTACGCCTTCAGC |
| | AGCTCTTGGATGAACTGGGTCAAGCAGCGGCCAGGAAAGGGCCTG |
| | GAATGGATCGGCAGAATCTACCCCGGCCACGGCGAGACAAACTAC |
| | AACGGCAAGTTCAAGGACAAGGCTACAGTGACCGCCGATAAAAGC |
| | TCTTCTACTACCTACATGCAGCTGAGCTCTCTGACCAGCGAGGACA |
| | GCGCCGTGTACTTCTGTGCCAGAGTGGATAATAGCGACCCTTATTA |
| | CTTTGACTACTGGGGCCAGGGCACAACCCTGACCGTGTCCAGC |
| | (SEQ ID NO: 78) |
| ZLA-1243- | CAAGTGCAGCTGGTGGAATCTGGCGGCGGACTGGTTCAGCCCGGC |
| A01 | GGCTCCCTGCGGCTGAGCTGTGCCGCTAGCGGCTTCACCTTCAGCT |
| | CTTATGCCATGAGCTGGGTGCGGCAGGCCCCTGGAAAAGGCCTGG |
| | AATGGGTCTCCGCCATCAGCGGCAGCGGAGGCAGCACATACTACG |
| | CTGATAGCGTGAAGGGCAGATTCACAATCTCTAGAGATAATAGCA |
| | AGAACACCCTGTACCTGCAGATGAACAGCCTGAGAGCCGAGGACA |
| | CCGCCGTGTACTACTGCGCCAAGGACAACGAGTGGACCCCTGGCT |
| | CTTTTTTCGACTACTGGGGCCAGGGCACCCTCGTGACAGTGTCCAG |
| | C (SEQ ID NO: 80) |
| ZLA-1243- | CAAGTGCAGCTGGTCCAGAGCGGCGGAGGCCTGGTGCAGCCCGGC |
| E01 | AGAAGCCTGAGACTGAGCTGTGCTGCTAGCGGCTTCACATTCGAC |
| | GACTACGCCATGCACTGGGTGCGGCAGGCCCCTGGCAAGGGCCTC |
| | GAGTGGGTTTCTGGCATCAGCTGGAATTCTGGCAGCATCGGCTACG |
| | CCGACAGCGTGAAGGGAAGATTCACCATCTCTAGAGATAACGCCA |
| | AGAACAGCCTGTATCTGCAGATGAACTCCCTGCGGGCCGAAGATA |
| | CAGCCCTGTACTACTGCGCCAAAGGACTGTGGCCTAGCTACGACCT |
| | GGACGCCTTTGATATCTGGGGCCAGGGCACCATGGTGACCGTGTC |
| | CAGC (SEQ ID NO: 82) |
| ZLA-1214- | GAGGTCCAGCTGGTGCAGAGCGGAGCCGAGGTGAAAAAGCCCGG |
| D08 | CGCTTCTGTGAAGGTGTCCTGCAAGGCCTCTGGATACACCTTCACC |
| | GGCTACTACATGCACTGGGTGCGGCAGGCCCCTGGCCAAGGCCTG |
| | GAATGGATGGGCAGGATCAATCCTAACAGCGGCGGAACAAACTAC |
| | GCCCAGAAGTTCCAGGGCCGGGTGACAATGACCAGAGATACCAGC |
| | ACAAGCACCGCCTACATGGAACTGAGCAGACTGAGAAGCGACGAC |
| | ACAGCTGTGTACTATTGTGCCAGAGTGATCAACGACGCCTTTGATA |
| | TGTGGGGCCAGGGCACCATGGTGACCGTGTCCAGC (SEQ ID NO: |
| | 84) |
| ZLA-1214- | CAAGTGCAGCTGGTGCAGAGCGGCGCCGAGGTGAAAAAGCCCGG |
| B07 | CAGCAGCGTGAAGGTGTCTTGTAAAGCCAGCGGCGGAACATTCAG |
| | CTCCTACGCCATCAGCTGGGTGCGGCAGGCCCCTGGCCAGGGACT |
| | GGAGTGGATGGGCGGCATCATCCCTATTTTCGGCACAGCCAACTA |
| | CGCCCAGAAGTTTCAGGGCAGAGTGACCATCACCGCCGACGAGAG |
| | CACCAGCACAGCTTATATGGAACTGAGCAGCCTGAGATCTGAAGA |
| | TACCGCTGTGTACTACTGCGCCAGAGATGGCGGACGGGACGGCTA |
| | CAACCCAAAGAAGATCCTGGAGTTCGACTACTGGGGCCAGGGCAC |
| | CCTGGTCACCGTGTCCTCT (SEQ ID NO: 86) |
| ZLA-1212- | CAAGTGCAGCTGGTTCAGAGCGGAGGAGGCGTGGTGCAGCCCGGC |
| E09 | AGAAGCCTGAGACTGAGCTGTGCTGCTTCTGGATTTACATTCAGCT |
| | CCTACGGCATGCACTGGGTGCGGCAGGCCCCTGGCAAAGGCCTGG |
| | AATGGGTCGCCGTGATCAGCTACGACGGCAGCAACAAGTACTACG |
| | CCGATAGCGTGAAGGGCAGATTCACCATCTCTAGAGATAATAGCA |
| | AGAACACCCTCTACCTGCAGATGAACAGCCTGCGGGCCGAGGACA |
| | CCGCCGTGTACTACTGCGCCAAGGACGACAGCGGCTACTATGATT |
| | CTAGCGGCTACGACTACTGGGGCCAGGGCACACTGGTGACCGTGT |
| | CCTCC (SEQ ID NO: 88) |
| ZLA-1212- | CAAGTGCAGCTGGTGCAGAGCGGAGGCGGAGTGGTGCAGCCCGG |
| B05 | AAGAAGCCTGCGGCTGAGCTGTGCTGCTTCTGGCTTTACCTTCAGC |
| | TCTTATGCCATGCACTGGGTGCGGCAGGCCCCTGGCAAAGGCCTG |
| | GAATGGGTCGCCGTGATCAGCTACGACGGCTCCAACAAGTACTAC |
| | GCCGATAGCGTGAAGGGCAGATTCACCATCTCTAGAGATAATAGC |
| | AAGAACACACTCTACCTGCAGATGAACAGCCTGCGCGCCGAGGAC |
| | ACCGCCGTGTACTACTGCGCCAGAGAGGGCGGCAGCTACTACGGC |
| | TACTTCGACTACTGGGGCCAGGGCACCCTGGTTACAGTGTCCAGC |
| | (SEQ ID NO: 90) |
| ZLA-1214- | CAAGTGCAGCTGGTCCAGAGCGGAGCCGAGGTGAAAAAGCCCGG |
| H05 | CGCTTCTGTGAAGGTGTCCTGCAAGGCCAGCGGCTACACCTTCACC |
| | AGCTACGGCATCAGCTGGGTGCGGCAGGCCCCTGGCCAGGGCCTG |
| | GAATGGATGGGCTGGATCTCCGCCTACAACGGCAATACCAACTAC |
| | GCCCAGAAGCTGCAGGGAAGAGTGACCATGACAACAGATACATCT |
| | ACCAGCACCGCTTACATGGAGCTGAGAAGCCTGCGGAGCGACGAC |
| | ACCGCCGTGTACTACTGTGCCAGAATGAGCGGATATTACGGCATG |
| | GACGTGTGGGGCCAGGGCACCCTGGTTACAGTGTCTAGC (SEQ ID |
| | NO: 92) |
| S15B1-VH1 | CAGGTTCAGTTGGTTCAGTCTGGCGCCGAGGTGAAGAAGCCTGGC |
| | GCCTCTGTGAAGGTGTCCTGCAAGGCCTCTGGCTACGCCTTCTCCA |
| | GCTCCTGGATGAACTGGGTCCGACAGGCTCCTGGACAAGGACTGG |
| | AATGGATCGGCAGAATCTACCCTGGCCATGGAGAAACAAACTACA |
| | ATGGCAAGTTCAAGGACAGAGTGACCATGACAGCTGACACCAGCA |
| | CCTCTACTGTGTACATGGAACTGTCCTCACTGAGGTCTGAGGACAC |
| | CGCCGTGTACTACTGTGCCAGAGTAGACAACAGCGACCCTTACTA |
| | CTTCGACTACTGGGGCCAGGGCACAACCGTGACCGTGTCCTCT |
| | (SEQ ID NO: 119) |
| S15B1-VH2 | CAGGTTCAGTTGGTTCAGTCTGGCGCCGAGGTGAAGAAGCCTGGC |
| | GCCTCTGTGAAGGTGTCCTGCAAGGCCTCTGGCTACGCCTTCTCCA |
| | GCTCCTGGATGAACTGGGTCCGACAGGCTCCTGGACAAGGACTGG |
| | AATGGATCGGCAGAATCTACCCTGGCCATGGAGAAACAAACTACA |
| | ATGGCAAGTTCAAGGACAGAGTGACCATGACAGCTGACACCAGCA |
| | CCTCTACTGTGTACATGGAACTGTCCTCACTGAGGTCTGAGGACAC |
| | CGCCGTGTACTTCTGCGCCAGAGTGGACAACAGCGACCCTTACTAC |
| | TTCGACTACTGGGGCCAGGGCACAACCGTGACCGTGTCCTCT (SEQ |
| | ID NO: 120) |
| S15B1-VH3 | CAGGTTCAGTTGGTTCAGTCTGGCGCCGAGGTGAAGAAGCCTGGC |
| | GCCTCTGTGAAGGTGTCCTGCAAGGCCTCTGGCTACGCCTTCTCCA |
| | GCAGCTGGATGAACTGGGTGAAGCAGGCTCCTGGCCAGGGCCTGG |
| | AATGGATCGGCAGAATCTACCCTGGCCATGGAGAAACAAACTACA |
| | ATGGAAAGTTCAAGGACAGAGCCACCATGACAGCAGACACCAGC |
| | ACCTCTACTGTGTACATGGAACTGTCCTCACTGAGGTCTGAGGACA |
| | CCGCCGTGTACTTCTGCGCCAGAGTGGACAACAGCGACCCTTACTA |
| | CTTCGACTACTGGGGCCAGGGCACAACCGTGACCGTGTCCTCT |
| | (SEQ NO ID: 121) |
| S15B1-VH4 | CAGGTTCAGTTGGTTCAGTCTGGCGCCGAGGTGAAGAAGCCTGGC |
| | GCCTCTGTGAAGGTGTCCTGCAAGGCCTCTGGCTACGCCTTCTCCA |
| | GCAGCTGGATGAACTGGGTGAAGCAGGCTCCTGGCCAGGGCCTGG |
| | AATGGATCGGCAGAATCTACCCTGGCCATGGAGAAACAAACTACA |
| | ATGGAAAGTTCAAGGACAGAGCCACCATGACCGCCGACAAGTCCA |
| | CCTCCACCGTGTATATGGAACTGTCCTCACTGAGGTCTGAGGACAC |
| | CGCCGTGTACTTCTGCGCCAGAGTGGACAACAGCGACCCTTACTAC |
| | TTCGACTACTGGGGCCAGGGCACAACCGTGACCGTGTCCTCT |
| | (SEQ: 122) |

Also provided herein are polynucleotides comprising a light chain variable region-encoding nucleotide sequence shown in Table 7, e.g., wherein an antibody or antigen-binding fragment thereof comprising the encoded light chain variable region binds to Siglec-15.

TABLE 7
Light chain variable region-encoding polynucleotide sequences

| Antibody | VL Sequence |
|---|---|
| 62E5 | GACATCGTGATGAGCCAGTCCCCTAGCTCCCTGGCCGTGTCCGTGG |
| | GCGAGAAGGTGACCATGTCTTGTAAAAGCTCTCAGTCTCTGCTGTA |
| | CAGCAGAAACCAGCAGAACTACCTGGCTTGGTTTCAGCAAAAGCC |
| | CGGCCAGAGCCCTGAGCTGCTGATCTACTGGGCCAGCACCCGGGA |
| | AAGCGGAGTGCCAGATAGATTCACAGGCAGCGGATCTGGCACCGA |
| | CTTCACCCTGACAATCAGCAGCGTGAAGGCCGAGGACCTGGCCGT |
| | CTACTACTGCCAGCAGTACTATAGCTACCCTCTGACCTTCGGCGCC |
| | GGCACAAAGCTGGAACTCAAG (SEQ ID NO: 77) |
| S15B1 | GACATCGTGCTGACCCAGAGCCCTGCCAGCCTGGCCGTGTCCCTGG |
| | GCCAGCGGGCCACAATCAGCTGTAGAGCCTCTAAAAGCGTCTCTA |
| | CAAGCGGCTACAGCTACATGCACTGGTATCAGCAGAAGCCCGGCC |
| | AACCTCCAAAGCTGCTGATCTACCTGGCTAGCAACCTGGAGAGCG |
| | GCGTGCCTGCTAGATTCAGCGGCAGCGGATCCGGCACCGACTTCA |
| | CCCTGAACATCCACCCCGTGGAAGAGGAAGATGCCGCCACCTACT |
| | ACTGCCAGCACGGCAGAGAGCTGCCTTTTACCTTCGGCTCTGGAAC |
| | AAAGCTCGAGATCAAG (SEQ ID NO: 79) |
| ZLA-1243- | CAAAGCGCCCTGACCCAGCCTGCTTCTGTGTCCGGATCTCCAGGCC |
| A01 | AGAGCATCACCATCTCTTGTACAGGCACAAGCAGCGACGTGGGCG |
| | GCTACAACTACGTGTCCTGGTATCAGCAGCACCCCGGCAAAGCCC |
| | CTAAGCTGATGATCTACGACGTCAGCAACCGGCCTAGCGGCGTGT |
| | CTAATAGATTCAGCGGATCTAAGAGCGGCAACACCGCCAGCCTGA |
| | CAATCAGCGGCCTGCAGGCCGAAGATGAGGCCGACTACTACTGCA |
| | GCAGCTACACCAGCAGCTCCACCAGAGTGTTCGGCGGAGGCACCA |
| | AGGTGACCGTGCTG (SEQ ID NO: 81) |
| ZLA-1243- | CAAAGCGCCCTGACACAGCCTGCCAGCGTGTCCGGATCTCCAGGC |
| E01 | CAGAGCATCACCCTGAGCTGTACCGGCACATCTAGCGACGTGGGC |
| | GCCTACAACTTCGTGTCTTGGTATCAGCAGCACCCTGGCACCGCCC |
| | CTAAGCTGATGATCTACGAGGTGTCCAACCGGCCTAGCGGCGTGT |
| | CCGACAGATTCAGCGGCAGCAAAAGCGGAAATACCGCTTCTCTGA |
| | CCATCAGCGGCCTGCAGGCTGAAGATGAGGCCGACTACTACTGCA |
| | GCAGCTACACCAGCACCATCCCCCCCTTCGTCTTTGGCACAGGCAC |
| | AAAGGTGACCGTGCTG (SEQ ID NO: 83) |
| ZLA-1214- | GATGTGGTGATGACACAGAGCCCCCTGAGCCTGCCTGTGACCCCT |
| D08 | GGCGAGCCTGCTTCTATCAGCTGTAGAAGCAGCCAGAGCCTGCTG |
| | CACAGCAACGGCTACAACTACCTGGACTGGTATCTCCAGAAACCT |
| | GGACAGTCCCCACAGCTGCTGATCTACCTGGGCTCTAATCGGGCCA |
| | GCGGCGTCCCCGATAGATTCAGCGGCAGCGGATCTGGCACCGACT |
| | TCACCCTGAAGATCTCCAGAGTGGAAGCCGAGGACGTGGGCGTGT |
| | ACTACTGCATGCAGGCCCTGCAGACCCAATACACCTTTGGCCAGG |
| | GCACAAAGGTGGACATCAAG (SEQ ID NO: 85) |
| ZLA-1214- | GAAATCGTGCTGACCCAGAGCCCTGGCACCCTCTCCCTGTCTCCAG |
| B07 | GCGAGCGGGCCACCCTGAGCTGTAGAGCTTCTCAGAGCGTGTCCT |
| | CCAGCTACCTGGCCTGGTATCAGCAGAAACCTGGCCAGGCCCCTA |
| | GACTGCTGATCTACGGCGCCAGCAGCAGAGCTACAGGCATTCCTG |
| | ATAGATTCAGCGGATCTGGCAGCGGCACAGACTTCACCCTGACAA |
| | TCAGCCGGCTGGAACCCGAGGACTTTGCCGTGTACTACTGCCAGC |
| | AATACGGCAGCAGCCCCATCACCTTCGGCCCTGGAACCAAGGTCG |
| | ACATCAAG (SEQ ID NO: 87) |
| ZLA-1212- | CAATCTGTGCTGACACAGCCCCCCTCCGTGTCCGGCGCTCCTGGCC |
| E09 | AGCGGGTGACCATCAGCTGCACCGGCTCTTCTTCCAACATCGGCGC |
| | CGGCTACGACGTGCACTGGTATCAGCAGCTGCCTGGAACAGCCCC |
| | TAAGCTGCTGATCTACGGCAACAGCAATAGACCTAGCGGCGTGCC |
| | AGATAGATTCAGCGGCAGCAAAAGCGGCACCAGCGCCAGCCTGGC |
| | CATCACCGGCCTGCAGGCTGAAGATGAGGCCGACTACTACTGTCA |
| | GAGCTACGACAGCTCTCTGAGCGGAGTCGTGTTCGGCGGAGGCAC |
| | AAAGCTGACCGTGCTC (SEQ ID NO: 89) |
| ZLA-1212- | CAAAGCGTTCTGACACAGCCTCCATCTGTGTCCGTCGCCCCCGGCA |
| B05 | AGACCGCTAGAATCACATGCGGCGGCAACAACATCGGCTCTAAGT |
| | CCGTGCACTGGTATCAGCAGAAACCTGGCCAGGCCCCTGTGCTGG |
| | TGATCTACGACGACTCTGATAGACCTAGCGGCATCCCCGAGCGGTT |
| | CAGCGGCAGCAACAGCGGCAATACCGCCACCCTGACCATCAGCAG |
| | AGTGGAAGCCGGAGATGAGGCCGACTACTACTGTCAGGTGTGGGA |
| | CAGCAGCAGCGACCACCGGGTGTTCGGCGGAGGAACAAAGCTGAC |
| | CGTGCTG (SEQ ID NO: 91) |
| ZLA-1214- | GATATTCAGCTGACCCAGAGCCCATCTAGCCTGAGCGCCAGCGTG |
| H05 | GGAGATAGAGTGACCATCACCTGTCAGGCTTCTCAGGGCATCAGC |
| | AACTACCTGAACTGGTATCAGCAGAAACCCGGCAAGGCCCCTAAG |
| | CTGCTGATCTACGACGCCTCTAATCTGGAAACCGGCGTGCCCAGCC |
| | GGTTCAGCGGCAGCGGCTCCGGCACCGACTTCACCTTTACCATCTC |
| | CAGCCTGCAGCCTGAGGACATCGCCACATACTACTGCCAGCAATA |
| | CGACAACCTGCCTCTGACATTCGGCGGCGGAACAAAGCTCGAGAT |
| | CAAG (SEQ ID NO: 93) |
| S15B1-VL1 | GATATCGTGCTGACCCAGTCTCCTGCCTCTCTGGCTGTGTCTCCTG |
| | GCCAGAGAGCCACCATCACCTGTAGAGCCTCCAAGTCCGTGTCTA |
| | CCTCAGGCTACTCCTACATGCACTGGTACCAGCAGAAGCCTGGCC |
| | AGCCTCCCAAGCTGCTGATCTACCTGGCCTCCAACCTGGAGAGTGG |
| | CGTGCCAGCCAGATTCTCCGGCTCTGGCTCTGGCACCGACTTCACC |
| | CTGACCATCAACCCAGTGGAAGCCGAGGACACAGCCAACTACTAC |
| | TGCCAGCATGGCAGAGAGCTGCCCTTCACCTTTGGCGGCGGCACC |
| | AAGCTGGAAATCAAG (SEQ ID NO: 123) |
| S15B1-VL2 | GATATCGTGCTGACCCAGTCTCCTGCCTCTCTGGCTGTGTCTCCTG |
| | GCCAGAGAGCCACCATCACCTGTAGAGCCTCCAAGTCCGTGTCTA |
| | CCTCAGGCTACTCCTACATGCACTGGTACCAGCAGAAGCCTGGCC |
| | AGCCTCCCAAGCTGCTGATCTACCTGGCCTCCAACCTGGAGAGTGG |
| | CGTGCCAGCCAGATTCTCCGGCTCTGGCTCTGGCACCGACTTCACC |
| | CTGACCATCAACCCAGTGGAAGCCGAGGACACCGCCACCTACTAC |
| | TGTCAGCACGGCAGAGAGCTGCCCTTCACCTTTGGCGGCGGCACC |
| | AAGCTGGAAATCAAG (SEQ ID NO: 124) |

Also provided herein are polynucleotides comprising a nucleotide sequence that encodes SEQ ID NO: 10, 11, 18, 19, 26, 27, 34, 35, 42, 43, 50, 51, 58, 59, 66, 67, 74, 75, 103, 104, 105, 106, 107, or 108 and is at least about 80%, 85%, or 90% identical to SEQ ID NO: 76, 77, 78, 79, 80, 81, 82, 83, 84, 85, 86, 87, 88, 89, 90, 91, 92, 93, 119, 120, 121, 122, 123, or 124, respectively.

Also provided herein are polynucleotides comprising a nucleotide sequence that encodes SEQ ID NO: 10, 11, 18, 19, 26, 27, 34, 35, 42, 43, 50, 51, 58, 59, 66, 67, 74, 75, 103, 104, 105, 106, 107, or 108 and is at least about 95% identical to SEQ ID NO: 76, 77, 78, 79, 80, 81, 82, 83, 84, 85, 86, 87, 88, 89, 90, 91, 92, 93, 119, 120, 121, 122, 123, or 124, respectively.

Also provided herein are polynucleotides comprising a nucleotide sequence that encodes SEQ ID NO: 10, 11, 18, 19, 26, 27, 34, 35, 42, 43, 50, 51, 58, 59, 66, 67, 74, 75, 103, 104, 105, 106, 107, or 108 and is at least about 96% identical to SEQ ID NO: 76, 77, 78, 79, 80, 81, 82, 83, 84, 85, 86, 87, 88, 89, 90, 91, 92, 93, 119, 120, 121, 122, 123, or 124, respectively.

Also provided herein are polynucleotides comprising a nucleotide sequence that encodes SEQ ID NO: 10, 11, 18, 19, 26, 27, 34, 35, 42, 43, 50, 51, 58, 59, 66, 67, 74, 75, 103, 104, 105, 106, 107, or 108 and is at least about 97% identical to SEQ ID NO: 76, 77, 78, 79, 80, 81, 82, 83, 84, 85, 86, 87, 88, 89, 90, 91, 92, 93, 119, 120, 121, 122, 123, or 124, respectively.

Also provided herein are polynucleotides comprising a nucleotide sequence that encodes SEQ ID NO: 10, 11, 18, 19, 26, 27, 34, 35, 42, 43, 50, 51, 58, 59, 66, 67, 74, 75, 103, 104, 105, 106, 107, or 108 and is at least about 98% identical to SEQ ID NO: 76, 77, 78, 79, 80, 81, 82, 83, 84, 85, 86, 87, 88, 89, 90, 91, 92, 93, 119, 120, 121, 122, 123, or 124, respectively.

Also provided herein are polynucleotides comprising a nucleotide sequence that encodes SEQ ID NO: 10, 11, 18, 19, 26, 27, 34, 35, 42, 43, 50, 51, 58, 59, 66, 67, 74, 75, 103, 104, 105, 106, 107, or 108 and is at least about 99% identical to SEQ ID NO: 76, 77, 78, 79, 80, 81, 82, 83, 84, 85, 86, 87, 88, 89, 90, 91, 92, 93, 119, 120, 121, 122, 123, or 124, respectively.

In particular embodiments, provided herein are polynucleotides comprising a nucleotide sequence encoding an anti-Siglec-15 antibody comprising three VH chain CDRs, e.g., containing VH CDR1, VH CDR2, VH CDR3 of any one of antibodies described herein (e.g., see Table 1). In specific embodiments, provided herein are polynucleotides comprising three VL chain CDRs, e.g., containing VL CDR1, VL CDR2, and VL CDR3 of any one of antibodies described herein (e.g., see Table 2). In specific embodiments, provided herein are polynucleotides comprising a nucleotide sequence encoding an anti-Siglec-15 antibody comprising three VH chain CDRs, e.g., containing VH CDR1, VH CDR2, and VH CDR3 of any one of antibodies described herein (e.g., see Table 1) and three VL chain CDRs, e.g., containing VL CDR1, VL CDR2, and VL CDR3 of any one of antibodies described herein (e.g., see Table 2).

In particular embodiments, provided herein are polynucleotides comprising a nucleotide sequence encoding an anti-Siglec-15 antibody or an antigen-binding fragment thereof or a fragment thereof comprising a VH domain, comprising an amino acid sequence described herein (e.g., see Table 1, e.g., the VH CDRs of a particular antibody identified by name in the tables). In specific embodiments, provided herein are polynucleotides comprising a nucleotide sequence encoding an anti-Siglec-15 antibody or antigen-binding fragment thereof or a fragment thereof comprising a VL domain, comprising an amino acid sequence described herein (e.g., see Table 2, e.g., the VL CDRs of a particular antibody identified by name in the Tables).

In certain embodiments, a polynucleotide described herein comprises a nucleotide sequence encoding a heavy chain variable region comprising an amino acid sequence described herein (e.g., SEQ ID NOs: 10, 18, 26, 34, 42, 50, 58, 66, 74, 103, 104, 105, or 106), wherein an antibody containing the heavy chain variable region immunospecifically binds to Siglec-15 (e.g., human Siglec-15). In a certain embodiment, a polynucleotide described herein comprises a heavy chain variable region-encoding sequence provided herein, wherein an antibody containing the heavy chain variable region immunospecifically binds to Siglec-15 (e.g., human Siglec-15).

In certain embodiments, a polynucleotide described herein comprises a nucleotide sequence encoding a light chain variable region comprising an amino acid sequence described herein (e.g., SEQ ID NOs: 11, 19, 27, 35, 43, 51, 59, 67, 75, 107, or 108), wherein an antibody containing the light chain variable region immunospecifically binds to Siglec-15 (e.g., human Siglec-15). In a certain embodiment, a polynucleotide described herein comprises a light chain variable region-encoding sequence provided herein, wherein an antibody containing the heavy chain variable region immunospecifically binds to Siglec-15 (e.g., human Siglec-15).

In a particular embodiment, a polynucleotide or combination of polynucleotides provided herein comprises a nucleotide sequence or combination of nucleotide sequences encoding an antibody or antigen-binding fragment thereof that immunospecifically binds to Siglec-15 (e.g., human Siglec-15), wherein the antibody or antigen-binding fragment thereof comprises a heavy chain, wherein the heavy chain comprises a heavy chain variable domain comprising an amino acid sequence set forth in Table 3 (e.g., SEQ ID NOs: 10, 18, 26, 34, 42, 50, 58, 66, 74, 103, 104, 105, or 106) and a constant region comprising the amino acid sequence of a human gamma (γ) heavy chain constant region.

In a particular embodiment, a polynucleotide or combination of polynucleotides provided herein comprises a nucleotide sequence or combination of nucleotide sequences encoding an antibody or antigen-binding fragment thereof that immunospecifically binds to Siglec-15 (e.g., human Siglec-15), wherein the antibody or antigen-binding fragment thereof comprises a light chain, wherein the light chain comprises a light chain variable domain comprising an amino acid sequence set forth in Table 4 (e.g., SEQ ID NOs: 11, 19, 27, 35, 43, 51, 59, 67, 75, 107, or 108) and a constant region comprising the amino acid sequence of a human kappa or lambda light chain constant region.

In a particular embodiment, a polynucleotide or combination of polynucleotides provided herein comprises a nucleotide sequence or combination of nucleotide sequences encoding an antibody or antigen-binding fragment thereof that immunospecifically binds to Siglec-15 (e.g., human Siglec-15), wherein the antibody or antigen-binding fragment thereof comprises (i) a heavy chain, wherein heavy chain comprises a heavy chain variable domain comprising an amino acid sequence set forth in Table 3 (e.g., SEQ ID NO: 10, 18, 26, 34, 42, 50, 58, 66, 74, 103, 104, 105, or 106) and a constant region comprising the amino acid sequence of a human gamma (γ) heavy chain constant region and (ii) a light chain, wherein light chain comprises a light chain variable domain comprising an amino acid sequence set forth in Table 4 (e.g., SEQ ID NO: 11, 19, 27, 35, 43, 51, 59, 67, 75, 107, or 108) and a constant region comprising the amino acid sequence of a human kappa or lambda light chain constant region.

In certain embodiments, provided herein are polynucleotides comprising a nucleotide sequence encoding a bispecific antibody binding to human Siglec-15 and human CD3. In some embodiments, the polynucleotides comprise a nucleotide sequence or combination of nucleotide sequences encoding an anti-Siglec-15 arm, the anti-Siglec-15 arm comprising a light chain set forth in SEQ ID NO: 127 and a heavy chain set forth in SEQ ID NO: 128, and an anti-CD3 arm, the anti-CD3 arm comprising a light chain set forth in SEQ ID NO: 130 and a heavy chain set forth in SEQ ID NO: 129. In some embodiments, the anti-Siglec-15 arm comprises a light chain set forth in SEQ ID NO: 131 and a heavy chain set forth in SEQ ID NO: 132, and the anti-CD3 arm comprises a light chain set forth in SEQ ID NO: 134 and a heavy chain set forth in SEQ ID NO: 133.

Also provided herein are polynucleotides comprising nucleotide sequences that encode the light chain of SEQ ID NO: 111 and the heavy chain of SEQ ID NO: 112, and the light chain of SEQ ID NO: 114 and the heavy chain of SEQ ID NO: 113, wherein the light chains and heavy chains encoded form a bispecific antibody binding to human Siglec-15 and human CD3.

Also provided herein are polynucleotides comprising nucleotide sequences that encode the light chain of SEQ ID NO: 115 and the heavy chain of SEQ ID NO: 116, and the light chain of SEQ ID NO: 118 and the heavy chain of SEQ ID NO: 117, wherein the light chains and heavy chains encoded form a bispecific antibody binding to human Siglec-15 and human CD3.

In a specific embodiment, provided herein are polynucleotides comprising a nucleotide sequence encoding an anti-Siglec-15 antibody or antigen-binding fragment thereof or a domain thereof, designated herein.

Also provided herein are polynucleotides encoding an anti-Siglec-15 antibody or antigen-binding fragment thereof described herein or a domain thereof that are optimized, e.g., by codon/RNA optimization, replacement with heterologous signal sequences, and elimination of mRNA instability elements. Methods to generate optimized nucleic acids encoding an anti-Siglec-15 antibody or antigen-binding fragment thereof or a domain thereof (e.g., heavy chain, light chain, VH domain, or VL domain) for recombinant expression by introducing codon changes (e.g., a codon change that encodes the same amino acid due to the degeneracy of the genetic code) and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, each of which is incorporated by reference herein, accordingly.

A polynucleotide encoding an antibody or antigen-binding fragment thereof described herein or a domain thereof can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody or antigen-binding fragment thereof. Such PCR amplification methods can be used to obtain nucleic acids comprising the sequence encoding the variable light chain region and/or the variable heavy chain region of an antibody or antigen-binding fragment thereof. The amplified nucleic acids can be cloned into vectors for expression in host cells and for further cloning, for example, to generate chimeric and humanized antibodies or antigen-binding fragments thereof.

Polynucleotides provided herein can be, e.g., in the form of RNA or in the form of DNA. DNA includes cDNA, genomic DNA, and synthetic DNA, and DNA can be double-stranded or single-stranded. If single stranded, DNA can be the coding strand or non-coding (anti-sense) strand. In certain embodiments, the polynucleotide is a cDNA or a DNA lacking one more endogenous introns. In certain embodiments, a polynucleotide is a non-naturally occurring polynucleotide. In certain embodiments, a polynucleotide is recombinantly produced. In certain embodiments, the polynucleotides are isolated. In certain embodiments, the polynucleotides are substantially pure. In certain embodiments, a polynucleotide is purified from natural components.

### 1.2.3 Chimeric Antigen Receptors

In some aspects, the present disclosure provides methods of preparing a cell expressing a chimeric antigen receptor comprising transfecting a cell with the polynucleotides disclosed herein (e.g., anti-Siglec-15 CAR construct). In some aspects, the cell comprises a T cell, a B cell, a regulatory T cell (Treg), a tumor infiltrating lymphocyte (TIL), a natural killer (NK) cell, a natural killer T (NKT) cell, a stem cell, an induced pluripotent stem cell, and any combination thereof.

The T cell can come from any source. For example, T cells can be differentiated in vitro from a stem cell population, or T cells can be obtained from a subject. T cells can also be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, T cells can be derived from one or more available T cell lines. T cells can also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748 (incorporated by reference in its entirety).

In some aspects, the CAR comprises an extracellular domain comprising an anti-Siglec-15 domain, a transmembrane domain, and a cytoplasmic domain. In some aspects, the CAR further comprises one or more linker sequences, a marker sequence, an extracellular spacer, an activatory domain, a co-stimulatory domain, a suicide gene, a secretable immunomodulatory factor, and/or a signal transduction unit. In some embodiments, the CAR is designed to have two, three, four, or more costimulatory domains. In some aspects, the signal transduction unit is CD3, CD28, 4-1BB, or OX40. In some aspects, the transmembrane domain is a transmembrane domain of CD28, 4-1BB, CD8 alpha, CD4, CD19, CD3, CD45, CD5, CD9, CD16, CD22, CD33, CD37, CD80, CD86, CD134, CD137, CD154, a T cell receptor, or any combination thereof.

In some aspects, the co-stimulatory domain is a signaling region of CD2, CD3 delta, CD3 epsilon, CD3 gamma, CD4, CD7, CD8α, CD8β, CD11a (ITGAL), CD11b (ITGAM), CD11c (ITGAX), CD11d (ITGAD), CD18 (ITGB2), CD19 (B4), CD27 (TNFRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKD80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll ligand receptor, and or fragments or combinations thereof.

In some aspects, the present disclosure provides a method of expanding a cell expressing a chimeric antigen receptor comprising culturing a cell comprising a polynucleotide disclosed herein or a vector disclosed herein or a polypeptide disclosed herein, under suitable conditions. In some aspects, the method further comprises isolating the cell.

### 1.2.4 Cells and Vectors

In certain aspects, provided herein are vectors (e.g., expression vectors) comprising polynucleotides comprising nucleotide sequences encoding anti-Siglec-15 antibodies and antigen-binding fragments thereof or a domain thereof for recombinant expression in host cells, preferably in mammalian cells. Also provided herein are cells, e.g. host cells, comprising such vectors for recombinantly expressing anti-Siglec-15 antibodies or antigen-binding fragments thereof described herein (e.g., human or humanized antibodies or antigen-binding fragments thereof). In a particular aspect, provided herein are methods for producing an antibody or antigen-binding fragments thereof described herein, comprising expressing such antibody or antigen-binding fragment thereof in a host cell.

In certain embodiments, recombinant expression of an antibody or antigen-binding fragment thereof or domain thereof described herein (e.g., a heavy or light chain described herein) that specifically binds to Siglec-15 (e.g., human Siglec-15) involves construction of an expression vector containing a polynucleotide that encodes the antibody or antigen-binding fragment thereof or domain thereof. Once a polynucleotide encoding an antibody or antigen-binding fragment thereof or domain thereof (e.g., heavy or light chain variable domain) described herein has been obtained, the vector for the production of the antibody or antigen-binding fragment thereof can be produced by recombinant DNA technology using techniques well known in the art. Thus, methods for preparing a protein by expressing a polynucleotide containing an antibody or antigen-binding fragment thereof or domain thereof (e.g., light chain or heavy chain) encoding nucleotide sequence are described herein. Methods which are well known to those skilled in the art can be used to construct expression vectors containing antibody or antigen-binding fragment thereof or domain thereof (e.g., light chain or heavy chain) coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding an antibody or antigen-binding fragment thereof described herein, a heavy or light chain, a heavy or light chain variable domain, or a heavy or light chain CDR, operably linked to a promoter. Such vectors can, for example, include the nucleotide sequence encoding the constant region of the antibody or antigen-binding fragment thereof (see, e.g., International Publication Nos. WO 86/05807 and WO 89/01036; and U.S. Pat. No. 5,122,464), and variable domains of the antibody or antigen-binding fragment thereof can be cloned into such a vector for expression of the entire heavy, the entire light chain, or both the entire heavy and light chains.

An expression vector can be transferred to a cell (e.g., host cell) by conventional techniques and the resulting cells can then be cultured by conventional techniques to produce an antibody or antigen-binding fragment thereof described herein (e.g., an antibody or antigen-binding fragment thereof comprising the six CDRs, the VH, the VL, the VH and the VL, the heavy chain, the light chain, or the heavy and the light chain) or a domain thereof (e.g., the VH, the VL, the VH and the VL, the heavy chain, or the light chain). Thus, provided herein are host cells containing a polynucleotide encoding an antibody or antigen-binding fragment thereof described herein (e.g., an antibody or antigen-binding fragment thereof comprising the six CDRs, the VH, the VL, the VH and the VL, the heavy chain, the light chain, or the heavy and the light chain) or a domain thereof (e.g., the VH, the VL, the VH and the VL, the heavy chain, or the light chain), operably linked to a promoter for expression of such sequences in the host cell. In certain embodiments, for the expression of double-chained antibodies or antigen-binding fragments thereof, vectors encoding both the heavy and light chains, individually, can be co-expressed in the host cell for expression of the entire immunoglobulin, as detailed below. In certain embodiments, a host cell contains a vector comprising a polynucleotide encoding both the heavy chain and light chain of an antibody described herein, or a domain thereof (e.g., the VH and the VL). In specific embodiments, a host cell contains two different vectors, a first vector comprising a polynucleotide encoding a heavy chain or a heavy chain variable region of an antibody or antigen-binding fragment thereof described herein, and a second vector comprising a polynucleotide encoding a light chain or a light chain variable region of an antibody described herein, or a domain thereof. In other embodiments, a first host cell comprises a first vector comprising a polynucleotide encoding a heavy chain or a heavy chain variable region of an antibody or antigen-binding fragment thereof described herein, and a second host cell comprises a second vector comprising a polynucleotide encoding a light chain or a light chain variable region of an antibody or antigen-binding fragment thereof described herein. In specific embodiments, a heavy chain/heavy chain variable region expressed by a first cell associated with a light chain/light chain variable region of a second cell to form an anti-Siglec-15 antibody or antigen-binding fragment thereof described herein. In certain embodiments, provided herein is a population of host cells comprising such first host cell and such second host cell.

In a particular embodiment, provided herein is a population of vectors comprising a first vector comprising a polynucleotide encoding a light chain/light chain variable region of an anti-Siglec-15 antibody or antigen-binding fragment thereof described herein, and a second vector comprising a polynucleotide encoding a heavy chain/heavy chain variable region of an anti-Siglec-15 antibody or antigen-binding fragment thereof described herein. Alternatively, a single vector can be used which encodes, and is capable of expressing, both heavy and light chain polypeptides.

A variety of host-expression vector systems can be utilized to express antibodies and antigen-binding fragments thereof described herein (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express an antibody or antigen-binding fragment thereof described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS 1 or COS), CHO, BHK, MDCK, HEK 293, NSO, PER.C6, VERO, CRL7030, HsS78Bst, HeLa, and NIH 3T3, HEK-293T, HepG2, SP210, R1.1, B-W, L-M, BSC1, BSC40, YB/20 and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In a specific embodiment, cells for expressing antibodies and antigen-binding fragments thereof described herein are CHO cells, for example CHO cells from the CHO GS System™ (Lonza). In a particular embodiment, cells for expressing antibodies described herein are human cells, e.g., human cell lines. In a specific embodiment, a mammalian expression vector is pOptiVEC™ or pcDNA3.3. In a particular embodiment, bacterial cells such as Escherichia coli, or eukaryotic cells (e.g., mammalian cells), especially for the expression of whole recombinant antibody molecule, are used for the expression of a recombinant antibody molecule. For example, mammalian cells such as Chinese hamster ovary (CHO) cells in conjunction with a vector such as the major intermediate early gene promoter element from human cytomegalovirus is an effective expression system for antibodies (Foecking M K & Hofstetter H (1986) Gene 45:101-105; and Cockett M I et al., (1990) Biotechnology 8:662-667). In certain embodiments, antibodies or antigen-binding fragments thereof described herein are produced by CHO cells or NSO cells (a murine myeloma cell line that does not endogenously produce any immunoglobulin chains).

In addition, a host cell strain can be chosen which modulates the expression of the inserted sequences, or modifies and processes the gene product in the specific fashion desired. Such modifications (e.g., glycosylation) and processing (e.g., cleavage) of protein products can contribute to the function of the protein. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NSO, CRL7030, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells.

Once an antibody or antigen-binding fragment thereof described herein has been produced by recombinant expression, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies or antigen-binding fragments thereof described herein can be fused to heterologous polypeptide sequences described herein or otherwise known in the art to facilitate purification.

In specific embodiments, an antibody or antigen-binding fragment thereof described herein is isolated or purified. Generally, an isolated antibody or antigen-binding fragment thereof is one that is substantially free of other antibodies or antigen-binding fragments thereof with different antigenic specificities than the isolated antibody or antigen-binding fragment thereof. For example, in a particular embodiment, a preparation of an antibody or antigen-binding fragment thereof described herein is substantially free of cellular material and/or chemical precursors.

### 1.3 Pharmaceutical Compositions

Provided herein are compositions comprising an antibody or antigen-binding fragment thereof, bispecific antibody, ADC or CAR-T cell described herein having the desired degree of purity in a physiologically acceptable carrier, excipient or stabilizer (Remington's Pharmaceutical Sciences (1990) Mack Publishing Co., Easton, Pa.). Acceptable carriers, excipients, or stabilizers are nontoxic to recipients at the dosages and concentrations employed.

In various embodiments, compositions comprising an anti-Siglec-15 antibody or antigen-binding fragment thereof, bispecific antibody, ADC or CAR-T cell are provided in formulations with a pharmaceutically acceptable carrier (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000))

Pharmaceutical compositions described herein can be useful in blocking the inhibitory activity of Siglec-15 against T cells and/or in ADCC, ADCP, or CDC-dependent depletion of Siglec-15 expressing cells. Pharmaceutical compositions described herein can be useful in treating a condition such as cancer. Examples of cancer that can be treated in accordance with the methods described herein include, but are not limited to, breast cancer (e.g., triple negative breast cancer, ductal carcinoma), endometrial carcinoma, ovarian cancer, and non-small cell lung cancer (e.g., squamous cell carcinoma), pancreatic cancer, thyroid cancer, kidney cancer (e.g., renal cell carcinoma), and bladder cancer (e.g., urothelial cell carcinoma). A non-small cell lung cancer can be, e.g., an adenocarcinoma. Additional examples of cancer that can be treated in accordance with the methods described herein include, but are not limited to, head and neck cancer, small cell lung cancer, gastric cancer, melanoma, and cholangiocarcinoma.

The pharmaceutical compositions described herein are in one embodiment for use as a medicament. The pharmaceutical compositions described herein are in one embodiment for use as a diagnostic, e.g., to detect the presence of Siglec-15 in a sample obtained from a patient (e.g., a human patient).

The compositions to be used for in vivo administration can be sterile. This is readily accomplished by filtration through, e.g., sterile filtration membranes.

In some embodiments, pharmaceutical compositions are provided, wherein the pharmaceutical composition comprises anti-Siglec-15 antibodies or antigen-binding fragments thereof, bispecific antibodies, ADCs or CAR-T cells described herein and a pharmaceutically acceptable carrier. In some embodiments, a pharmaceutical composition comprises (i) an isolated antibody or antigen-binding fragment thereof that specifically binds to human Siglec-15, comprising (a) the heavy chain variable region (VH) complementarity determining region (CDR) 1, VH CDR2, VH CDR3 and light chain variable region (VL) CDR1, CDR2, and CDR3 sequences of SEQ ID NOs: 4, 5, 6, 7, 8, and 8, respectively, 12, 13, 14, 15, 16, and 17, respectively, 20, 21, 22, 23, 24, and 25, respectively, 28, 29, 30, 31, 32, and 33, respectively, 36, 37, 38, 39, 40, and 41, respectively, 44, 45, 46, 47, 48, and 49, respectively, 52, 53, 54, 55, 56, and 57, respectively, 60, 61, 62, 63, 64, and 65, respectively, or 68, 69, 70, 71, 72, and 73, respectively, or (b) a variable heavy chain region comprising the amino acid sequence of SEQ ID NO: 10, 18, 26, 34, 42, 50, 58, 66, 74, 104, or 105 and a variable light chain region comprising the amino acid sequence of SEQ ID NO: 11, 19, 27, 35, 43, 51, 59, 67, 75, 107, or 108, and (ii) a pharmaceutically acceptable excipient.

### 1.4 Uses and Methods

In one aspect, presented herein are methods for modulating one or more immune functions in a subject, comprising administering to a subject in need thereof an anti-Siglec-15 antibody or antigen-binding fragment thereof, bispecific antibody, ADC, or CAR-T cell described herein, or a pharmaceutical composition thereof as described above and herein.

In a certain embodiment, provided herein are methods of treating a cancer, e.g., a Siglec-15 expressing cancer. The method of treating cancer can comprise administering an anti-Siglec-15 antibody or antigen-binding fragment thereof, bispecific antibody, ADC, or CAR-T cell provided herein or a pharmaceutical composition comprising an anti-Siglec-15 antibody or antigen-binding fragment thereof, bispecific antibody, ADC, or CAR-T cell provided herein to a patient (e.g., a human patient) in need thereof.

In a certain embodiment, provided herein are methods of treating a cancer selected from the group consisting of: oral and pharynx cancer (tongue, mouth, pharynx, head and neck), digestive system cancers (esophagus, stomach, small intestine, colon, rectum, anus, liver, interhepatic bile duct, gallbladder, pancreas), respiratory system cancers (larynx, lung and bronchus, e.g., non-small cell lung cancer), bones and joint cancers (e.g., osteosarcoma, Ewing's sarcoma, chondrosarcoma, enchondroma, osteochondroma, undifferentiated pleomorphic sarcoma, fibrosarcoma, giant cell tumor, chordoma, myeloma), soft tissue cancers, skin cancers (melanoma, basal and squamous cell carcinoma), pediatric tumors (neuroblastoma, rhabdomyosarcoma, osteosarcoma, Ewing's sarcoma), tumors of the central nervous system (brain, astrocytoma, glioblastoma, glioma), and cancers of the breast (e.g., triple negative breast cancer, ductal carcinoma), the genital system (uterine cervix, uterine corpus, ovary, vulva, vagina, prostate, testis, penis, endometrium), the urinary system (urinary bladder, kidney and renal pelvis, ureter, e.g., renal cell carcinoma, urothelial cell carcinoma), the eye and orbit, the endocrine system (thyroid), liquid cancers (leukemia, lymphoma), and the brain and other nervous system, or any combination thereof. In a certain embodiment, provided herein are methods of treating a non-small cell lung cancer that is an adenocarcinoma. In some embodiments, such methods comprise administering an anti-Siglec-15 antibody or antigen-binding fragment thereof provided herein or a pharmaceutical composition comprising an anti-Siglec-15 antibody or antigen-binding fragment thereof provided herein to a patient (e.g., a human patient) in need thereof. In some embodiments, the cancer is a Siglec-15 expressing cancer. In some embodiments, such methods comprise administering an anti-Siglec-15 bispecific antibody, ADC or CAR-T cell of the invention to the patient.

In some aspects, the cancer can include, but is not limited to, adrenal cortical cancer, advanced cancer, anal cancer, aplastic anemia, bileduct cancer, bladder cancer, bone cancer, bone metastasis, brain tumors, brain cancer, breast cancer, childhood cancer, cancer of unknown primary origin, Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, liver cancer, non-small cell lung cancer, small cell lung cancer, lung carcinoid tumor, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment.

In certain aspects, the cancer or tumor is stage 0, such that, e.g., the cancer or tumor is very early in development and has not metastasized. In some aspects, the cancer or tumor is stage I, such that, e.g., the cancer or tumor is relatively small in size, has not spread into nearby tissue, and has not metastasized. In some aspects, the cancer or tumor is stage II or stage III, such that, e.g., the cancer or tumor is larger than in stage 0 or stage I, and it has grown into neighboring tissues but it has not metastasized, except potentially to the lymph nodes. In some aspects, the cancer or tumor is stage IV, such that, e.g., the cancer or tumor has metastasized. Stage IV can also be referred to as advanced or metastatic cancer.

In some aspects, the tumor is a solid tumor. A “solid tumor” includes, but is not limited to, sarcoma, melanoma, carcinoma, or other solid tumor cancer. Sarcomas include, but are not limited to, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

In some embodiments, provided herein is a method of reducing or slowing tumor growth in a subject. In some embodiments, such methods comprise administering an anti-Siglec-15 antibody or antigen-binding fragment thereof, bispecific antibody, ADC, CAR-T cell, or pharmaceutical composition thereof of the invention to the patient. In some embodiments, tumor growth is 5-100% inhibited in the subject. In some embodiments, administration of an anti-Siglec-15 antibody or antigen-binding fragment thereof, bispecific antibody, ADC, CAR-T cell, or pharmaceutical composition thereof results in about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% tumor growth inhibition in the subject. In some embodiments, administration of an anti-Siglec-15 antibody or antigen-binding fragment thereof, bispecific antibody, ADC, CAR-T cell, or pharmaceutical composition thereof results in at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% tumor growth inhibition in the subject.

In some embodiments, administration of an anti-Siglec-15 antibody or antigen-binding fragment thereof, bispecific antibody, ADC, CAR-T cell, or pharmaceutical composition thereof results in about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% less tumor volume relative to administration of a control. In some embodiments, administration of an anti-Siglec-15 antibody or antigen-binding fragment thereof, bispecific antibody, ADC, CAR-T cell, or pharmaceutical composition thereof results in at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% less tumor volume relative to administration of a control.

In some embodiments, provided herein is a method of treating a cancer that is a refractory cancer non-responsive to one or more prior therapies. In some embodiments, a prior therapy is chemotherapy, immunotherapy, neo-adjuvant therapy, or adjuvant therapy. In some embodiments, the immunotherapy is a checkpoint inhibitor. In a certain embodiment, provided herein is a method of treating a cancer that is an inadequate responder to one or more therapies. A cancer that is an inadequate responder, may have previously responded to a therapy, but may have become less responsive to the therapy, or the cancer may have never responded to the therapy. Inadequate response to a therapy means that aspects of the cancer that would be expected to improve following a standard dose of the therapy do not improve, and/or improvement only occurs if greater than a standard dose is administered. In some embodiments, a subject with a cancer that is an inadequate responder has experienced, or is experiencing, an inadequate response to the therapy after receiving a standard dose for at least two weeks, at least three weeks, at least four weeks, at least six weeks, or at least twelve weeks. A “standard” dose is determined by a medical professional, and may depend on the subject's age, weight, healthy history, severity of disease, the frequency of dosing, etc. In some embodiments, subject with a cancer that is an inadequate responder has experienced, or is experiencing, an inadequate response to a chemotherapy. In some embodiments, subject with a cancer that is an inadequate responder has experienced, or is experiencing, an inadequate response to an immunotherapy. In some embodiments, subject with a cancer that is an inadequate responder has experienced, or is experiencing, an inadequate response to a checkpoint inhibitor. In some embodiments, subject with a cancer that is an inadequate responder has experienced, or is experiencing, an inadequate response to a checkpoint inhibitor selected from a PD-1 inhibitor, a PD-L1 inhibitor, a LAG-3 inhibitor, and a CTLA-4 inhibitor. In some embodiments, a subject with cancer that is a checkpoint inhibitor inadequate responder has experienced, or is experiencing, an inadequate response to a checkpoint inhibitor selected from ipilimumab, tremelimumab, relatlimab, cemiplimab, durvalumab, nivolumab, pembrolizumab, and atezolizumab.

In further embodiments, an anti-Siglec-15 antibody or antigen-binding fragment thereof, bispecific antibody, ADC, CAR-T cell, or pharmaceutical composition, is administered to a patient as provided above, and further in combination with an additional therapeutic agent, e.g., a chemotherapeutic agent or an immune stimulating agent, such as a T cell checkpoint inhibitor.

In an exemplary embodiment, the additional therapeutic agent is a PD-1 antagonist, such as an antagonistic PD-1 antibody. Suitable PD-1 antibodies include, for example, OPDIVO (nivolumab), KEYTRUDA (pembrolizumab), or MEDI-0680 (AMP-514; WO2012/145493). Suitable PD-1 antibodies also include, for example, camrelizumab (SHR-1210), tislelizumab (BGB-A317), or spartalizumab (NPVPDR001, NVS240118, PDR001). The additional therapeutic agent may also include pidilizumab (CT-011). A recombinant protein composed of the extracellular domain of PD-L2 (B7-DC) fused to the Fc portion of IgG1, called AMP-224, can also be used to antagonize the PD-1 receptor.

In another exemplary embodiment, the additional therapeutic agent is a PD-L1 antagonist, such as an antagonistic PD-L1 antibody. Suitable PD-L1 antibodies include, for example, TECENTRIQ (atezolizumab), durvalumab (MEDI4736), BMS-936559 (WO2007/005874), MSB0010718C (WO2013/79174) or rHigM12B7.

Usually, the patient is a human but non-human mammals including transgenic mammals can also be treated.

In some embodiments, the present invention relates to an antibody or antigen-binding fragment thereof or pharmaceutical composition provided herein for use as a medicament. In some aspects, the present invention relates to an antibody or antigen-binding fragment thereof or pharmaceutical composition provided herein, for use in a method for the treatment of cancer. In some aspects, the present invention relates to an antibody or antigen-binding fragment thereof or pharmaceutical composition provided herein, for use in a method for the treatment of cancer in a subject, comprising administering to the subject an effective amount of an antibody or antigen-binding fragment thereof or pharmaceutical composition provided herein.

In a certain embodiment, provided herein are methods of diagnosis, detection, monitoring, prevention, and treatment of bone loss or bone resorption, or to determine or assist in the determination or identification of suitable patient populations or profiles. The detection or diagnosis of a disease, disorder or infection can include: (a) assaying the expression of Siglec-15 or derivatives thereof in cells, serum, plasma, blood or in a tissue sample (e.g., a tumor sample) of a subject using one or more antibodies (or fragments thereof) that immunospecifically bind to such antigens; and (b) comparing the level of the antigen with a control level, e.g., levels in normal tissue samples, whereby an increase in the assayed level of antigen compared to the control level of the antigen is indicative of the disease, disorder or infection. Such antibodies and fragments can be employed in immunoassays, such as the enzyme linked immunosorbent assay (ELISA), the radioimmunoassay (RIA) and fluorescence-activated cell sorting (FACS).

In some embodiments, the antibodies or fragments are used for IHC analysis in cells of an in vitro or in situ tissue sample or in vivo. Thus, the antibodies and fragments can be used in the detection and diagnosis of a disease, disorder, or infection in a human. In one embodiment, such diagnosis includes: a) administering to a subject (for example, parenterally, subcutaneously, or intraperitoneally) an effective amount of a labeled antibody or antigen-binding fragment of the invention; b) waiting for a time interval following the administration for permitting the labeled molecule to preferentially concentrate at sites in the subject where Siglec-15 is expressed (and for unbound labeled molecule to be cleared to background level); c) determining background level; and d) detecting the labeled antibody in the subject, such that localized detection of labeled antibody above or below the background level indicates that the subject has the disease, disorder, or infection and/or shows the location and relative expression level of Siglec-15+tissue. In accordance with this embodiment, the antibody can be labeled with an imaging moiety which is detectable in vivo using an imaging system known to one of skill in the art. Background level can be determined by various methods including, comparing the amount of labeled molecule detected to a standard value previously determined for a particular system.

Other methods include, for example, monitoring the progression of a disease, disorder or infection, by (a) assaying the expression of Siglec-15 in cells or in a tissue sample of a subject obtained at a first time point and later time point using a Siglec-15-binding molecule and (b) comparing the level of expression of Siglec-15 in the cells or in the tissue sample of the subject at the first and later time points, wherein an increase in the assayed level of Siglec-15 at the later time point compared to the first time point is indicative of the progression of disease, disorder or infection.

A method for monitoring a response to a treatment, can include, (a) assaying the expression of Siglec-15 in cells or in a tissue sample of a subject prior and after the treatment using a Siglec-15-binding molecule; and (b) comparing the level of Siglec-15 over time, whereby a decrease in the assayed level of Siglec-15 after treatment compared to the level of Siglec-15 prior to treatment is indicative of a favorable response to the treatment.

It will be understood in the art that the size of the subject and the imaging system used will determine the quantity of imaging moiety needed to produce diagnostic images. Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

In one embodiment, monitoring of a disease, disorder or infection is carried out by repeating the method for diagnosing the disease, disorder or infection, for example, one month after initial diagnosis, six months after initial diagnosis, one year after initial diagnosis, etc.

Presence of the labeled molecule can be detected in the subject using methods known in the art for in vivo scanning. These methods depend upon the type of label used. Skilled artisans will be able to determine the appropriate method for detecting a particular label. Methods and devices that can be used in the diagnostic methods include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

In some embodiments, the bone loss or resorption is associated with bone-related disease and/or an increase in osteoclast differentiation or activity. In some embodiments, the bone-related disease may be osteoporosis, osteopenia, osteomalacia, hyperparathyroidism, hypothyroidism, hyperthyroidism, hypogonadism, thyrotoxicosis, systemic mastocytosis, adult hypophosphatasia, hyperadrenocorticism, osteogenesis imperfecta, Paget's disease, Cushing's disease/syndrome, Turner's syndrome, Gaucher disease, Ehlers-Danlos syndrome, Marfan's syndrome, Menkes' syndrome, Fanconi's syndrome, multiple myeloma, hypercalcemia, hypocalcemia, arthritides, periodontal disease, rickets (including vitamin D dependent, type I and II, and x-linked hypophosphatemic rickets), fibrogenesis imperfecta ossium, osteosclerotic disorders such as pycnodysostosis and damage caused by macrophage-mediated inflammatory processes. In some embodiments, the bone loss or resorption is associated with a cancer treatment. In some embodiments, the bone loss or resorption is associated with a bone cancer (e.g., osteosarcoma, Ewing's sarcoma, chondrosarcoma, enchondroma, osteochondroma, undifferentiated pleomorphic sarcoma, fibrosarcoma, giant cell tumor, chordoma, myeloma) or metastatic cancer that has spread to the bone.

In some embodiments, the present invention relates to an antibody or antigen-binding fragment thereof or pharmaceutical composition provided herein, for use in a method of the treatment of bone loss or resorption in a subject, comprising administering to the subject an effective amount of an antibody or antigen-binding fragment thereof of pharmaceutical composition provided herein (e.g., In some aspects, the antibody or antigen-binding fragment thereof inhibits the differentiation of osteoclasts and/or inhibits bone resorption. In certain instances, the anti-Siglec-15 antibodies and antigen binding fragment thereof, bispecific antibodies, ADCs, or CAR-T cells may be administered concurrently in combination with other treatments given for the same condition. As such, the antibodies may be administered with anti-resorptives (e.g., bisphosphonates) that are known to those skilled in the art. Additionally, the antibodies may be administered with anti-mitotics (e.g., taxanes), platinum-based agents (e.g., cisplatin), DNA damaging agents (e.g. Doxorubicin), and other cytotoxic therapies that are known to those skilled in the art. In other instances, the anti-Siglec-15 antibodies and immunologically functional fragments therein may be administered with other therapeutic antibodies. These include, but are not limited to, antibodies that target RANKL, EGFR, CD-20, and Her2.

### 1.4.1.1 Routes of Administration & Dosage

An antibody or antigen-binding fragment thereof or composition described herein can be delivered to a subject by a variety of routes, such as parenteral, subcutaneous, intravenous, intradermal, transdermal, transmucosal, intramuscular, intranasal, intratumoral, and administration to a tumor draining lymph node. In one embodiment, the antibody or antigen-binding fragment thereof or composition is administered by an intravenous route.

The amount of an antibody or antigen-binding fragment thereof or composition which will be effective in the treatment of a condition will depend on the nature of the disease. The precise dose to be employed in a composition will also depend on the route of administration, and the seriousness of the disease.

### 1.4.2 Detection & Diagnostic Uses

The Siglec-15 agents of the present disclosure, such as the isolated antibodies of the present disclosure (e.g., an anti-Siglec-15 antibody described herein) also have diagnostic utility. This disclosure therefore provides for methods of using the antibodies of this disclosure, or functional fragments thereof, for diagnostic purposes, such as the detection of a Siglec-15 protein in an individual or in tissue samples derived from an individual. In some embodiments, the anti-Siglec-15 antibody 62E5 or antigen-binding fragments thereof is useful for such diagnostic purposes.

In some embodiments, the individual is a human. In some embodiments, the individual is a human patient suffering from, or at risk for developing a disease, disorder, or injury of the present disclosure. In some embodiments, the diagnostic methods involve detecting a Siglec-15 protein in a biological sample, such as a biopsy specimen, a tissue, or a cell. A Siglec-15 agent of the present disclosure (e.g., an anti-Siglec-15 antibody described herein) is contacted with the biological sample and antigen-bound antibody is detected. For example, a biopsy specimen may be stained with an anti-Siglec-15 antibody described herein in order to detect and/or quantify disease-associated cells. The detection method may involve quantification of the antigen-bound antibody. Antibody detection in biological samples may occur with any method known in the art, including immunofluorescence microscopy, immunocytochemistry, immunohistochemistry, ELISA, FACS analysis, immunoprecipitation, Western blotting, or micro-positron emission tomography. In certain embodiments, the antibody is radiolabeled, for example with 18F and subsequently detected utilizing micro-positron emission tomography analysis. Antibody-binding may also be quantified in a patient by non-invasive techniques such as positron emission tomography (PET), X-ray computed tomography, single-photon emission computed tomography (SPECT), computed tomography (CT), and computed axial tomography (CAT).

Assaying for the expression level of Siglec-15 protein is intended to include qualitatively or quantitatively measuring or estimating the level of a Siglec-15 protein in a first biological sample either directly (e.g., by determining or estimating absolute protein level) or relatively (e.g. by comparing to the protein level in a second biological sample). Siglec-15 protein expression level in the first biological sample can be measured or estimated and compared to a standard Siglec-15 protein level, the standard being taken from a second biological sample obtained from an individual not having a disease or disorder or being determined by averaging levels from a population of individuals not having a disease or disorder.

An anti-Siglec-15 antibody described herein can be used for prognostic, diagnostic, monitoring and screening applications, including in vitro and in vivo applications well known and standard to the skilled artisan and based on the present description. Prognostic, diagnostic, monitoring and screening assays and kits for in vitro assessment and evaluation of immune system status and/or immune response may be utilized to predict, diagnose and monitor to evaluate patient samples including those known to have or suspected of having an immune system-dysfunction or cancer. In vivo applications include directed cell therapy and immune system modulation and radio imaging of immune responses.

Anti-Siglec-15 antibodies and antigen-binding fragments thereof described herein can carry a detectable or functional label. When fluorescence labels are used, currently available microscopy and fluorescence-activated cell sorter analysis (FACS) or combination of both methods procedures known in the art may be utilized to identify and to quantitate the specific binding members. Anti-Siglec-15 antibodies or antigen-binding fragments thereof described herein can carry a fluorescence label. Exemplary fluorescence labels include, for example, reactive and conjugated probes, e.g., Aminocoumarin, Fluorescein and Texas red, Alexa Fluor dyes, Cy dyes and DyLight dyes. An anti-Siglec-15 antibody can carry a radioactive label, such as the isotopes 3H, 14C, 32P, 35S, 36C1, 51Cr, 57Co, 58Co, 59Fe, 67Cu, 90Y, 99Tc, 111 In, 117Lu, 121I, 124I, 125I, 131I, 198Au, 211At, 213Bi, 225Ac and 186Re. When radioactive labels are used, currently available counting procedures known in the art may be utilized to identify and quantitate the specific binding of anti-Siglec-15 antibody or antigen-binding fragment to Siglec-15 (e.g., human Siglec-15). In the instance where the label is an enzyme, detection may be accomplished by any of the presently utilized colorimetric, spectrophotometric, fluorospectrophotometric, amperometric or gasometric techniques as known in the art. This can be achieved by contacting a sample or a control sample with an anti-Siglec-15 antibody or antigen-binding fragment thereof under conditions that allow for the formation of a complex between the antibody or antigen-binding fragment thereof and Siglec-15. Any complexes formed between the antibody or antigen-binding fragment thereof and Siglec-15 are detected and compared in the sample and the control. In light of the specific binding of the antibodies or antigen-binding fragments thereof described herein for Siglec-15, the antibodies or antigen-binding fragments thereof can be used to specifically detect Siglec-15 expression on the surface of cells. The antibodies or antigen-binding fragments thereof described herein can also be used to purify Siglec-15 via immunoaffinity purification.

Also included herein is an assay system which may be prepared in the form of a test kit for the quantitative analysis of the extent of the presence of, for instance, Siglec-15. The system or test kit may comprise a labeled component, e.g., a labeled antibody or antigen-binding fragment, and one or more additional immunochemical reagents.

In some aspects, methods for in vitro detecting Siglec-15 in a sample, comprising contacting said sample with an antibody or antigen-binding fragment thereof, are provided herein. In some aspects, provided herein is the use of an antibody or antigen-binding fragment thereof provided herein, for in vitro detecting Siglec-15 in a sample. In one aspect, provided herein is an antibody or antigen-binding fragment thereof or pharmaceutical composition provided herein for use in the detection of Siglec-15 in a subject or a sample obtained from a subject. In one aspect, provided herein is an antibody or antigen-binding fragment thereof or pharmaceutical composition provided herein for use as a diagnostic. In one preferred embodiment, the antibody comprises a detectable label. In one preferred embodiment, Siglec-15 is human Siglec-15. In one preferred embodiment, the subject is a human.

### 1.5 Kits

Provided herein are kits comprising one or more antibodies or antigen-binding fragments thereof described herein or conjugates thereof. In a specific embodiment, provided herein is a pharmaceutical pack or kit comprising one or more containers filled with one or more of the ingredients of the pharmaceutical compositions described herein, such as one or more antibodies or antigen-binding fragments thereof provided herein. Optionally associated with such container(s) can be a notice in the form prescribed by a governmental agency regulating the manufacture, use, or sale of pharmaceuticals or biological products, which notice reflects approval by the agency of manufacture, use or sale for human administration.

Also provided herein are kits that can be used in diagnostic methods. In one embodiment, a kit comprises an antibody or antigen-binding fragment thereof described herein, preferably a purified antibody or antigen-binding fragment thereof, in one or more containers. In a specific embodiment, kits described herein contain a substantially isolated Siglec-15 antigen (e.g., human Siglec-15) that can be used as a control. In another specific embodiment, the kits described herein further comprise a control antibody or antigen-binding fragment thereof which does not react with a Siglec-15 antigen. In another specific embodiment, kits described herein contain one or more elements for detecting the binding of an antibody or antigen-binding fragment thereof to a Siglec-15 antigen (e.g., the antibody or antigen-binding fragment thereof can be conjugated to a detectable substrate such as a fluorescent compound, an enzymatic substrate, a radioactive compound or a luminescent compound, or a second antibody or antigen-binding fragment thereof which recognizes the first antibody or antigen-binding fragment thereof can be conjugated to a detectable substrate). In specific embodiments, a kit provided herein can include a recombinantly produced or chemically synthesized Siglec-15 antigen. The Siglec-15 antigen provided in the kit can also be attached to a solid support. In a more specific embodiment, the detecting means of the above described kit includes a solid support to which a Siglec-15 antigen is attached. Such a kit can also include a non-attached reporter-labeled anti-human antibody or antigen-binding fragment thereof or anti-mouse/rat antibody or antigen-binding fragment thereof. In this embodiment, binding of the antibody or antigen-binding fragment thereof to the Siglec-15 antigen can be detected by binding of the said reporter-labeled antibody or antigen-binding fragment thereof.

### EXAMPLES

The examples in this Section are offered by way of illustration, and not by way of limitation.

### Example 1: Characterization of Tumor Growth and Metastatic Spread in Siglec-15 Knockout Mice

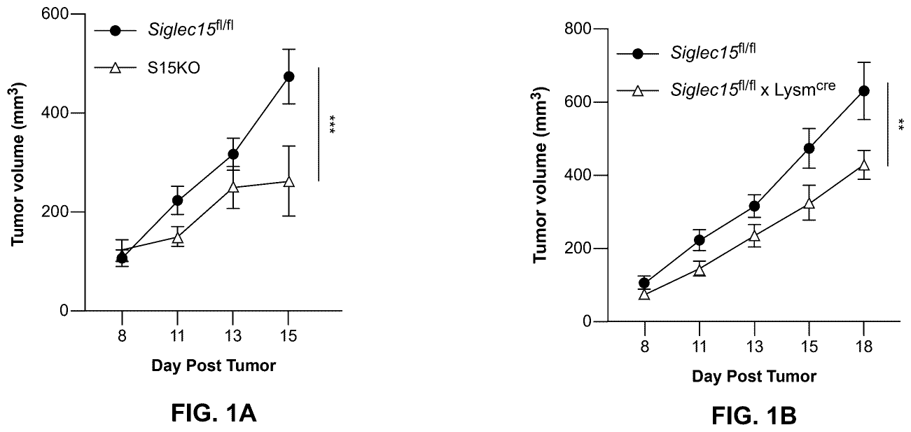

Siglec-15 knockout (KO) mice were utilized to observe the impact of Siglec-15 deficiency on tumor progression. Female C57BL/6 wild-type (WT) and Siglec-15 knockout (KO) mice were injected with EO771 murine mammary cancer cells. EO771 breast tumors demonstrated a significantly slower tumor growth rate in Siglec-15 KO mice relative to WT mice (FIG. 1A), and the final tumor weight was significantly lower in Siglec-KO mice (FIG. 1B). Further, analysis of metastatic spread revealed a significant decrease in metastasis in Siglec-15 KO mice relative to WT mice (FIG. 1C). Collectively, these findings implicate Siglec-15 as a critical modulator of both tumor growth and metastatic dissemination.

### Materials and Methods

Female C57BL/6 wild-type (WT) and Siglec-15 knockout (KO) mice, aged 6-8 weeks, were housed under specific pathogen-free conditions with a 12-hour light/dark cycle. EO771 mouse breast cancer cells were cultured in DMEM (Dulbecco's Modified Eagle Medium) supplemented with 10% fetal bovine serum (FBS) and 1% penicillin-streptomycin at 37° C. in a humidified atmosphere with 5% CO2. Cells were harvested at 80-90% confluency using 0.25% trypsin-EDTA solution. Prior to injection, E0771 cells were washed with phosphate-buffered saline (PBS), counted, and resuspended in PBS. The final concentration was adjusted to 1×10\hat{ }6 cells in 100 μL for each injection. Mice were anesthetized with a ketamine-xylazine mixture (100 mg/kg and 10 mg/kg body weight, respectively) administered intraperitoneally. Carprofen (5 mg/kg) was given subcutaneously for analgesia prior to surgery and every 24 hours for 48 hours post-procedure. The area around the fourth mammary fat pad was sterilized with 70% ethanol. A small incision was made in the skin overlaying the mammary fat pad, and 100 μL of the cell-PBS suspension was carefully injected into the fat pad using a 27-gauge needle. The incision was closed with surgical glue or sutures. Mice were monitored until fully recovered from anesthesia and then daily for any signs of distress or infection. Tumor growth was assessed by palpation twice weekly, and tumor size was measured using calipers once the tumor became palpable.

The experimental endpoint was determined either by ethical tumor size (1 cm\hat{ }3), signs of ulceration, or significant health decline. Mice were euthanized, and tumors were excised for weighing, fixation, and histological analysis. Lungs and other organs were also collected for the evaluation of metastatic spread if applicable.

Metastasis was assessed by detecting GFP-positive cells in the peripheral blood and organs. Blood and homogenized tissue samples were subjected to flow cytometry in a BD FACSymphony analyzer. Counting beads were added to each sample to quantify the number of GFP-positive cells per million total cells. The growth curves were analyzed using repeated measures ANOVA to compare tumor growth over time between the two groups. The tumor weights and metastatic counts were compared using an unpaired t-test or Mann-Whitney U test, depending on the data distribution. A p-value of less than 0.05 was considered statistically significant.

### Example 2: Development of Novel Anti-Siglec-15 Monoclonal Antibodies with Hybridoma Technology

BALB/c and Siglec-15 knockout (KO) C57/BL6 mice were immunized using full-length recombinant human Siglec-15 protein to elicit an immune response. Following immunization, spleen cells from the mice were fused with myeloma cells to generate hybridomas, which were subsequently screened for their ability to produce antibodies that recognize human Siglec-15.

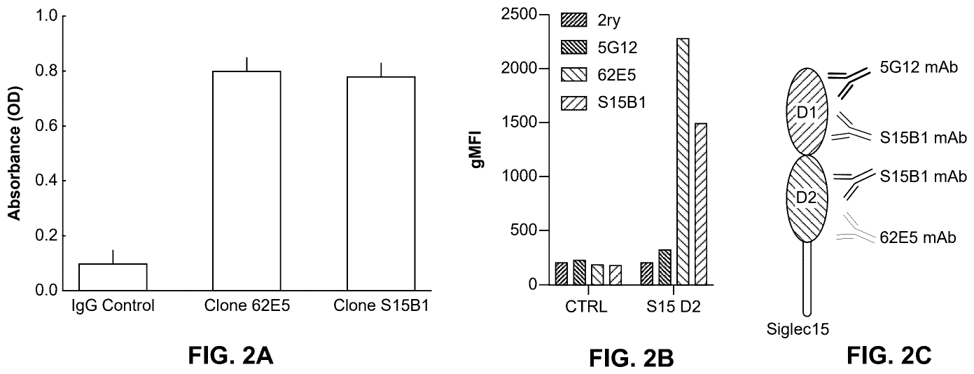

An enzyme-linked immunosorbent assay (ELISA) targeting the full-length recombinant human Siglec-15 protein was used to identify hybridomas producing antibodies with the desired specificity, resulting in the selection of two promising clones: 62E5 and S15B1 (FIG. 2A). Flow cytometry was used to interrogate the binding of anti-Siglec-15 monoclonal antibodies to cells expressing only a truncated version of Siglec-15 comprising solely of the second domain (D2). This strategy was useful to identify clones that recognize epitopes outside of the canonical sialic acid binding site located in the first domain (D1).

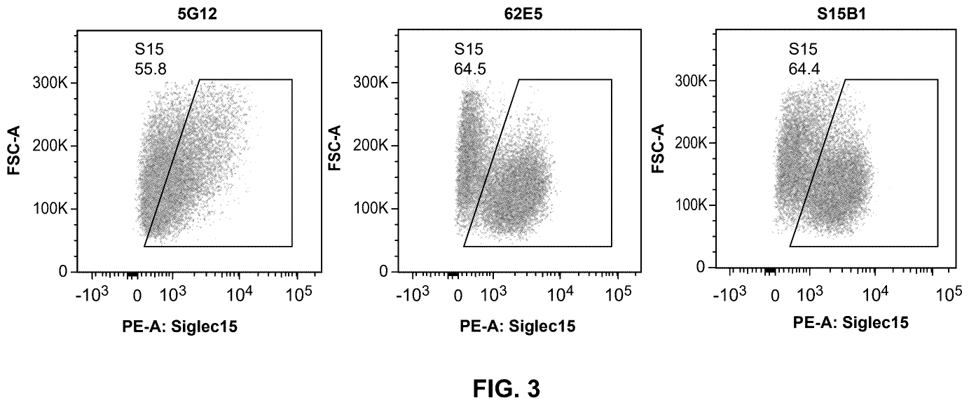

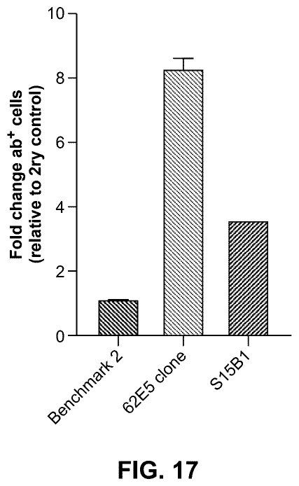

Clones 62E5 and S15B1 displayed binding affinity for D2, distinguishing them from previously characterized antibodies that primarily target the sialic acid binding site within D1, such as the 5G12 antibody developed by Nextcure (FIG. 2B and FIG. 2C). Unlike 5G12, which does not interact with D2, clones 62E5 and S15B1 exhibit a unique binding profile that may confer distinct therapeutic advantages. Clones 62E5 and S15B1 demonstrated approximately 8- and 4-fold increase in antibody-positive cells measured by flow cytometry, indicating significant binding to D2 compared to a second benchmark, antibody clone A2A5C7E8 (FIG. 17). Clones 62E5 and S15B1 demonstrated superior binding to Siglec-15 on human macrophages compared to the control clone 5G12 (FIG. 3).

### Materials and Methods

Human Siglec-15 ectodomain was produced in HEK 293T cells through transient transfection using a suitable expression vector. Following the expression period, the culture supernatant was harvested and subjected to a series of purification steps, including affinity chromatography utilizing a HIS-trap column, followed by dialysis to remove any residual binding reagents. The purity and integrity of the ectodomain were verified by SDS-PAGE and Western blot analysis.

Four female mice (two BALB/c and two Siglec-15 knockout C56/bl6 mice) were immunized following the Euromabnet guidelines. The immunization schedule was as follows:

Initial immunization with 100 μg of purified recombinant Siglec-15 ectodomain emulsified in complete Freund's adjuvant, delivered subcutaneously. A second dose of 100 μg of the ectodomain in incomplete Freund's adjuvant was administered subcutaneously 10 days later. A third subcutaneous injection of the ectodomain in PBS was given after another 10 days. On day 40, a booster dose of 200 μg of the ectodomain in PBS was administered intraperitoneally.

Splenocytes from the immunized mice were harvested and fused with SP2/0-Ag14 myeloma cells using a 1:2 ratio. Fusion was facilitated using PEG1500 at 37° C. The resulting hybridomas were then cultured in HAT Medium in a 96-well plate to select for successful hybridoma growth. After two weeks, ELISA was performed to identify hybridomas secreting anti-Siglec-15 antibodies. Positive clones were expanded in HT medium and subjected to limiting dilution cloning to ensure monoclonality.

96-well plates were coated with the Siglec-15 ectodomain at 2 ug/mL in a carbonate-bicarbonate coating buffer, and plates were blocked with BSA in PBS. Hybridoma supernatants were added and incubated. Plates were washed and incubated with an HRP-conjugated secondary anti-mouse IgG antibody. After washing, TMB substrate was added, followed by a stop solution. The OD at 450 nm was measured in a multi-plate reader (Perkin Elmer Nivo) to assess binding.

Positive hybridoma clones were sequenced. The variable regions of the heavy and light chains of the antibodies were sequenced to determine the sequence of the antibodies.

### Example 3: Cytotoxicity of Anti-Siglec-15 CAR-T Cells

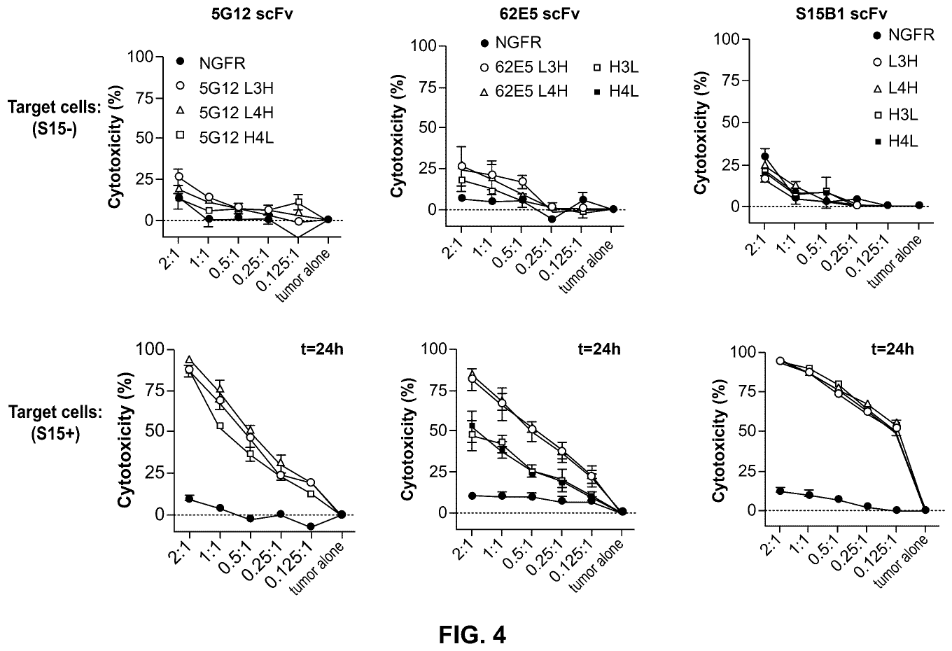

The variable regions of the anti-Siglec-15 antibodies were engineered into different CAR constructs with the objective of assessing their potential to deplete target cells. These CAR-T cells were then challenged in vitro to evaluate their cytotoxic response against cell targets engineered to express Siglec-15 (FIG. 4). Three anti-Siglec-15 CAR clones (5G12, 62E5, S15B1) and four linker variations (L3H, L4H, H3L, H4L) were evaluated to determine the most effective construct. The cytotoxic potential of anti-Siglec-15 CAR-T engineered with the generated anti-Siglec-15 monoclonal antibodies cells was quantified. Notably, CAR-T cells incorporating clone S15B1 exhibited enhanced cytotoxic effects against Siglec-15 expressing cells, surpassing those engineered with both the novel clone 62E5 and the pre-existing clone 5G12. These results underscore the promising therapeutic capacity of clone S15B1 in targeting Siglec-15 positive cells within the tumor microenvironment.

Based on these results, clone S15B1 was prioritized for development into CAR-T cells due to its potent cytotoxic activity and specificity for human Siglec-15. Expanding upon the binding characteristics of clone S15B1, its potential was further evaluated through cytotoxicity assays using anti-Siglec-15 CAR-T cells against primary human macrophages, a relevant component of the tumor microenvironment where Siglec-15 expression contributes to immune evasion. The cytotoxic potential of the CAR-T cells engineered with clone S15B1 was quantified. To this end, we first studied the phenotypic profiles of macrophages differentiated from human monocytes and polarized under various conditions.

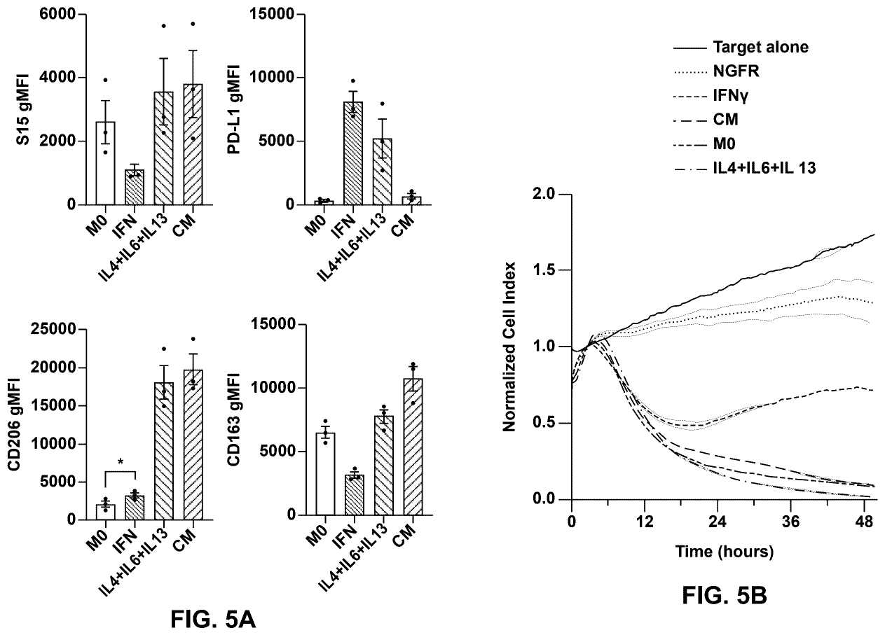

Using flow cytometry, we assessed the expression of Siglec-15 alongside macrophage polarization markers such as PD-L1, CD206, and CD163. These markers helped to confirm the polarization states: M0 (baseline), M1 (pro-inflammatory, induced by IFNγ), M2 (anti-inflammatory, induced by IL4, IL6, and IL13), and those conditioned by MDA-MB-231 tumor cell media (FIG. 5A). Then, the efficacy of CAR-T cells expressing clone S15B1 in targeting and killing Siglec-15 positive macrophages was evaluated (FIG. 5B), showing minimal activity of CAR-T cells against IFNγ-treated macrophages, which lack Siglec-15 expression and thus are not recognized by the CAR construct. The impedance-based cytotoxicity readout reflects the specificity and real-time killing capacity of the CAR-T cells, with an evident preferential targeting of macrophages polarized towards an immunosuppressive phenotype (FIGS. 5A and 5B). This data supports clone S15B1's features as a potent immunotherapeutic agent and underscores the precision with which S15B1-equipped CAR-T cells can target and deplete Siglec-15 positive cells. Such specificity is relevant for reducing off-target effects and maximizing anti-tumor activity in the clinical setting.

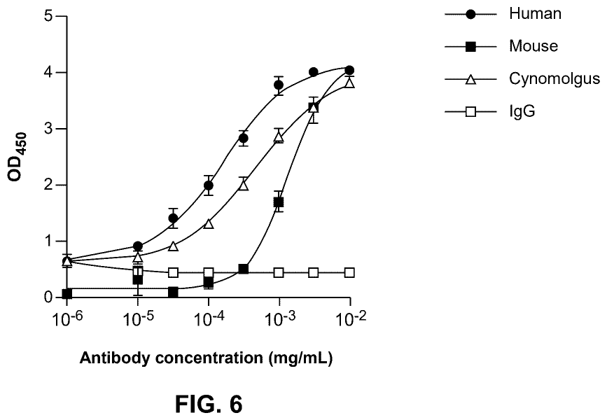

We then assessed clone S15B1 for its cross-reactivity with Siglec-15 from different species to support its translational potential in diverse models (FIG. 6). These results highlight the potential of Siglec-15 as a therapeutic target and underscores the efficacy of our antibodies in promoting target cell depletion. The exploration of these antibodies in various CAR constructs and their substantial cytotoxic activity mark a significant advancement in the development of targeted cancer therapies. These novel anti-Siglec-15 antibodies may offer a broader therapeutic window and improved clinical outcomes for patients suffering from solid tumors, such as breast, lung, and liver cancers, by mitigating the immunosuppressive tactics of malignant cells.

### Materials and Methods

MOLM-13 cells were genetically modified to stably overexpress human Siglec-15 using a lentiviral system. Cells were selected with appropriate antibiotics to ensure stable integration. Untransduced MOLM-13 cells were used as a control.

Peripheral blood mononuclear cells (PBMCs) were obtained from healthy donors. CD8+ T cells were then isolated using the Stemcell Robosep CD8 T Cell Isolation Kit, following the manufacturer's protocol to achieve high-purity negative selection. The isolated CD8+ T cells were activated to enhance their responsiveness to genetic manipulation and subsequent CAR expression. This was achieved using anti-CD3/CD28 beads, which effectively stimulate T cells by cross-linking the CD3 and CD28 receptors, mimicking antigenic stimulation. Lentiviral vectors encoding anti-Siglec-15 CAR constructs were prepared. These constructs included different antibody clones (5G12, 62E5, S15B1) to target Siglec-15. Various linker sequences and arrangements (L3H, L4H, H3L, H4L) were incorporated to assess the optimal configuration for CAR functionality. Each CAR construct also included a sequence encoding for the nerve growth factor receptor (NGFR). This serves as a marker for transduction efficiency, enabling easy tracking and quantification of successfully transduced T cells. Activated CD8+ T cells were transduced with lentiviral particles. Transduced T cells were assessed for CAR expression using flow cytometry, focusing on the NGFR marker. This step was crucial to confirm successful transduction and to estimate the proportion of T cells expressing the CAR.

CAR-T cells were cocultured with target MOLM-13 cells at various E: T ratios. After 24 hours, cells were harvested and stained with antibodies against NGFR (to identify CAR-T cells) and DAPI (to detect dead cells). Flow cytometry was used to assess the percentage of alive GFP+target cells, with the loss of target cell viability indicating CAR-T cell-mediated cytotoxicity. Control experiments included cocultures of untransduced MOLM-13 cells with CAR-T cells and cocultures of MOLM-13-Siglec-15+ cells with non-CAR expressing NGFR+ T cells. Percent cytotoxicity was calculated with the formula: Cytotoxicity (percentage)=(1-number of alive GFP+target cells in experimental condition/number of alive GFP+target cells in untreated condition)×100. Data from three independent donors were analyzed statistically to evaluate the effectiveness and specificity of each anti-Siglec-15 CAR construct.

Human peripheral blood mononuclear cells (PBMCs) were isolated from blood donors, who provided informed consent, using density gradient centrifugation. Monocytes were then purified from PBMCs utilizing a CD14 selection kit (Stemcell Robosep) according to the manufacturer's instructions. The isolated monocytes were differentiated into macrophages by culturing in RPMI 1640 medium supplemented with 10% FBS and 50 ng/ml macrophage colony-stimulating factor (M-CSF) for 5 days. Following differentiation, macrophages were washed and incubated in fresh media under four different conditions to induce specific polarization states:
M0 (control): Macrophages were maintained without additional treatment.
M1: Macrophages were treated with 100 ng/ml interferon-gamma (IFNγ).
M2: Macrophages were treated with a combination of 20 μg/mL each of interleukin 4 (IL4), interleukin 6 (IL6), and interleukin 13 (IL13).

Tumor-associated macrophages (TAMs): Macrophages were incubated with 50% MDA-MB-231 conditioned media (CM) for the specified duration.

Post-polarization, the macrophages were harvested, washed using Flow Cytometry Staining Buffer (00-4222-26, Thermo Fisher Scientific), and subsequently incubated with fluorochrome-conjugated antibodies against Siglec-15, PD-L1, CD206, and CD163. This step was performed for 30 minutes at 4° C. in a dark environment to prevent photobleaching of the fluorochromes and to maintain the integrity of the antibodies.

Following incubation, the cells underwent a second washing step and then resuspended in 200 μL of Flow Cytometry Staining Buffer containing DAPI (1:10,000) (D1306, Invitrogen). DAPI is a nuclear stain that facilitates the identification of viable cells and enables the exclusion of dead cells or debris during analysis. The stained cells were analyzed using a FACSymphony flow cytometer (BD Biosciences). The flow cytometry data were then processed and analyzed using FlowJo software (BD Biosciences).

An xCELLigence impedance system was used to assess the cytotoxicity of anti-Siglec-15 CAR-T cells against polarized macrophages. Macrophages were seeded in xCELLigence E-Plates and allowed to adhere. CAR-T cells were prepared as described previously and then added to the macrophages at a predetermined effector-to-target ratio. The system monitored electrical impedance across interdigitated micro-electrodes integrated on the bottom of the E-Plates, providing a real-time, label-free measurement of cell lysis. The change in impedance correlates with the number of living cells attached to the plate, thereby allowing quantification of CAR-T cell-mediated cytotoxicity.

To assess the cross-reactivity of the monoclonal antibody S15B1, we performed an ELISA with recombinant Siglec-15 proteins from mouse, human, and Cynomolgus monkey. The steps were as follows:

Coating: High-binding ELISA plates were coated with 100 ng per well of recombinant Siglec-15 proteins from the respective species diluted in a carbonate-bicarbonate coating buffer, pH 9.6, and incubated overnight at 4° C.

Blocking: The plates were washed with PBST (PBS with 0.05% Tween-20) and blocked with 1% BSA in PBS for 1 hour at room temperature to prevent non-specific binding.

Antibody Incubation: Diluted monoclonal antibody S15B1 was added to the wells and incubated for 2 hours at room temperature to allow binding to the coated proteins.

Detection: After washing the plates to remove unbound antibodies, an HRP-conjugated anti-mouse IgG secondary antibody was added to the wells and incubated for 1 hour at room temperature. Plates were washed again, and TMB substrate was added to each well. The colorimetric reaction was allowed to proceed for a controlled time before adding the stop solution. The optical density at 450 nm (OD450) was measured using a microplate reader (Perkin Elmer Nivo) to determine the binding affinity of S15B1 for each species' Siglec-15.

### Example 4: Development of Novel Anti-Siglec-15 Monoclonal Antibodies with Phage Display Screening

Seven fully human anti-Siglec-15 antibodies were generated utilizing the phage display technology. This approach has yielded fully human antibodies that exhibit cross-reactivity with both human and mouse Siglec-15. Notably, certain antibodies within this panel have shown the capability to enhance the cytotoxic activity of T cells in functional assays—a promising attribute for potential cancer immunotherapy applications. Three parallel phage display strategies were executed, each designed to isolate antibodies with unique binding characteristics to Siglec-15 (Table 8).

TABLE 8

| | Counter | Positive | Positive | Positive | Positive |
|---|---|---|---|---|---|
| | selection | selection | selection | selection | selection |
| Strategy | in PR1-PR4 | in PR1 | in PR2 | in PR3 | in PR4 |
| S1 | hlgG1-Fc | Rec. Siglec- | Rec. Siglec- | Rec. Siglec- | Rec. Siglec- |
| | | 15-bio | 15-bio | 15-bio | 15-bio |
| S2 | Molm13-wt | Rec. Siglec- | Molm13 | Molm13 | Molm13 |
| | | 15-bio | S15ATM | S15ATM | S15ATM |
| S3 | Molm13-wt | Molm13 | Molm13 | Molm13 | Molm13 |
| | | S15ATM | S15ATM | S15ATM | S15ATM |

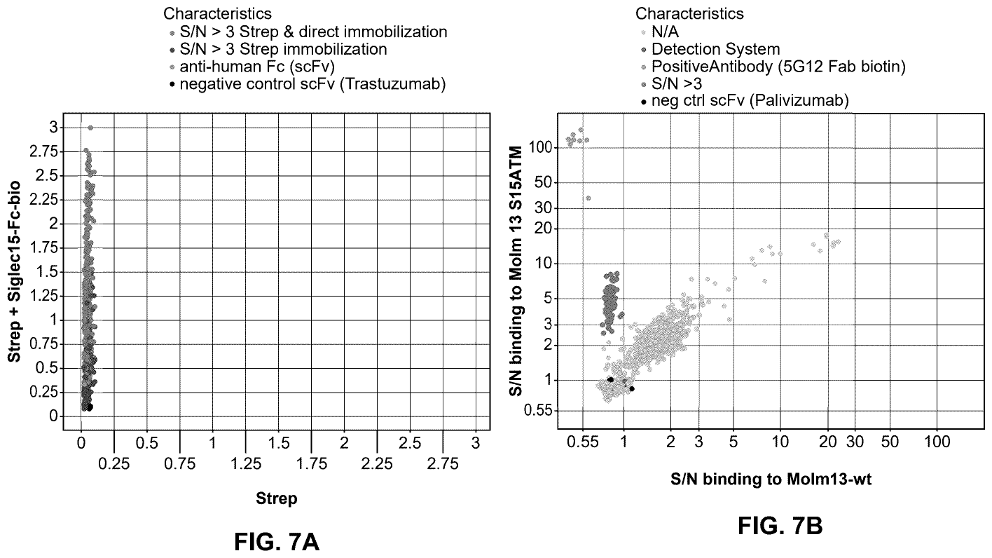

Strategy One (S1) focused on using recombinant human Siglec-15 Fc fusion protein (rS15Fc) as a bait for isolating phages from a naive human single-chain variable fragment (scFv) library. Phages with a high affinity for rS15Fc were enriched through iterative rounds of biopanning. Positive hits were identified via ELISA, revealing a diverse panel of scFvs with specific binding to rS15Fc (FIG. 7A). This specificity was further validated by retesting the positive scFvs against rS15Fc in ELISA, ensuring the reproducibility of the interaction.

Strategy Two (S2) mirrored the initial targeting and validation steps of S1, using the same rS15Fc protein to capture specific binders. However, the validation of unique phage clones was enhanced by employing flow cytometry, which allowed for the assessment of binding to Siglec-15 in a more physiologically relevant cellular context. This strategy provided additional insights into the ability of the antibodies to recognize and bind to the native conformation of Siglec-15 presented on cell surfaces.

Strategy Three (S3) sought to mimic the native conformational epitopes of Siglec-15 by utilizing MOLM-13 cells stably expressing the target protein. This approach aimed to capture phages capable of recognizing Siglec-15 as it naturally occurs on cell surfaces, a crucial aspect for therapeutic antibody development. The screening was conducted using flow cytometry to detect direct phage binding to cells. The final validation step involved confirming the specific interaction of these phages with Siglec-15-expressing cells, again using flow cytometry, to ascertain the relevance of the identified antibodies to native Siglec-15 (FIG. 7B). Subsequent sequencing of the positive clones provided a comprehensive understanding of the binding diversity.

Each sublibrary underwent four rounds of selection, known as biopanning, to enrich for phages displaying scFvs with the highest affinity for the Siglec-15 antigen. This dual approach of using recombinant protein and antigen-expressing cells, coupled with the separate kappa and lambda sublibrary screenings, was designed to ensure a comprehensive probe of the library's diversity. The result was a diverse panel of antibodies with varying specificities and affinities, ready for further characterization and potential development into therapeutic agents. The number of identified hits with each strategy are detailed in Table 9.

TABLE 9

| Strategy | PR3 | PR4 | Total |
|---|---|---|---|
| S1 | 265 | 244 | 509 |
| S2 | 65 | 12 | 77 |
| S3 | 3 | 0 | 3 |
| total | 333 | 256 | 589 |

During the advanced stages of the phage display antibody discovery campaign, DNA sequencing analysis was performed to determine the uniqueness of the antibody clones obtained from the three different strategies. From Strategy One (S1), 83 unique antibodies were successfully identified. This remarkable diversity underscores the effectiveness of the biopanning strategy against the recombinant form of Siglec-15. Further analysis of 80 additional clones that emerged from Strategies Two (S2) and Three (S3) revealed 4 additional unique antibodies. Notably, these 4 unique clones accounted for 21 out of the 80 hits identified, signifying a substantial contribution to the pool of specific binders. It is also noteworthy that the remaining hits were not new entities but rather duplicates of three clones previously discovered in Strategy One (S1). These clones, which included ZLA-1214-D08, ZLA-1214-H05, and ZLA-1214-B07, were not only identified in the initial protein panning but were also confirmed as cell binders in the subsequent validation screenings via flow cytometry. The repeated identification of these clones in two independent discovery campaigns—one focused on the recombinant protein and the other on cell-expressed antigens—strongly suggests that these antibodies exhibit specific binding to the Siglec-15 target. The cross-validation of these clones, through distinct and independent methodologies, provides a robust foundation for their potential as therapeutically relevant anti-Siglec-15 antibodies.

Identified antibody sequences were converted into full-length human IgG1 format with silenced Fc regions using amino acid substitutions E233P, L234V, L235A, G236 deletion, D265G, A327Q, A330S, and C-terminus lysine deletion relative to wild-type IgG1 to facilitate in-depth functional studies. This process involved the cloning of the antibody sequences into mammalian expression vectors for the production of human IgG1 antibodies. Upon preparation of the transfection-grade DNA, which included sequence verification to ensure fidelity, we carried out the transient transfection of HEK 293T cells. The expressed antibodies were then harvested and subjected to protein A purification, resulting in a collection of fully human IgG1 antibodies. Quality control of these purified antibodies was performed using UV/VIS spectrometry for concentration determination and SDS-PAGE under reducing conditions to confirm purity and integrity.

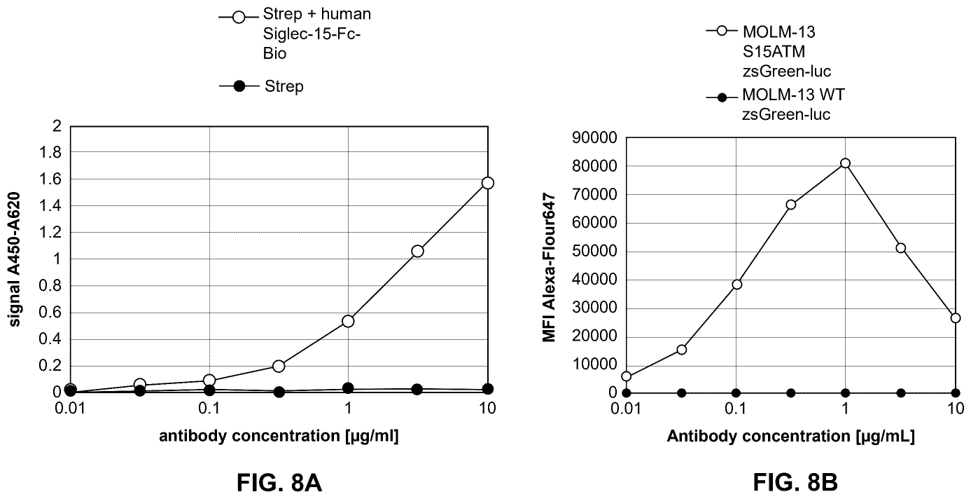

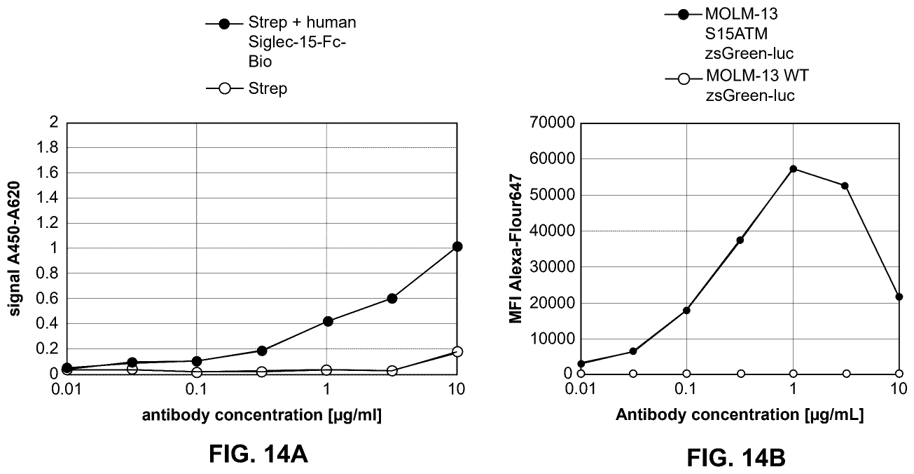

The binding efficacy of the converted full-length IgG1 anti-Siglec-15 antibodies was then evaluated using both ELISA and flow cytometry. By immobilizing the antigen and titrating the antibodies starting at a concentration of 10 μg/ml, binding kinetics were determined via ELISA, using an anti-human IgG Fab-specific secondary antibody for detection. Palivizumab served as the negative control in these assays (FIG. 8A). Complementary to the ELISA, flow cytometry assays provided insight into the binding behavior of the antibodies on the cell surface. Here, titration of the antibodies began at the same concentration of 10 μg/ml, using MOLM-13 human Siglec-15 and MOLM-13 wild-type cells as a negative control, with detection performed using an anti-human Fc secondary antibody (FIG. 8B). Clone ZLA-1243-A01 is exemplified in an ELISA binding curve (FIG. 14A) and in a MOLM-13 flow cytometry analysis (FIG. 14B). From these assays, EC50 values were calculated, offering a quantitative measure of the antibodies' affinity towards Siglec-15, and leading to the selection of 7 unique hits (Table 10).

Based on the strategy employed for biopanning and the binding characteristics of full-length antibodies to Siglec-15 expressed on the cellular surface, 7 antibodies were selected for downstream characterization. Binding data, together with the screening strategy employed, is summarized in Table 10.

TABLE 10

| | | EC50 | 95% | |
|---|---|---|---|---|
| | Screening | (ng/mL) | CI EC50 | R |
| Antibody | strategy | hS15 | [ng/ml] | squared |
| ZLA-1243-A01 | S3 | 180.6 | 132.1 to 245.1 | 0.9867 |
| ZLA-1243-E01 | S2 | 174.3 | 47.36 to 994.0 | 0.8183 |
| ZLA-1214-D08 | S1 | 105.6 | 33.45 to 382.8 | 0.8551 |
| ZLA-1214-B07 | S1 | 132.6 | 103.1 to 170.2 | 0.9914 |
| ZLA-1212-E09 | S1 | 177.6 | 114.9 to 272.4 | 0.9715 |
| ZLA-1212-B05 | S1 | 214.7 | 172.0 to 266.2 | 0.9932 |
| ZLA-1214-H05 | S1 | 592.8 | 481.5 to 726.8 | 0.9936 |

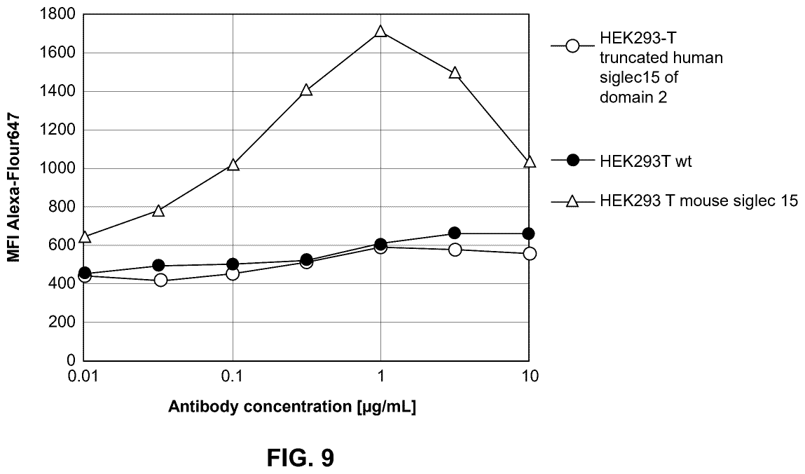

Cross-species reactivity and domain-specific binding of the full-length anti-Siglec-15 antibody candidates were evaluated. The antibodies were titrated against HEK293T cells expressing mouse Siglec-15 or HEK293T cells expressing a truncated human Siglec-15 consisting solely of domain 2 (FIG. 9). Despite the initial concentration of 10 μg/ml, none of the seven antibodies demonstrated binding to the truncated domain 2. However, the binding to mouse Siglec-15 was evident for all tested antibodies, with EC50 values calculated to rank the affinity of the antibodies. The data is summarized in Table 11.

TABLE 11

| | | EC50 | 95% | |
|---|---|---|---|---|
| | Screening | (ng/mL) | CI EC50 | R |
| Antibody | strategy | mS15 | [ng/ml] | squared |
| ZLA-1243-A01 | S3 | 75.28 | 42.91 to 132.6 | 0.9599 |
| ZLA-1243-E01 | S2 | 84.88 | 34.95 to 198.5 | 0.9131 |
| ZLA-1214-D08 | S1 | 102.9 | 56.06 to 183.3 | 0.9606 |
| ZLA-1214-B07 | S1 | 101.8 | 64.41 to 156.1 | 0.9721 |
| ZLA-1212-E09 | S1 | 191.4 | 118.3 to 302.8 | 0.9712 |
| ZLA-1212-B05 | S1 | 168.2 | 46.06 to 640.3 | 0.8321 |
| ZLA-1214-H05 | S1 | 561.5 | 117.0 to 1804 | 0.8325 |

### Materials and Methods

The identification and selection of anti-Siglec-15 antibodies were performed through the screening of a naive human single-chain variable fragment (scFv) phage display library, derived from B cells. The library screening was carried out following three distinct strategies.

### Strategy One (S1):

Targeting: We employed recombinant human Siglec-15 fused to an Fc protein (rS15Fc) to capture specific phages.

Validation: Phages binding to rS15Fc were identified via ELISA, and subsequent sequencing of these phages provided insights into the diversity of the binders.

Assessment: Selected phages were tested for their specific binding to rS15Fc in an ELISA setup, validating the interaction.

### Strategy Two (S2):

Targeting and Validation: Similar to S1, the rS15Fc protein served as the target for phage binding, followed by ELISA validation.

Assessment: Unique phage clones were further validated for binding specificity, this time via flow cytometry, offering a different modality to confirm the interaction with Siglec-15 expressed on the surface of cells.

### Strategy Three (S3):

Targeting: The target in this strategy was a cell line (MOLM-13 S15) stably expressing Siglec-15, aiming to identify phages that could recognize the native conformation of Siglec-15 on the cell surface.

Validation and Assessment: Binding phages were isolated using flow cytometry, sequenced for diversity analysis, and further validated through specific binding assays employing flow cytometry.

Antibody conversion was carried out through several steps. First, the antibody sequences were cloned into mammalian expression vectors designed specifically for the expression of human IgG1 with silenced Fc regions. Transfection-grade DNA was then prepared, with the sequence integrity confirmed by sequencing analysis. Following this, HEK cells were transiently transfected with the constructs. After an appropriate expression period, the antibodies were purified using protein A affinity chromatography, a technique suited for isolating human IgG1 antibodies. Finally, the quality control of the purified antibodies was carried out using UV/VIS spectrometry, which provided accurate concentration measurements, and SDS-PAGE under reducing conditions, which ensured the molecular integrity of the antibodies. This approach ensured the production of high-quality antibodies for further functional characterization.

For the ELISA, antigens were immobilized on high-binding plates, and the full-length IgG1 anti-Siglec-15 antibodies were titrated beginning at a concentration of 10 μg/ml. After washing, an anti-human IgG Fab-specific HRP-conjugated secondary antibody was applied for detection. Palivizumab was included as the negative control. Optical densities were measured to generate binding curves from which EC50 values were derived. In the flow cytometry assay, MOLM-13 cells expressing human Siglec-15 and MOLM-13 wild-type cells (negative control) were incubated with titrated antibodies starting at 10 μg/ml. After incubation, cells were washed and incubated with an anti-human Fc-specific secondary antibody conjugated with a fluorescent marker. Flow cytometric analysis was conducted, and fluorescence intensity was used to calculate EC50 values, providing a quantitative assessment of the antibodies' cell surface binding efficacy. HEK293T cells were genetically engineered to express full-length mouse Siglec-15 and a truncated version of human Siglec-15 encompassing only domain 2. Antibodies were titrated starting at a concentration of 10 μg/ml and detected with an anti-human Fc-specific secondary antibody to detect bound primary antibodies. The half-maximal effective concentration (EC50) was calculated for each antibody to determine its binding affinity to mouse Siglec-15. Results were analyzed and ranked based on the EC50 values.

### Example 5: Functional Characterization of Anti-Siglec-15 Antibodies

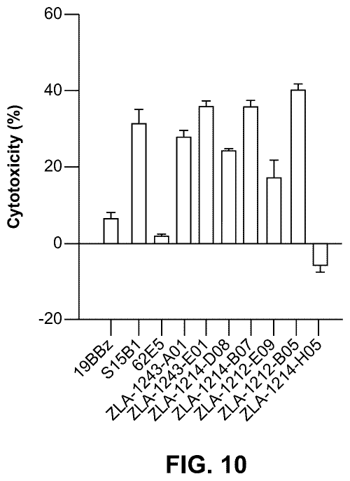

The functional capacity of the generated anti-Siglec-15 monoclonal antibodies, derived from both hybridoma technology and phage display, to modulate T cell cytotoxicity was assessed. An in vitro cytotoxicity assay to evaluate the restoration of T cell antitumor activity in the presence of the antibodies was performed utilizing THP1 cells engineered to overexpress Siglec-15 and the model antigen CD19. This model is particularly relevant as it mirrors the immunosuppressive macrophage environment observed in many solid tumors. Results demonstrate a marked increase in CAR-T cell cytotoxicity against THP1 cells in the presence of antibody clone S15B1 (generated by phage display) and six out of seven tested anti-Siglec-15 antibodies generated by phage display (FIG. 10), highlighting their potential to counteract the immunosuppressive effects of Siglec-15. Conversely, one antibody (clone ZLA-1214-H05) demonstrated a suppressive effect on CAR-T cell cytotoxicity, suggesting a distinct functional profile. Clone 62E5 shown no significant effect neither promoting nor suppressing cytotoxicity. These findings validate the diverse functional capabilities of the generated antibodies and underscore their therapeutic potential in enhancing T cell-mediated tumor cell eradication.

### Materials and Methods

T cells were obtained from buffy coats of healthy donors, provided by Biobanco Vasco, under the ethical approval protocol PI+CES-BIOEF 2019-08. Peripheral Blood Mononuclear Cells (PBMCs) were isolated using Ficoll-Histopaque (17-1440-03, Fisher Scientific) density gradient centrifugation. CD8+ T cells were then purified through negative selection using the EasySep™ Human T Cell Enrichment Kit (Stemcell Technologies), following the manufacturer's instructions. This step ensures a high purity (>95%) of the T cell population, as confirmed by subsequent flow cytometry analysis. The purified T cells were activated using anti-CD3/CD28 Dynabeads (11131D, Thermo Fisher Scientific) in CST OpTimizer T Cell Expansion Serum-Free Medium (A1048501, Gibco) supplemented with IL-2 at a concentration of 100 IU/mL (130-097-743, Miltenyi Biotec). This process is crucial for inducing T cell proliferation and preparing them for effective CAR-T cell formation.

Human full-length Siglec-15 and CD19 genes were synthesized and cloned into the pLV-MSCV lentiviral vector (Genscript), which also contained P2A-Blasticidin and P2A-Puromycin cassettes for selection purposes. To produce lentiviral particles, 5×10\hat{ }6 HEK 293T cells were seeded in a 100 mm dish. After 24 hours, the cells were transfected using a mixture of lentiviral plasmids (5 μg transfer plasmid, 4 μg psPAX2 [Addgene #12260], and 1.5 μg VSV-G [Addgene #8454]) with the jetPEI transfection reagent (101-10 N, Polyplus Transfection), following the manufacturer's guidelines. The supernatant, containing lentiviral particles, was collected 48 hours post-transfection, filtered through a 0.45 μm filter (514-0063, VWR), and concentrated using LentiX Concentrator (631232, Takara) at a 3:1 ratio overnight at 4° C. The concentrated viral particles were then centrifuged at 1500×g for 45 minutes, aliquoted, and stored at −80° C. until further use.

THP1 cells, engineered to overexpress Siglec-15 and CD19, were pre-incubated with each anti-Siglec-15 antibody at a concentration of 10 μg/mL in 150 μL AIMV serum-free media. These pre-treated THP1 cells were then cocultured with anti-CD19 CAR-T cells at an effector-to-target ratio of 0.00625:1. After 36 hours of coculture, the viability of target cells was assessed using an anti-His antibody (dilution 1:1000) to detect cells expressing the His-tagged Siglec-15. The results of this assay provided a quantitative measure of the impact of each anti-Siglec-15 antibody on the cytotoxicity of CAR-T cells, demonstrating their potential to either enhance or suppress T cell-mediated killing of cancer cells.

### Example 6: Binding Characterization of Anti-Siglec-15 Antibodies

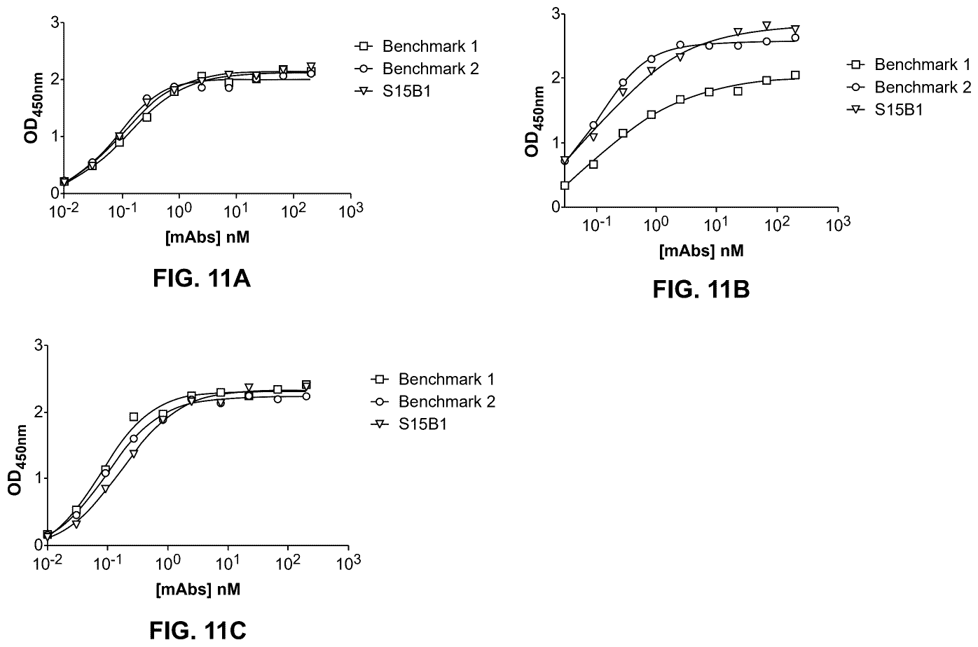

The binding characteristics of the generated anti-Siglec-15 monoclonal antibodies were assessed. ELISA experiments were performed to evaluate the cross-species binding affinity of monoclonal antibody S15B1 to recombinant Siglec-15 proteins derived from humans (FIG. 11A), cynomolgus monkeys (FIG. 11B), and mice (FIG. 11C) compared to benchmark antibodies 5G12 and A2A5C7E8-3. Antibody S15B1 demonstrated a similar binding profile as A2A5C7E8-3 (benchmark 2) across all three species, and both S15B1 and A2A5C7E8-3 bound cynomolgous Siglec-15 to a higher extent than 5G12.

Epitope mapping of the generated anti-Siglec-15 monoclonal antibodies was performed by ELISA of overlapping synthetic peptides. The binding region results are shown in bold below in Table 12, with domain 1 of human Siglec-15 underlined and domain 2 in italics.

TABLE 12
Epitope Mapping synthetic of anti-siglec-15 antibodies, performed
by ELISA of overlapping synthetic peptides.

| Clone | Human Siglec-15 sequence |
|---|---|
| 62E5 | MEKSIWLLACLAWVLPTGSFVRTKIDTTENLLNTEVHSSPAQRWSMQVPPE |
| | VSAEAGDAAVLPCTFTHPHRHYDGPLTAIWRAGEPYAGPQVFRCAAARGSE |
| | LCQTALSLHGRFRLLGNPRRNDLSLRVERLALADDRRYFCRVEFAGDVHDR |
| | YESRHGVRLHVTAAPRIVNISVLPSPAHAFRALCTAEGEPPPALAWSGPALGNS |
| | LAAVRSPREGHGHLVTAELPALTHDGRYTCTAANSLGRSEASVYLFRFHGASGAS |
| | TVALLLGALGFKALLLLGVLAARAARRRPEHLDTPDTPPRSQAQESNYENLSQM |
| | NPRSPPATMCSP |
| S15B1 | MEKSIWLLACLAWVLPTGSFVRTKIDTTENLLNTEVHSSPAQRWSMQVPP |
| | EVSAEAGDAAVLPCTFTHPHRHYDGPLTAIWRAGEPYAGPQVFRCAAARG |
| | SELCQTALSLHGRFRLLGNPRRNDLSLRVERLALADDRRYFCRVEFAGDV |
| | HDRYESRHGVRLHVTAAPRIVNISVLPSPAHAFRALCTAEGEPPPALAWSGPA |
| | LGNSLAAVRSPREGHGHLVTAELPALTHDGRYTCTAANSLGRSEASVYLFRFHG |
| | ASGASTVALLLGALGFKALLLLGVLAARAARRRPEHLDTPDTPPRSQAQESNYE |
| | NLSOMNPRSPPATMCSP |
| ZLA-1243-A01 | MEKSIWLLACLAWVLPTGSFVRTKIDTTENLLNTEVHSSPAQRWSMQVPP |
| | EVSAEAGDAAVLPCTFTHPHRHYDGPLTAIWRAGEPYAGPQVFRCAAAR |
| | GSELCQTALSLHGRFRLLGNPRRNDLSLRVERLALADDRRYFCRVEFAGD |
| | VHDRYESRHGVRLHVTAAPRIVNISVLPSPAHAFRALCTAEGEPPPALAWSG |
| | PALGNSLAAVRSPREGHGHLVTAELPALTHDGRYTCTAANSLGRSEASVYLFR |
| | FHGASGASTVALLLGALGFKALLLLGVLAARAARRRPEHLDTPDTPPRSQAQE |
| | SNYENLSOMNPRSPPATMCSP |
| ZLA-1212-B05 | MEKSIWLLACLAWVLPTGSFVRTKIDTTENLLNTEVHSSPAQRWSMQVP |
| | PEVSAEAGDAAVLPCTFTHPHRHYDGPLTAIWRAGEPYAGPQVFRCAAARG |
| | SELCQTALSLHGRFRLLGNPRRNDLSLRVERLALADDRRYFCRVEFAGDV |
| | HDRYESRHGVRLHVTAAPRIVNISVLPSPAHAFRALCTAEGEPPPALAWSGPA |
| | LGNSLAAVRSPREGHGHLVTAELPALTHDGRYTCTAANSLGRSEASVYLFRFHG |
| | ASGASTVALLLGALGFKALLLLGVLAARAARRRPEHLDTPDTPPRSQAQESNYE |
| | NLSOMNPRSPPATMCSP |
| ZLA-1214-B07 | MEKSIWLLACLAWVLPTGSFVRTKIDTTENLLNTEVHSSPAQRWSMQVP |
| | PEVSAEAGDAAVLPCTFTHPHRHYDGPLTAIWRAGEPYAGPQVFRCAAAR |
| | GSELCQTALSLHGRFRLLGNPRRNDLSLRVERLALADDRRYFCRVEFAGD |
| | VHDRYESRHGVRLHVTAAPRIVNISVLPSPAHAFRALCTAEGEPPPALAWSGP |
| | ALGNSLAAVRSPREGHGHLVTAELPALTHDGRYTCTAANSLGRSEASVYLFRFH |
| | GASGASTVALLLGALGFKALLLLGVLAARAARRRPEHLDTPDTPPRSQAQESNY |
| | ENLSQMNPRSPPATMCSP |
| ZLA-1214-D08 | MEKSIWLLACLAWVLPTGSFVRTKIDTTENLLNTEVHSSPAQRWSMQVPPE |
| | VSAEAGDAAVLPCTFTHPHRHYDGPLTAIWRAGEPYAGPQVFRCAAARGSE |
| | LCQTALSLHGRFRLLGNPRRNDLSLRVERLALADDRRYFCRVEFAGDVHD |
| | RYESRHGVRLHVTAAPRIVNISVLPSPAHAFRALCTAEGEPPPALAWSGPALG |
| | NSLAAVRSPREGHGHLVTAELPALTHDGRYTCTAANSLGRSEASVYLFRFHGAS |
| | GASTVALLLGALGFKALLLLGVLAARAARRRPEHLDTPDTPPRSQAQESNYENLS |
| | QMNPRSPPATMCSP |
| ZLA-1243-E01 | MEKSIWLLACLAWVLPTGSFVRTKIDTTENLLNTEVHSSPAQRWSMQVPPE |
| | VSAEAGDAAVLPCTFTHPHRHYDGPLTAIWRAGEPYAGPQVFRCAAARGSE |
| | LCQTALSLHGRFRLLGNPRRNDLSLRVERLALADDRRYFCRVEFAGDVHD |
| | RYESRHGVRLHVTAAPRIVNISVLPSPAHAFRALCTAEGEPPPALAWSGPALGNS |
| | LAAVRSPREGHGHLVTAELPALTHDGRYTCTAANSLGRSEASVYLFRFHGASGAS |
| | TVALLLGALGFKALLLLGVLAARAARRRPEHLDTPDTPPRSQAQESNYENLSQM |
| | NPRSPPATMCSP |
| ZLA-1212-E09 | MEKSIWLLACLAWVLPTGSFVRTKIDTTENLLNTEVHSSPAQRWSMQVPP |
| | EVSAEAGDAAVLPCTFTHPHRHYDGPLTAIWRAGEPYAGPQVFRCAAARG |
| | SELCQTALSLHGRFRLLGNPRRNDLSLRVERLALADDRRYFCRVEFAGDV |
| | HDRYESRHGVRLHVTAAPRIVNISVLPSPAHAFRALCTAEGEPPPALAWSGPA |
| | LGNSLAAVRSPREGHGHLVTAELPALTHDGRYTCTAANSLGRSEASVYLFRFHG |
| | ASGASTVALLLGALGFKALLLLGVLAARAARRRPEHLDTPDTPPRSQAQESNYEN |
| | LSQMNPRSPPATMCSP |

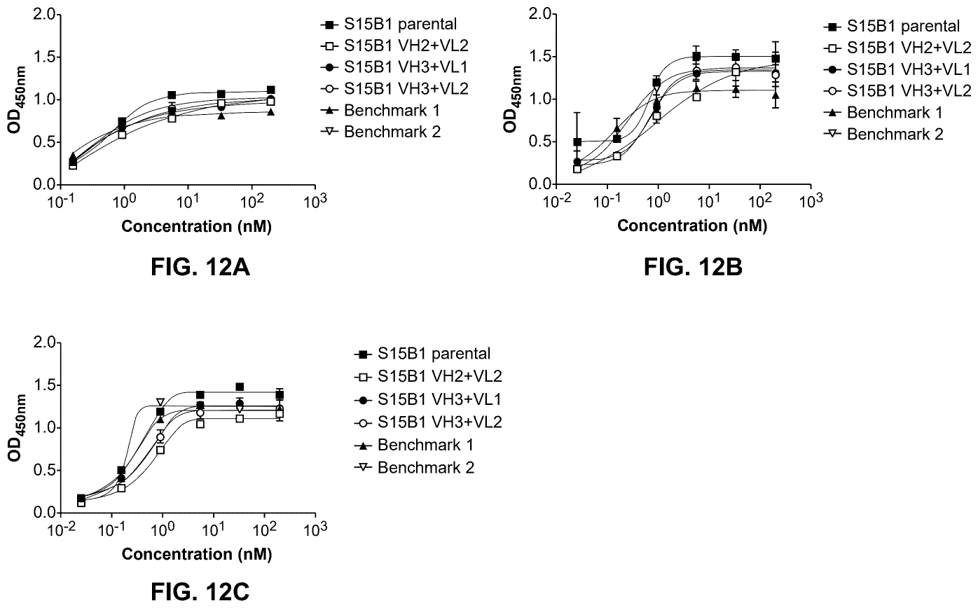

Humanized versions of antibody S15B1 were generated using humanized VH sequences VH1, VH2, VH3, and VH4 in combination with humanized VL sequences VL1 and VL2, shown in Tables 3 and 4. ELISA experiments were performed on antibodies generated using the combinations of humanized S15B1 VH2+VL2, VH3+VL1, and VH3+VL2 to evaluate the binding of each to recombinant Siglec-15 proteins derived from humans (FIG. 12A), cynomolgus monkeys (FIG. 12B), and mice (FIG. 12C) compared to the parental S15B1 antibody and benchmark antibodies 5G12 and A2A5C7E8-3. The humanized S15B1 antibodies demonstrated a similar binding profile as the parental S15B1 antibody, showing that humanization did not disrupt binding capability. The human Siglec-15 binding affinity parameters of the humanized S15B1 antibodies and parental S15B1 antibody, as measured by SPR, are shown in Table 13 below.

TABLE 13
Binding affinity parameters of different combinations of VH +
VL of humanized huS15B1 antibodies and the parental mouse S15B1
antibody to recombinant human Siglec-15 protein, measured by SPR.

| Ligand | Analyte | Chi² (RU²) | ka (1/Ms) | kd (1/s) | KD (M) |
|---|---|---|---|---|---|
| S15B1-VH + VL | human Siglec-15 | 1.33E−01 | 4.46E+06 | 2.33E−04 | 5.22E−11 |
| (Parental) | | | | | |
| S15B1-VH1 + VL1 | human Siglec-15 | 8.11E−02 | 3.35E+06 | 4.55E−04 | 1.36E−10 |
| S15B1-VH1 + VL2 | human Siglec-15 | 6.97E−02 | 3.54E+06 | 5.63E−04 | 1.59E−10 |
| S15B1-VH2 + VL1 | human Siglec-15 | 1.01E−01 | 3.80E+06 | 4.73E−04 | 1.25E−10 |
| S15B1-VH2 + VL2 | human Siglec-15 | 1.05E−01 | 3.44E+06 | 3.26E−04 | 9.46E−11 |
| S15B1-VH3 + VL1 | human Siglec-15 | 9.04E−02 | 3.73E+06 | 2.57E−04 | 6.90E−11 |
| S15B1-VH3 + VL2 | human Siglec-15 | 1.03E−01 | 4.49E+06 | 2.93E−04 | 6.53E−11 |
| S15B1-VH4 + VL1 | human Siglec-15 | 1.03E−01 | 4.10E+06 | 3.85E−04 | 9.38E−11 |
| S15B1-VH4 + VL2 | human Siglec-15 | 2.55E−01 | 1.69E+06 | 2.34E−04 | 1.38E−10 |

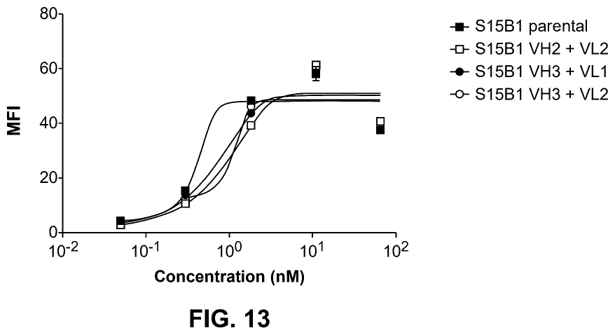

To assess Siglec-15 binding capability on cells, the S15B1 parental and humanized antibody variants were bound to HEK293T cells expressing human Siglec-15 proteins. Flow cytometry was used to quantify the antibodies bound to the HEK293T cells. The S15B1 humanized antibody variants demonstrated a similar binding profile as the S15B1 parental antibody (FIG. 13).

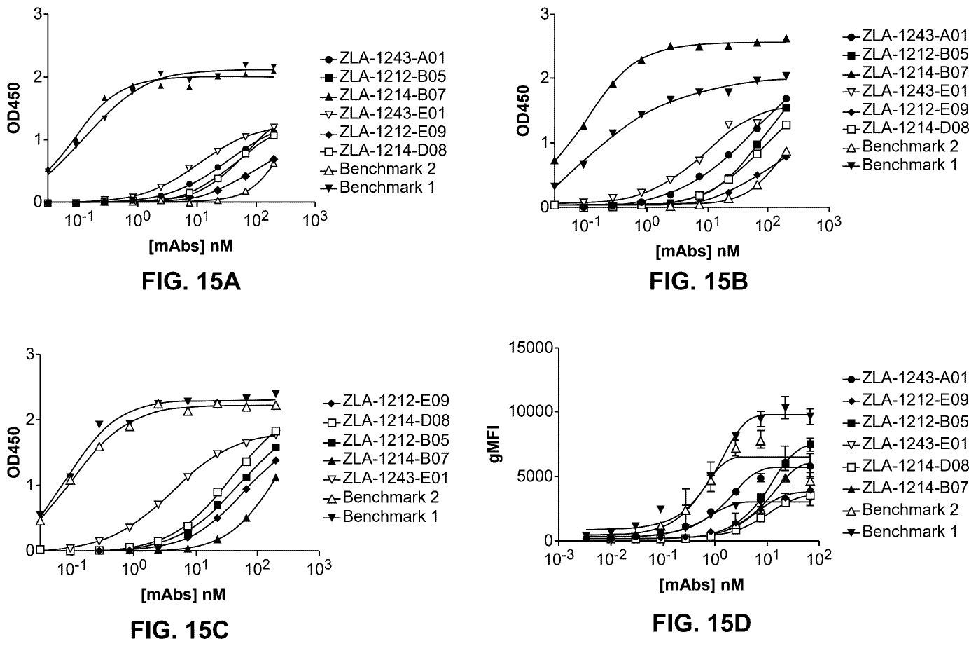

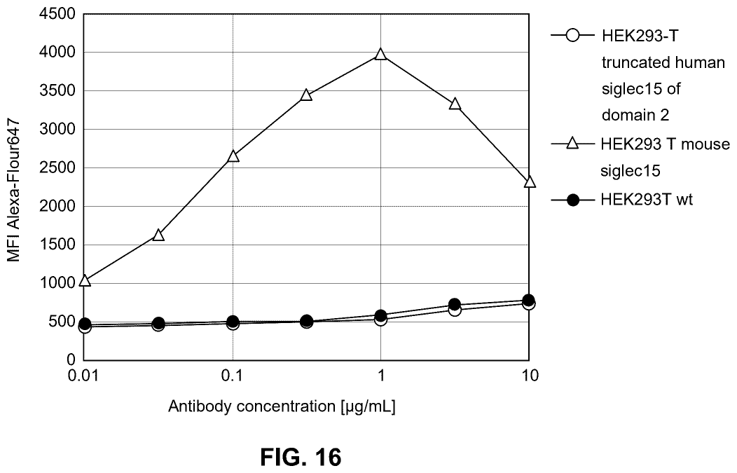

The cross-species binding experiments were repeated on fully human anti-Siglec-15 antibodies and compared to the 5G12 and A2A5C7E8-3 benchmarks. Binding affinity to human Siglec-15 (FIG. 15A), cynomolgus Siglec-15 (FIG. 15B), mouse Siglec-15 (FIG. 15C), and HEK293T cells expressing human Siglec-15 (FIG. 15D) was lower for the fully human anti-Siglec-15 antibodies than for either benchmark antibody. These antibodies were further tested for binding to HEK293T cells expressing mouse Siglec-15 or a truncated version of human Siglec-15 comprising only domain 2 (FIG. 16; clone ZLA-1243-A01 is shown). Binding was demonstrated for mouse Siglec-15 but not domain 2 of human Siglec-15.

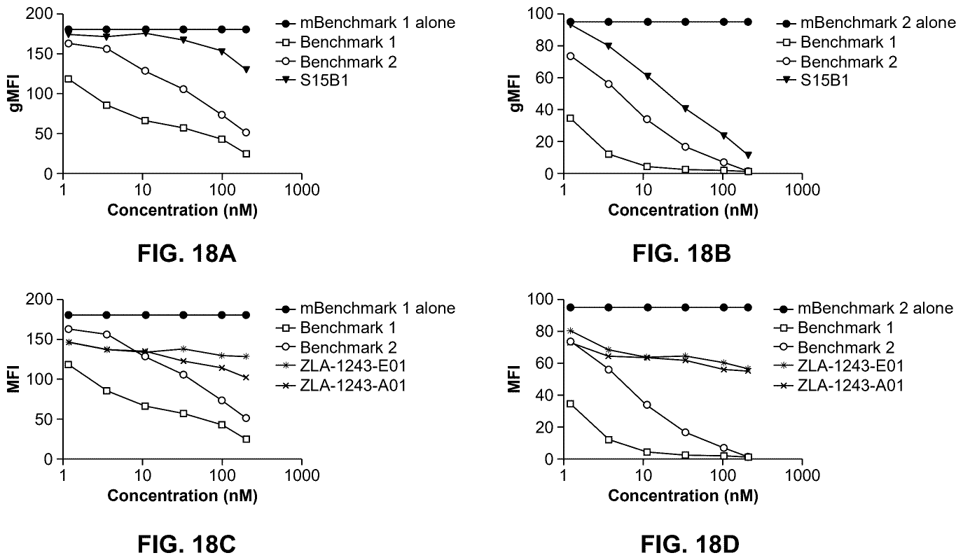

Antibodies S15B1, ZLA-1243-E01, and ZLA-1243-A01 were tested for their ability to inhibit binding of antibodies 5G12 (benchmark 1) and A2A5C7E8 (benchmark 2) in a competitive flow cytometry assay. Antibody S15B1 outperformed A2A5C7E8 in inhibiting binding of 5G12 (FIG. 18A) and outperformed 5G12 in inhibiting binding of A2A5C7E8 (FIG. 18B). Antibodies ZLA-1243-E01 and ZLA-1243-A01 outperformed A2A5C7E8 in inhibiting 5G12 at concentrations above 10 nM (FIG. 18C), and outperformed 5G12 in inhibiting A2A5C7E8 across all concentrations (FIG. 18D).

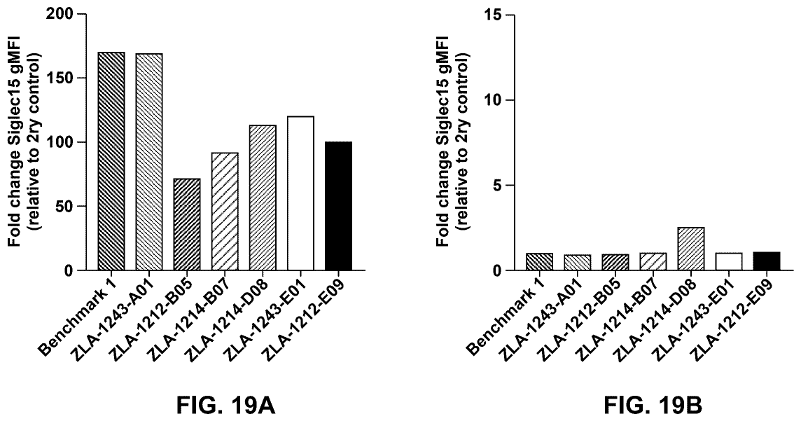

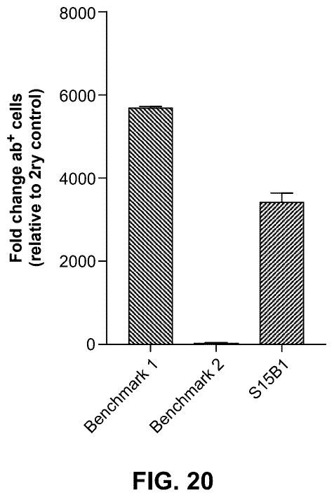

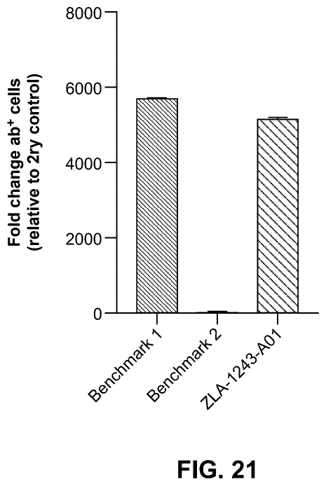

The fully human antibody clones were tested for binding to HEK293T cells expressing full-length (containing domain 1 and domain 2) or domain 2-only human Siglec-15 in comparison to 5G12. All fully human antibody clones demonstrated significant binding to cells expressing full-length Siglec-15 (FIG. 19A) but not to domain 2 of Siglec-15 (FIG. 19B), demonstrating specificity for domain 1 of Siglec-15. Antibody clones were further tested for binding affinity to a mutated version of human Siglec-15 containing an R143A amino acid substitution. Clones S15B1 (FIG. 20) and ZLA-1243-A01 (FIG. 21) demonstrated binding to HEK293T cells expressing mutated Siglec-15, as did the 5G12 control (benchmark 1). The R143A substitution completely eliminated binding of clone A2A5C7E8-3 (benchmark 2).

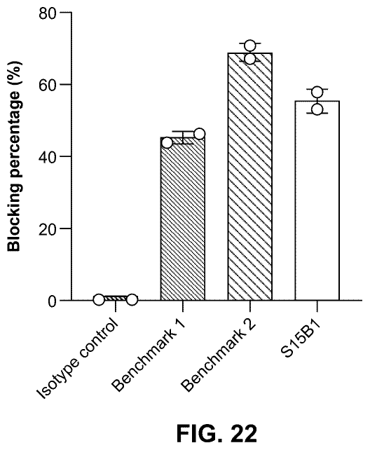

To investigate the efficacy of the described anti-Siglec-15 antibodies in blocking Siglec-15 binding, antibody S15B1 was tested for the ability to block binding of human Siglec-15 to Jurkat cells in a flow cytometry assay. S15B1 was able to block approximately 60% of binding, compared to approximately 40% with the 5G12 clone (benchmark 1) and approximately 70% with the A2A5C7E8-3 clone (benchmark 2) (FIG. 22).

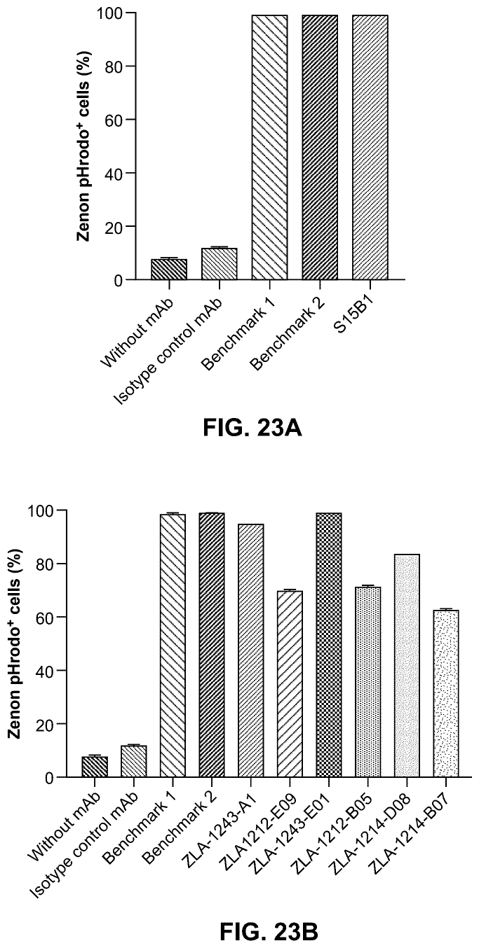

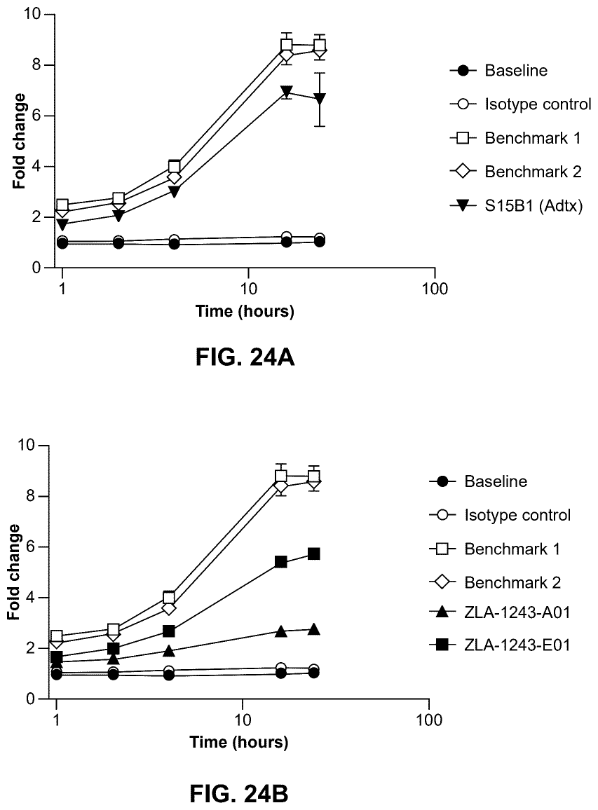

The described anti-Siglec-15 antibodies were then labeled with Zenon pHrodo iFL Green and incubated with cells for 24 hours, followed by quantification to determine the extent to which each antibody was internalized. Antibody S15B1 was internalized in 100% of cells, as were control antibodies 5G12 (benchmark 1) and A2A5C7E8-3 (benchmark 2) (FIG. 23A). The fully human anti-Siglec-15 antibody clones exhibited internalization in 60-100% of cells (FIG. 23B). The internalization kinetics of anti-Siglec-15 antibodies over 1, 2, 4, 16, and 24 hours demonstrated an approximately 6-fold increase over an isotype control for antibodies S15B1 and ZLA-1243-E01, and approximately 2-fold increase for ZLA-1243-A01 (FIGS. 24A and 24B).

### Materials and Methods

Binding to human, cynomolgus and mouse Siglec-15 by ELISA: 96-well micro plates were coated with 50 μl of 2 μg/ml cynomolgus Siglec-15-his protein (Bonopusbio, CW70-50 ug), or 2 μg/ml mouse Siglec-15-his protein (Bonopusbio, CW71-50 ug), or 2 μg/ml human Siglec-15-his protein (Bonopusbio, CW37-50 ug) in carbonate/bicarbonate buffer (pH 9.6) overnight at 4° C. ELISA plates were washed once with wash buffer (PBS+0.1% v/v Tween-20, PBST) and then blocked with 200 μl/well blocking buffer (3% w/v non-fatty milk in PBST) 1 hour at room temperature. Plates were washed 3 times and incubated with 100 pl serially diluted anti-Siglec-15 antibodies of the disclosure or controls (starting at 198 nM, 3-fold serial dilution in PBST with 1% w/v non-fatty milk) for 2 hours at room temperature. ELISA plates were washed 3 times again and incubated with Peroxidase Goat anti-Human IgG Fc Secondary Antibody (100 μl, 1:5000 dilution in PBST buffer, A18817 Thermo Scientific) for 1 hour at room temperature. After another 3 washes, plates were incubated with 100 μl/well TMB (N301, Thermo Scientific). The reaction was stopped 2 minutes later at room temperature with 50 μl of STOP solution (N600, Thermo Scientific), and the absorbance of each well was read on a NIVO microplate reader at 450. OD values were plotted against antibody concentration. Data was analyzed using Graphpad Prism software.

Binding affinity determination of anti-Siglec-15 monoclonal antibodies using BIACORE surface plasmon resonance: The purified anti-Siglec-15 monoclonal antibodies (mAbs) were characterized for binding affinity and binding kinetics by Biacore T200 system (GE healthcare, Pittsburgh, PA, USA). Anti-Siglec-15 antibodies were covalently linked to a CM5 chip (carboxy methyl dextran coated chip, GE healthcare, Cat #BR-1005-30) via primary amines, using a standard amine coupling kit provided by Biacore (GE healthcare, Pittsburgh, PA, USA). Un-reacted moieties on the biosensor surface were blocked with ethanolamine. Then, serially diluted recombinant human Siglec-15-his protein (Bonopusbio, CW70-50 ug), 2-fold serial dilution in HBS-EP buffer starting at 80 nM, were respectively flown onto the chip at a flow rate of 30 pL/min. The antigen-antibody association kinetics was followed for 2 minutes, and the dissociation kinetics was followed for 10 minutes. The association and dissociation curves were fit to a 1:1 Langmuir binding model using BIAcore evaluation software. The dissociation rate (kd) and association constant (ka) rate constants were obtained using Evaluation Software. The equilibrium dissociation constant (KD) was calculated from the ratio of kd over ka.

Assessment of the binding of anti-Siglec-15 antibodies to HEK293T cells expressing full length human Siglec-15, or mutant human Siglec-15 (R143A) or the domain 2 of human Siglec-15 by Flow cytometry: Siglec-15+, Siglec-15+R143A, and Siglec-15 Domain 2 (D2) HEK293T cells were detached using tryple and washed once with PBS, resuspended in 1× flow cytometry buffer (PBS with 1% FBS) at approximately 1-2×10\hat{ }6 cells/mL. Then, anti-Siglec-15 mAbs were added to the cell suspension at the desired concentration, starting at 10 μg/mL and performing 1/3 dilutions. Cells were incubated with the indicated antibodies for 30-60 minutes at 4° C. in the dark to prevent degradation of the fluorescent signal. After the primary antibody incubation, cells were washed twice with PBS or flow cytometry buffer by centrifuging at 300-500× g for 5 minutes and the pellet resuspended in fresh buffer. The following antibody was added: anti-human IgG-PE at a 1:500 dilution. Cells were incubated with the secondary antibody for 30 minutes at 4° C. in the dark. Finally, cells were washed twice with PBS or flow cytometry buffer to remove any excess secondary antibody and acquired in a flow cytometer.

Epitope Mapping by synthetic epitopes via ELISA: Peptide Synthesis: To reconstruct epitopes of the target molecule, a library of peptide-based mimics was synthesized using Fmoc-based solid-phase peptide synthesis. The process begins with grafting an amino-functionalized polypropylene support with a proprietary hydrophilic polymer formulation, followed by reaction with butyloxycarbonyl-t-hexamethylenediamine (BocHMDA) using dicyclohexylcarbodiimide (DCC) and N-hydroxybenzotriazole (HOBt). The Boc-groups are then cleaved using trifluoroacetic acid (TFA). Standard Fmoc-peptide synthesis was used to synthesize peptides on the amino-functionalized solid support using custom-modified JANUS liquid handling stations (Perkin Elmer). If constrained peptides were involved in the study, Biosynth's proprietary Chemically Linked Peptides on Scaffolds (CLIPS) technology was employed. This technology enables the structure of peptides into various configurations, including single loops, double loops, triple loops, sheet-like folds, helix-like folds, or combinations thereof. The CLIPS scaffold is coupled to cysteine residues, with the side-chains of multiple cysteines in the peptides being linked to a CLIPS scaffold containing two or three reactive groups. For example, a 0.5 mM solution of the P2 CLIPS (2,6-bis(bromomethyl)pyridine) is dissolved in ammonium bicarbonate (20 mM, pH 7.8) and acetonitrile (1: 3 (v/v)). This solution is then added to the peptide arrays. The CLIPS template binds to side-chains of two cysteines introduced in the solid-phase bound peptides on a 455-well plate (3 μl per well). The arrays are gently shaken in this solution for 30-60 minutes. Afterward, the peptide arrays are extensively washed with excess water and sonicated in disrupt-buffer (1% SDS/0.1% 2,2′-(Ethylenedioxy) diethanethiol in PBS, pH 7.2) at 70° C. for 30 minutes, followed by additional sonication in water for another 45 minutes. Peptides carrying T3 CLIPS are synthesized similarly, but with three cysteines.

ELISA Screening: The binding of antibodies to each of the synthesized peptides was tested using an ELISA based on the Biosynth platform. The peptide arrays were incubated with primary antibody solution under two conditions: either overnight at 4° C. with a small amount of solution on top of the array (NORMAL) or at room temperature in a larger volume with agitation (BOX). After washing, the peptide arrays were incubated for one hour at 25° C. with a 1/1000 dilution of an appropriate peroxidase-conjugated secondary antibody—either goat anti-human HRP conjugate (Southern Biotech, 2010-05) or rabbit anti-mouse IgG (H+L) HRP conjugate (Southern Biotech, 6175-05). Following another wash, the peroxidase substrate, ABTS (2,2′-azino-di-3-ethylbenzthiazoline sulfonate), and 20 μl/ml of 3% hydrogen peroxide (H2O2) were added. After an hour, the color development was measured and quantified using a charge-coupled device (CCD) camera and an image processing system. This approach allows for a comprehensive evaluation of antibody binding to synthetic peptide epitopes, enabling effective epitope mapping.

Epitope mapping assay by Flow cytometry: Competition with benchmark mouse IgG antibodies: 50,000 cells/well of THP-1 S15 OE were added to a round bottom 96 well plate, and centrifuged at 450×G for 5 minutes, and the supernatant was discarded. Human anti-siglec-15 IgG antibodies (starting at 198 nM, 3-fold serial dilution in staining buffer) were added to 100 μl of staining buffer and cells were resuspended with it and incubated for 40 minutes on ice. 100 μl of staining buffer were added to wells, centrifuged at 450× G for 5 minutes and the supernatants discarded. Benchmark mouse IgG antibodies were diluted at 33.3 nM and cells resuspended in 100 μl of the mix and incubated for 40 minutes on ice. Then, 100 μl of staining buffer were added to wells, centrifuged at 450×G for 5 minutes and the supernatants discarded. The secondary antibody anti-mouse IgG-Alexa647 was prepared at 1:200 dilution in 100 μl of staining buffer and added to each well, and cells were incubated for 40 minutes on ice. Cells were washed with 100 μl of staining buffer and centrifuged at 450×G for 5 minutes to discard the supernatant. Cells were resuspended in 150 μl of staining buffer and acquired in a flow cytometer.

Blocking assay (Flow cytometry): Briefly, the anti-Siglec-15 antibodies of the disclosure or controls were diluted with human Siglec-15-human Fc protein (Sinobiologicals, 13976-H02H1), at 66.67 nM, and incubated at room temperature for 40 minutes. Then, Jurkat T cells were harvested from cell culture flasks, washed twice, and re-suspended in PBS containing 2% v/v Bovine serum albumin (FACS buffer). Then, 0.5×10⁵ cells per well in 96 well-plates were incubated in 100 μL of the antibody/Siglec-15-human Fc mixtures for 40 minutes at 4° C. The plates were washed twice with FACS buffer, and then added and incubated for 40 minutes at 4° C. in dark with 100 μl/well Goat Anti-human IgG (H+L) (1:200 dilution in FACS buffer, Thermofisher). Cells were washed twice and re-suspended in FACS buffer. Fluorescence was measured in a flow cytometer. Data was analyzed using Flowjo and Graphpad Prism software.

Internalization assay (Zhenon pHrodo): Sufficient volumes of 4× working solution of anti-Siglec-15 antibodies (10 nM) were prepared in cell culture medium for a total of 25 μL for each well of a 96-well plate. A working solution of Zenon™ pHrodo™ iFL IgG Labeling Reagent was prepared at 30 nM and 25 μL were added to each well. The mix was incubated for 5 minutes at room temperature to allow the labeling complexes to form. 5 mL of suspension cells at 2×10⁶ cells/mL in cell culture medium were prepared and 50 μL of cells were added to each well of the 96-well plate containing the mix of anti-Siglec-15 antibody and the Zenon™ labeling reagent. Cells were incubated with the labeling complex for different timepoints (1-24 hours) under standard cell culture conditions. Fluorescence was analyzed by flow cytometry.

### Example 7: Characterization of Anti-Siglec-15 Antibody-Drug Conjugates (ADCs) and Bispecific Antibodies

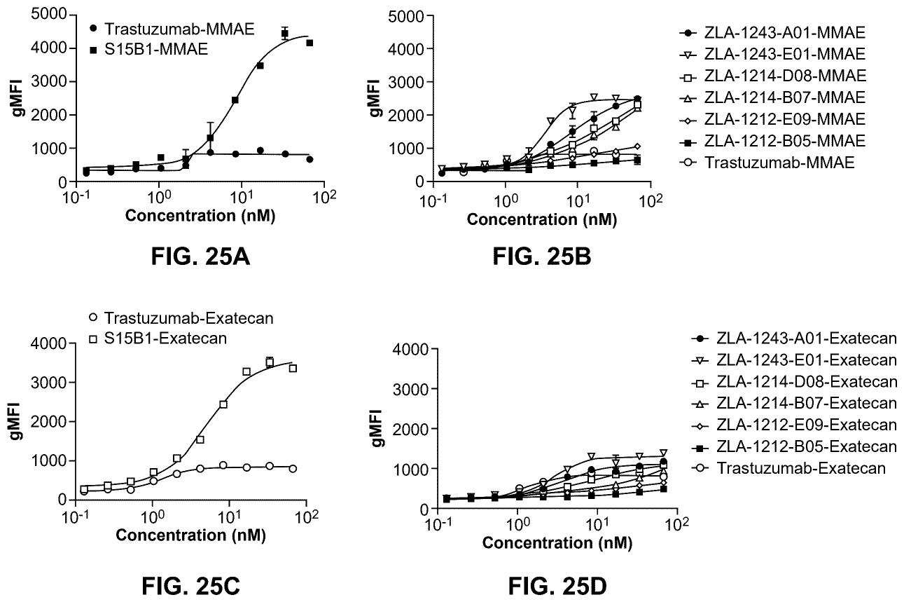

Antibody-drug conjugates (ADCs) with anti-Siglec-15 antibodies and MMAE or Exatecan were prepared and tested for the ability to bind THP-1 cells overexpressing human Siglec-15. The S15B1-MMAE ADC demonstrated dramatically increased binding capacity to THP-1 cells relative to a Trastuzumab-based ADC negative control, as measured by flow cytometry (FIG. 25A). Fully human clone-based ADCs ZLA-1243-A01-MMAE, ZLA-1243-E01-MMAE, ZLA-1243-D08-MMAE, and ZLA-1243-B07-MMAE also demonstrated increased binding capacity relative to a trastuzumab-based ADC negative control (FIG. 25B). Exatecan-containing ADCs demonstrated similar binding patterns, albeit slightly reduced relative to trastuzumab (FIGS. 25C and 25D).

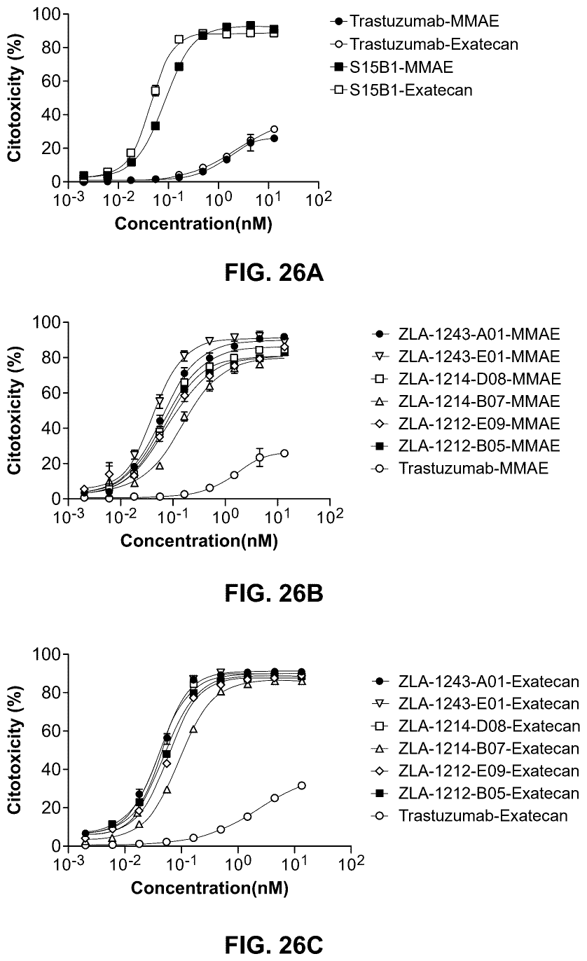

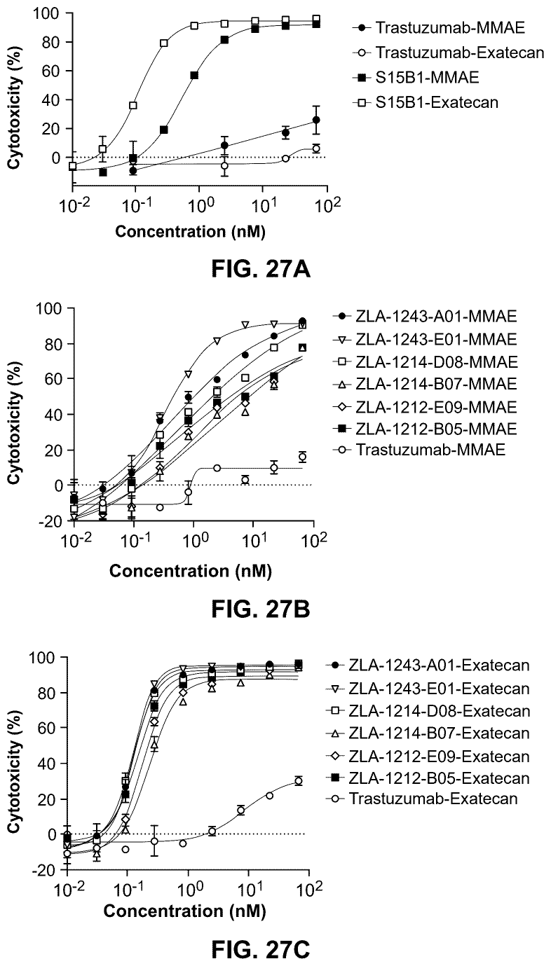

Cytotoxic effects of the ADCs were assessed by counting DAPI-positive THP-1 cells via flow cytometry after 48 hours of culture. S15B1-MMAE and S15B1-Exatecan demonstrated approximately 90% cytotoxicity, compared to approximately 20% with trastuzumab controls (FIG. 26A). Fully human antibody-based ADCs similarly demonstrated approximately 80-90% cytoxicity with MMAE (FIG. 26B) and Exatecan (FIG. 26C) payloads. Cytotoxicity was also measured by an ATP assay after 48 hours of culture. S15B1-based ADCs demonstrated nearly 100% cytotoxicity, with S15B1-Exatecan achieving these results at a lower concentration than S15B1-MMAE (FIG. 27A). Fully human antibody ADCs demonstrated approximately 70-95% cytotoxicity with an MMAE payload (FIG. 27B), and approximately 90-100% cytotoxicity with an Exatecan payload at lower concentrations (FIG. 27C).

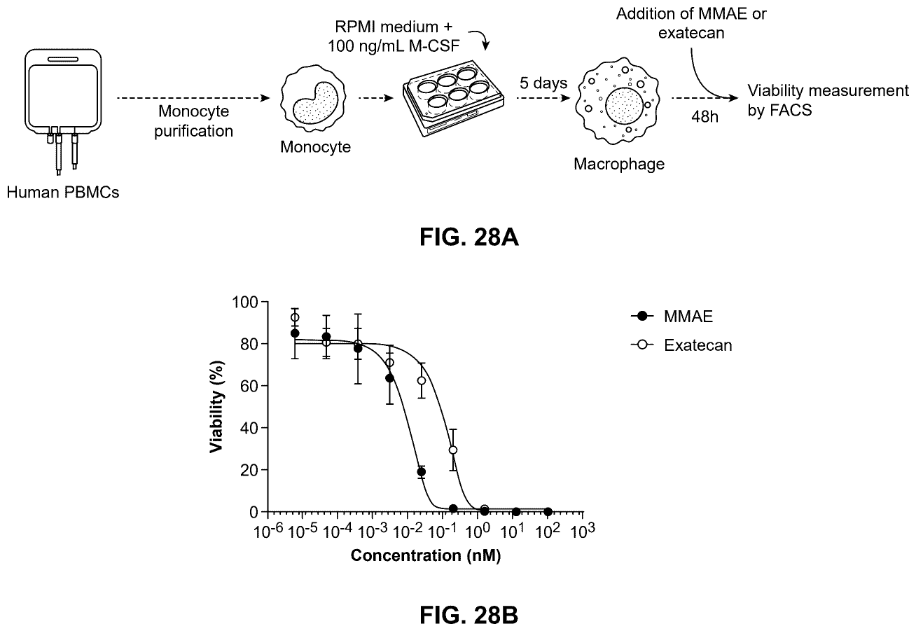

The MMAE and Exatecan payloads were then tested in human macrophages. Human PBMCs were obtained, purified for monocytes, and differentiated into macrophages prior to incubation with MMAE or Exatecan (FIG. 28A). Viability of macrophages after a 48-hour incubation was assessed via flow cytometry counting of calcein-positive and Ethidium Homodimer-1 negative cells. MMAE produced loss of cell viability at a lower concentration than Exatecan.

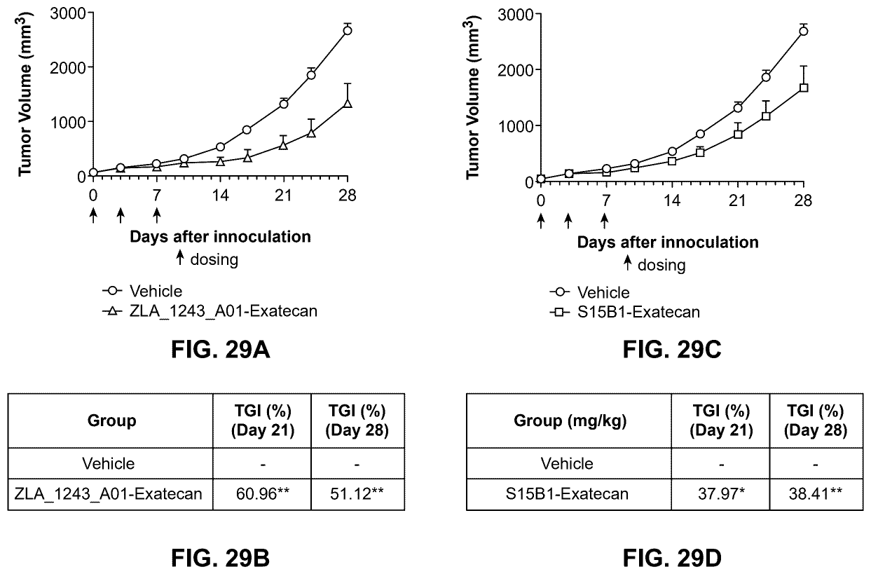

Balb/c mice with subcutaneous EMT-6 breast tumor model cells were treated with anti-Siglec-15 ADCs or a vehicle control. Administration of three doses of the ZLA-1243-A01-Exatecan ADC resulted in less than half the tumor volume compared to administration of the vehicle control by day 28 (FIG. 29A) and significantly higher tumor growth inhibition percentages (FIG. 29B). The S15B1-Exatecan ADC also produced reduced tumor volume (FIG. 29C) and significantly increased tumor growth inhibition percentages (FIG. 29D) relative to the vehicle control.

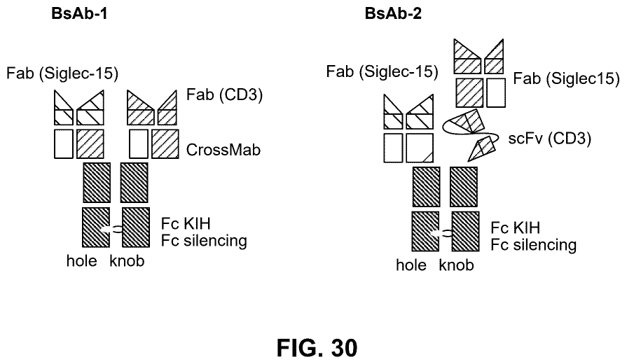

Two formats of bispecific antibodies to Siglec-15 and CD3, BsAb-1 and BsAb-2, were constructed (FIG. 30). Clone SP34, a mouse anti-CD3 monoclonal antibody, was humanized for use in bispecific Siglec-15 and CD3 antibodies. Sequences of the humanized anti-human CD3 antibody used in the bispecific constructs are shown below in Table 14, and the sequences of BsAb-1 and BsAb-2 are shown in Table 15 and Table 16.

TABLE 14
Sequences of the anti-human CD3 humanized antibody used
for the bispecific constructs.
Humanized SP34 CD3 antibody

| | |
|---|---|
| >VH-3-15 | EVOLVESGGGLVKPGGSLRLSCAASGFTFNTYAMNWVRQAPGKG |
| | LEWVARIRSKYNNYATYYADSVKDRFTISRDDSKNTLYLQMNSLK |
| | TEDTAVYYCVRHGNFGNSYVSWFAYWGQGTLVTVSS (SEQ ID |
| | NO: 109) |
| >VL-8-61 | QTVVTQEPSFSVSPGGTVTLTCRSSTGAVTTSNYANWVQQTPGQA |
| | PRGLIGGTNKRAPGVPARFSGSILGNKAALTITGAQADDEAEYFCA |
| | LWYSNLWVFGGGTKLTVL (SEQ ID NO: 110) |
| >VH-3-15 | GAAGTGCAACTAGTGGAAAGTGGTGGTGGTCTCGTGAAACCCG |
| DNA | GCGGATCTCTGAGACTGTCCTGTGCTGCTTCTGGCTTCACCTTCA |
| | ACACCTACGCCATGAACTGGGTCAGACAGGCTCCTGGCAAGGG |
| | CCTGGAATGGGTGGCCAGAATCCGGTCCAAGTACAACAACTAC |
| | GCTACATATTACGCCGACTCCGTGAAGGACCGGTTTACCATCTC |
| | CAGAGATGACTCTAAGAACACACTGTACCTGCAGATGAATAGC |
| | CTGAAGACCGAGGACACCGCCGTGTACTACTGCGTGCGGCACG |
| | GCAACTTCGGCAACTCCTACGTGTCTTGGTTCGCCTACTGGGGC |
| | CAGGGCACCCTGGTGACCGTGTCCAGC (SEQ ID NO: 125) |
| >VL-8-61 | CAAACTGTGGTGACTCAAGAACCAAGTTTTTCCGTGTCCCCAGG |
| DNA | AGGTACCGTGACACTGACCTGTCGGAGCTCTACCGGCGCCGTGA |
| | CCACATCTAACTACGCCAACTGGGTCCAACAGACCCCTGGCCAG |
| | GCTCCTAGAGGCCTGATCGGCGGCACCAATAAACGGGCCCCTG |
| | GCGTGCCCGCTAGATTTTCTGGCTCCATCCTGGGCAACAAGGCC |
| | GCTCTCACCATCACCGGCGCTCAGGCCGACGATGAAGCCGAGT |
| | ACTTCTGCGCCCTGTGGTACTCCAACCTGTGGGTGTTCGGCGGC |
| | GGAACCAAGCTGACAGTGCTG (SEQ ID NO: 126) |

TABLE 15
Sequences of the S15 × CD3 bispecific antibodies (BsAb-1 and BsAb-2).

| Name | Format | Chain | Chain | Sequence |
|---|---|---|---|---|
| BsAb-1 | Format | Siglec 15 | VL1 + | DIVLTQSPASLAVSLGQRATISCRASKSVSTSGYS |
| | -1: KIH | Fab hole | CL | YMHWYQQKPGQPPKLLIYLASNLESGVPARFSG |
| | & | | | SGSGTDFTLNIHPVEEEDAATYYCQHGRELPFTF |
| | CrossmAb | | | GSGTKLEIKRTVAAPSVFIFPPSDEQLKSGTASVV |
| | | | | CLLNNFYPREAKVQWKVDNALQSGNSQESVTE |
| | | | | QDSKDSTYSLSSTLTLSKADYEKHKVYACEVTH |
| | | | | QGLSSPVTKSFNRGEC (SEQ ID NO: 111) |
| | | | VH1 + | QVQLQQSGPELVKPGASVKISCKASGYAFSSSW |
| | | | CH | MNWVKQRPGKGLEWIGRIYPGHGETNYNGKFK |
| | | | | DKATVTADKSSSTTYMQLSSLTSEDSAVYFCAR |
| | | | | VDNSDPYYFDYWGQGTTLTVSSASTKGPSVFPL |
| | | | | APSSKSTSGGTAALGCLVKDYFPEPVTVSWNSG |
| | | | | ALTSGVHTFPAVLQSSGLYSLSSVVTVPSSSLGT |
| | | | | QTYICNVNHKPSNTKVDKKVEPKSCDKTHTCPP |
| | | | | CPAPEAAGGPSVFLFPPKPKDTLMISRTPEVTCV |
| | | | | VVDVSHEDPEVKFNWYVDGVEVHNAKTKPREE |
| | | | | QYNSTYRVVSVLTVLHQDWLNGKEYKCKVSNK |
| | | | | ALPAPIEKTISKAKGQPREPQVCTLPPSRDELTKN |
| | | | | QVSLSCAVKGFYPSDIAVEWESNGQPENNYKTT |
| | | | | PPVLDSDGSFFLVSKLTVDKSRWQQGNVFSCSV |
| | | | | MHEALHNHYTQKSLSLSPGK (SEQ ID NO: 112) |
| | | CD3 | VH2 + | EVQLVESGGGLVKPGGSLRLSCAASGFTFNTYA |
| | | knob | CH | MNWVRQAPGKGLEWVARIRSKYNNYATYYAD |
| | | | | SVKDRFTISRDDSKNTLYLQMNSLKTEDTAVYY |
| | | | | CVRHGNFGNSYVSWFAYWGQGTLVTVSSGQPK |
| | | | | AAPSVTLFPPSSEELQANKATLVCLISDFYPGAVT |
| | | | | VAWKADSSPVKAGVETTTPSKQSNNKYAASSYL |
| | | | | SLTPEQWKSHRSYSCQVTHEGSTVEKTVAPTECS |
| | | | | DKTHTCPPCPAPEAAGGPSVFLFPPKPKDTLMISR |
| | | | | TPEVTCVVVDVSHEDPEVKFNWYVDGVEVHNA |
| | | | | KTKPREEQYNSTYRVVSVLTVLHQDWLNGKEY |
| | | | | KCKVSNKALPAPIEKTISKAKGQPREPQVYTLPP |
| | | | | CRDELTKNQVSLWCLVKGFYPSDIAVEWESNGQ |
| | | | | PENNYKTTPPVLDSDGSFFLYSKLTVDKSRWQQ |
| | | | | GNVFSCSVMHEALHNHYTQKSLSLSPGK (SEQ |
| | | | | ID NO: 113) |
| | | | VL2 + | QTVVTQEPSFSVSPGGTVTLTCRSSTGAVTTSNY |
| | | | CL | ANWVQQTPGQAPRGLIGGTNKRAPGVPARFSGS |
| | | | | ILGNKAALTITGAQADDEAEYFCALWYSNLWVF |
| | | | | GGGTKLTVLASTKGPSVFPLAPSSKSTSGGTAAL |
| | | | | GCLVKDYFPEPVTVSWNSGALTSGVHTFPAVLQ |
| | | | | SSGLYSLSSVVTVPSSSLGTQTYICNVNHKPSNTK |
| | | | | VDKKVEPKSC (SEQ ID NO: 114) |
| BsAb-2 | Format | Siglec 15 | VL1 + | DIVLTQSPASLAVSLGQRATISCRASKSVSTSGYS |
| | -2: KIH | Fab hole | CL | YMHWYQQKPGQPPKLLIYLASNLESGVPARFSG |
| | & scFv | | | SGSGTDFTLNIHPVEEEDAATYYCQHGRELPFTF |
| | (2 + 1) | | | GSGTKLEIKRTVAAPSVFIFPPSDEQLKSGTASVV |
| | | | | CLLNNFYPREAKVQWKVDNALQSGNSQESVTE |
| | | | | QDSKDSTYSLSSTLTLSKADYEKHKVYACEVTH |
| | | | | QGLSSPVTKSFNRGEC (SEQ ID NO: 115) |
| | | | VH1 + | QVQLQQSGPELVKPGASVKISCKASGYAFSSSW |
| | | | VH | MNWVKQRPGKGLEWIGRIYPGHGETNYNGKFK |
| | | | | DKATVTADKSSSTTYMQLSSLTSEDSAVYFCAR |
| | | | | VDNSDPYYFDYWGQGTTLTVSSASTKGPSVFPL |
| | | | | APSSKSTSGGTAALGCLVKDYFPEPVTVSWNSG |
| | | | | ALTSGVHTFPAVLQSSGLYSLSSVVTVPSSSLGT |
| | | | | QTYICNVNHKPSNTKVDKKVEPKSCDKTHTCPP |
| | | | | CPAPEAAGGPSVFLFPPKPKDTLMISRTPEVTCV |
| | | | | VVDVSHEDPEVKFNWYVDGVEVHNAKTKPREE |
| | | | | QYNSTYRVVSVLTVLHQDWLNGKEYKCKVSNK |
| | | | | ALPAPIEKTISKAKGQPREPQVCTLPPSRDELTKN |
| | | | | QVSLSCAVKGFYPSDIAVEWESNGQPENNYKTT |
| | | | | PPVLDSDGSFFLVSKLTVDKSRWQQGNVFSCSV |
| | | | | MHEALHNHYTQKSLSLSPGK (SEQ ID NO: 116) |
| | | CD3 | VH2 + | QVQLQQSGPELVKPGASVKISCKASGYAFSSSW |
| | | knob | VH | MNWVKQRPGKGLEWIGRIYPGHGETNYNGKFK |
| | | | | DKATVTADKSSSTTYMQLSSLTSEDSAVYFCAR |
| | | | | VDNSDPYYFDYWGQGTTLTVSSASTKGPSVFPL |
| | | | | APSSKSTSGGTAALGCLVKDYFPEPVTVSWNSG |
| | | | | ALTSGVHTFPAVLQSSGLYSLSSVVTVPSSSLGT |
| | | | | QTYICNVNHKPSNTKVDKKVEPKSCGGGGSGGG |
| | | | | GSGGGGSQTVVTQEPSFSVSPGGTVTLTCRSSTG |
| | | | | AVTTSNYANWVQQTPGQAPRGLIGGTNKRAPG |
| | | | | VPARFSGSILGNKAALTITGAQADDEAEYFCAL |
| | | | | WYSNLWVFGGGTKLTVLGGGGSGGGGSGGGGS |
| | | | | GGGGSEVQLVESGGGLVKPGGSLRLSCAASGFT |
| | | | | FNTYAMNWVRQAPGKGLEWVARIRSKYNNYAT |
| | | | | YYADSVKDRFTISRDDSKNTLYLQMNSLKTEDT |
| | | | | AVYYCVRHGNFGNSYVSWFAYWGQGTLVTVSS |
| | | | | GGGGSGGGGSGGGGSDKTHTCPPCPAPEAAGGP |
| | | | | SVFLFPPKPKDTLMISRTPEVTCVVVDVSHEDPE |
| | | | | VKFNWYVDGVEVHNAKTKPREEQYNSTYRVVS |
| | | | | VLTVLHQDWLNGKEYKCKVSNKALPAPIEKTIS |
| | | | | KAKGQPREPQVYTLPPCRDELTKNQVSLWCLVK |
| | | | | GFYPSDIAVEWESNGQPENNYKTTPPVLDSDGSF |
| | | | | FLYSKLTVDKSRWQQGNVFSCSVMHEALHNHY |
| | | | | TQKSLSLSPGK (SEQ ID NO: 117) |
| | | | VL2 + | DIVLTQSPASLAVSLGQRATISCRASKSVSTSGYS |
| | | | CL | YMHWYQQKPGQPPKLLIYLASNLESGVPARFSG |
| | | | | SGSGTDFTLNIHPVEEEDAATYYCQHGRELPFTF |
| | | | | GSGTKLEIKRTVAAPSVFIFPPSDEQLKSGTASVV |
| | | | | CLLNNFYPREAKVQWKVDNALQSGNSQESVTE |
| | | | | QDSKDSTYSLSSTLTLSKADYEKHKVYACEVTH |
| | | | | QGLSSPVTKSFNRGEC (SEQ ID NO: 118) |

TABLE 16
DNA Sequences of the S15 x CD3 bispecific antibodies (BsAb-1 and BsAb-2).

| Name | Format | Chain | Chain | Sequence |
|---|---|---|---|---|
| BsAb-1 | Format | Siglec 15 | VL1 + | GATATCGTGCTAACTCAAAGTCCAGCAAGTC |
| | -1: KIH & | Fab hole | CL | TGGCCGTGTCTCTGGGCCAACGGGCCACCAT |
| | CrossmAb | | | CAGCTGTAGAGCCTCTAAGTCCGTGTCCACC |
| | | | | TCTGGCTACTCCTACATGCACTGGTATCAGC |
| | | | | AGAAGCCTGGCCAGCCTCCAAAACTGCTGAT |
| | | | | CTACCTGGCTTCCAACCTGGAATCTGGAGTC |
| | | | | CCCGCTCGGTTCTCCGGCAGCGGCTCTGGAA |
| | | | | CAGATTTTACCCTGAACATCCATCCTGTGGA |
| | | | | AGAGGAGGACGCCGCTACCTACTACTGCCAG |
| | | | | CACGGCAGAGAGCTGCCTTTCACCTTCGGCT |
| | | | | CCGGCACCAAGCTCGAGATCAAGAGGACAG |
| | | | | TGGCCGCCCCAAGCGTGTTCATCTTTCCCCCT |
| | | | | TCCGACGAGCAGCTGAAGTCTGGCACCGCCA |
| | | | | GCGTGGTGTGCCTGCTGAACAACTTCTACCC |
| | | | | TCGGGAGGCCAAGGTCCAGTGGAAGGTGGA |
| | | | | TAACGCCCTGCAGTCTGGCAATAGCCAGGAG |
| | | | | TCCGTGACCGAGCAGGACTCTAAGGATAGCA |
| | | | | CATATTCCCTGTCTAGCACCCTGACACTGAG |
| | | | | CAAGGCCGATTACGAGAAGCACAAGGTGTA |
| | | | | TGCCTGTGAAGTCACCCATCAGGGGCTGTCA |
| | | | | TCACCCGTCACTAAGTCATTCAATCGCGGAG |
| | | | | AATGC (SEQ ID NO: 127) |
| | | | VH1 + | CAAGTGCAACTACAACAAAGTGGTCCAGAA |
| | | | CH | CTGGTGAAGCCTGGAGCTTCTGTGAAGATCT |
| | | | | CCTGCAAGGCCTCTGGCTACGCCTTCTCCTCT |
| | | | | TCTTGGATGAATTGGGTCAAGCAGCGGCCTG |
| | | | | GCAAAGGCCTGGAATGGATCGGCAGAATCT |
| | | | | ACCCCGGACACGGCGAGACCAACTACAACG |
| | | | | GCAAGTTCAAGGACAAAGCTACAGTGACCG |
| | | | | CTGATAAGTCCAGCTCTACCACCTACATGCA |
| | | | | GCTGTCCTCTCTGACCTCCGAGGACTCCGCC |
| | | | | GTGTATTTTTGTGCCAGAGTGGACAACTCCG |
| | | | | ATCCTTACTACTTCGACTACTGGGGCCAGGG |
| | | | | CACAACCCTGACCGTGTCCAGCGCCAGCACC |
| | | | | AAAGGCCCCTCCGTGTTCCCCCTGGCCCCTA |
| | | | | GCTCCAAGTCTACCTCTGGCGGTACTGCTGC |
| | | | | CCTGGGCTGCCTGGTGAAGGACTACTTCCCT |
| | | | | GAGCCTGTGACAGTGTCCTGGAACTCCGGAG |
| | | | | CTCTGACCTCCGGCGTGCATACCTTTCCTGCT |
| | | | | GTGCTGCAATCCTCTGGACTGTACTCCCTGTC |
| | | | | CTCTGTGGTGACCGTGCCATCTTCCAGCCTG |
| | | | | GGCACCCAGACCTACATCTGCAACGTGAACC |
| | | | | ACAAGCCTTCCAACACCAAGGTGGACAAGA |
| | | | | AAGTGGAACCTAAGTCCTGCGATAAGACTCA |
| | | | | TACTTGCCCACCATGCCCAGCTCCTGAGGCT |
| | | | | GCTGGTGGCCCTTCTGTGTTTCTGTTCCCTCC |
| | | | | TAAGCCCAAGGACACCCTGATGATCTCTCGG |
| | | | | ACCCCTGAAGTGACCTGCGTGGTCGTGGACG |
| | | | | TGTCTCACGAGGACCCCGAGGTGAAGTTCAA |
| | | | | CTGGTATGTGGATGGCGTGGAAGTGCACAAC |
| | | | | GCCAAGACCAAGCCTCGGGAAGAGCAGTAC |
| | | | | AACTCCACCTACAGAGTGGTGTCCGTGCTGA |
| | | | | CCGTTCTGCACCAGGACTGGCTGAACGGCAA |
| | | | | AGAGTACAAGTGCAAGGTGTCCAACAAGGC |
| | | | | TCTGCCTGCCCCTATCGAGAAGACCATCTCC |
| | | | | AAGGCCAAAGGACAGCCTAGAGAGCCTCAA |
| | | | | GTGTGCACACTGCCACCATCTCGCGACGAGC |
| | | | | TGACAAAGAACCAGGTGTCTCTCTCCTGTGC |
| | | | | CGTCAAAGGCTTCTACCCCTCTGATATCGCC |
| | | | | GTGGAATGGGAATCCAATGGCCAGCCCGAG |
| | | | | AACAACTACAAGACCACACCTCCTGTGCTGG |
| | | | | ACTCCGATGGCAGCTTCTTCCTGGTGAGCAA |
| | | | | GCTGACCGTGGACAAGAGCAGATGGCAGCA |
| | | | | GGGCAACGTCTTCTCCTGCTCCGTGATGCAC |
| | | | | GAGGCCCTGCATAATCACTACACCCAGAAGT |
| | | | | CCCTGAGCCTGTCTCCTGGCAAG (SEQ ID NO: |
| | | | | 128) |
| | | CD3 | VH2 + | GAAGTGCAACTAGTGGAAAGTGGTGGTGGT |
| | | knob | CH | CTCGTGAAACCCGGCGGATCTCTGAGACTGT |
| | | | | CCTGTGCTGCTTCTGGCTTCACCTTCAACACC |
| | | | | TACGCCATGAACTGGGTCAGACAGGCTCCTG |
| | | | | GCAAGGGCCTGGAATGGGTGGCCAGAATCC |
| | | | | GGTCCAAGTACAACAACTACGCTACATATTA |
| | | | | CGCCGACTCCGTGAAGGACCGGTTTACCATC |
| | | | | TCCAGAGATGACTCTAAGAACACACTGTACC |
| | | | | TGCAGATGAATAGCCTGAAGACCGAGGACA |
| | | | | CCGCCGTGTACTACTGCGTGCGGCACGGCAA |
| | | | | CTTCGGCAACTCCTACGTGTCTTGGTTCGCCT |
| | | | | ACTGGGGCCAGGGCACCCTGGTGACCGTGTC |
| | | | | CAGCGGACAGCCCAAGGCTGCTCCTTCCGTG |
| | | | | ACCCTGTTCCCTCCATCCTCCGAGGAACTGC |
| | | | | AGGCCAATAAGGCCACCCTGGTGTGCCTGAT |
| | | | | CTCCGACTTCTACCCTGGCGCCGTGACCGTG |
| | | | | GCCTGGAAGGCCGACTCTTCTCCTGTGAAGG |
| | | | | CTGGCGTGGAGACAACCACCCCCTCCAAGCA |
| | | | | GTCCAACAACAAGTACGCCGCTTCTTCTTAC |
| | | | | CTGTCTCTGACCCCTGAGCAGTGGAAGTCTC |
| | | | | ATAGATCCTACTCCTGTCAAGTGACCCACGA |
| | | | | GGGCTCCACCGTGGAAAAAACCGTGGCCCCT |
| | | | | ACCGAGTGCTCCGACAAAACCCACACATGTC |
| | | | | CTCCTTGTCCTGCTCCTGAGGCCGCCGGCGG |
| | | | | CCCCTCTGTGTTTCTGTTCCCCCCTAAGCCTA |
| | | | | AAGATACCCTGATGATCTCTAGAACCCCAGA |
| | | | | AGTTACCTGTGTCGTGGTGGACGTGAGCCAC |
| | | | | GAGGATCCTGAAGTGAAGTTCAACTGGTACG |
| | | | | TGGACGGCGTGGAAGTGCACAACGCCAAGA |
| | | | | CCAAGCCCCGGGAAGAGCAGTACAACTCCA |
| | | | | CCTACAGAGTGGTCTCCGTGCTGACCGTGCT |
| | | | | GCACCAGGACTGGCTGAACGGAAAAGAGTA |
| | | | | CAAGTGCAAGGTGTCTAACAAGGCCCTCCCT |
| | | | | GCCCCTATCGAGAAGACCATCTCGAAGGCTA |
| | | | | AAGGCCAACCTAGAGAGCCTCAAGTGTACA |
| | | | | CACTTCCACCTTGCCGCGACGAGCTGACCAA |
| | | | | GAACCAGGTGTCTCTGTGGTGCCTGGTGAAG |
| | | | | GGCTTCTACCCTTCCGACATCGCCGTGGAGT |
| | | | | GGGAGTCCAATGGCCAGCCAGAGAACAACT |
| | | | | ACAAGACTACCCCTCCTGTGCTGGACTCCGA |
| | | | | TGGCTCCTTCTTCCTGTACTCCAAGCTGACAG |
| | | | | TGGATAAGTCTCGGTGGCAGCAGGGCAACGT |
| | | | | GTTCTCCTGCAGCGTGATGCACGAAGCCCTG |
| | | | | CATAATCACTACACCCAGAAGTCCCTGTCTC |
| | | | | TGAGCCCTGGCAAG (SEQ ID NO: 129) |
| | | | VL2 + | CAAACTGTGGTGACTCAAGAACCAAGTTTTT |
| | | | CL | CCGTGTCCCCAGGAGGTACCGTGACACTGAC |
| | | | | CTGTCGGAGCTCTACCGGCGCCGTGACCACA |
| | | | | TCTAACTACGCCAACTGGGTCCAACAGACCC |
| | | | | CTGGCCAGGCTCCTAGAGGCCTGATCGGCGG |
| | | | | CACCAATAAACGGGCCCCTGGCGTGCCCGCT |
| | | | | AGATTTTCTGGCTCCATCCTGGGCAACAAGG |
| | | | | CCGCTCTCACCATCACCGGCGCTCAGGCCGA |
| | | | | CGATGAAGCCGAGTACTTCTGCGCCCTGTGG |
| | | | | TACTCCAACCTGTGGGTGTTCGGCGGCGGAA |
| | | | | CCAAGCTGACAGTGCTGGCCTCCACAAAGGG |
| | | | | CCCAAGCGTGTTCCCTCTGGCTCCTTCCTCCA |
| | | | | AGTCCACCTCTGGCGGCACCGCCGCCCTGGG |
| | | | | CTGCCTGGTGAAGGACTACTTCCCCGAGCCT |
| | | | | GTGACCGTGTCCTGGAACTCCGGCGCTCTGA |
| | | | | CCTCCGGCGTGCACACCTTTCCTGCTGTGCTG |
| | | | | CAAAGCAGCGGACTGTACTCCCTGTCCTCCG |
| | | | | TGGTGACCGTGCCATCTTCCAGCCTGGGAAC |
| | | | | CCAGACATACATCTGCAACGTGAACCACAAG |
| | | | | CCCTCTAATACCAAGGTGGACAAGAAAGTG |
| | | | | GAACCTAAGTCTTGC (SEQ ID NO: 130) |
| BsAb-2 | Format | Siglec 15 | VL1 + | GATATCGTGCTAACTCAAAGTCCAGCAAGTC |
| | -2: KIH | Fab hole | CL | TGGCCGTGTCTCTGGGCCAACGGGCCACCAT |
| | & scFv | | | CAGCTGTAGAGCCTCTAAGTCCGTGTCCACC |
| | (2 + 1) | | | TCTGGCTACTCCTACATGCACTGGTATCAGC |
| | | | | AGAAGCCTGGCCAGCCTCCAAAACTGCTGAT |
| | | | | CTACCTGGCTTCCAACCTGGAATCTGGAGTC |
| | | | | CCCGCTCGGTTCTCCGGCAGCGGCTCTGGAA |
| | | | | CAGATTTTACCCTGAACATCCATCCTGTGGA |
| | | | | AGAGGAGGACGCCGCTACCTACTACTGCCAG |
| | | | | CACGGCAGAGAGCTGCCTTTCACCTTCGGCT |
| | | | | CCGGCACCAAGCTCGAGATCAAGAGGACAG |
| | | | | TGGCCGCCCCAAGCGTGTTCATCTTTCCCCCT |
| | | | | TCCGACGAGCAGCTGAAGTCTGGCACCGCCA |
| | | | | GCGTGGTGTGCCTGCTGAACAACTTCTACCC |
| | | | | TCGGGAGGCCAAGGTCCAGTGGAAGGTGGA |
| | | | | TAACGCCCTGCAGTCTGGCAATAGCCAGGAG |
| | | | | TCCGTGACCGAGCAGGACTCTAAGGATAGCA |
| | | | | CATATTCCCTGTCTAGCACCCTGACACTGAG |
| | | | | CAAGGCCGATTACGAGAAGCACAAGGTGTA |
| | | | | TGCCTGTGAAGTCACCCATCAGGGGCTGTCA |
| | | | | TCACCCGTCACTAAGTCATTCAATCGCGGAG |
| | | | | AATGC (SEQ ID NO: 131) |
| | | | VH1 + | CAAGTGCAACTACAACAAAGTGGTCCAGAA |
| | | | VH | CTGGTGAAGCCTGGAGCTTCTGTGAAGATCT |
| | | | | CCTGCAAGGCCTCTGGCTACGCCTTCTCCTCT |
| | | | | TCTTGGATGAATTGGGTCAAGCAGCGGCCTG |
| | | | | GCAAAGGCCTGGAATGGATCGGCAGAATCT |
| | | | | ACCCCGGACACGGCGAGACCAACTACAACG |
| | | | | GCAAGTTCAAGGACAAAGCTACAGTGACCG |
| | | | | CTGATAAGTCCAGCTCTACCACCTACATGCA |
| | | | | GCTGTCCTCTCTGACCTCCGAGGACTCCGCC |
| | | | | GTGTATTTTTGTGCCAGAGTGGACAACTCCG |
| | | | | ATCCTTACTACTTCGACTACTGGGGCCAGGG |
| | | | | CACAACCCTGACCGTGTCCAGCGCCAGCACC |
| | | | | AAAGGCCCCTCCGTGTTCCCCCTGGCCCCTA |
| | | | | GCTCCAAGTCTACCTCTGGCGGTACTGCTGC |
| | | | | CCTGGGCTGCCTGGTGAAGGACTACTTCCCT |
| | | | | GAGCCTGTGACAGTGTCCTGGAACTCCGGAG |
| | | | | CTCTGACCTCCGGCGTGCATACCTTTCCTGCT |
| | | | | GTGCTGCAATCCTCTGGACTGTACTCCCTGTC |
| | | | | CTCTGTGGTGACCGTGCCATCTTCCAGCCTG |
| | | | | GGCACCCAGACCTACATCTGCAACGTGAACC |
| | | | | ACAAGCCTTCCAACACCAAGGTGGACAAGA |
| | | | | AAGTGGAACCTAAGTCCTGCGATAAGACTCA |
| | | | | TACTTGCCCACCATGCCCAGCTCCTGAGGCT |
| | | | | GCTGGTGGCCCTTCTGTGTTTCTGTTCCCTCC |
| | | | | TAAGCCCAAGGACACCCTGATGATCTCTCGG |
| | | | | ACCCCTGAAGTGACCTGCGTGGTCGTGGACG |
| | | | | TGTCTCACGAGGACCCCGAGGTGAAGTTCAA |
| | | | | CTGGTATGTGGATGGCGTGGAAGTGCACAAC |
| | | | | GCCAAGACCAAGCCTCGGGAAGAGCAGTAC |
| | | | | AACTCCACCTACAGAGTGGTGTCCGTGCTGA |
| | | | | CCGTTCTGCACCAGGACTGGCTGAACGGCAA |
| | | | | AGAGTACAAGTGCAAGGTGTCCAACAAGGC |
| | | | | TCTGCCTGCCCCTATCGAGAAGACCATCTCC |
| | | | | AAGGCCAAAGGACAGCCTAGAGAGCCTCAA |
| | | | | GTGTGCACACTGCCACCATCTCGCGACGAGC |
| | | | | TGACAAAGAACCAGGTGTCTCTCTCCTGTGC |
| | | | | CGTCAAAGGCTTCTACCCCTCTGATATCGCC |
| | | | | GTGGAATGGGAATCCAATGGCCAGCCCGAG |
| | | | | AACAACTACAAGACCACACCTCCTGTGCTGG |
| | | | | ACTCCGATGGCAGCTTCTTCCTGGTGAGCAA |
| | | | | GCTGACCGTGGACAAGAGCAGATGGCAGCA |
| | | | | GGGCAACGTCTTCTCCTGCTCCGTGATGCAC |
| | | | | GAGGCCCTGCATAATCACTACACCCAGAAGT |
| | | | | CCCTGAGCCTGTCTCCTGGCAAG (SEQ ID |
| | | | | NO: 132) |
| | | CD3 | VH2 + | CAAGTGCAACTACAACAAAGTGGTCCAGAA |
| | | knob | VH | CTCGTGAAGCCTGGCGCATCCGTCAAGATCT |
| | | | | CCTGCAAGGCCTCCGGCTACGCCTTCTCCTC |
| | | | | CTCCTGGATGAACTGGGTCAAGCAGAGACCT |
| | | | | GGAAAAGGACTGGAGTGGATCGGTAGAATC |
| | | | | TACCCTGGCCACGGCGAGACCAATTACAACG |
| | | | | GCAAGTTCAAGGACAAGGCTACCGTGACCG |
| | | | | CCGATAAGTCTTCCTCCACCACCTACATGCA |
| | | | | GCTGTCTTCTCTGACATCTGAGGACTCTGCC |
| | | | | GTTTACTTCTGCGCCAGAGTGGACAACTCGG |
| | | | | ACCCCTACTACTTCGACTACTGGGGCCAGGG |
| | | | | CACCACCCTGACAGTGTCCTCTGCCTCTACC |
| | | | | AAGGGCCCATCTGTGTTTCCTCTGGCTCCTTC |
| | | | | TTCCAAAAGCACCTCCGGCGGGACCGCTGCT |
| | | | | CTGGGCTGCCTGGTGAAGGATTATTTCCCCG |
| | | | | AGCCTGTGACAGTGTCCTGGAACTCCGGCGC |
| | | | | TCTGACCAGCGGCGTCCACACCTTTCCAGCC |
| | | | | GTGCTGCAGTCTTCTGGCCTGTATTCTCTGAG |
| | | | | CTCCGTGGTGACAGTGCCCTCCTCTTCTCTGG |
| | | | | GAACGCAGACCTACATCTGCAACGTGAACCA |
| | | | | CAAGCCTAGCAATACCAAGGTGGACAAGAA |
| | | | | AGTGGAACCTAAGTCCTGTGGCGGAGGTGGC |
| | | | | TCTGGAGGCGGTGGATCTGGTGGTGGCGGAT |
| | | | | CACAAACCGTGGTGACCCAGGAACCCTCCTT |
| | | | | CTCCGTGTCCCCTGGAGGAACCGTTACCCTG |
| | | | | ACCTGCCGGAGTAGCACCGGCGCTGTGACCA |
| | | | | CCTCTAACTACGCCAACTGGGTACAGCAGAC |
| | | | | CCCTGGCCAGGCCCCTAGAGGACTGATCGGC |
| | | | | GGCACCAACAAGAGAGCCCCTGGCGTGCCT |
| | | | | GCTCGGTTCTCTGGCTCTATCCTCGGCAACA |
| | | | | AGGCCGCTCTGACCATTACCGGCGCCCAGGC |
| | | | | TGACGACGAGGCCGAGTACTTTTGTGCCCTG |
| | | | | TGGTACTCCAACCTGTGGGTGTTCGGAGGCG |
| | | | | GTACCAAACTGACAGTGCTGGGTGGAGGCG |
| | | | | GGAGCGGAGGCGGCGGTAGTGGTGGAGGAG |
| | | | | GCTCTGGCGGTGGTGGAAGCGAGGTGCAGCT |
| | | | | GGTCGAATCCGGCGGCGGCCTGGTGAAACCC |
| | | | | GGCGGCAGCCTGAGACTGAGTTGTGCTGCCT |
| | | | | CCGGCTTCACCTTCAACACCTATGCTATGAA |
| | | | | TTGGGTGCGGCAGGCTCCTGGCAAGGGCCTG |
| | | | | GAATGGGTGGCCCGGATCAGGTCTAAGTACA |
| | | | | ACAACTACGCCACCTACTACGCCGACTCCGT |
| | | | | GAAGGACAGATTCACCATCTCTCGGGACGAT |
| | | | | TCCAAGAACACACTGTACCTCCAGATGAACT |
| | | | | CCCTGAAGACTGAGGATACAGCCGTGTACTA |
| | | | | CTGCGTGCGCCATGGCAACTTCGGCAACTCC |
| | | | | TACGTGAGCTGGTTCGCCTACTGGGGCCAAG |
| | | | | GCACACTGGTGACCGTGTCCTCCGGTGGCGG |
| | | | | CGGATCTGGCGGAGGAGGTTCAGGTGGAGG |
| | | | | TGGATCTGACAAAACCCACACATGTCCTCCT |
| | | | | TGTCCTGCTCCTGAGGCCGCCGGCGGCCCCT |
| | | | | CTGTGTTTCTGTTCCCCCCTAAGCCTAAAGAT |
| | | | | ACCCTGATGATCTCTAGAACCCCAGAAGTTA |
| | | | | CCTGTGTCGTGGTGGACGTGAGCCACGAGGA |
| | | | | TCCTGAAGTGAAGTTCAACTGGTACGTGGAC |
| | | | | GGCGTGGAAGTGCACAACGCCAAGACCAAG |
| | | | | CCCCGGGAAGAGCAGTACAACTCCACCTACA |
| | | | | GAGTGGTCTCCGTGCTGACCGTGCTGCACCA |
| | | | | GGACTGGCTGAACGGAAAAGAGTACAAGTG |
| | | | | CAAGGTGTCTAACAAGGCCCTCCCTGCCCCT |
| | | | | ATCGAGAAGACCATCTCGAAGGCTAAAGGC |
| | | | | CAACCTAGAGAGCCTCAAGTGTACACACTTC |
| | | | | CACCTTGCCGCGACGAGCTGACCAAGAACCA |
| | | | | GGTGTCTCTGTGGTGCCTGGTGAAGGGCTTC |
| | | | | TACCCTTCCGACATCGCCGTGGAGTGGGAGT |
| | | | | CCAATGGCCAGCCAGAGAACAACTACAAGA |
| | | | | CTACCCCTCCTGTGCTGGACTCCGATGGCTC |
| | | | | CTTCTTCCTGTACTCCAAGCTGACAGTGGAT |
| | | | | AAGTCTCGGTGGCAGCAGGGCAACGTGTTCT |
| | | | | CCTGCAGCGTGATGCACGAAGCCCTGCATAA |
| | | | | TCACTACACCCAGAAGTCCCTGTCTCTGAGC |
| | | | | CCTGGCAAG (SEQ ID NO: 133) |
| | | | VL2 + | GATATCGTGCTAACTCAAAGTCCAGCAAGTC |
| | | | CL | TGGCCGTGTCTCTGGGCCAACGGGCCACCAT |
| | | | | CAGCTGTAGAGCCTCTAAGTCCGTGTCCACC |
| | | | | TCTGGCTACTCCTACATGCACTGGTATCAGC |
| | | | | AGAAGCCTGGCCAGCCTCCAAAACTGCTGAT |
| | | | | CTACCTGGCTTCCAACCTGGAATCTGGAGTC |
| | | | | CCCGCTCGGTTCTCCGGCAGCGGCTCTGGAA |
| | | | | CAGATTTTACCCTGAACATCCATCCTGTGGA |
| | | | | AGAGGAGGACGCCGCTACCTACTACTGCCAG |
| | | | | CACGGCAGAGAGCTGCCTTTCACCTTCGGCT |
| | | | | CCGGCACCAAGCTCGAGATCAAGAGGACAG |
| | | | | TGGCCGCCCCAAGCGTGTTCATCTTTCCCCCT |
| | | | | TCCGACGAGCAGCTGAAGTCTGGCACCGCCA |
| | | | | GCGTGGTGTGCCTGCTGAACAACTTCTACCC |
| | | | | TCGGGAGGCCAAGGTCCAGTGGAAGGTGGA |
| | | | | TAACGCCCTGCAGTCTGGCAATAGCCAGGAG |
| | | | | TCCGTGACCGAGCAGGACTCTAAGGATAGCA |
| | | | | CATATTCCCTGTCTAGCACCCTGACACTGAG |
| | | | | CAAGGCCGATTACGAGAAGCACAAGGTGTA |
| | | | | TGCCTGTGAAGTCACCCATCAGGGGCTGTCA |
| | | | | TCACCCGTCACTAAGTCATTCAATCGCGGAG |
| | | | | AATGC (SEQ ID NO: 134) |

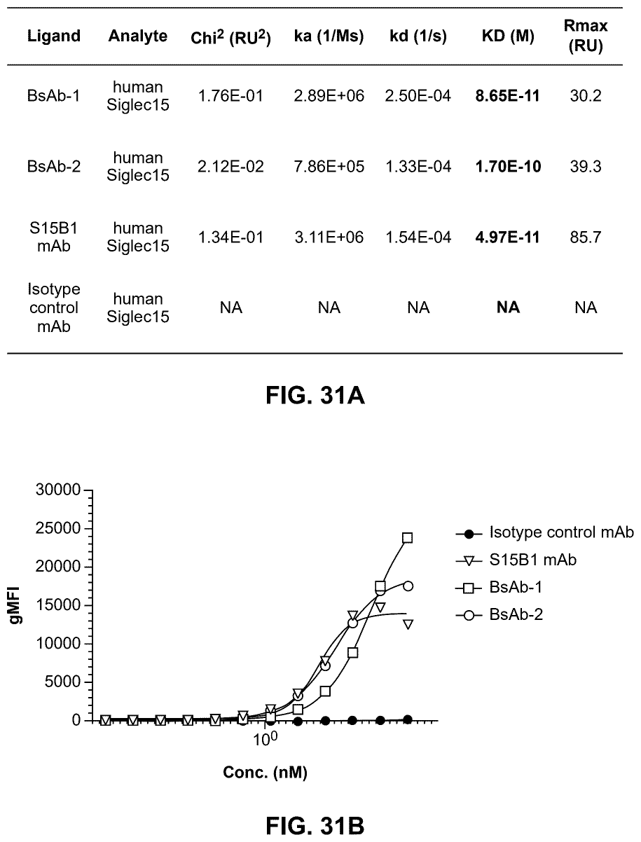

The binding affinity to recombinant Siglec-15 of BsAb-1 and BsAb-2 compared to S15B1 and an isotype control was measured with BLI (FIG. 31A). The bispecific antibodies were further assessed for binding affinity to CHO cells expressing Siglec-15 by flow cytometry (FIG. 31B). BsAb-1 and BsAb-2 demonstrated similar binding profiles as S15B1 to Siglec-15 in both assays.

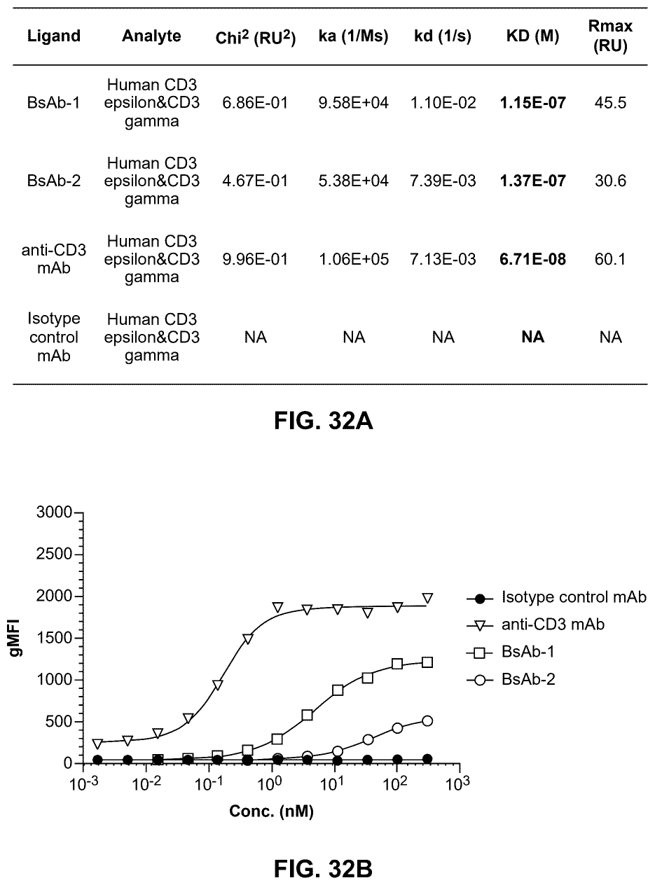

BsAb-1 and BsAb-2 were then tested for binding to recombinant CD3 and to Jurkat cells expressing CD3 compared to the parental anti-CD3 antibody and an isotype control. BsAb-1 and BsAb-2 demonstrated lower binding affinity for recombinant CD3 than the parental anti-CD3 antibody as measured by BLI (FIG. 32A). Both bispecific antibodies also exhibited lower binding affinity for CD3-expressing Jurkat cells than the parental anti-CD3, with BsAb-1 having higher affinity than BsAb-2 (FIG. 32B).

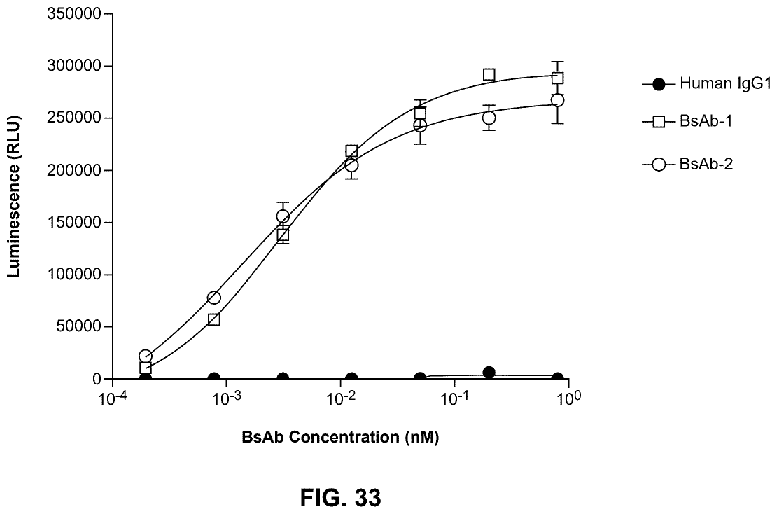

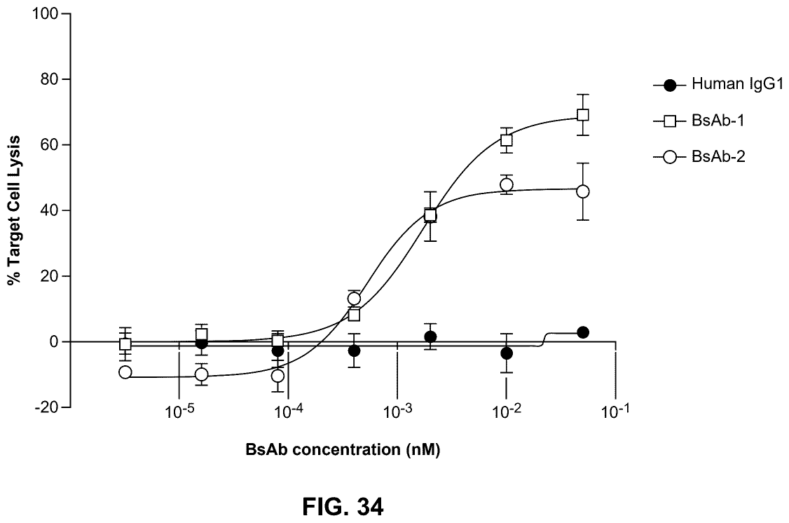

BsAb-1 and BsAb-2 demonstrated similar activation of Jurkat T-cells after incubation, as measured by a luminescence-based reporter assay (FIG. 33). BsAb-1 and BsAb-2 produced approximately 70% and 40% lysis, respectively, of target cells expressing Siglec-15 in the presence of human T-cells (FIG. 34).

### Materials and Methods

Production and characterization of ADCs: DNA was codon optimized, synthesized and cloned into production vectors. Detailed sequence analysis (in silico analysis) was carried out including, but not limited to the correctness of the sequence, potential N-glycosylation sites, potential O-glycosylation sites, non-paired Cys residues, and potential deamidation and oxidation sites. After verification of the construct by sequencing, a corresponding amount of plasmid was prepared for subsequent transfection. After transfection, the CHO cell culture was scaled up to 500 mL for 14 days fed batch. Cell culture supernatant was collected for purification and antibodies were purified with a protein A column and SEC/IEX column to deliver at least 98% purity and endotoxin levels <0.2 EU/mg. The protein was formulated in a buffer with pH between 5-6 at >5 mg/ml concentration. The payload linker was analyzed by collecting purity by HPLC, molecule weight by LCMS to ensure the quality of payload linker. At least 60 mg vc-MMAE and 120 mg MC-GGFG-Exatecan were used. At least 100 mg for each 12 antibody drug conjugates were generated.

Binding of anti-Siglec-15 ADCs to THP-1 S15+ cells: 50,000 cells/well of THP-1 S15 OE cells were added to a round bottom 96 well plate, centrifuged at 450×G for 5 minutes and the supernatant discarded. Human anti-siglec-15 IgG ADCs were prepared (starting at 198 nM, 3-fold serial dilution in staining buffer) and the cells were resuspended in 100 μl of ADC. Cells were incubated for 40 minutes on ice and 100 μl of staining buffer were added to each well, followed by centrifugation at 450×G for 5 minutes and supernatants were discarded. The secondary antibody was prepared (anti-human IgG-PE at 1:500 dilution) and added 100 μl to each well. Cells were incubated for 40 minutes on ice and 100 μl of staining buffer were added to each well followed by centrifugation at 450× G for 5 minutes to then discard the supernatant. Cells were resuspended in 150 μl of staining buffer and acquired in a flow cytometer.

ADC Cytotoxicity assay (readout by flow cytometry): 50,000 THP-1 S15 OE cells were added to each well of a 96 round-bottom well plate. 100 μl of anti-Siglec-15 and trastuzumab ADCs were prepared (starting at 13.3 nM, 3-fold serial dilution in RPMI 10% Fetal Bovine Serum 1% Penicillin/streptomycin), added to the cells and incubated for 48 hours at 37° C. 5% CO2. For the assessment of ADC cytotoxicity by flow cytometry, cells were centrifuged at 600×G for 5 minutes, the supernatant was discarded and cells were resuspended in 200 μl of staining buffer with 1:20000 dilution of DAPI. Cells were acquired in a flow cytometer. For the assessment of ADC cytotoxicity by luminescence (CellTiter-Glo), a staining mix was prepared containing a volume of CellTiter-Glo® Reagent and an equal volume of cell culture medium. This mix was added to the ADC-treated cells and incubated for 2 minutes on an orbital shaker to induce cell lysis. The plate was the incubated at room temperature for 10 minutes to stabilize the luminescent signal. The luminescence signal corresponding to alive cells was measured in a multimode plate reader (PerkinElmer Victor Nivo).

Macrophage differentiation from CD14+ monocytes: Human peripheral blood mononuclear cells (PBMCs) were isolated from blood donors, who provided informed consent, using density gradient centrifugation. Monocytes were then purified from PBMCs utilizing a CD14 selection kit (Stemcell) according to the manufacturer's instructions. The isolated monocytes were differentiated into macrophages by culturing in RPMI 1640 medium supplemented with 10% FBS and 100 ng/mL macrophage colony-stimulating factor (M-CSF) for 5 days.

Viability assay with Calcein AM: To prepare the viability assay with Calcein AM for flow cytometry, 5 μL of Calcein AM (Component A) and 20 μL of Ethidium Homodimer-1 (Component B) was added to 10 mL of DPBS. Cells were centrifuged at 450×G for 5 minutes to remove the culture medium, and 100-200 μL of the staining solution was added directly to the cells and incubated for 30 minutes at room temperature (20-25° C.). After the incubation, cells were centrifuged at 450×G for 5 minutes to remove the supernatant, and resuspended in 150 μL of DPBS. Cells were analyzed by flow cytometry.

In vivo efficacy study on EMT-6 subcutaneous syngeneic tumor model: Female Balb/c mice (8 mice per group) were used to evaluate the efficacy of anti-Siglec-15 ADCs in the EMT-6 subcutaneous syngeneic breast cancer model. ADC i.v. administration started when the tumor reached about 40-80 mm³. Tumor volume and body weight were measured twice a week for 3-4 weeks. ADCs were injected at 10 mg/kg. Mice were sacrificed when tumor volume reached 2500 mm³.

Bispecific antibody production and affinity validation: A) BsAb Design and Recombinant Production: To produce bispecific antibodies (BsAbs), a set of BsAbs and anti-CD3 monoclonal antibody (mAb) will be transiently transfected into Expi CHO-S cells. The transfections were conducted at a 300 ml culture scale. After transfection, antibodies were purified using either a protein A affinity column or his-tag purification resin, depending on the affinity and properties of the expressed antibodies. To further purify the antibodies, size-exclusion chromatography (SEC-HPLC) was employed as a second-step purification, based on the initial purity analysis. B) BsAb Affinity Validation: Once the antibodies were purified, the following steps were performed for affinity validation: 1-Biacore SPR Analysis for Siglec15 Binding: Surface plasmon resonance (SPR) analysis using a Biacore system was conducted for all BsAbs and parental mAbs against Siglec15 to assess binding affinity. 2-Biacore SPR Analysis for CD3 Binding: Similarly, Biacore SPR analysis was conducted for all BsAbs and the parental anti-CD3 mAb to evaluate their binding affinity to CD3. 3-FACS EC50 Analysis for Cell Surface Siglec15 Binding: Flow cytometry (FACS) analysis was conducted to determine the effective concentration (EC50) of the BsAbs and parental mAbs for binding to Siglec15 on the surface of either overexpressing cell lines or tumor cell lines. A concentration gradient (10 points) was used for the EC50 determination.

TDCC Reporter Gene Assay: 20.000 THP-1 S15 OE cells were harvested, gently resuspended in the corresponding complete culture medium, and added to a 96-well assay plate. The bispecific antibody samples were diluted with assay medium, starting at a concentration of 0.8 nM, with a 4-fold dilution factor, and transferred to the appropriate wells of the assay plate. The plate was incubated at room temperature for approximately 30 minutes. Subsequently, 20,000 TDCC reporter cells were harvested and added to the assay plate, followed by incubation for approximately 6 hours. After incubation, Bio-Lite Luciferase working solution was added to the corresponding wells. Luminescence values were measured using a PHERAstar FSX system, and the data were recorded. Dose-response curves were constructed by plotting the relative luminescence units (RLU) against the concentration of the test samples.

PBMC based TDCC assay: 5000 target cells (THP-1 S15 OE) were harvested, gently resuspended in the corresponding assay buffer, and added to a 96-well assay plate. The bispecific antibody samples were diluted in assay medium, starting at a concentration of 0.8 nM with a 4-fold dilution factor, and transferred to the appropriate wells of the assay plate. The plate was incubated at room temperature for approximately 30 minutes. Effector cells (PBMCs) were then harvested, resuspended in assay buffer, and their density was adjusted to achieve the desired effector-to-target (E/T) ratio. The effector cell suspension (100 μL per well) was added to the corresponding wells of the assay plate. Following incubation at 37° C. with 5% CO2 for 24 hours, the assay plate was removed, and 50 μL of supernatant from each well was transferred to a new 96-well assay plate. A LDH assay mixture (50 μL per well) was prepared and added to the corresponding wells. Absorbance values were measured at 492 nm and 650 nm using the PHERAstar system. Dose-response curves were generated by plotting the percentage of target cell lysis against the sample concentration. The percentage of target cell lysis was calculated using the formula: %

$$Target cell lysis = 100 * ( ODSample data - ODTarget cells plus effector cells ) / ( ODMaximum release - ODMinimum release ) .$$

### Example 8: Anti-Siglec-15 Antibody Immunohistochemistry and Osteoclast Assays

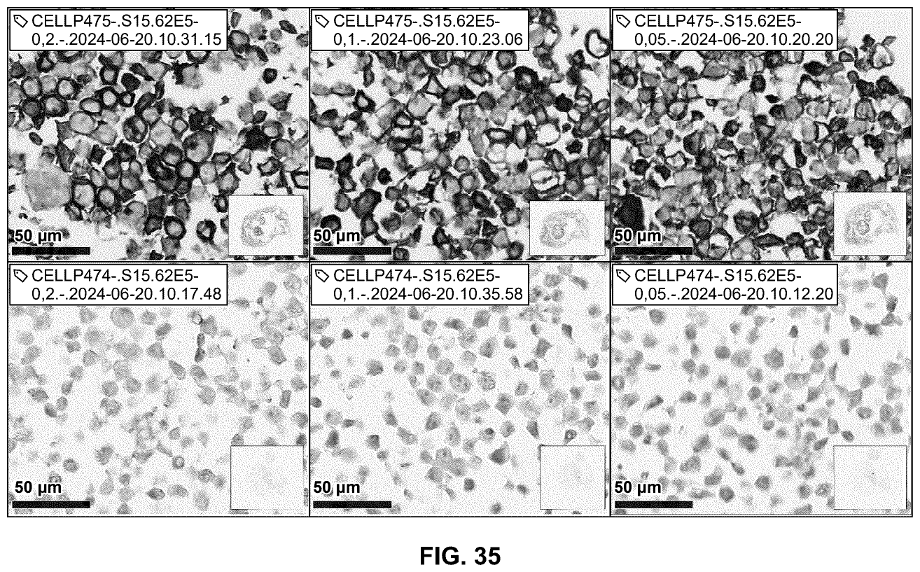

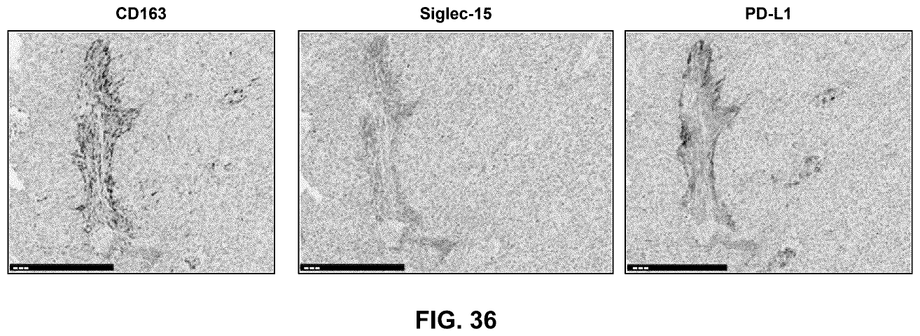

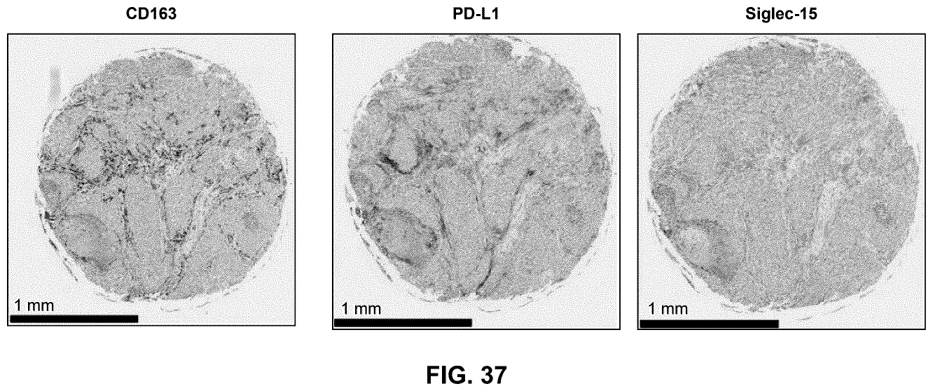

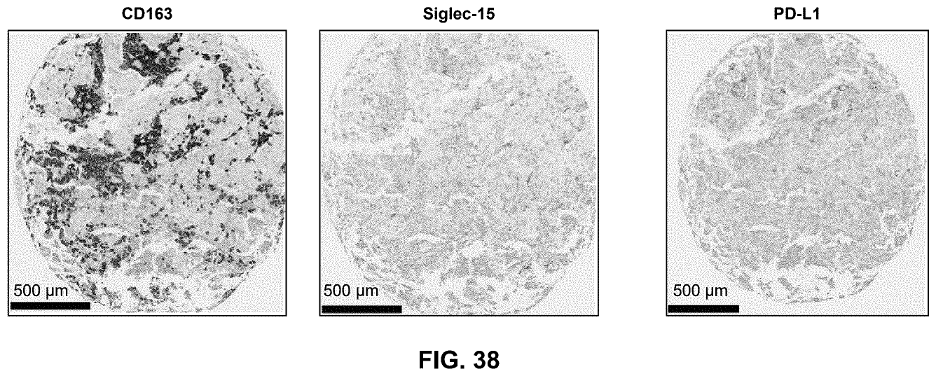

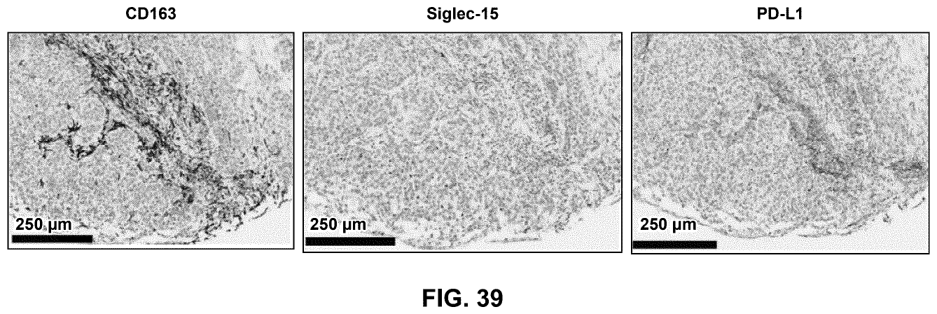

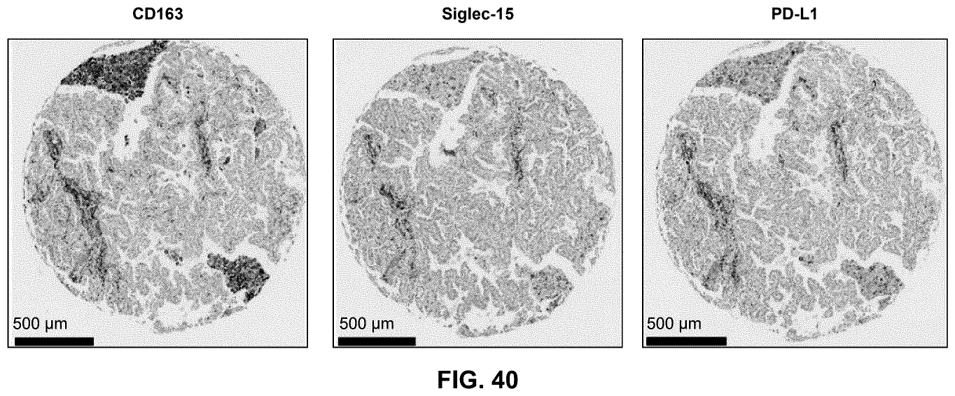

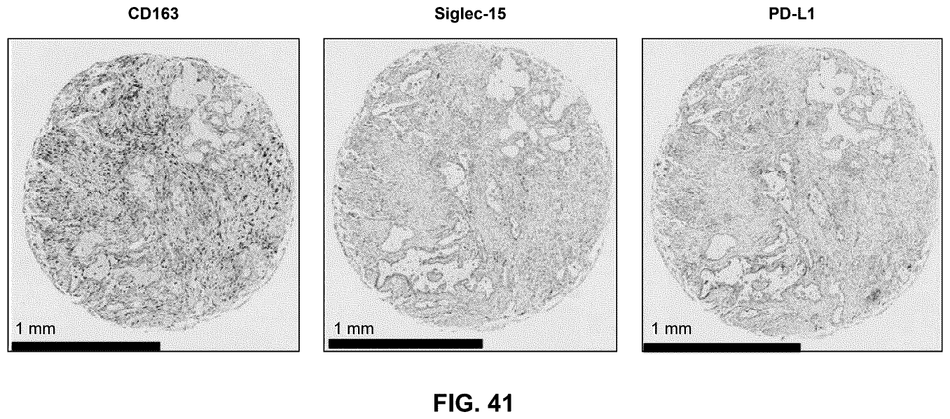

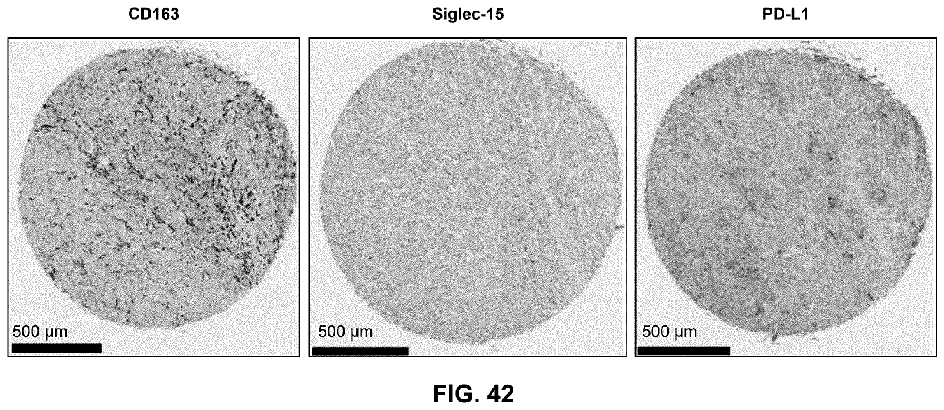

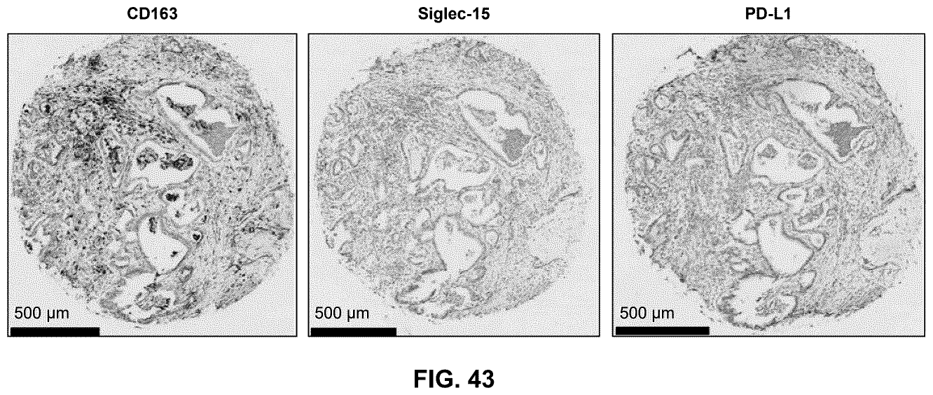

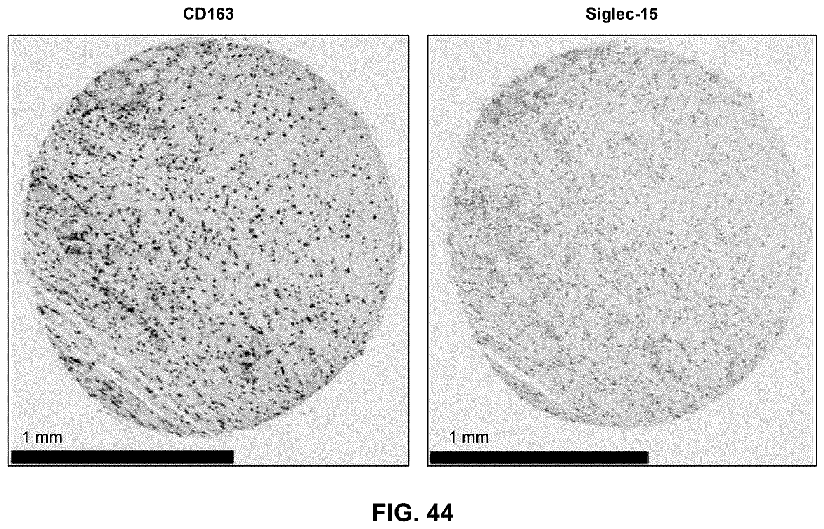

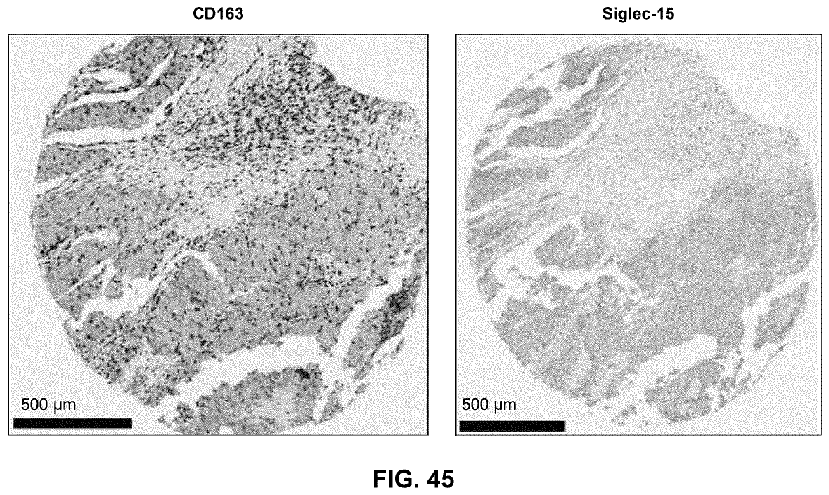

A series of immunohistochemistry (IHC) experiments were conducted using the described anti-Siglec-15 antibodies. IHC using antibody 62E5 showed binding in Siglec-15 positive HEK293T cell pellets but not Siglec-15 negative cells, demonstrating specificity and sensitivity of the antibody (FIG. 35). Binding of an anti-CD163 antibody, clone 62E5, and an anti-PD-L1 antibody are shown in a human breast cancer sample in FIG. 36, FIG. 37, FIG. 38, and FIG. 39. Binding of these antibodies to a human lung cancer sample are shown in FIG. 40 and FIG. 41. FIG. 42 shows binding of the anti-CD163 antibody, clone 62E5, and anti-PD-L1 antibody in a human liver cancer sample. FIG. 43 shows binding of the anti-CD163 antibody, clone 62E5, and anti-PD-L1 antibody to a human pancreatic cancer sample. FIG. 44 and FIG. 45 show an anti-CD163 antibody and clone 62E5 in a human osteosarcoma sample.

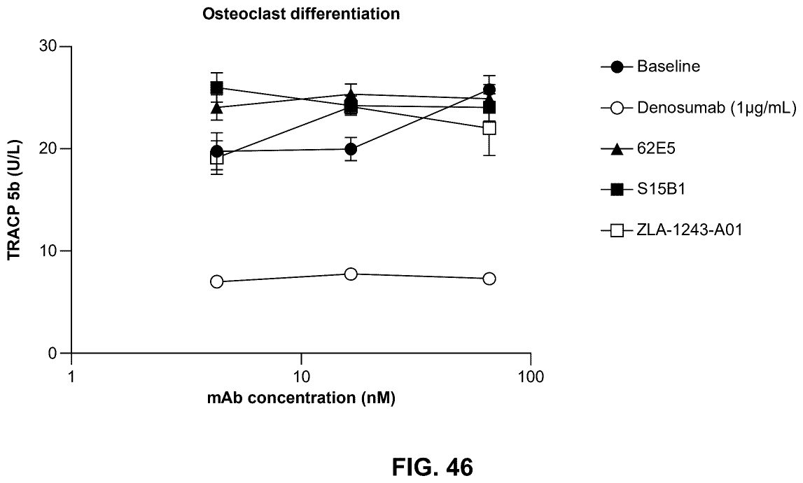

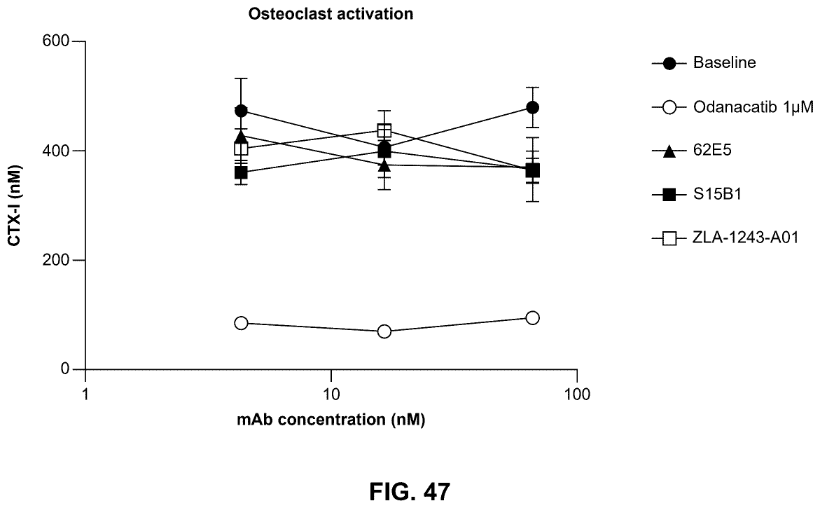

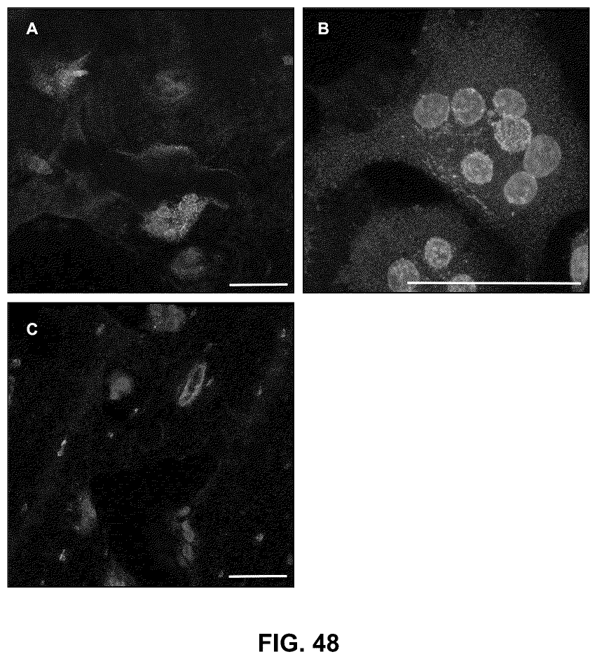

The effects of antibodies S15B1, 62E5 and ZLA-1243-A01 on osteoclast differentiation and activation were tested in vitro by measuring TRACP 5b and CTX-I in the culture medium. Antibodies S15B1, 62E5 and ZLA-1243-A01 did not significantly affect osteoclast differentiation relative to baseline levels, while denosumab reduced differentiation (FIG. 46). Similarly, antibodies S15B1, 62E5 and ZLA-1243-A01 did not significantly affect osteoclast activation relative to baseline, while an odanacatib control reduced activation (FIG. 47). FIG. 48A and FIG. 48B show immunofluorescence staining with clone 62E5 at day 7 of osteoclast differentiation, while FIG. 48C shows staining with a secondary antibody control. Collectively, these results suggest antibodies S15B1, 62E5 and ZLA-1243-A01 do not have an effect on osteoclast activity.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes.

1. An antibody or antigen-binding fragment thereof that specifically binds to Siglec-15, wherein the antibody or antigen-binding fragment thereof comprises a heavy chain variable region (VH) comprising complementarity determining regions CDRH1, CDRH2, and CDRH3, and a light chain variable region (VL) comprising complementarity determining regions CDRL1, CDRL2, and CDRL3, wherein the CDRH1, CDRH2, CDRH3, CDRL1, CDRL2, and CDRL3 comprise the CDRH1, CDRH2, CDRH3, CDRL1, CDRL2, and CDRL3 amino acid sequences, respectively, set forth in:
(i) SEQ ID NOs: 4, 5, 6, 7, 8, and 9;
(ii) SEQ ID NOs: 12, 13, 14, 15, 16, and 17;
(iii) SEQ ID NOs: 20, 21, 22, 23, 24, and 25;
(iv) SEQ ID NOs: 28, 29, 30, 31, 32, and 33;
(v) SEQ ID NOs: 36, 37, 38, 39, 40, and 41;
(vi) SEQ ID NOs: 44, 45, 46, 47, 48, and 49;
(vii) SEQ ID NOs: 52, 53, 54, 55, 56, and 57;
(viii) SEQ ID NOs: 60, 61, 62, 63, 64, and 65; or
(ix) SEQ ID NOs: 68, 69, 70, 71, 72, and 73.

2. (canceled)

3. The antibody or antigen-binding fragment thereof of claim 1, wherein the heavy chain variable region (VH) comprises the amino acid sequence of:
(i) SEQ ID NO: 10;
(ii) SEQ ID NO: 18;
(iii) SEQ ID NO: 26;
(iv) SEQ ID NO: 34;
(v) SEQ ID NO: 42;
(vi) SEQ ID NO: 50;
(vii) SEQ ID NO: 58;
(viii) SEQ ID NO: 66;
(ix) SEQ ID NO: 74;
(x) SEQ ID NO: 103;
(xi) SEQ ID NO: 104;
(xii) SEQ ID NO: 105; or
(xiii) SEQ ID NO: 106.

4. The antibody or antigen-binding fragment thereof of claim 1, wherein the light chain variable region (VL) comprises the amino acid sequence of:
(i) SEQ ID NO: 11;
(ii) SEQ ID NO: 19;
(iii) SEQ ID NO: 27;
(iv) SEQ ID NO: 35;
(v) SEQ ID NO: 43;
(vi) SEQ ID NO: 51;
(vii) SEQ ID NO: 59;
(viii) SEQ ID NO: 67;
(ix) SEQ ID NO: 75;
(x) SEQ ID NO: 107; or
(xi) SEQ ID NO: 108.

5-6. (canceled)

7. An antibody or antigen-binding fragment thereof that specifically binds to Siglec-15, wherein the antibody or antigen-binding fragment thereof comprises a heavy chain variable region and a light chain variable region comprising the amino acid sequences, respectively, of:
(i) SEQ ID NOs: 10 and 11;
(ii) SEQ ID NO: 18 and 19;
(iii) SEQ ID NO: 26 and 27;
(iv) SEQ ID NO: 34 and 35;
(v) SEQ ID NO: 42 and 43;
(vi) SEQ ID NO: 50 and 51;
(vii) SEQ ID NO: 58 and 59;
(viii) SEQ ID NO: 66 and 67;
(ix) SEQ ID NO: 74 and 75;
(x) SEQ ID NO: 104 and 108;
(xi) SEQ ID NO: 105 and 107; or
(xii) SEQ ID NO: 105 and 108.

8. (canceled)

9. The antibody or antigen-binding fragment thereof of claim 1, wherein the Siglec-15 is human Siglec-15.

10. The antibody or antigen-binding fragment thereof of claim 1, wherein the antibody or antigen-binding fragment thereof further comprises a heavy chain constant region, wherein the heavy chain constant region is selected from the group consisting of IgG1, IgG2, IgG3, and IgG4, or a variant thereof.

11-12. (canceled)

13. The antibody or antigen-binding fragment thereof of claim 10, wherein the heavy chain constant region comprises a silenced Fc region, wherein the silenced Fc region comprises one or more modifications selected from the group consisting of NA, AAA, L234A/L235A, IgG4-PE, RR, GA, FES, IgG2m4, L234AL235AP329G, L234F/L235E/P331S, E233P/L234V/L235A, IgG2c4d, and FEA.

14-16. (canceled)

17. The antibody or antigen-binding fragment thereof of claim 1, wherein the antibody or antigen-binding fragment further comprises a light chain constant region, wherein the light chain constant region is lambda or kappa.

18-19. (canceled)

20. The antibody or antigen-binding fragment thereof of claim 1, wherein the antibody or antigen-binding fragment thereof binds the D2 domain of Siglec-15.

21-23. (canceled)

24. The antibody or antigen-binding fragment thereof of claim 1, wherein the antibody or antigen-binding fragment thereof is a monoclonal antibody, a fully human antibody, a murine antibody, a humanized antibody, a nanobody, a single-domain antibody, or a chimeric antibody or antigen-binding fragment thereof.

25. (canceled)

26. The antibody or antigen-binding fragment thereof of claim 1, wherein the antibody is a bispecific antibody comprising a first binding domain that specifically binds to Siglec-15, further comprising a second binding domain that specifically binds to a cell-surface molecule selected from the group consisting of CD3, PD-L1, PD-L2, Siglec-9, Siglec-10, SIRP1a, CD47, TREM2, and a second Siglec-15.

27-30. (canceled)

31. The antibody or antigen-binding fragment thereof of claim 1, wherein the antibody or antigen-binding fragment is conjugated to an immunomodulatory agent, a cytotoxic agent, a therapeutic agent, a nucleic acid, a radiolabeled agent, a linker, a prodrug, or any combination thereof.

32. A pharmaceutical composition comprising the antibody or antigen-binding fragment thereof of claim 1, and a carrier.

33. The pharmaceutical composition of claim 32, further comprising an additional therapeutic agent, wherein the additional therapeutic agent is selected from the group consisting of an anti-checkpoint inhibitor antibody, an immunotherapeutic agent, a chemotherapeutic agent, a radiotherapeutic agent, a CAR T-cell therapeutic, and tumor infiltrating lymphocytes (TIL).

34-36. (canceled)

37. An isolated nucleic acid molecule encoding the heavy chain variable region (VH) and/or light chain variable region (VL) of the antibody or antigen-binding fragment thereof of claim 1.

38. A vector comprising the nucleic acid molecule of claim 37.

39. (canceled)

40. A host cell comprising the vector of claim 38.

41. A chimeric antigen receptor (CAR) comprising a Siglec-15 binding domain comprising complementarity determining regions CDRH1, CDRH2, CDRH3, CDRL1, CDRL2, and CDRL3 comprising the amino acid sequences, respectively, of:
(i) SEQ ID NOs: 4, 5, 6, 7, 8, and 9;
(ii) SEQ ID NOs: 12, 13, 14, 15, 16, and 17;
(iii) SEQ ID NOs: 20, 21, 22, 23, 24, and 25;
(iv) SEQ ID NOs: 28, 29, 30, 31, 32, and 33;
(v) SEQ ID NOs: 36, 37, 38, 39, 40, and 41;
(vi) SEQ ID NOs: 44, 45, 46, 47, 48, and 49;
(vii) SEQ ID NOs: 52, 53, 54, 55, 56, and 57;
(viii) SEQ ID NOs: 60, 61, 62, 63, 64, and 65; or
(ix) SEQ ID NOs: 68, 69, 70, 71, 72, and 73.

42-46. (canceled)

47. An engineered cell comprising the CAR of claim 41.

48-49. (canceled)

50. A method of producing an antibody or antigen-binding fragment thereof that specifically binds to Siglec-15 comprising (i) culturing a cell comprising the vector of claim 38 under conditions that express the antibody or antigen-binding fragment thereof; and (ii) recovering the antibody or antigen-binding fragment thereof.

51-55. (canceled)

56. A method of depleting cells expressing Siglec-15 in a subject comprising contacting the cells expressing Siglec-15 with the antibody or antigen-binding fragment thereof of claim 1.

57-59. (canceled)

60. A method of treating, preventing, alleviating a symptom of, or delaying the progression of a cancer in a subject in need thereof comprising administering the antibody or antigen-binding fragment thereof of claim 1, wherein the cancer is a bladder cancer, a bone cancer, a breast cancer, a carcinoid, a cervical cancer, a colon cancer, an endometrial cancer, a glioma, a head and neck cancer, a liver cancer, a lung cancer, a lymphoma, a melanoma, an osteosarcoma, an ovarian cancer, a pancreatic cancer, a prostate cancer, a renal cancer, a sarcoma, a skin cancer, a stomach cancer, a testis cancer, a thyroid cancer, a urogenital cancer, or a urothelial cancer.

61-73. (canceled)


SEQUENCE LISTING (134 sequences)

SEQ ID NO: 1 - protein, 328 aa, Homo sapiens
  source 1..328: note = Human Siglec-15
MEKSIWLLACLAWVLPTGSFVRTKIDTTENLLNTEVHSSPAQRWSMQVPPEVSAEAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRHGVRLHVTAAPRIVNISVLPSPAHAFRALCTAEGEPPPALAWSGPALGNSLAAVRSPREGHGHLVTAELPALTHDGRYTCTAANSLGRSEASVYLFRFHGASGASTVALLLGALGFKALLLLGVLAARAARRRPEHLDTPDTPPRSQAQESNYENLSQMNPRSPPATMCSP

SEQ ID NO: 2 - protein, 327 aa, Macaca fascicularis
  source 1..327: note = Cynomolgus monkey Siglec-15
MESSIRLLACLACVLPTGSFVRTKIDTTENLLNTEVHSSPVQRWSMQVPAEVSAAAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRHGVRLHVTAAPRIINISVLPGPAHAFRALCTTEGEPPPALAWSGPALGNGSAAVPSSGQGHGHLVTAELPALNHDGRYTCTAANSLGRSEASVYLFRFHGASASTVALLLGALGLKALLLLGVLAAGVARHRPEHLNTPDTPPRSQAQESNYENLSQMNPRSPPAAMCSP

SEQ ID NO: 3 - protein, 342 aa, Mus musculus
  source 1..342: note = Murine Siglec-15
MEGSLQLLACLACVLQMGSLVKTRRDASGDLLNTEAHSAPAQRWSMQVPAEVNAEAGDAAVLPCTFTHPHRHYDGPLTAIWRSGEPYAGPQVFRCTAAPGSELCQTALSLHGRFRLLGNPRRNDLSLRVERLALADSGRYFCRVEFTGDAHDRYESRHGVRLRVTAAAPRIVNISVLPGPAHAFRALCTAEGEPPPALAWSGPAPGNSSAALQGQGHGYQVTAELPALTRDGRYTCTAANSLGRAEASVYLFRFHGAPGTSTLALLLGALGLKALLLLGILGARATRRRLDHLVPQDTPPRSQAQESNYENLSQMSPPGHQLPRVCCEELLSHHHLVIHHEK

SEQ ID NO: 4 - protein, 5 aa, synthetic construct
  source 1..5: note = Clone 62E5 CDRH1
GYWMS

SEQ ID NO: 5 - protein, 17 aa, synthetic construct
  source 1..17: note = Clone 62E5 CDRH2
EIIPDSSTINYTPSLKD

SEQ ID NO: 6 - protein, 8 aa, synthetic construct
  source 1..8: note = Clone 62E5 CDRH3
RDGYWFAY

SEQ ID NO: 7 - protein, 17 aa, synthetic construct
  source 1..17: note = Clone 62E5 CDRL1
KSSQSLLYSRNQQNYLA

SEQ ID NO: 8 - protein, 7 aa, synthetic construct
  source 1..7: note = Clone 62E5 CDRL2
WASTRES

SEQ ID NO: 9 - protein, 9 aa, synthetic construct
  source 1..9: note = Clone 62E5 CDRL3
QQYYSYPLT

SEQ ID NO: 10 - protein, 117 aa, synthetic construct
  source 1..117: note = Clone 62E5 heavy chain (VH)
EVKLLESGGGLVQPGGSLKLSCAASGFDFSGYWMSWVRQAPGKGLEWIGEIIPDSSTINYTPSLKDKFIISRDNAKNTLFLQMRKVRSEDTALYYCARRDGYWFAYWGQGTLVTVSA

SEQ ID NO: 11 - protein, 113 aa, synthetic construct
  source 1..113: note = Clone 62E5 light chain (VL)
DIVMSQSPSSLAVSVGEKVTMSCKSSQSLLYSRNQQNYLAWFQQKPGQSPELLIYWASTRESGVPDRFTGSGSGTDFTLTISSVKAEDLAVYYCQQYYSYPLTFGAGTKLELK

SEQ ID NO: 12 - protein, 5 aa, synthetic construct
  source 1..5: note = Clone S15B1 CDRH1
SSWMN

SEQ ID NO: 13 - protein, 17 aa, synthetic construct
  source 1..17: note = Clone S15B1 CDRH2
RIYPGHGETNYNGKFKD

SEQ ID NO: 14 - protein, 11 aa, synthetic construct
  source 1..11: note = Clone S15B1 CDRH3
VDNSDPYYFDY

SEQ ID NO: 15 - protein, 15 aa, synthetic construct
  source 1..15: note = Clone S15B1 CDRL1
RASKSVSTSGYSYMH

SEQ ID NO: 16 - protein, 7 aa, synthetic construct
  source 1..7: note = Clone S15B1 CDRL2
LASNLES

SEQ ID NO: 17 - protein, 9 aa, synthetic construct
  source 1..9: note = Clone S15B1 CDRL3
QHGRELPFT

SEQ ID NO: 18 - protein, 120 aa, synthetic construct
  source 1..120: note = Clone S15B1 VH
QVQLQQSGPELVKPGASVKISCKASGYAFSSSWMNWVKQRPGKGLEWIGRIYPGHGETNYNGKFKDKATVTADKSSSTTYMQLSSLTSEDSAVYFCARVDNSDPYYFDYWGQGTTLTVSS

SEQ ID NO: 19 - protein, 111 aa, synthetic construct
  source 1..111: note = Clone S15B1 VL
DIVLTQSPASLAVSLGQRATISCRASKSVSTSGYSYMHWYQQKPGQPPKLLIYLASNLESGVPARFSGSGSGTDFTLNIHPVEEEDAATYYCQHGRELPFTFGSGTKLEIK

SEQ ID NO: 20 - protein, 5 aa, synthetic construct
  source 1..5: note = ZLA-1243-A01 CDRH1
SYAMS

SEQ ID NO: 21 - protein, 17 aa, synthetic construct
  source 1..17: note = ZLA-1243-A01 CDRH2
AISGSGGSTYYADSVKG

SEQ ID NO: 22 - protein, 12 aa, synthetic construct
  source 1..12: note = ZLA-1243-A01 CDRH3
DNEWTPGSFFDY

SEQ ID NO: 23 - protein, 14 aa, synthetic construct
  source 1..14: note = ZLA-1243-A01 CDRL1
TGTSSDVGGYNYVS

SEQ ID NO: 24 - protein, 7 aa, synthetic construct
  source 1..7: note = ZLA-1243-A01 CDRL2
DVSNRPS

SEQ ID NO: 25 - protein, 10 aa, synthetic construct
  source 1..10: note = ZLA-1243-A01 CDRL3
SSYTSSSTRV

SEQ ID NO: 26 - protein, 121 aa, synthetic construct
  source 1..121: note = ZLA-1243-A01 VH
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDNEWTPGSFFDYWGQGTLVTVSS

SEQ ID NO: 27 - protein, 110 aa, synthetic construct
  source 1..110: note = ZLA-1243-A01 VL
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGGGTKVTVL

SEQ ID NO: 28 - protein, 5 aa, synthetic construct
  source 1..5: note = ZLA-1243-E01 CDRH1
DYAMH

SEQ ID NO: 29 - protein, 17 aa, synthetic construct
  source 1..17: note = ZLA-1243-E01 CDRH2
GISWNSGSIGYADSVKG

SEQ ID NO: 30 - protein, 13 aa, synthetic construct
  source 1..13: note = ZLA-1243-E01 CDRH3
GLWPSYDLDAFDI

SEQ ID NO: 31 - protein, 14 aa, synthetic construct
  source 1..14: note = ZLA-1243-E01 CDRL1
TGTSSDVGAYNFVS

SEQ ID NO: 32 - protein, 7 aa, synthetic construct
  source 1..7: note = ZLA-1243-E01 CDRL2
EVSNRPS

SEQ ID NO: 33 - protein, 11 aa, synthetic construct
  source 1..11: note = ZLA-1243-E01 CDRL3
SSYTSTIPPFV

SEQ ID NO: 34 - protein, 122 aa, synthetic construct
  source 1..122: note = ZLA-1243-E01 VH
QVQLVQSGGGLVQPGRSLRLSCAASGFTFDDYAMHWVRQAPGKGLEWVSGISWNSGSIGYADSVKGRFTISRDNAKNSLYLQMNSLRAEDTALYYCAKGLWPSYDLDAFDIWGQGTMVTVSS

SEQ ID NO: 35 - protein, 111 aa, synthetic construct
  source 1..111: note = ZLA-1243-E01 VL
QSALTQPASVSGSPGQSITLSCTGTSSDVGAYNFVSWYQQHPGTAPKLMIYEVSNRPSGVSDRFSGSKSGNTASLTISGLQAEDEADYYCSSYTSTIPPFVFGTGTKVTVL

SEQ ID NO: 36 - protein, 5 aa, synthetic construct
  source 1..5: note = ZLA-1214-D08 CDRH1
GYYMH

SEQ ID NO: 37 - protein, 17 aa, synthetic construct
  source 1..17: note = ZLA-1214-D08 CDRH2
RINPNSGGTNYAQKFQG

SEQ ID NO: 38 - protein, 8 aa, synthetic construct
  source 1..8: note = ZLA-1214-D08 CDRH3
VINDAFDM

SEQ ID NO: 39 - protein, 16 aa, synthetic construct
  source 1..16: note = ZLA-1214-D08 CDRL1
RSSQSLLHSNGYNYLD

SEQ ID NO: 40 - protein, 7 aa, synthetic construct
  source 1..7: note = ZLA-1214-D08 CDRL2
LGSNRAS

SEQ ID NO: 41 - protein, 9 aa, synthetic construct
  source 1..9: note = ZLA-1214-D08 CDRL3
MQALQTQYT

SEQ ID NO: 42 - protein, 117 aa, synthetic construct
  source 1..117: note = ZLA-1214-D08 VH
EVQLVQSGAEVKKPGASVKVSCKASGYTFTGYYMHWVRQAPGQGLEWMGRINPNSGGTNYAQKFQGRVTMTRDTSTSTAYMELSRLRSDDTAVYYCARVINDAFDMWGQGTMVTVSS

SEQ ID NO: 43 - protein, 112 aa, synthetic construct
  source 1..112: note = ZLA-1214-D08 VL
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTQYTFGQGTKVDIK

SEQ ID NO: 44 - protein, 5 aa, synthetic construct
  source 1..5: note = ZLA-1214-B07 CDRH1
SYAIS

SEQ ID NO: 45 - protein, 17 aa, synthetic construct
  source 1..17: note = ZLA-1214-B07 CDRH2
GIIPIFGTANYAQKFQG

SEQ ID NO: 46 - protein, 17 aa, synthetic construct
  source 1..17: note = ZLA-1214-B07 CDRH3
DGGRDGYNPKKILEFDY

SEQ ID NO: 47 - protein, 12 aa, synthetic construct
  source 1..12: note = ZLA-1214-B07 CDRL1
RASQSVSSSYLA

SEQ ID NO: 48 - protein, 7 aa, synthetic construct
  source 1..7: note = ZLA-1214-B07 CDRL2
GASSRAT

SEQ ID NO: 49 - protein, 9 aa, synthetic construct
  source 1..9: note = ZLA-1214-B07 CDRL3
QQYGSSPIT

SEQ ID NO: 50 - protein, 126 aa, synthetic construct
  source 1..126: note = ZLA-1214-B07 VH
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDGGRDGYNPKKILEFDYWGQGTLVTVSS

SEQ ID NO: 51 - protein, 108 aa, synthetic construct
  source 1..108: note = ZLA-1214-B07 VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGPGTKVDIK

SEQ ID NO: 52 - protein, 5 aa, synthetic construct
  source 1..5: note = ZLA-1212-E09 CDRH1
SYGMH

SEQ ID NO: 53 - protein, 17 aa, synthetic construct
  source 1..17: note = ZLA-1212-E09 CDRH2
VISYDGSNKYYADSVKG

SEQ ID NO: 54 - protein, 13 aa, synthetic construct
  source 1..13: note = ZLA-1212-E09 CDRH3
DDSGYYDSSGYDY

SEQ ID NO: 55 - protein, 14 aa, synthetic construct
  source 1..14: note = ZLA-1212-E09 CDRL1
TGSSSNIGAGYDVH

SEQ ID NO: 56 - protein, 7 aa, synthetic construct
  source 1..7: note = ZLA-1212-E09 CDRL2
GNSNRPS

SEQ ID NO: 57 - protein, 11 aa, synthetic construct
  source 1..11: note = ZLA-1212-E09 CDRL3
QSYDSSLSGVV

SEQ ID NO: 58 - protein, 122 aa, synthetic construct
  source 1..122: note = ZLA-1212-E09 VH
QVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCAKDDSGYYDSSGYDYWGQGTLVTVSS

SEQ ID NO: 59 - protein, 111 aa, synthetic construct
  source 1..111: note = ZLA-1212-E09 VL
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGVVFGGGTKLTVL

SEQ ID NO: 60 - protein, 5 aa, synthetic construct
  source 1..5: note = ZLA-1212-B05 CDRH1
SYAMH

SEQ ID NO: 61 - protein, 17 aa, synthetic construct
  source 1..17: note = ZLA-1212-B05 CDRH2
VISYDGSNKYYADSVKG

SEQ ID NO: 62 - protein, 11 aa, synthetic construct
  source 1..11: note = ZLA-1212-B05 CDRH3
EGGSYYGYFDY

SEQ ID NO: 63 - protein, 11 aa, synthetic construct
  source 1..11: note = ZLA-1212-B05 CDRL1
GGNNIGSKSVH

SEQ ID NO: 64 - protein, 7 aa, synthetic construct
  source 1..7: note = ZLA-1212-B05 CDRL2
DDSDRPS

SEQ ID NO: 65 - protein, 11 aa, synthetic construct
  source 1..11: note = ZLA-1212-B05 CDRL3
QVWDSSSDHRV

SEQ ID NO: 66 - protein, 120 aa, synthetic construct
  source 1..120: note = ZLA-1212-B05 VH
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCAREGGSYYGYFDYWGQGTLVTVSS

SEQ ID NO: 67 - protein, 108 aa, synthetic construct
  source 1..108: note = ZLA-1212-B05 VL
QSVLTQPPSVSVAPGKTARITCGGNNIGSKSVHWYQQKPGQAPVLVIYDDSDRPSGIPERFSGSNSGNTATLTISRVEAGDEADYYCQVWDSSSDHRVFGGGTKLTVL

SEQ ID NO: 68 - protein, 5 aa, synthetic construct
  source 1..5: note = ZLA-1214-H05 CDRH1
SYGIS

SEQ ID NO: 69 - protein, 17 aa, synthetic construct
  source 1..17: note = ZLA-1214-H05 CDRH2
WISAYNGNTNYAQKLQG

SEQ ID NO: 70 - protein, 9 aa, synthetic construct
  source 1..9: note = ZLA-1214-H05 CDRH3
MSGYYGMDV

SEQ ID NO: 71 - protein, 11 aa, synthetic construct
  source 1..11: note = ZLA-1214-H05 CDRL1
QASQGISNYLN

SEQ ID NO: 72 - protein, 7 aa, synthetic construct
  source 1..7: note = ZLA-1214-H05 CDRL2
DASNLET

SEQ ID NO: 73 - protein, 9 aa, synthetic construct
  source 1..9: note = ZLA-1214-H05 CDRL3
QQYDNLPLT

SEQ ID NO: 74 - protein, 118 aa, synthetic construct
  source 1..118: note = ZLA-1214-H05 VH
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARMSGYYGMDVWGQGTLVTVSS

SEQ ID NO: 75 - protein, 107 aa, synthetic construct
  source 1..107: note = ZLA-1214-H05 VL
DIQLTQSPSSLSASVGDRVTITCQASQGISNYLNWYQQKPGKAPKLLIYDASNLETGVPSRFSGSGSGTDFTFTISSLQPEDIATYYCQQYDNLPLTFGGGTKLEIK

SEQ ID NO: 76 - DNA, 351 bp, synthetic construct
  source 1..351: mol_type = other DNA; note = Clone 62E5 heavy chain (VH)
gaggtgaaactgctggaatctggcggaggcctggtgcagcccggcggcagcctgaaactgagctgtgctgcttctggatttgacttcagcggctactggatgagctgggtcagacaggcccctggcaagggcctcgagtggatcggagaaatcattccagatagcagcaccatcaactacacccctagcctgaaggacaagttcatcatcagcagagataatgccaagaacacactgttcctgcaaatgagaaaggtgcggtccgaggacaccgccctgtactactgcgccagacgggacggctattggttcgcctactggggccagggcacactggtgaccgtgtccgcc

SEQ ID NO: 77 - DNA, 339 bp, synthetic construct
  source 1..339: mol_type = other DNA; note = Clone 62E5 light chain (VL)
gacatcgtgatgagccagtcccctagctccctggccgtgtccgtgggcgagaaggtgaccatgtcttgtaaaagctctcagtctctgctgtacagcagaaaccagcagaactacctggcttggtttcagcaaaagcccggccagagccctgagctgctgatctactgggccagcacccgggaaagcggagtgccagatagattcacaggcagcggatctggcaccgacttcaccctgacaatcagcagcgtgaaggccgaggacctggccgtctactactgccagcagtactatagctaccctctgaccttcggcgccggcacaaagctggaactcaag

SEQ ID NO: 78 - DNA, 360 bp, synthetic construct
  source 1..360: mol_type = other DNA; note = Clone S15B1 VH
caagtgcagctgcagcagagcggacctgaactggtgaagcctggcgctagcgtgaaaatcagctgcaaggcctccggctacgccttcagcagctcttggatgaactgggtcaagcagcggccaggaaagggcctggaatggatcggcagaatctaccccggccacggcgagacaaactacaacggcaagttcaaggacaaggctacagtgaccgccgataaaagctcttctactacctacatgcagctgagctctctgaccagcgaggacagcgccgtgtacttctgtgccagagtggataatagcgacccttattactttgactactggggccagggcacaaccctgaccgtgtccagc

SEQ ID NO: 79 - DNA, 333 bp, synthetic construct
  source 1..333: mol_type = other DNA; note = Clone S15B1 VL
gacatcgtgctgacccagagccctgccagcctggccgtgtccctgggccagcgggccacaatcagctgtagagcctctaaaagcgtctctacaagcggctacagctacatgcactggtatcagcagaagcccggccaacctccaaagctgctgatctacctggctagcaacctggagagcggcgtgcctgctagattcagcggcagcggatccggcaccgacttcaccctgaacatccaccccgtggaagaggaagatgccgccacctactactgccagcacggcagagagctgccttttaccttcggctctggaacaaagctcgagatcaag

SEQ ID NO: 80 - DNA, 363 bp, synthetic construct
  source 1..363: mol_type = other DNA; note = ZLA-1243-A01 VH
caagtgcagctggtggaatctggcggcggactggttcagcccggcggctccctgcggctgagctgtgccgctagcggcttcaccttcagctcttatgccatgagctgggtgcggcaggcccctggaaaaggcctggaatgggtctccgccatcagcggcagcggaggcagcacatactacgctgatagcgtgaagggcagattcacaatctctagagataatagcaagaacaccctgtacctgcagatgaacagcctgagagccgaggacaccgccgtgtactactgcgccaaggacaacgagtggacccctggctcttttttcgactactggggccagggcaccctcgtgacagtgtccagc

SEQ ID NO: 81 - DNA, 330 bp, synthetic construct
  source 1..330: mol_type = other DNA; note = ZLA-1243-A01 VL
caaagcgccctgacccagcctgcttctgtgtccggatctccaggccagagcatcaccatctcttgtacaggcacaagcagcgacgtgggcggctacaactacgtgtcctggtatcagcagcaccccggcaaagcccctaagctgatgatctacgacgtcagcaaccggcctagcggcgtgtctaatagattcagcggatctaagagcggcaacaccgccagcctgacaatcagcggcctgcaggccgaagatgaggccgactactactgcagcagctacaccagcagctccaccagagtgttcggcggaggcaccaaggtgaccgtgctg

SEQ ID NO: 82 - DNA, 366 bp, synthetic construct
  source 1..366: mol_type = other DNA; note = ZLA-1243-E01 VH
caagtgcagctggtccagagcggcggaggcctggtgcagcccggcagaagcctgagactgagctgtgctgctagcggcttcacattcgacgactacgccatgcactgggtgcggcaggcccctggcaagggcctcgagtgggtttctggcatcagctggaattctggcagcatcggctacgccgacagcgtgaagggaagattcaccatctctagagataacgccaagaacagcctgtatctgcagatgaactccctgcgggccgaagatacagccctgtactactgcgccaaaggactgtggcctagctacgacctggacgcctttgatatctggggccagggcaccatggtgaccgtgtccagc

SEQ ID NO: 83 - DNA, 333 bp, synthetic construct
  source 1..333: mol_type = other DNA; note = ZLA-1243-E01 VL
caaagcgccctgacacagcctgccagcgtgtccggatctccaggccagagcatcaccctgagctgtaccggcacatctagcgacgtgggcgcctacaacttcgtgtcttggtatcagcagcaccctggcaccgcccctaagctgatgatctacgaggtgtccaaccggcctagcggcgtgtccgacagattcagcggcagcaaaagcggaaataccgcttctctgaccatcagcggcctgcaggctgaagatgaggccgactactactgcagcagctacaccagcaccatcccccccttcgtctttggcacaggcacaaaggtgaccgtgctg

SEQ ID NO: 84 - DNA, 351 bp, synthetic construct
  source 1..351: mol_type = other DNA; note = ZLA-1214-D08 VH
gaggtccagctggtgcagagcggagccgaggtgaaaaagcccggcgcttctgtgaaggtgtcctgcaaggcctctggatacaccttcaccggctactacatgcactgggtgcggcaggcccctggccaaggcctggaatggatgggcaggatcaatcctaacagcggcggaacaaactacgcccagaagttccagggccgggtgacaatgaccagagataccagcacaagcaccgcctacatggaactgagcagactgagaagcgacgacacagctgtgtactattgtgccagagtgatcaacgacgcctttgatatgtggggccagggcaccatggtgaccgtgtccagc

SEQ ID NO: 85 - DNA, 336 bp, synthetic construct
  source 1..336: mol_type = other DNA; note = ZLA-1214-D08 VL
gatgtggtgatgacacagagccccctgagcctgcctgtgacccctggcgagcctgcttctatcagctgtagaagcagccagagcctgctgcacagcaacggctacaactacctggactggtatctccagaaacctggacagtccccacagctgctgatctacctgggctctaatcgggccagcggcgtccccgatagattcagcggcagcggatctggcaccgacttcaccctgaagatctccagagtggaagccgaggacgtgggcgtgtactactgcatgcaggccctgcagacccaatacacctttggccagggcacaaaggtggacatcaag

SEQ ID NO: 86 - DNA, 378 bp, synthetic construct
  source 1..378: mol_type = other DNA; note = ZLA-1214-B07 VH
caagtgcagctggtgcagagcggcgccgaggtgaaaaagcccggcagcagcgtgaaggtgtcttgtaaagccagcggcggaacattcagctcctacgccatcagctgggtgcggcaggcccctggccagggactggagtggatgggcggcatcatccctattttcggcacagccaactacgcccagaagtttcagggcagagtgaccatcaccgccgacgagagcaccagcacagcttatatggaactgagcagcctgagatctgaagataccgctgtgtactactgcgccagagatggcggacgggacggctacaacccaaagaagatcctggagttcgactactggggccagggcaccctggtcaccgtgtcctct

SEQ ID NO: 87 - DNA, 324 bp, synthetic construct
  source 1..324: mol_type = other DNA; note = ZLA-1214-B07 VL
gaaatcgtgctgacccagagccctggcaccctctccctgtctccaggcgagcgggccaccctgagctgtagagcttctcagagcgtgtcctccagctacctggcctggtatcagcagaaacctggccaggcccctagactgctgatctacggcgccagcagcagagctacaggcattcctgatagattcagcggatctggcagcggcacagacttcaccctgacaatcagccggctggaacccgaggactttgccgtgtactactgccagcaatacggcagcagccccatcaccttcggccctggaaccaaggtcgacatcaag

SEQ ID NO: 88 - DNA, 366 bp, synthetic construct
  source 1..366: mol_type = other DNA; note = ZLA-1212-E09 VH
caagtgcagctggttcagagcggaggaggcgtggtgcagcccggcagaagcctgagactgagctgtgctgcttctggatttacattcagctcctacggcatgcactgggtgcggcaggcccctggcaaaggcctggaatgggtcgccgtgatcagctacgacggcagcaacaagtactacgccgatagcgtgaagggcagattcaccatctctagagataatagcaagaacaccctctacctgcagatgaacagcctgcgggccgaggacaccgccgtgtactactgcgccaaggacgacagcggctactatgattctagcggctacgactactggggccagggcacactggtgaccgtgtcctcc

SEQ ID NO: 89 - DNA, 333 bp, synthetic construct
  source 1..333: mol_type = other DNA; note = ZLA-1212-E09 VL
caatctgtgctgacacagcccccctccgtgtccggcgctcctggccagcgggtgaccatcagctgcaccggctcttcttccaacatcggcgccggctacgacgtgcactggtatcagcagctgcctggaacagcccctaagctgctgatctacggcaacagcaatagacctagcggcgtgccagatagattcagcggcagcaaaagcggcaccagcgccagcctggccatcaccggcctgcaggctgaagatgaggccgactactactgtcagagctacgacagctctctgagcggagtcgtgttcggcggaggcacaaagctgaccgtgctc

SEQ ID NO: 90 - DNA, 360 bp, synthetic construct
  source 1..360: mol_type = other DNA; note = ZLA-1212-B05 VH
caagtgcagctggtgcagagcggaggcggagtggtgcagcccggaagaagcctgcggctgagctgtgctgcttctggctttaccttcagctcttatgccatgcactgggtgcggcaggcccctggcaaaggcctggaatgggtcgccgtgatcagctacgacggctccaacaagtactacgccgatagcgtgaagggcagattcaccatctctagagataatagcaagaacacactctacctgcagatgaacagcctgcgcgccgaggacaccgccgtgtactactgcgccagagagggcggcagctactacggctacttcgactactggggccagggcaccctggttacagtgtccagc

SEQ ID NO: 91 - DNA, 324 bp, synthetic construct
  source 1..324: mol_type = other DNA; note = ZLA-1212-B05 VL
caaagcgttctgacacagcctccatctgtgtccgtcgcccccggcaagaccgctagaatcacatgcggcggcaacaacatcggctctaagtccgtgcactggtatcagcagaaacctggccaggcccctgtgctggtgatctacgacgactctgatagacctagcggcatccccgagcggttcagcggcagcaacagcggcaataccgccaccctgaccatcagcagagtggaagccggagatgaggccgactactactgtcaggtgtgggacagcagcagcgaccaccgggtgttcggcggaggaacaaagctgaccgtgctg

SEQ ID NO: 92 - DNA, 354 bp, synthetic construct
  source 1..354: mol_type = other DNA; note = ZLA-1214-H05 VH
caagtgcagctggtccagagcggagccgaggtgaaaaagcccggcgcttctgtgaaggtgtcctgcaaggccagcggctacaccttcaccagctacggcatcagctgggtgcggcaggcccctggccagggcctggaatggatgggctggatctccgcctacaacggcaataccaactacgcccagaagctgcagggaagagtgaccatgacaacagatacatctaccagcaccgcttacatggagctgagaagcctgcggagcgacgacaccgccgtgtactactgtgccagaatgagcggatattacggcatggacgtgtggggccagggcaccctggttacagtgtctagc

SEQ ID NO: 93 - DNA, 321 bp, synthetic construct
  source 1..321: mol_type = other DNA; note = ZLA-1214-H05 VL
gatattcagctgacccagagcccatctagcctgagcgccagcgtgggagatagagtgaccatcacctgtcaggcttctcagggcatcagcaactacctgaactggtatcagcagaaacccggcaaggcccctaagctgctgatctacgacgcctctaatctggaaaccggcgtgcccagccggttcagcggcagcggctccggcaccgacttcacctttaccatctccagcctgcagcctgaggacatcgccacatactactgccagcaatacgacaacctgcctctgacattcggcggcggaacaaagctcgagatcaag

SEQ ID NO: 94 - protein, 119 aa, Homo sapiens
  source 1..119: note = Human Siglec-15 D1
PAQRWSMQVPPEVSAEAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRH

SEQ ID NO: 95 - protein, 84 aa, Homo sapiens
  source 1..84: note = Human Siglec-15 D2
PRIVNISVLPSPAHAFRALCTAEGEPPPALAWSGPALGNSLAAVRSPREGHGHLVTAELPALTHDGRYTCTAANSLGRSEASVY

SEQ ID NO: 96 - protein, 106 aa, Homo sapiens
  source 1..106: note = Human lambda CL v1
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 97 - protein, 105 aa, Homo sapiens
  source 1..105: note = Human lambda CL v2
GQPKAAPSVTLFPPSSEELQANKATLVCLISDFYPGAVTVAWKADSSPVKAGVETTTPSKQSNNKYAASSYLSLTPEQWKSHRSYSCQTHEGSTVEKTVAPTECS

SEQ ID NO: 98 - protein, 105 aa, Homo sapiens
  source 1..105: note = Human lambda CL v3
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 99 - protein, 106 aa, Homo sapiens
  source 1..106: note = Human lambda CL v4
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 100 - protein, 106 aa, Homo sapiens
  source 1..106: note = Human lambda CL v5
GQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 101 - protein, 106 aa, Homo sapiens
  source 1..106: note = Human kappa CL v1
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 107 aa, Homo sapiens [sequence omitted]

SEQ ID NO: 103 - protein, 120 aa, Synthetic construct
  source 1..120: note = S15B1-VH1
QVQLVQSGAEVKKPGASVKVSCKASGYAFSSSWMNWVRQAPGQGLEWIGRIYPGHGETNYNGKFKDRVTMTADTSTSTVYMELSSLRSEDTAVYFCARVDNSDPYYFDYWGQGTTVTVSS

SEQ ID NO: 104 - protein, 120 aa, Synthetic construct; identical to SEQ ID NO: 103
  source 1..120: note = S15B1-VH2

SEQ ID NO: 105 - protein, 120 aa, Synthetic construct
  source 1..120: note = S15B1-VH3
QVQLVQSGAEVKKPGASVKVSCKASGYAFSSSWMNWVKQAPGQGLEWIGRIYPGHGETNYNGKFKDRATMTADTSTSTVYMELSSLRSEDTAVYFCARVDNSDPYYFDYWGQGTTVTVSS

SEQ ID NO: 106 - protein, 120 aa, Synthetic construct
  source 1..120: note = S15B1-VH4
QVQLVQSGAEVKKPGASVKVSCKASGYAFSSSWMNWVKQAPGQGLEWIGRIYPGHGETNYNGKFKDRATMTADKSTSTVYMELSSLRSEDTAVYFCARVDNSDPYYFDYWGQGTTVTVSS

SEQ ID NO: 107 - protein, 111 aa, Synthetic construct
  source 1..111: note = S15B1-VL1
DIVLTQSPASLAVSPGQRATITCRASKSVSTSGYSYMHWYQQKPGQPPKLLIYLASNLESGVPARFSGSGSGTDFTLTINPVEAEDTANYYCQHGRELPFTFGGGTKLEIK

SEQ ID NO: 108 - protein, 111 aa, Synthetic construct
  source 1..111: note = S15B1-VL2
DIVLTQSPASLAVSPGQRATITCRASKSVSTSGYSYMHWYQQKPGQPPKLLIYLASNLESGVPARFSGSGSGTDFTLTINPVEAEDTATYYCQHGRELPFTFGGGTKLEIK

SEQ ID NO: 109 - protein, 125 aa, Synthetic construct
  source 1..125: note = VH-3-15
EVQLVESGGGLVKPGGSLRLSCAASGFTFNTYAMNWVRQAPGKGLEWVARIRSKYNNYATYYADSVKDRFTISRDDSKNTLYLQMNSLKTEDTAVYYCVRHGNFGNSYVSWFAYWGQGTLVTVSS

SEQ ID NO: 110 - protein, 109 aa, Synthetic construct
  source 1..109: note = VL-8-61
QTVVTQEPSFSVSPGGTVTLTCRSSTGAVTTSNYANWVQQTPGQAPRGLIGGTNKRAPGVPARFSGSILGNKAALTITGAQADDEAEYFCALWYSNLWVFGGGTKLTVL

SEQ ID NO: 111 - protein, 218 aa, Synthetic construct
  source 1..218: note = BsAb-1 VL1 + CL
DIVLTQSPASLAVSLGQRATISCRASKSVSTSGYSYMHWYQQKPGQPPKLLIYLASNLESGVPARFSGSGSGTDFTLNIHPVEEEDAATYYCQHGRELPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 450 aa, Synthetic construct
  source 1..450: note = BsAb-1 VH1 + CH
QVQLQQSGPELVKPGASVKISCKASGYAFSSSWMNWVKQRPGKGLEWIGRIYPGHGETNYNGKFKDKATVTADKSSSTTYMQLSSLTSEDSAVYFCARVDNSDPYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 113 - protein, 458 aa, Synthetic construct
  source 1..458: note = BsAb-1 VH2 + CH
EVQLVESGGGLVKPGGSLRLSCAASGFTFNTYAMNWVRQAPGKGLEWVARIRSKYNNYATYYADSVKDRFTISRDDSKNTLYLQMNSLKTEDTAVYYCVRHGNFGNSYVSWFAYWGQGTLVTVSSGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 212 aa, Synthetic construct
  source 1..212: note = BsAb-1 VL2 + CL
QTVVTQEPSFSVSPGGTVTLTCRSSTGAVTTSNYANWVQQTPGQAPRGLIGGTNKRAPGVPARFSGSILGNKAALTITGAQADDEAEYFCALWYSNLWVFGGGTKLTVLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 115 - protein, 218 aa, Synthetic construct; identical to SEQ ID NO: 111
  source 1..218: note = BsAb-2 VL1 + CL

SEQ ID NO: 116 - protein, 450 aa, Synthetic construct; identical to SEQ ID NO: 112
  source 1..450: note = BsAb-2 VH1 + CH

SEQ ID NO: 117 - protein, 734 aa, Synthetic construct
  source 1..734: note = BsAb-2 VH2 + CH
QVQLQQSGPELVKPGASVKISCKASGYAFSSSWMNWVKQRPGKGLEWIGRIYPGHGETNYNGKFKDKATVTADKSSSTTYMQLSSLTSEDSAVYFCARVDNSDPYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSQTVVTQEPSFSVSPGGTVTLTCRSSTGAVTTSNYANWVQQTPGQAPRGLIGGTNKRAPGVPARFSGSILGNKAALTITGAQADDEAEYFCALWYSNLWVFGGGTKLTVLGGGGSGGGGSGGGGSGGGGSEVQLVESGGGLVKPGGSLRLSCAASGFTFNTYAMNWVRQAPGKGLEWVARIRSKYNNYATYYADSVKDRFTISRDDSKNTLYLQMNSLKTEDTAVYYCVRHGNFGNSYVSWFAYWGQGTLVTVSSGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 218 aa, Synthetic construct; identical to SEQ ID NO: 111
  source 1..218: note = BsAb-2 VL2 + CL

SEQ ID NO: 119 - DNA, 360 bp, Synthetic construct
  source 1..360: mol_type = other DNA; note = S15B1-VH1
caggttcagttggttcagtctggcgccgaggtgaagaagcctggcgcctctgtgaaggtgtcctgcaaggcctctggctacgccttctccagctcctggatgaactgggtccgacaggctcctggacaaggactggaatggatcggcagaatctaccctggccatggagaaacaaactacaatggcaagttcaaggacagagtgaccatgacagctgacaccagcacctctactgtgtacatggaactgtcctcactgaggtctgaggacaccgccgtgtactactgtgccagagtagacaacagcgacccttactacttcgactactggggccagggcacaaccgtgaccgtgtcctct

SEQ ID NO: 120 - DNA, 360 bp, Synthetic construct
  source 1..360: mol_type = other DNA; note = S15B1-VH2
caggttcagttggttcagtctggcgccgaggtgaagaagcctggcgcctctgtgaaggtgtcctgcaaggcctctggctacgccttctccagctcctggatgaactgggtccgacaggctcctggacaaggactggaatggatcggcagaatctaccctggccatggagaaacaaactacaatggcaagttcaaggacagagtgaccatgacagctgacaccagcacctctactgtgtacatggaactgtcctcactgaggtctgaggacaccgccgtgtacttctgcgccagagtggacaacagcgacccttactacttcgactactggggccagggcacaaccgtgaccgtgtcctct

SEQ ID NO: 121 - DNA, 360 bp, Synthetic construct
  source 1..360: mol_type = other DNA; note = S15B1-VH3
caggttcagttggttcagtctggcgccgaggtgaagaagcctggcgcctctgtgaaggtgtcctgcaaggcctctggctacgccttctccagcagctggatgaactgggtgaagcaggctcctggccagggcctggaatggatcggcagaatctaccctggccatggagaaacaaactacaatggaaagttcaaggacagagccaccatgacagcagacaccagcacctctactgtgtacatggaactgtcctcactgaggtctgaggacaccgccgtgtacttctgcgccagagtggacaacagcgacccttactacttcgactactggggccagggcacaaccgtgaccgtgtcctct

SEQ ID NO: 122 - DNA, 360 bp, Synthetic construct
  source 1..360: mol_type = other DNA; note = S15B1-VH4
caggttcagttggttcagtctggcgccgaggtgaagaagcctggcgcctctgtgaaggtgtcctgcaaggcctctggctacgccttctccagcagctggatgaactgggtgaagcaggctcctggccagggcctggaatggatcggcagaatctaccctggccatggagaaacaaactacaatggaaagttcaaggacagagccaccatgaccgccgacaagtccacctccaccgtgtatatggaactgtcctcactgaggtctgaggacaccgccgtgtacttctgcgccagagtggacaacagcgacccttactacttcgactactggggccagggcacaaccgtgaccgtgtcctct

SEQ ID NO: 123 - DNA, 333 bp, Synthetic construct
  source 1..333: mol_type = other DNA; note = S15B1-VL1
gatatcgtgctgacccagtctcctgcctctctggctgtgtctcctggccagagagccaccatcacctgtagagcctccaagtccgtgtctacctcaggctactcctacatgcactggtaccagcagaagcctggccagcctcccaagctgctgatctacctggcctccaacctggagagtggcgtgccagccagattctccggctctggctctggcaccgacttcaccctgaccatcaacccagtggaagccgaggacacagccaactactactgccagcatggcagagagctgcccttcacctttggcggcggcaccaagctggaaatcaag

SEQ ID NO: 124 - DNA, 333 bp, Synthetic construct
  source 1..333: mol_type = other DNA; note = S15B1-VL2
gatatcgtgctgacccagtctcctgcctctctggctgtgtctcctggccagagagccaccatcacctgtagagcctccaagtccgtgtctacctcaggctactcctacatgcactggtaccagcagaagcctggccagcctcccaagctgctgatctacctggcctccaacctggagagtggcgtgccagccagattctccggctctggctctggcaccgacttcaccctgaccatcaacccagtggaagccgaggacaccgccacctactactgtcagcacggcagagagctgcccttcacctttggcggcggcaccaagctggaaatcaag

SEQ ID NO: 125 - DNA, 375 bp, Synthetic construct
  source 1..375: mol_type = other DNA; note = VH-3-15
gaagtgcaactagtggaaagtggtggtggtctcgtgaaacccggcggatctctgagactgtcctgtgctgcttctggcttcaccttcaacacctacgccatgaactgggtcagacaggctcctggcaagggcctggaatgggtggccagaatccggtccaagtacaacaactacgctacatattacgccgactccgtgaaggaccggtttaccatctccagagatgactctaagaacacactgtacctgcagatgaatagcctgaagaccgaggacaccgccgtgtactactgcgtgcggcacggcaacttcggcaactcctacgtgtcttggttcgcctactggggccagggcaccctggtgaccgtgtccagc

SEQ ID NO: 126 - DNA, 327 bp, Synthetic construct
  source 1..327: mol_type = other DNA; note = VL-8-61
caaactgtggtgactcaagaaccaagtttttccgtgtccccaggaggtaccgtgacactgacctgtcggagctctaccggcgccgtgaccacatctaactacgccaactgggtccaacagacccctggccaggctcctagaggcctgatcggcggcaccaataaacgggcccctggcgtgcccgctagattttctggctccatcctgggcaacaaggccgctctcaccatcaccggcgctcaggccgacgatgaagccgagtacttctgcgccctgtggtactccaacctgtgggtgttcggcggcggaaccaagctgacagtgctg

SEQ ID NO: 127 - DNA, 654 bp, Synthetic construct
  source 1..654: mol_type = other DNA; note = BsAb-1 VL1 + CL
gatatcgtgctaactcaaagtccagcaagtctggccgtgtctctgggccaacgggccaccatcagctgtagagcctctaagtccgtgtccacctctggctactcctacatgcactggtatcagcagaagcctggccagcctccaaaactgctgatctacctggcttccaacctggaatctggagtccccgctcggttctccggcagcggctctggaacagattttaccctgaacatccatcctgtggaagaggaggacgccgctacctactactgccagcacggcagagagctgcctttcaccttcggctccggcaccaagctcgagatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgc

SEQ ID NO: 128 - DNA, 1350 bp, Synthetic construct
  source 1..1350: mol_type = other DNA; note = BsAb-1 VH1 + CH
caagtgcaactacaacaaagtggtccagaactggtgaagcctggagcttctgtgaagatctcctgcaaggcctctggctacgccttctcctcttcttggatgaattgggtcaagcagcggcctggcaaaggcctggaatggatcggcagaatctaccccggacacggcgagaccaactacaacggcaagttcaaggacaaagctacagtgaccgctgataagtccagctctaccacctacatgcagctgtcctctctgacctccgaggactccgccgtgtatttttgtgccagagtggacaactccgatccttactacttcgactactggggccagggcacaaccctgaccgtgtccagcgccagcaccaaaggcccctccgtgttccccctggcccctagctccaagtctacctctggcggtactgctgccctgggctgcctggtgaaggactacttccctgagcctgtgacagtgtcctggaactccggagctctgacctccggcgtgcatacctttcctgctgtgctgcaatcctctggactgtactccctgtcctctgtggtgaccgtgccatcttccagcctgggcacccagacctacatctgcaacgtgaaccacaagccttccaacaccaaggtggacaagaaagtggaacctaagtcctgcgataagactcatacttgcccaccatgcccagctcctgaggctgctggtggcccttctgtgtttctgttccctcctaagcccaaggacaccctgatgatctctcggacccctgaagtgacctgcgtggtcgtggacgtgtctcacgaggaccccgaggtgaagttcaactggtatgtggatggcgtggaagtgcacaacgccaagaccaagcctcgggaagagcagtacaactccacctacagagtggtgtccgtgctgaccgttctgcaccaggactggctgaacggcaaagagtacaagtgcaaggtgtccaacaaggctctgcctgcccctatcgagaagaccatctccaaggccaaaggacagcctagagagcctcaagtgtgcacactgccaccatctcgcgacgagctgacaaagaaccaggtgtctctctcctgtgccgtcaaaggcttctacccctctgatatcgccgtggaatgggaatccaatggccagcccgagaacaactacaagaccacacctcctgtgctggactccgatggcagcttcttcctggtgagcaagctgaccgtggacaagagcagatggcagcagggcaacgtcttctcctgctccgtgatgcacgaggccctgcataatcactacacccagaagtccctgagcctgtctcctggcaag

SEQ ID NO: 129 - DNA, 1374 bp, Synthetic construct
  source 1..1374: mol_type = other DNA; note = BsAb-1 VH2 + CH
gaagtgcaactagtggaaagtggtggtggtctcgtgaaacccggcggatctctgagactgtcctgtgctgcttctggcttcaccttcaacacctacgccatgaactgggtcagacaggctcctggcaagggcctggaatgggtggccagaatccggtccaagtacaacaactacgctacatattacgccgactccgtgaaggaccggtttaccatctccagagatgactctaagaacacactgtacctgcagatgaatagcctgaagaccgaggacaccgccgtgtactactgcgtgcggcacggcaacttcggcaactcctacgtgtcttggttcgcctactggggccagggcaccctggtgaccgtgtccagcggacagcccaaggctgctccttccgtgaccctgttccctccatcctccgaggaactgcaggccaataaggccaccctggtgtgcctgatctccgacttctaccctggcgccgtgaccgtggcctggaaggccgactcttctcctgtgaaggctggcgtggagacaaccaccccctccaagcagtccaacaacaagtacgccgcttcttcttacctgtctctgacccctgagcagtggaagtctcatagatcctactcctgtcaagtgacccacgagggctccaccgtggaaaaaaccgtggcccctaccgagtgctccgacaaaacccacacatgtcctccttgtcctgctcctgaggccgccggcggcccctctgtgtttctgttcccccctaagcctaaagataccctgatgatctctagaaccccagaagttacctgtgtcgtggtggacgtgagccacgaggatcctgaagtgaagttcaactggtacgtggacggcgtggaagtgcacaacgccaagaccaagccccgggaagagcagtacaactccacctacagagtggtctccgtgctgaccgtgctgcaccaggactggctgaacggaaaagagtacaagtgcaaggtgtctaacaaggccctccctgcccctatcgagaagaccatctcgaaggctaaaggccaacctagagagcctcaagtgtacacacttccaccttgccgcgacgagctgaccaagaaccaggtgtctctgtggtgcctggtgaagggcttctacccttccgacatcgccgtggagtgggagtccaatggccagccagagaacaactacaagactacccctcctgtgctggactccgatggctccttcttcctgtactccaagctgacagtggataagtctcggtggcagcagggcaacgtgttctcctgcagcgtgatgcacgaagccctgcataatcactacacccagaagtccctgtctctgagccctggcaag

SEQ ID NO: 130 - DNA, 636 bp, Synthetic construct
  source 1..636: mol_type = other DNA; note = BsAb-1 VL2 + CL
caaactgtggtgactcaagaaccaagtttttccgtgtccccaggaggtaccgtgacactgacctgtcggagctctaccggcgccgtgaccacatctaactacgccaactgggtccaacagacccctggccaggctcctagaggcctgatcggcggcaccaataaacgggcccctggcgtgcccgctagattttctggctccatcctgggcaacaaggccgctctcaccatcaccggcgctcaggccgacgatgaagccgagtacttctgcgccctgtggtactccaacctgtgggtgttcggcggcggaaccaagctgacagtgctggcctccacaaagggcccaagcgtgttccctctggctccttcctccaagtccacctctggcggcaccgccgccctgggctgcctggtgaaggactacttccccgagcctgtgaccgtgtcctggaactccggcgctctgacctccggcgtgcacacctttcctgctgtgctgcaaagcagcggactgtactccctgtcctccgtggtgaccgtgccatcttccagcctgggaacccagacatacatctgcaacgtgaaccacaagccctctaataccaaggtggacaagaaagtggaacctaagtcttgc

SEQ ID NO: 131 - DNA, 654 bp, Synthetic construct; identical to SEQ ID NO: 127
  source 1..654: mol_type = other DNA; note = BsAb-2 VL1 + CL

SEQ ID NO: 132 - DNA, 1350 bp, Synthetic construct; identical to SEQ ID NO: 128
  source 1..1350: mol_type = other DNA; note = BsAb-2 VH1 + CH

SEQ ID NO: 133 - DNA, 2202 bp, Synthetic construct
  source 1..2202: mol_type = other DNA; note = BsAb-2 VH2 + CH
caagtgcaactacaacaaagtggtccagaactcgtgaagcctggcgcatccgtcaagatctcctgcaaggcctccggctacgccttctcctcctcctggatgaactgggtcaagcagagacctggaaaaggactggagtggatcggtagaatctaccctggccacggcgagaccaattacaacggcaagttcaaggacaaggctaccgtgaccgccgataagtcttcctccaccacctacatgcagctgtcttctctgacatctgaggactctgccgtttacttctgcgccagagtggacaactcggacccctactacttcgactactggggccagggcaccaccctgacagtgtcctctgcctctaccaagggcccatctgtgtttcctctggctccttcttccaaaagcacctccggcgggaccgctgctctgggctgcctggtgaaggattatttccccgagcctgtgacagtgtcctggaactccggcgctctgaccagcggcgtccacacctttccagccgtgctgcagtcttctggcctgtattctctgagctccgtggtgacagtgccctcctcttctctgggaacgcagacctacatctgcaacgtgaaccacaagcctagcaataccaaggtggacaagaaagtggaacctaagtcctgtggcggaggtggctctggaggcggtggatctggtggtggcggatcacaaaccgtggtgacccaggaaccctccttctccgtgtcccctggaggaaccgttaccctgacctgccggagtagcaccggcgctgtgaccacctctaactacgccaactgggtacagcagacccctggccaggcccctagaggactgatcggcggcaccaacaagagagcccctggcgtgcctgctcggttctctggctctatcctcggcaacaaggccgctctgaccattaccggcgcccaggctgacgacgaggccgagtacttttgtgccctgtggtactccaacctgtgggtgttcggaggcggtaccaaactgacagtgctgggtggaggcgggagcggaggcggcggtagtggtggaggaggctctggcggtggtggaagcgaggtgcagctggtcgaatccggcggcggcctggtgaaacccggcggcagcctgagactgagttgtgctgcctccggcttcaccttcaacacctatgctatgaattgggtgcggcaggctcctggcaagggcctggaatgggtggcccggatcaggtctaagtacaacaactacgccacctactacgccgactccgtgaaggacagattcaccatctctcgggacgattccaagaacacactgtacctccagatgaactccctgaagactgaggatacagccgtgtactactgcgtgcgccatggcaacttcggcaactcctacgtgagctggttcgcctactggggccaaggcacactggtgaccgtgtcctccggtggcggcggatctggcggaggaggttcaggtggaggtggatctgacaaaacccacacatgtcctccttgtcctgctcctgaggccgccggcggcccctctgtgtttctgttcccccctaagcctaaagataccctgatgatctctagaaccccagaagttacctgtgtcgtggtggacgtgagccacgaggatcctgaagtgaagttcaactggtacgtggacggcgtggaagtgcacaacgccaagaccaagccccgggaagagcagtacaactccacctacagagtggtctccgtgctgaccgtgctgcaccaggactggctgaacggaaaagagtacaagtgcaaggtgtctaacaaggccctccctgcccctatcgagaagaccatctcgaaggctaaaggccaacctagagagcctcaagtgtacacacttccaccttgccgcgacgagctgaccaagaaccaggtgtctctgtggtgcctggtgaagggcttctacccttccgacatcgccgtggagtgggagtccaatggccagccagagaacaactacaagactacccctcctgtgctggactccgatggctccttcttcctgtactccaagctgacagtggataagtctcggtggcagcagggcaacgtgttctcctgcagcgtgatgcacgaagccctgcataatcactacacccagaagtccctgtctctgagccctggcaag

SEQ ID NO: 134 - DNA, 654 bp, Synthetic construct; identical to SEQ ID NO: 127
  source 1..654: mol_type = other DNA; note = BsAb-2 VL2 + CL